(19) United States
(12) Patent Application Publication
(10) Pub. No.: US 2006/0223792 A1
(43) Pub. Date: Oct. 5, 2006

(54) Phenyl and pyridyl LTA4H modulators

(76) Inventors: Christopher R. Butler, Urbana, IL (US); James P. Edwards, San Diego, CA (US); Anne M. Fourie, San Diego, CA (US); Cheryl A. Grice, Carlsbad, CA (US); Lars Karlsson, San Diego, CA (US); Brad M. Savall, San Diego, CA (US); Kevin L. Tays, Cardiff, CA (US); Jianmei Wei, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/393,532

(22) Filed: Mar. 30, 2006

Related U.S. Application Data
(60) Provisional application No. 60/667,199, filed on Mar. 31, 2005.

(51) Int. Cl. A61K 31/4545; A61K 31/454; C07D 211/30; C07D 211/68; C07D 403/02; C07D 401/02

(52) U.S. Cl. 514/217.11; 514/317; 514/316; 514/318; 514/326; 514/319; 514/567; 546/194; 546/209; 546/190; 546/205

(57) ABSTRACT

Leukotriene A4 hydrolase (LTA4H) inhibitors, compositions containing them, and methods of use for the inhibition of LTA4H enzyme activity and the treatment, prevention or inhibition of inflammation and inflammatory conditions.

This application claims the benefit of U.S. provisional patent application Ser. No. 60/667,199, filed on Mar. 31, 2005, the contents of which is incorporated herein by reference.

### FIELD OF THE INVENTION

This invention relates to leukotriene A4 hydrolase (LTA4H) inhibitors for the treatment of inflammation. More particularly, this invention relates to certain phenyl and pyridyl amine compounds useful as selective inhibitors of the LTA4H enzyme for the treatment of inflammatory conditions.

### BACKGROUND OF THE INVENTION

Inflammation is normally an acute response by the immune system to invasion by microbial pathogens, chemicals or physical injury. In some cases, however, the inflammatory response can progress to a chronic state, and be the cause of inflammatory disease. Therapeutic control of inflammation in diverse diseases is a major medical need.

Leukotrienes (LT) are biologically active metabolites of arachidonic acid (Samuelsson, B. Science 1983, 220(4597):568-575) that have been implicated in inflammatory diseases, including asthma (Munafo, D. A., et al. J. Clin. Invest. 1994, 93(3), 1042-1050), inflammatory bowel disease (IBD) (Sharon, P. et al. Gastroenterology 1984, 86(3), 453-460), chronic obstructive pulmonary disease (COPD) (Barnes, P. J. Respiration 2001, 68(5), 441-448), arthritis (Griffiths, R. J., et al. Proc. Natl. Acad. Sci. U.S.A. 1995, 92(2), 517-521; Tsuji, F., et al. Life Sci. 1998, 64(3), L51-L56), psoriasis (Ikai, K. J. Dermatol. Sci. 1999, 21(3), 135-146; Zhu, Y. I. et al. Skin Pharmacol. Appl. Skin Physiol. 2000, 13(5), 235-245) and atherosclerosis (Friedrich, E. B., et al. Arterioscler. Thromb. Vasc. Biol. 2003, 23,1761-7; Subbarao, K., et al. Arterioscler. Thromb. Vasc. Biol. 2004, 24, 369-75; Helgadottir, A., et al. Nat. Genet. 2004, 36(3), 233-9; Jala, V. R. et al. Trends Immunol. 2004, 25(6), 315-322). The synthesis of leukotrienes is initiated by the conversion of arachidonic acid to an unstable epoxide intermediate, leukotriene A4 (LTA4), by 5-lipoxygenase (5-LO) (Ford-Hutchinson, F. A., et al. Annu. Rev. Biochem. 1994, 63, 383-347). This enzyme is expressed predominantly by cells of myeloid origin, particularly neutrophils, eosinophils, monocytes/macrophages and mast cells (Reid, G. K., et al. J. Biol. Chem. 1990, 265(32), 19818-19823). LTA4 can either be conjugated with glutathione by leukotriene C4 (LTC4) synthase to produce the cysteinyl leukotriene, LTC4, or hydrolyzed to the diol, leukotriene B4 (LTB4) (Samuelsson, B., 1983). LTC4 and its metabolites, LTD4 and LTE4, induce smooth muscle contraction, broncho-constriction and vascular permeability, while LTB4 is a potent chemo-attractant and activator of neutrophils.

The stereospecific hydrolysis of LTA4 to LTB4 is catalyzed by leukotriene A4 hydrolase (LTA4H), a zinc-containing, cytosolic enzyme. This enzyme is ubiquitously expressed, with high levels in small intestinal epithelial cells, lung, and aorta (Samuelsson, B. et al. J. Biol. Chem. 1989, 264(33), 19469-19472). Moderate expression of LTA4H is observed in leukocytes, particularly neutrophils (Yokomizo, T., et al. J. Lipid Mediat. Cell Signal. 1995, 12(2,3), 321-332).

Leukotriene B4 is a key pro-inflammatory mediator, able to recruit inflammatory cells, such as neutrophils and eosinophils, as well as activate neutrophils (Fitzpatrick, F. A., et al. Ann. N. Y. Acad. Sci. 1994, 714, 64-74; Crooks, S. W. et al. Int. J. Biochem. Cell Biol. 1998, 30(2), 173-178; Klein, A., et al. J. Immunol. 2000, 164(8), 4271-4276). LTB4 mediates its pro-inflammatory effects by binding to G protein-coupled receptors, leukotriene B4 receptor 1 (BLT1) and leukotriene B4 receptor 2 (BLT2) (Yokomizo, T., et al. Arch. Biochem. Biophys. 2001, 385(2), 231-241). The receptor first identified, BLT1, binds LTB₄ with high affinity, leading to intracellular signaling and chemotaxis. BLT1 is expressed mainly in peripheral leukocytes, particularly neutrophils, eosinophils, macrophages (Huang, W. W., et al. J. Exp. Med. 1998, 188(6), 1063-74) and monocytes (Yokomizo, T., et al. Life Sci. 2001, 68, 2207-12). The murine receptor is also expressed on effector T cells and was recently shown to mediate LTB₄-dependent migration of effector CD8⁺ T cells (Goodarzi, K., et al. Nat. Immunol. 2003, 4(10), 965-73; Ott, V. L. et al. Nat. Immunol. 2003, 4(10), 974-81), early effector CD4⁺ T helper type 1 (T_{H}1) and T_{H}2 chemotaxis and adhesion to endothelial cells, as well as early effector CD4⁺ and CD8⁺ T cell recruitment in an asthma animal model (Tager, A. M., et al. Nat. Immunol. 2003, 4(10), 982-90). LTB4 receptor BLT2 (Wang, S., et al. J. Biol. Chem. 2000, 275(52), 40686-40694; Yokomizo, T., et al. J. Exp. Med. 2000,192(3), 421-431) shares 42% amino acid homology with BLT1, but is more broadly expressed, including in peripheral tissues such as the spleen, ovary and liver, as well as in leukocytes. BLT2 binds LTB4 with lower affinity than BLT1 does, mediates chemotaxis at higher concentrations of LTB4, and differs from BLT1 in its affinity for certain antagonists. While LTB4 receptor antagonists may differ in their affinity for BLT1 versus BLT2, blocking the production of LTB4 using LTA4H inhibitors is expected to inhibit the downstream events mediated through both BLT1 and BLT2.

Studies have shown that introduction of exogenous LTB4 into normal tissues can induce inflammatory symptoms (Camp, R. D. R., et al. Br. J. Pharmacol. 1983, 80(3), 497-502; Camp, R., et al. J. Invest. Dermatol. 1984, 82(2), 202-204). Elevated levels of LTB4 have been observed in a number of inflammatory diseases including IBD, COPD, psoriasis, rheumatoid arthritis (RA), cystic fibrosis and asthma (Crooks, S. W. et al. Int. J. Biochem. Cell Biol. 1998, 30(2), 173-178). Therefore, reduction of LTB4 production by an inhibitor of LTA4H activity is expected to have therapeutic potential in a wide range of diseases.

Support for these effects includes studies of LTA4H-deficient mice that, while otherwise healthy, exhibited markedly decreased neutrophil influx in arachidonic acid-induced ear inflammation and zymosan-induced peritonitis models (Byrum, R. S., et al. J. Immunol. 1999, 163(12), 6810-6819). Furthermore, LTA4H inhibitors have been shown to be effective anti-inflammatory agents in pre-clinical studies. For example, oral administration of LTA4H inhibitor SC57461 caused inhibition of ionophore-induced LTB4 production in mouse blood ex vivo, and in rat peritoneum in vivo (Kachur, J. K., et al. J. Pharmacol. Exp. Ther. 2002, 300(2), 583-587). Eight weeks of treatment with the same inhibitor compound significantly improved colitis symptoms in cotton top tamarins (Penning, T. D. Curr. Pharm. Des. 2001, 7(3), 163-179). The spontaneous colitis that develops in these animals is very similar to human IBD. The results therefore indicate that LTA4H inhibitors would have therapeutic utility in this and other human inflammatory diseases.

Events that elicit the inflammatory response include the formation of the pro-inflammatory mediator leukotriene B4. Hydrolase LTA4H catalyzes the formation of this mediator, and LTA4H inhibitors block the production of the pro-inflammatory mediator LTB4, thus providing the ability to prevent and/or treat leukotriene-mediated conditions, such as inflammation. The inflammatory response is characterized by pain, increased temperature, redness, swelling, or reduced function, or by a combination of two or more of these symptoms. Regarding the onset and evolution of inflammation, inflammatory diseases or inflammation-mediated diseases or conditions include, but are not limited to, acute inflammation, allergic inflammation, and chronic inflammation.

Examples of textbooks on the subject of inflammation include J. I. Gallin and R. Snyderman, Inflammation: Basic Principles and Clinical Correlates, 3^{rd} Edition, (Lippincott Williams & Wilkins, Philadelphia, 1999); V. Stvrtinova, J. Jakubovsky and I. Hulin, “Inflammation and Fever”, Pathophysiology Principles of Diseases (Textbook for Medical Students, Academic Press, 1995); Cecil et al., Textbook Of Medicine, 18 Edition (W. B. Saunders Company, 1988); and Steadmans Medical Dictionary.

Background and review material on inflammation and conditions related with inflammation can be found in articles such as the following: Nathan, C. Nature 2002, 420(6917), 846-852; Tracey, K. J. Nature 2002, 420(6917), 853-859; Coussens, L. M. et al. Nature 2002, 420(6917), 860-867; Libby, P. Nature 2002, 420(6917), 868-874; Benoist, C. et al. Nature 2002, 420(6917), 875-878; Weiner, H. L. et al. Nature 2002, 420(6917), 879-884; Cohen, J. Nature 2002, 420(6917), 885-891; Steinberg, D. Nat. Med. 2002, 8(11), 1211-1217. Cited references are incorporated herein by reference.

Inflammation is due to any one of a plurality of conditions, such as asthma, chronic obstructed pulmonary disease (COPD), atherosclerosis, rheumatoid arthritis, multiple sclerosis, inflammatory bowel diseases (including Crohn's disease and ulcerative colitis), or psoriasis, which are each characterized by excessive or prolonged inflammation at some stage of the disease.

Leukotriene modifiers are expected to have a beneficial role in the cardiovascular field by blocking aspects of the inflammatory component of cardiovascular diseases. It is to be noted in this regard that inflammation and immune mechanisms are important in atherosclerosis, and studies in the field support the rationale for blocking inflammation as a means for improving clinical cardiovascular conditions. Several studies have outlined an important function of leukotrienes in the development and progression of atherosclerosis, a disease that is now recognized as an inflammatory disease. Based on the role of LTA4H inhibitors in inflammation, and on evidence linking the leukotriene pathway to cardiovascular disease, LTA4H inhibitors are also likely to be useful in treating cardiovascular diseases that have an inflammatory component. LTA4H inhibitors are likely to be useful in treating, for example, myocardial infarction, aortic aneurysm, ischemia reperfusion, and stroke (Funk, C. D., Nat. Rev. Drug Disc. 2005, 4, 664-672; Jala, V. R. et al., 2004).

Applicants have discovered phenyl and pyridyl amine compounds and derivatives thereof; their use as inhibitors of enzymes, such as the LTA4H enzyme, in the formation of pro-inflammatory mediators, such as the LTB4 mediator; also their use for the treatment of inflammatory conditions; and the preparation of pharmaceutical compositions for the treatment of inflammation. Alkoxyphenylalkylamine derivatives having an antipsychotic action have been disclosed in U.S. Pat. No. 5,495,046. Phenylalkyl amine derivatives having anti-ischaemic activity have been disclosed in EP application 89202383.9.

### SUMMARY OF THE INVENTION

There are provided by the present invention compounds which have the following general formula (I): 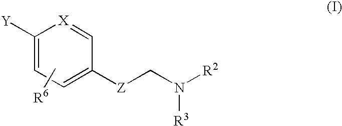
wherein
X is selected from the group consisting of CH and N;
Y is selected from the group consisting of R¹(CH₂)₂₋₃O—, R⁷N(R⁸)CO₂—, R⁷N(R⁸)C(O)N(R⁸)—, R⁷N(R⁸)CO₂CH₂—, R⁷N(R⁸)C(O)CH₂—, R¹OC(O)N(R⁸)—, R¹OCO₂—, R¹CO₂—, R¹CH(R⁹)CO₂—, R¹C(O)CH(R¹⁰)O—, and R¹CH(R⁹)CH(R¹⁰)O—, provided that when one of R⁹ and R¹⁰ in R¹CH(R⁹)CH(R¹⁰)O— is —H, then the other is not —H;
R¹ is a moiety selected from the group consisting of phenyl, thienyl, pyrrolyl, furanyl, oxazolyl, imidazolyl, thiazolyl, indolyl, indanyl, and tetrahydronaphthyl, wherein R¹ is substituted with 0, 1, or 2 substituents R⁴;
R⁴ is selected from the group consisting of —H, —OCH₃, —Cl, —F, —Br, —I, —OH, —NH₂, —CN, —CF₃, and —CH₃;
R⁷ is —C₁₋₄alkyl or is selected from the group consisting of phenyl, thienyl, pyrrolyl, furanyl, oxazolyl, imidazolyl, thiazolyl, indolyl, indanyl, and tetrahydronaphthyl, wherein R⁷ is substituted with 0, 1, or 2 substituents R⁴;
R⁸ is —H or —C₁₋₄alkyl;
or, R⁷ and R⁸ are taken together with the nitrogen member to which they are attached to form pyrrolidinyl, piperidinyl, morpholinyl, or thiomorpholinyl;
R⁹ is —H, —C₁₋₄alkyl, —Cl, or —OH;
R¹⁰ is —H, —C₁₋₄alkyl or is taken together with one of R⁴ to form a 5- or 6-membered carbocyclic ring;
Z is selected from the group consisting of bond, —CH₂—, —OCH₂—, —OCH₂CH(R¹¹)—, and —CH₂CH(R¹¹)—;
R¹¹ is —H or —OH;
provided that when Z is bond, then Y is one of R¹(CH₂)₂₋₃O—, R¹CO₂—, R¹CH(R⁹)CO₂—, R¹C(O)CH(R¹⁰)O—, and R¹CH(OH)CH(R¹⁰)O—;
R⁶ is —H or —F; and
R² and R³ are each independently selected from the group consisting of
   A) —H, —C₁₋₇alkyl, —C₃₋₇alkenyl, wherein the carbon in said alkenyl that is attached to the nitrogen member has only single bonds, —C₃₋₇alkynyl, wherein the carbon in said alkynyl that is attached to the nitrogen member has only single bonds, —C₃₋₇cycloalkyl optionally benzo fused, —C₅₋₇cycloalkenyl, —C₃₋₇cycloalkylC₁₋₇alkyl, —C₁₋₇alkylC₃₋₇cycloalkyl and phenyl, wherein each of the substituents A) is independently substituted with 0, 1, or 2 substituents R^{Q}, and each of said R^{Q} is a substituent at a carbon member that is at least one carbon member removed from the nitrogen member;
   B) a 4-7 membered saturated heterocyclic ring HetR^{a}, said 4-7 membered saturated heterocyclic ring HetR^{a}, having 0 or 1 double bonds, having a carbon member point of attachment and containing a member >NR^{M} as a heteroatom member, and said heteroatom member being separated from said carbon member point of attachment by at least one additional carbon member;
   C) —C₁₋₇alkylC(O)R^{x}, optionally substituted with CH₂R^{Ar} or CH₂R^{Ar′};
   D) —C₂₋₅alkylC(O)R^{x}, wherein two valence allowed carbon members in the C₂₋₅alkyl of said —C₂₋₅alkylC(O)R^{x} are part of a saturated C₃₋₆carbocycle;
   E) —C₂₋₅alkylOH wherein two valence allowed carbon members in the C₂₋₅alkyl of said —C₂₋₅alkylOH are part of a saturated C₃₋₆carbocycle;
   F) —C₀₋₄alkylphenyl, wherein the phenyl in said —C₀₋₄alkylphenyl is fused at two adjacent carbon members in said phenyl to R^{f}, or is benzofused;
   G) —C₀₋₄alkylAr⁶, where Ar⁶ is a 6-membered heteroaryl having a carbon member point of attachment and having 1 or 2 —N═ heteroatom members, and benzofused;
   H) —C₀₋₄alkylAr⁵, where Ar⁵ is a 5-membered heteroaryl, having one heteroatom member selected from the group consisting of O, S, and >NR^{Y}, and having 0 or 1 —N═ additional heteroatom member, optionally containing 1 or 2 carbonyl groups, and optionally benzofused;
   I) —C₁₋₄alkylAr^{5′}, where Ar^{5′} is a 5-membered heteroaryl containing 3 or 4 nitrogen members, optionally substituted with R^{Y}, and having a valence allowed site as a point of attachment;
   J) —C₀₋₄alkylAr⁶⁻⁶, where Ar⁶⁻⁶ is a C₀₋₄alkyl-attached phenyl fused at valence allowed sites to a 6-membered heteroaryl, wherein said 6-membered heteroaryl has 1 or 2-N═ heteroatom members;
   K) —C₀₋₄alkylAr⁶⁻⁵, where Ar⁶⁻⁵ is a C₀₋₄alkyl-attached phenyl fused at valence allowed sites to a 5-membered heteroaryl, said 5-membered heteroaryl having one heteroatom member selected from the group consisting of O, S, and >NR^{Y}, and said 5-membered heteroaryl having 0 or 1 additional heteroatom member which is —N═;
   L) one of 2-(4-ethyl-phenoxy)-benzothiazole, 2-(4-ethyl-phenoxy)-benzooxazole, and 2-(4-ethyl-phenoxy)-1H-benzoimidazole; and
   M) —SO₂C₁₋₄alkyl;
alternatively R² and R³ are taken together with the nitrogen to which they are attached to form a heterocyclic ring that contains at least one heteroatom member that is said attachment nitrogen, said heterocyclic ring being selected from the group consisting of
   i) a 4-7 membered saturated heterocyclic ring HetR^{b}, said 4-7 membered saturated heterocyclic ring HetR^{b} having one heteroatom member that is said attachment nitrogen, and being substituted with 0, 1, or 2 substituents at the same or at different ring members, said substituents being selected from the group consisting of —R^{Y}, —CN, —C(O)R^{Y}, —C₀₋₄alkylCO₂R^{Y}, —CO₀₋₄alkylC(O)CO₂R^{Y}, —C₀₋₄alkylOR^{Y}, —C₀₋₄alkylC(O)NR^{Y}R^{Z}, —C₀₋₄alkylNR^{Y}C(O)R^{Z}, —C(O)NR^{Z}OR^{Y}, —C₀₋₄alkylNR^{Y}C(O)CH₂OR^{Y}, —C₀₋₄alkylNR^{Y}C(O)CH₂C(O)R^{Y}, —C₀₋₄alkylNR^{Y}CO₂R^{Y}, —C₀₋₄alkylNR^{Y}C(O)NR^{Y}R^{Z}, —C₀₋₄alkylNR^{Y}C(S)NR^{Y}R^{Z}, —NR^{Y}C(O)CO₂R^{Y}, —NR^{Y}R^{Z}, —C₀₋₄alkylNR^{W}SO₂R^{Y}, 1,3-dihydro-indol-2-one-1-yl, 1,3-dihydro-benzoimidazol-2-one-1-yl, tetrazol-5-yl, 1-R^{Y}-1H-tetrazol-5-yl, R^{Y}-triazolyl, 2-R^{Y}-2H-tetrazol-5-yl, pyrrolidine-2-thion-1-yl, piperidine-2-thion-1-yl, —C₀₋₄alkylC(O)N(R^{Y})(SO₂R^{Y}), —C₀₋₄alkylN(R^{Y})(SO₂)NR^{Y}R^{Y}, —C₀₋₄alkylN(R^{Y})(SO₂)NR^{Y}CO₂R^{Y}, halo, 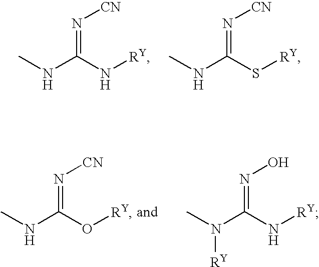
   ii) a 5-7 membered saturated heterocyclic ring HetR^{c}, said 5-7 membered saturated heterocyclic ring HetR^{c} having one additional heteroatom member separated from said attachment nitrogen by at least one carbon member, said additional heteroatom member being selected from the group consisting of O, S(═O)₀₋₂, and >NR^{M}, said 5-7 membered saturated heterocyclic ring HetR^{c} having 0 or 1 carbonyl members, and being substituted with 0, 1, or 2 substituents at the same or at different carbon ring members, said substituents being selected from the group consisting of —C(O)R^{Y}, —CO₂R^{Y}, —C₃₋₄alkylCO₂R^{Y} and R^{Z};
   iii) one of imidazolidin-1-yl, 2-imidazolin-1-yl, pyrazol-1-yl, imidazol-1-yl, 2H-tetrazol-2-yl, 1H-tetrazol-1-yl, pyrrol-1-yl, 2-pyrrolin-1-yl, and 3-pyrrolin-1-yl, wherein each of said 2H-tetrazol-2-yl and 1H-tetrazol-1-yl is substituted at the carbon member Y with 0 or 1 of —C₀₋₄alkylR^{Z}, —C₀₋₄alkylSR^{Y}, —C₀₋₄alkylCO₂R^{Y}, and substituent HetR^{a}; and
   iv) one of 1,2,3,4-tetrahydro-quinolin-1-yl, 1,2,3,4-tetrahydro-isoquinolin-2-yl, indol-1-yl, isoindol-2-yl, indolin-1-yl, benzimidazol-1-yl, 2,8-diaza-spiro[4.5]decan-1-one-8-yl, 4-{[(2-tert-butoxycarbonylamino-cyclobutanecarbonyl)-amino]-methyl}-piperidin-1-yl, 4-{[(2-amino-cyclobutanecarbonyl)-amino]-methyl}-piperidin-1-yl, 3,9-diaza-spiro[5.5]undecane-3-carboxylic acid-9-yl tert-butyl ester, 4-oxo-1-phenyl-1,3,8-triaza-spiro[4.5]dec-8-yl, and 4-oxo-1,3,8-triaza-spiro[4.5]dec-8-yl;
      wherein
R^{K} is selected from the group consisting of —H, —C₁₋₄alkyl and —C₀₋₄alkylR^{Ar}, each of said —C₁₋₄alkyl and —C₀₋₄alkylR^{Ar} being optionally substituted with 1, 2, or 3 substituents R^{N};
R^{L} is selected from the group consisting of —CO₂R^{S} and —C(O)NR^{S}R^{S′};
R^{M} is selected from the group consisting of R^{Z}, indol-7-yl, —SO₂R^{Y}, —C₃₋₄alkylCO₂R^{Y}, —CO₂R^{Y}, —C(O)NR^{Z}OR^{Y}, —C(O)R^{Y}, —C(O)C₁₋₄alkylOR^{Y}, —C₀₋₄alkylC(O)NR^{S}R^{S′}, C⁰⁻⁴alkylC(O)CO₂R^{Y}, 1,3-dihydro-indol-2-one-1-yl, 1,3-dihydro-benzoimidazol-2-one-1-yl, tetrazol-5-yl, 1-R^{Y}-1H-tetrazol-5-yl, R^{Y}-triazolyl, 2-R^{Y}-2H-tetrazol-5-yl and —C₀₋₄alkylC(O)N(R^{Y})(SO₂R^{Y}) each of said R^{M} that is not —H being optionally substituted with 1, 2, or 3 substituents R^{N};
R^{N} is selected from the group consisting of —OCH₃, —Cl, —F, —Br, —I, —OH, —NH₂, —CN, —CF₃, —CH₃, —OC(O)CH₃, and —NO₂;
R^{Q} is selected from the group consisting of —Cl, —F, —Br, —I, —CF₃, —CCl₃, —CN, —C₁₋₄alkyl, —C₀₋₄alkylR^{Ar}, —C₀₋₄alkylR^{Ar′}, —C₀₋₄alkylOR^{Y}, —C₀₋₄alkylCO₂R^{Y}, —C₀₋₄alkylNR^{Y}R^{Z}, —C₀₋₄alkylNR^{Y}COR^{Y}, —C₀₋₄alkylNR^{Y}CONR^{Y}R^{Z}, —C₀₋₄alkylNR^{Y}SO₂R^{Y}, and —C₀₋₄alkylSR^{Y}; R^{S} and R^{S′} are independently selected from the group consisting of —H, —C₁₋₄alkyl, and —C₀₋₄alkylphenyl; alternatively, R^{S} and R^{S′} are taken together with the nitrogen member to which said R^{S} and R^{S′} are attached to form a 4-7 membered heterocyclic ring having 0 or 1 additional heteroatom member selected from the group consisting of O, S, and >NR^{Y}, provided that said additional heteroatom member is separated by at least two carbon members from said nitrogen member to which said R^{S} and R^{S′} are attached, and provided that where R^{Y} is C₀₋₄alkylR^{Ar}, then R^{Ar} is not substituted with R^{L};
R^{W} is selected from the group consisting of R^{Y}, and —C₃₋₇cycloalkyl;
R^{X} is selected from the group consisting of —OR^{Y}, —NR^{Y}R^{Z}, —C₁₋₄alkyl, and —C₀₋₄alkylR^{Ar};
R^{Y} is selected from the group consisting of —H, —C₁₋₄alkyl, —C₀₋₄alkylR^{Ar} and —C₀₋₄alkylR^{Ar′}, each of said R^{Y} that is not —H being optionally substituted with 1, 2, or 3 substituents R^{N};
R^{Z} is selected from the group consisting of R^{Y}, —C₂₋₄alkylOR^{Y}, —C₁₋₂alkylCO₂R^{Y}, —C₁₋₂alkylC(O)NR^{S}R^{S′}, and —C₂₋₄alkylNR^{S}R^{S′}; provided that when R^{Y} and R^{Z} are attached to a nitrogen member, then R^{Y} and R^{Z} are selected as defined above, or R^{Y} and R^{Z} are taken together with the R^{Y}- and R^{Z}-attached nitrogen member to form a 4-7 membered heterocyclic ring HetR^{d} having 0 or 1 additional heteroatom members selected from the group consisting of O, S, and >NR^{M}, said 4-7 membered heterocyclic ring HetR^{d} having 0 or 1 carbonyl members, and said 4-7 membered heterocyclic ring HetR^{d} having 0 or 1 valence allowed carbon members substituted with at least one of R^{M}, —CO₂H, and —C₀₋₁alkylOR^{Y}; R^{Ar} is a moiety with a carbon member attachment point and said R^{Ar} is selected from the group consisting of phenyl, pyridyl, pyrimidyl, and pyrazinyl, wherein each valence allowed carbon member in each of said R^{Ar} is independently substituted with at least one of 0, 1, 2, or 3 substituents R^{N}, and 0 or 1 substituent R^{L};
R^{Ar′} is a 3-8 membered ring having 0, 1, or 2 heteroatom members selected from the group consisting of O, S, N, and >NR^{Y}, said R^{Ar′} having 0, 1, or 2 unsaturated bonds and having 0 or 1 carbonyl members, wherein each valence allowed member in each of said R^{Ar′} ring is independently substituted with 0, 1, or 2 substituents R^{K}; and
R^{f} is a linear 3- to 5-membered hydrocarbon moiety having 0 or 1 unsaturated carbon-carbon bonds and having 0 or 1 carbonyl members; and enantiomers, diasteromers, racemates, tautomers, hydrates, solvates, and pharmaceutically acceptable salts, esters, and amides thereof. Embodiments of compounds of formula (I) are LTA4H modulators. Embodiments of compounds of formula (I) are LTA4H inhibitors. Embodiments of this invention comprise mixtures of compounds of formula (I).

Embodiments of the present invention comprise compounds that have the following general formula (II), and enantiomers, diasteromers, racemates, tautomers, hydrates, solvates, and pharmaceutically acceptable salts, esters, and amides thereof: 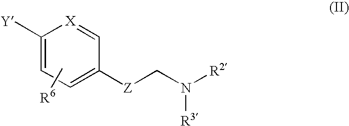
wherein
X is selected from the group consisting of CH and N;
Y is selected from the group consisting of R¹(CH₂)₂₋₃O—, R⁷N(R⁸)CO₂—, R⁷N(R⁸)C(O)N(R⁸)—, R⁷N(R⁸)CO₂CH₂—, R⁷N(R⁸)C(O)CH₂—, R¹OC(O)N(R⁸)—, R¹OCO₂—, R¹CO₂—, R¹CH(R⁹)CO₂—, R¹C(O)CH(R¹⁰)O—, and R¹CH(R⁹)CH(R¹⁰)O—, provided that when one of R⁹ and R¹⁰ in R¹CH(R⁹)CH(R¹⁰)O— is —H, then the other is not —H;
R¹ is a moiety selected from the group consisting of phenyl, thienyl, pyrrolyl, furanyl, oxazolyl, imidazolyl, thiazolyl, indolyl, indanyl, and tetrahydronaphthyl, wherein R¹ is substituted with 0, 1, or 2 substituents R⁴;
R⁴ is selected from the group consisting of —H, —OCH₃, —Cl, —F, —Br, —I, —OH, —NH₂, —CN, —CF₃, and —CH₃;
R⁷ is —C₁₋₄alkyl or is selected from the group consisting of phenyl, thienyl, pyrrolyl, furanyl, oxazolyl, imidazolyl, thiazolyl, indolyl, indanyl, and tetrahydronaphthyl, wherein R⁷ is substituted with 0, 1, or 2 substituents R⁴;
R⁸ is —H or —C₁₋₄alkyl;
or, R⁷ and R⁸ are taken together with the nitrogen member to which they are attached to form pyrrolidinyl, piperidinyl, morpholinyl, or thiomorpholinyl;
R⁹ is —H, —C₁₋₄alkyl, —Cl, or —OH;
R¹⁰ is —H, —C₁₋₄alkyl or is taken together with one of R⁴ to form a 5- or 6-membered carbocyclic ring;
R¹¹ is —H or —OH;
Z is selected from the group consisting of bond, —CH₂—, —OCH₂—, —OCH₂CH(R¹¹)—, and —CH₂CH(R¹¹)—;
provided that when Z is bond, then Y is one of R¹(CH₂)₂₋₃O—, R¹CO₂—, R¹CH(R⁹)CO₂—, R¹C(O)CH(R¹⁰)O—, and R¹CH(OH)CH(R¹⁰)O—;
R⁶ is —H or —F; and
R^{2′} and R^{3′} are each independently selected from the group consisting of
   A) H, C₁₋₇alkyl, C₃₋₇alkenyl, wherein the carbon in said alkenyl that is attached to the nitrogen member has only single bonds, C₃₋₇alkynyl, wherein the carbon in said alkynyl that is attached to the nitrogen member has only single bonds, C₃₋₇cycloalkyl optionally benzofused, C₅₋₇cycloalkenyl, C₃₋₇cycloalkylC₁₋₇alkyl, C₁₋₇alkylC₃₋₇cycloalkyl and phenyl, wherein each of the substituents A) is independently substituted with 0, 1, or 2 substituents R^{Q}, and each of said R^{Q} is a substituent at a carbon member that is at least one carbon member removed from the nitrogen member;
   B) a 4-7 membered saturated heterocyclic ring HetR^{a}, said 4-7 membered saturated heterocyclic ring HetR^{a}, having 0 or 1 double bonds, having a carbon member point of attachment and containing a member >NR^{M} as a heteroatom member, and said heteroatom member being separated from said carbon member point of attachment by at least one additional carbon member;
   C) C₁₋₇alkylC(O)R^{x}, optionally substituted with CH₂R^{Ar} or CH₂R^{Ar′};
   D) C₂₋₅alkylC(O)R^{x}, wherein two valence allowed carbon members in the C₂₋₅alkyl of said —C₂₋₅alkylC(O)R^{x} are part of a saturated C₃₋₆carbocycle;
   E) —C₂₋₅alkylOH wherein two valence allowed carbon members in the C₂₋₅alkyl of said —C₂₋₅alkylOH are part of a saturated C₃₋₆carbocycle;
   F) —C₀₋₄alkylphenyl, wherein the phenyl in said —C₀₋₄alkylphenyl is fused at two adjacent carbon members in said phenyl to R^{f}, or is benzofused;
   G) —C₀₋₄alkylAr⁶, where Ar⁶ is a 6-membered heteroaryl having a carbon member point of attachment and having 1 or 2 —N═ heteroatom members, and benzofused;
   H) —C₀₋₄alkylAr⁵, where Ar⁵ is a 5-membered heteroaryl, having one heteroatom member selected from the group consisting of O, S, and >NR^{Y}, and having 0 or 1 —N═ additional heteroatom member, optionally containing 1 or 2 carbonyl groups, and optionally benzofused;
   I) —C₁₋₄alkylAr^{5′}, where Ar^{5′} is a 5-membered heteroaryl containing 3 or 4 nitrogen members, optionally substituted with R^{Y}, and having a valence allowed site as a point of attachment;
   J) —C₀₋₄alkylAr⁶⁻⁶, where Ar⁶⁻⁶ is a C₀₋₄alkyl-attached phenyl fused at valence allowed sites to a 6-membered heteroaryl, wherein said 6-membered heteroaryl has 1 or 2 —N═ heteroatom members;
   K) —C₀₋₄alkylAr⁶⁻⁵, where Ar⁶⁻⁵ is a C₀₋₄alkyl-attached phenyl fused at valence allowed sites to a 5-membered heteroaryl, said 5-membered heteroaryl having one heteroatom member selected from the group consisting of O, S, and >NR^{Y}, and said 5-membered heteroaryl having 0 or 1 additional heteroatom member which is —N═;
   L) one of 2-(4-ethyl-phenoxy)-benzothiazole, 2-(4-ethyl-phenoxy)-benzooxazole, and 2-(4-ethyl-phenoxy)-1H-benzoimidazole; and
   M) —SO₂C₁₋₄alkyl;
      alternatively R^{2′} and R^{3′} are taken together with the nitrogen to which they are attached to form a heterocyclic ring that contains at least one heteroatom member that is said attachment nitrogen, said heterocyclic ring being selected from the group consisting of
   i) a 4-7 membered saturated heterocyclic ring HetR^{b}, said 4-7 membered saturated heterocyclic ring HetR^{b} having one heteroatom member that is said attachment nitrogen, and being substituted with 0, 1, or 2 substituents at the same or at different ring members, said substituents being selected from the group consisting of —R^{Y}, —CN, —C(O)R^{Y}, —C₀₋₄alkylC(O)CO₂R^{Y}, —CO₀₋₄alkylC(O)CO₂R^{Y}, —C₀₋₄alkylOR^{Y}, —C₀₋₄alkylC(O)NR^{Y}R^{Z}, —C₀₋₄alkylNR^{Y}C(O)R^{Z}, —C(O)NR^{Z}OR^{Y}, —C₀₋₄alkylNR^{Y}C(O)CH₂OR^{Y}, —C₀₋₄alkylNR^{Y}C(O)CH₂C(O)R^{Y}, —C₀₋₄alkylNR^{Y}CO₂R^{Y}, —C₀₋₄alkylNR^{Y}C(O)NR^{Y}R^{Z}, —C₀₋₄alkylNR^{Y}C(S)NR^{Y}R^{Z}, —NR^{Y}C(O)CO₂R^{Y}, —NR^{Y}R^{Z}, —C₀₋₄alkylNR^{W}SO₂R^{Y}, 1,3-dihydro-indol-2-one-1-yl, 1,3-dihydro-benzoimidazol-2-one-1-yl, tetrazol-5-yl, 1-R^{Y}-1H-tetrazol-5-yl, R^{Y}-triazolyl, 2-R^{Y}-2H-tetrazol-5-yl, pyrrolidine-2-thion-1-yl, piperidine-2-thion-1-yl, —C₀₋₄alkylC(O)N(R^{Y})(SO₂R^{Y}), —C₀₋₄alkylN(R^{Y})(SO₂)NR^{Y}R^{Y}, —C₀₋₄alkylN(R^{Y})(SO₂)NR^{Y}CO₂R^{Y}, halo, 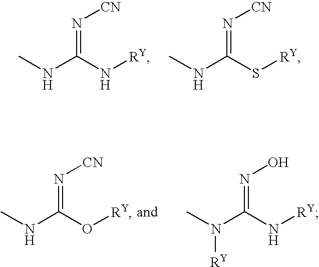
   ii) a 5-7 membered saturated heterocyclic ring HetR^{c}, said 5-7 membered saturated heterocyclic ring HetR^{c} having one additional heteroatom member separated from said attachment nitrogen by at least one carbon member, said additional heteroatom member being selected from the group consisting of O, S(═O)₀₋₂, and >NR^{M}, said 5-7 membered saturated heterocyclic ring HetR^{c} having 0 or 1 carbonyl members, and being substituted with 0, 1, or 2 substituents at the same or at different carbon ring members, said substituents being selected from the group consisting of —C(O)R^{Y}, —CO₂R^{Y}, —C₃₋₄alkylCO₂R^{Y} and R^{Z};
   iii) one of imidazolidin-1-yl, 2-imidazolin-1-yl, pyrazol-1-yl, imidazol-1-yl, 2H-tetrazol-2-yl, 1H-tetrazol-1-yl, pyrrol-1-yl, 2-pyrrolin-1-yl, and 3-pyrrolin-1-yl, wherein each of said 2H-tetrazol-2-yl and 1H-tetrazol-1-yl is substituted at the carbon member Y with 0 or 1 of —C₀₋₄alkylR^{Z}, —C₀₋₄alkylSR^{Y}, —C₀₋₄alkylCO₂R^{Y}, and substituent HetR^{a} ; and
   iv) one of 1,2,3,4-tetrahydro-quinolin-1-yl, 1,2,3,4-tetrahydro-isoquinolin-2-yl, indol-1-yl, isoindol-2-yl, indolin-1-yl, benzimidazol-1-yl, 2,8-diaza-spiro[4.5]decan-1-one-8-yl, 4-{[(2-tert-butoxycarbonylamino-cyclobutanecarbonyl)-amino]-methyl}-piperidin-1-yl, 4-{[(2-amino-cyclobutanecarbonyl)-amino]-methyl}-piperidin-1-yl, 3,9-diaza-spiro[5.5]undecane-3-carboxylic acid-9-yl tert-butyl ester, 4-oxo-1-phenyl-1,3,8-triaza-spiro[4.5]dec-8-yl, and 4-oxo-1,3,8-triaza-spiro[4.5]dec-8-yl;
      wherein
R^{K} is selected from the group consisting of —H, —C₁₋₄alkyl and —C₀₋₄alkylR^{Ar}, each of said —C₁₋₄alkyl and —C₀₋₄alkylR^{Ar} being optionally substituted with 1, 2, or 3 substituents R^{N};
R^{L} is selected from the group consisting of —CO₂R^{S} and —C(O)NR^{S}R^{S′};
R^{M} is selected from the group consisting of R^{Z}, indol-7-yl, —SO₂R^{Y}, —C₃₋₄alkylCO₂R^{Y}, —CO₂R^{Y}, —C(O)NR^{Z}OR^{Y}, —C(O)R^{Y}, —C(O)C₁₋₄alkylOR^{Y}, —C₀₋₄alkylC(O)NR^{S}R^{S′}, C⁰⁻⁴alkylC(O)CO₂R^{Y}, 1,3-dihydro-indol-2-one-1-yl, 1,3-dihydro-benzoimidazol-2-one-1-yl, tetrazol-5-yl, 1-R^{Y}-1H-tetrazol-5-yl, R^{Y}-triazolyl, 2-R^{Y}-2H-tetrazol-5-yl and —C₀₋₄alkylC(O)N(R^{Y})(SO₂R^{Y}) each of said R^{M} that is not —H being optionally substituted with 1, 2, or 3 substituents R^{N};
R^{N} is selected from the group consisting of —OCH₃, —Cl, —F, —Br, —I, —OH, —NH₂, —CN, —CF₃, —CH₃, —OC(O)CH₃, and —NO₂;
R^{Q} is selected from the group consisting of —Cl, —F, —Br, —I, —CF₃, —CCl₃, —CN, —C₁₋₄alkyl, —C₀₋₄alkylR^{Ar}, —C₀₋₄alkylR^{Ar′}, —C₀₋₄alkylOR^{Y}, —C₀₋₄alkylCO₂R^{Y}, —C₀₋₄alkylNR^{Y}R^{Z}, —C₀₋₄alkylNR^{Y}COR^{Y}, —C₀₋₄alkylNR^{Y}CONR^{Y}R^{Z}, —C₀₋₄alkylNR^{Y}SO₂R^{Y}, and —C₀₋₄alkylSR^{Y};
R^{S} and R^{S′} are independently selected from the group consisting of —H, —C₁₋₄alkyl, and —C₀₋₄alkylphenyl; alternatively, R^{S} and R^{S′} are taken together with the nitrogen member to which said R^{S} and R^{S′} are attached to form a 4-7 membered heterocyclic ring having 0 or 1 additional heteroatom member selected from the group consisting of O, S, and >NR^{Y}, provided that said additional heteroatom member is separated by at least two carbon members from said nitrogen member to which said R^{S} and R^{S′} are attached, and provided that where R^{Y} is C₀₋₄alkylR^{Ar}, then R^{Ar} is not substituted with R^{L};
R^{W} is selected from the group consisting of R^{Y}, and —C₃₋₇cycloalkyl;
R^{X} is selected from the group consisting of —OR^{Y}, —NR^{Y}R^{Z}, —C₁₋₄alkyl, and —C₀₋₄alkylR^{Ar};
R^{Y} is selected from the group consisting of —H, —C₁₋₄alkyl, —C₀₋₄alkylR^{Ar} and —C₀₋₄alkylR^{Ar′}, each of said R^{Y} that is not —H being optionally substituted with 1, 2, or 3 substituents R^{N};
R^{Z} is selected from the group consisting of R^{Y}, —C₂₋₄alkylOR^{Y}, —C₁₋₂alkylCO₂R^{Y}, —C₁₋₂alkylC(O)NR^{S}R^{S′}, and —C₂₋₄alkylNR^{S}R^{S′}; provided that when R^{Y} and R^{Z} are attached to a nitrogen member, then R^{Y} and R^{Z} are selected as defined above, or R^{Y} and R^{Z} are taken together with the R^{Y}- and R^{Z}-attached nitrogen member to form a 4-7 membered heterocyclic ring HetR^{d} having 0 or 1 additional heteroatom members selected from the group consisting of O, S, and >NR^{M}, said 4-7 membered heterocyclic ring HetR^{d} having 0 or 1 carbonyl members, and said 4-7 membered heterocyclic ring HetR^{d} having 0 or 1 valence allowed carbon members substituted with at least one of R^{M}, —CO₂H, and —C₀₋₁alkylOR^{Y}; R^{Ar} is a moiety with a carbon member attachment point and said R^{Ar} is selected from the group consisting of phenyl, pyridyl, pyrimidyl, and pyrazinyl, wherein each valence allowed carbon member in each of said R^{Ar} is independently substituted with at least one of 0, 1, 2, or 3 substituents R^{N}, and 0 or 1 substituent R^{L};
R^{Ar′} is a 3-8 membered ring having 0, 1, or 2 heteroatom members selected from the group consisting of O, S, N, and >NR^{Y}, said R^{Ar′} having 0, 1, or 2 unsaturated bonds and having 0 or 1 carbonyl members, wherein each valence allowed member in each of said R^{Ar′} ring is independently substituted with 0, 1, or 2 substituents R^{K}; and
R^{f} is a linear 3- to 5-membered hydrocarbon moiety having 0 or 1 unsaturated carbon-carbon bonds and having 0 or 1 carbonyl members; provided that when
   (c1) Y′ is R¹(CH₂)₂₋₃O—,
   (c2) Z is —CH₂—, and
   (c3) X is CH,
      then R^{2′} and R^{3′} independently are not —H, —C₁₋₇alkyl, or unsubstituted —C₁₋₇alkylC(O)R^{x}; or R^{2′} and R^{3′} taken together with the nitrogen member to which they are attached do not form HetR^{b} or HetR^{c} where R^{Y} or R^{M} are phenyl, pyridyl, or pyrimidyl.

Embodiments of compounds of formula (II) are LTA4H modulators. Embodiments of compounds of formula (II) are LTA4H inhibitors.

Isomeric forms of the compounds of formulae (I) and (II), and of their pharmaceutically acceptable salts, amides and esters, are encompassed within the present invention, and reference herein to one of such isomeric forms is meant to refer to at least one of such isomeric forms. One of ordinary skill in the art will recognize that compounds according to this invention may exist, for example in a single isomeric form whereas other compounds may exist in the form of an isomeric mixture.

Whether stated explicitly or not in any part of the written description and claims, it is understood that each substituent and member assignment in the context of this invention is made independently of any other member and substituent assignment, unless stated otherwise. By way of a first example on substituent terminology, if substituent S¹ₑₓₐₘₚₗₑ is one of S₁ and S₂, and substituent S²ₑₓₐₘₚₗₑ example is one of S₃ and S₄, then these assignments refer to embodiments of this invention given according to the choices S¹ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in-generic terms, is meant to illustrate the various substituent R assignments described herein. The foregoing convention given herein for substituents extends, when applicable, to members such as X and Z, and to any index if applicable.

Furthermore, when more than one assignment is given for any member or substituent, embodiments of this invention comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent R assignments described herein. The foregoing convention given herein for substituents extends, when applicable, to members such as X and Z, and to any index if applicable.

The nomenclature “Cᵢ₋ⱼ” with j>i, when applied herein to a class of substituents, is meant to refer to embodiments of this invention for which each and every one of the number of carbon members, from i to j, including i and j, is independently realized. By way of example, the term C₁₋₃ refers independently to embodiments that have one carbon member (C₁), embodiments that have two carbon members (C₂), and embodiments that have three carbon members (C₃).

The term Cₙ₋ₘalkyl refers to an aliphatic chain, whether straight or branched, with a total number N of carbon members in the chain that satisfies n≦N≦m, with m>n.

When any variable referring to a substituent, compound member or index, occurs more than once, the full range of assignments is meant to apply to each occurrence, independently of the specific assignment(s) to any other occurrence of such variable.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

Any linker referred to herein is meant to encompass the various attachment possibilities when more than one of such possibilities are allowed. For example, reference to linker -A-B—, where A≠B, refers herein to such member with A attached to a first terminus and B attached to a second terminus, and it also refers to such linker with A attached to the second terminus and B attached to the first terminus. Examples of such linker are provided by Z assignments such as —OCH₂—, —OCH₂CH(R¹¹)—, and —CH₂CH(R¹¹)—.

The present invention also features methods for inhibiting LTA4H enzyme activity with such compounds, pharmaceutical compositions containing such compounds, and methods of using such compositions in the treatment or prevention of conditions that are mediated by LTA4H enzyme activity.

Pharmaceutical compositions according to the present invention include at least one of the compounds of the present invention. If more than one of such compounds is included in a composition, the therapeutically effective amount may be a jointly effective amount. As such inhibitors of the LTA4H enzyme, compounds and compositions according to the present invention are useful in the prevention, inhibition, or treatment of inflammation.

The invention also features a pharmaceutical composition for treating or preventing an LTA4H-mediated condition in a subject, comprising a therapeutically effective amount of at least one LTA4H modulator selected from compounds of formulae (I) and (II), enantiomers, diastereomers, racemates, tautomers, hydrates, solvates thereof, pharmaceutically acceptable salts, amides and esters thereof. In addition, the invention features a pharmaceutical composition for inhibiting inflammatory response in a subject, comprising a therapeutically effective amount of at least one LTA4H inhibitor selected from compounds of formulae (I) and (II), enantiomers, diastereomers, racemates, tautomers, hydrates, solvates thereof, pharmaceutically acceptable salts, amides and esters thereof. The invention additionally features an anti-inflammatory composition, comprising a therapeutically effective amount of at least one anti-inflammatory compound selected from compounds of formulae (I) and (II), enantiomers, diastereomers, racemates, tautomers, hydrates, solvates thereof, pharmaceutically acceptable salts, amides and esters thereof.

The invention features methods for treating or preventing inflammation in a subject, comprising administering to the subject in connection with an inflammatory response a pharmaceutical composition that comprises a therapeutically effective amount of at least one anti-inflammatory compound selected from compounds of formulae (I) and (II), enantiomers, diastereomers, racemates, tautomers, hydrates, solvates thereof, pharmaceutically acceptable salts, amides and esters thereof. The invention also features methods for treating or preventing an LTA4H-mediated condition in a subject, comprising administering to the subject a pharmaceutical composition that comprises a therapeutically effective amount of at least one LTA4H modulator selected from compounds of formulae (I) and (II), enantiomers, diastereomers, racemates, tautomers, hydrates, solvates thereof, pharmaceutically acceptable salts, amides and esters thereof. Furthermore, the invention features methods for inhibiting inflammation in a subject, comprising administering to the subject a pharmaceutical composition that comprises a therapeutically effective amount of at least one LTA4H inhibitor selected from compounds of formulae (I) and (II), enantiomers, diastereomers, racemates, tautomers, hydrates, solvates thereof, pharmaceutically acceptable salts, amides and esters thereof.

This invention features methods for the treatment, prevention and/or inhibition of conditions that are associated with and/or cause inflammation, such as any one or a plurality of the following conditions: Asthma, chronic obstructed pulmonary disease (COPD), atherosclerosis, rheumatoid arthritis, multiple sclerosis, inflammatory bowel diseases (including Crohn's disease and ulcerative colitis), or psoriasis, which are each characterized by excessive or prolonged inflammation at some stage of the disease.

In addition, this invention features methods for the treatment, prevention, and/or inhibition of cardiovascular disease with an inflammatory component, such as myocardial infarction, aortic aneurysm, ischemia reperfusion, or stroke, comprising administering to the subject a pharmaceutical composition that comprises a therapeutically effective amount of at least one LTA4H modulator selected from compounds of formula (I), formula (II), enantiomers, diastereomers, racemates, tautomers, hydrates, solvates thereof, pharmaceutically acceptable salts, amides and esters thereof.

Additional features and advantages of the invention will become apparent from the detailed description below, including examples, and the appended claims.

### DETAILED DESCRIPTION OF THE INVENTION

The present invention is directed to compounds of formula (I) and (II), as herein defined, enantiomers, diastereomers, racemates, tautomers, hydrates, solvates thereof, pharmaceutically acceptable salts, amides and esters thereof, pharmaceutical compositions that contain at least one of such compounds, methods of using, including treatment and/or prevention of conditions such as those that are mediated by LTA4H, and methods of making such pharmaceutical compositions.

The following terms are defined below, and by their usage throughout the disclosure.

“Alkyl” includes straight chain and branched hydrocarbons with at least one hydrogen removed to form a radical group. Alkyl groups include methyl, ethyl, propyl, isopropyl, butyl, isobutyl, t-butyl, 1-methylpropyl, pentyl, isopentyl, sec-pentyl, hexyl, heptyl, octyl, and so on. Alkyl does not include cycloalkyl.

“Alkenyl” includes straight chain and branched hydrocarbon radicals as above with at least-one carbon-carbon double bond (sp²). Unless indicated otherwise by the prefix that indicates the number of carbon members, alkenyls include ethenyl (or vinyl), prop-1-enyl, prop-2-enyl (or allyl), isopropenyl (or 1-methylvinyl), but-1-enyl, but-2-enyl, butadienyls, pentenyls, hexa-2,4-dienyl, and so on.

“Alkynyl” includes straight chain and branched hydrocarbon radicals as above with at least one carbon-carbon triple bond (sp). Unless indicated otherwise by the prefix that indicates the number of carbon members, alkynyls include ethynyl, propynyls, butynyls, and pentynyls. Hydrocarbon radicals having a mixture of double bonds and triple bonds, such as 2-penten-4-ynyl, are grouped as alkynyls herein.

“Alkoxy” includes a straight chain or branched alkyl group with a terminal oxygen linking the alkyl group to the rest of the molecule. Alkoxy includes methoxy, ethoxy, propoxy, isopropoxy, butoxy, t-butoxy, pentoxy and so on. “Aminoalkyl”, “thioalkyl”, and “sulfonylalkyl” are analogous to alkoxy, replacing the terminal oxygen atom of alkoxy with, respectively, NH (or NR), S, and SO₂.

Unless indicated otherwise by the prefix that indicates the number of carbon members, “cycloalkyl” includes cyclopropyl, cyclobutyl, cyclopentyl, cyclohexyl, cycloheptyl, cyclooctyl, and so on.

Unless indicated otherwise by the prefix that indicates the number of members in the cyclic structure, “heterocyclyl”, “heterocyclic” or “heterocycle” is a 3- to 8-member aromatic, saturated, or partially saturated single or fused ring system that comprises carbon atoms wherein the heteroatoms are selected from N, O, and S. Examples of heterocyclyls include thiazoylyl, furyl, pyranyl, isobenzofuranyl, pyrrolyl, imidazolyl, pyrazolyl, isothiazolyl, isoxazolyl, pyridyl, pyrazinyl, pyrimidinyl, pyridazinyl, indolizinyl, isoindolyl, indolyl, indazolyl, purinyl, quinolyl, furazanyl, pyrrolidinyl, pyrrolinyl, imidazolidinyl, imidazolinyl, pyrazolidinyl, pyrazolinyl, piperidyl, piperazinyl, indolinyl, and morpholinyl. For example, preferred heterocyclyls or heterocyclic radicals include morpholinyl, piperazinyl, pyrrolidinyl, pyridyl, cyclohexylimino, cycloheptylimino, and more preferably, piperidyl.

“Aryl” includes phenyl, naphthyl, biphenylyl, tetrahydronaphthyl, and so on, any of which may be optionally substituted. Aryl also includes arylalkyl groups such as benzyl, phenethyl, and phenylpropyl. Aryl includes a ring system containing an optionally substituted 6-membered carbocyclic aromatic ring, said system may be bicyclic, bridged, and/or fused. The system may include rings that are aromatic, or partially or completely saturated. Examples of ring systems include indenyl, pentalenyl, 1-4-dihydronaphthyl, indanyl, benzimidazolyl, benzothiophenyl, indolyl, benzofuranyl, isoquinolinyl, and so on. Unless indicated otherwise, the terms “heteroaryl” or “heteroaromatic” refer to those heterocycles that are aromatic in nature. Examples illustrating heteroaryl are thienyl, furanyl, pyrrolyl, imidazolyl, oxazolyl, thiazolyl, benzothienyl, benzofuranyl, benzimidazolyl, benzoxazolyl, benzothiazolyl, pyridyl, and pyrimidinyl.

“Halo” includes fluoro, chloro, bromo, and iodo, and is preferably fluoro or chloro.

The term “carbonyl” refers to a >C═O moiety, such that when this term is characterized as being part of a chain or cyclic structure, the carbon member in the carbonyl group is taken as being one of the carbon members of such chain or cyclic structure.

The terms “carbocycle” and “carbocyclic” refer to a cycloalkyl or a partially saturated cycloalkyl that is not benzo

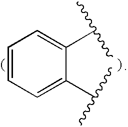

As in standard chemical nomenclature, the group phenyl is herein referred to as “phenyl” or as “Ph”.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum mass of the same entity that could be obtained under the particular stoichiometric conditions. Concentrations that are given as percentages refer to mass ratios, unless indicated differently.

It is understood that substitutions and combinations of substitutions recited herein, whether stated explicitly or not, refer to substitutions that are consistent with the valency of the member being substituted. Terms such as “valence allowed site,” “valence allowed member,” and morphological variations thereof are used in this sense. For example, “valence allowed” when applied to a carbon member refers to the tetravalency of C; it refers to the trivalency of N when applied to a nitrogen member; and it refers to the bonding of a nitrogen member that is conventionally characterized with a positive electric charge or that is in a quaternary form. The present invention also encompasses compounds as described herein and equivalents thereof with at least one valence allowed nitrogen member, including but not limited to a quaternary nitrogen member and a nitrogen oxide, each of which may be prepared according to methods known in the art (see J. March, Advanced Organic Chemistry, 4th ed., 1991, pp. 411-412, 1200-1201; R. C. Larock, Comprehensive Organic Transformations, 1989, pp. 397-400, 421-425; and references cited therein).

Particular preferred compounds of the invention comprise a compound of formula (I) or (II), or an enantiomer, diastereomer, racemate, tautomer, hydrate, solvate thereof, or a pharmaceutically acceptable salt, amide or ester thereof, wherein Y, Y′, X, R⁶, Z, R², R³, R^{2′} and R^{3′} have any of the meanings defined hereinabove and equivalents thereof, or at least one of the following assignments and equivalents thereof. Such assignments may be used where appropriate with any of the definitions, claims or embodiments defined herein:
X is CH;
Y′ is selected from the group consisting of R⁷N(R⁸)CO₂—, R⁷N(R⁸)C(O)N(R⁸)—, R⁷N(R⁸)CO₂CH₂—, R⁷N(R⁸)C(O)CH₂—, R¹OC(O)N(R⁸)—, R¹OCO₂—, R¹CO₂—, R¹CH(R⁹)CO₂—, R¹C(O)CH(R¹⁰)O—, and R¹CH(R⁹)CH(R¹⁰)O—, provided that when one of R⁹ and R¹⁰ in R¹CH(R⁹)CH(R¹⁰)O— is —H, then the other is not —H;
Y′ is R¹(CH₂)₂₋₃O—;
R¹ is selected from the group consisting of phenyl, thienyl, indolyl, and tetrahydronaphthyl, and R is substituted with 0, 1, or 2 substituents selected from the group consisting of —H, —OCH₃, —Cl, —F, —Br, —I, —OH, —NH₂, —CN, —CF₃, and —CH₃;
R¹ is phenyl;
R⁴ is selected from the group consisting of —H, —Cl, —F, and —OH;
R⁴ is —H;
R⁷ is —C₁₋₄alkyl;
R⁷ is methyl or ethyl;
R⁷ is selected from the group consisting of phenyl, thienyl, pyrrolyl, furanyl, oxazolyl, imidazolyl, thiazolyl, indolyl, indanyl, and tetrahydronaphthyl;
R⁷ is selected from the group consisting of phenyl, thienyl, indolyl, indanyl, and tetrahydronaphthyl;
R⁷ is phenyl;
R⁸is —C₁₋₄alkyl;
R⁸ is methyl or ethyl;
R⁷ and R⁸ are taken together with the nitrogen member to which they are attached to form pyrrolidinyl, piperidinyl, morpholinyl, or thiomorpholinyl;
R⁷ and R⁸ are taken together with the nitrogen member to which they are attached to form piperidinyl;
R⁹ is —H, —Cl, methyl, ethyl, or —OH;
R⁹ is —H, methyl, or —OH;
R⁹ is methyl;
R¹⁰ is —H, methyl, ethyl, isopropyl, or butyl;
R¹⁰ is —H;
R¹¹ is —H;
Z is selected from the group consisting of bond, —CH₂—, —OCH₂—, —OCH₂CH₂—, and —CH₂CH₂—;
Z is bond, then Y′ is one of R¹(CH₂)₂₋₃O—, R¹CO₂—, R¹CH(R⁹)CO₂—,
R¹C(O)CH(R¹⁰)O—, or R¹CH(R⁹)CH(R¹⁰)O—; provided that when one of R⁹ and R¹⁰ in R¹CH(R⁹)CH(R¹⁰)O— is —H, then the other is not —H;
Z is bond, and Y′ is R¹(CH₂)₂₋₃O—;
R⁶ is —H;
R^{2′} and R^{3′} are each independently selected from the group consisting of —H, —C₁₋₇alkyl, —C₃₋₇alkenyl, —C₃₋₇alkynyl, —C₃₋₇cycloalkyl optionally benzofused, —C₅₋₇cycloalkenyl, —C₃₋₇cycloalkylC₁₋₇alkyl, —C₁₋₇alkylC₃₋₇cycloalkyl, and phenyl;
Y′ is R¹(CH₂)₂₋₃O— and R^{2′} and R^{3′} are each independently selected from the group consisting of —C₃₋₇alkenyl, —C₃₋₇alkynyl, —C₃₋₇cycloalkyl optionally benzofused, —C₅₋₇cycloalkenyl, —C₃₋₇cycloalkylC₁₋₇alkyl, —C₁₋₇alkylC₃₋₇cycloalkyl, and phenyl;
R^{2′} and R^{3′} are each independently selected from the group consisting of a 4-7 membered saturated heterocyclic ring HetR^{a}, said 4-7 membered saturated heterocyclic ring HetR^{a}, having 0 or 1 double bonds, having a carbon member point of attachment and containing a member >NR^{M} as a heteroatom member, and said heteroatom member being separated from said carbon member point of attachment by at least one additional carbon member;
R^{2′} and R^{3′} are each independently selected from the group consisting of —C₁₋₇alkylC(O)R^{x}, optionally substituted with CH₂R^{Ar} or CH₂R^{Ar′};
Y′ is R¹(CH₂)₂₋₃O— and said R^{2′} and R^{3′} are each independently selected from the group consisting of —C₁₋₇alkylC(O)R^{x}, substituted with CH₂R^{Ar} or CH₂R^{Ar′};
R^{2′} and R^{3′} are each independently selected from the group consisting of —C₂₋₅alkylC(O)R^{x}, wherein two valence allowed carbon members in the C₂₋₅alkyl of said —C₂₋₅alkylC(O)R^{x} are part of a saturated C₃₋₆carbocycle;
R^{2′} and R^{3′} are each independently selected from the group consisting of —C₂₋₅alkylOH, wherein two valence allowed carbon members in the C₂₋₅alkyl of said —C₂₋₅alkylOH are part of a saturated C₃₋₆carbocycle;
R^{2′} and R^{3′} are each independently —C₁₋₄alkylAr^{5′}, where Ar^{5′} is a 5-membered heteroaryl containing 3 or 4 nitrogen members, optionally substituted with R^{Y}, and having a valence allowed site as a point of attachment; p1 R^{2′} and R^{3′} are taken together with the nitrogen member to which they are attached to form azetidinyl, pyrrolidinyl, piperidinyl, or homopiperidinyl; R and R are taken together with the nitrogen member to which they are attached to form piperidinyl;
Y′ is R¹(CH₂)₂₋₃O—, and said R^{2′} and R^{3′} are together with the nitrogen member to which they are attached to form piperidinyl, said piperidinyl being substituted with 1 or 2 substituents at the same or at different ring members, said substituents being selected from the group consisting of being selected from the group consisting of —R^{Y}, —CN, —C(O)R^{Y}, —C₀₋₄alkylC(O)CO₂R^{Y}, —C₀₋₄alkylC(O)CO₂R^{Y}, —C₀₋₄alkylOR^{Y}, —C₀₋₄alkylC(O)NR^{Y}R^{Z}, —C₀₋₄alkylNR^{Y}C(O)R^{Z}, —C(O)NR^{Z}OR^{Y}, —C₀₋₄alkylNR^{Y}C(O)CH₂OR^{Y}, —C₀₋₄alkylNR^{Y}C(O)CH₂C(O)R^{Y}, —C₀₋₄alkylNR^{Y}CO₂R^{Y}, —C₀₋₄alkylNR^{Y}C(O)NR^{Y}R^{Z}, —C₀₋₄alkylNR^{Y}C(S)NR^{Y}R^{Z}, —NR^{Y}C(O)CO₂R^{Y}, —NR^{Y}R^{Z}, —C₀₋₄alkylNR^{W}SO₂R^{Y}, 1,3-dihydro-indol-2-one-1-yl, 1,3-dihydro-benzoimidazol-2-one-1-yl, tetrazol-5-yl, 1-R^{Y}-1H-tetrazol-5-yl, R^{Y}-triazolyl, 2-R^{Y}-2H-tetrazol-5-yl, pyrrolidine-2-thion-1-yl, piperidine-2-thion-1-yl, —C₀₋₄alkylC(O)N(R^{Y})(SO₂R^{Y}), —C₀₋₄alkylN(R^{Y})(SO₂)NR^{Y}R^{Y}, —C₀₋₄alkylN(R^{Y})(SO₂)NR^{Y}CO₂R^{Y}, halo, 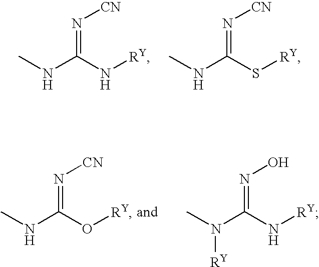
   R^{2′} and R^{3′} are taken together with the nitrogen member to which they are attached to form piperazinyl or piperazinonyl;

Compounds of formula (I) or (II) comprise compounds that satisfy any one of the combinations of definitions given herein and equivalents thereof.

It is understood that some compounds referred to herein are chiral and/or have geometric isomeric centers, for example E- and Z-isomers. The present invention encompasses all such optical isomers, including diasteroisomers and racemic mixtures, and geometric isomers that possess the activity that characterizes the compounds of this invention. In addition, certain compounds referred to herein can exist in solvated as well as unsolvated forms. It is understood that this invention encompasses all such solvated and unsolvated forms that possess the activity that characterizes the compounds of this invention. Compounds according to the present invention that have been modified to be detectable by some analytic technique are also within the scope of this invention. An example of such compounds is an isotopically labeled compound, such as an ¹⁸F isotopically labeled compound that may be used as a probe in detection and/or imaging techniques, such as positron emission tomography (PET) and single-photon emission computed tomography (SPECT). Another example of such compounds is an isotopically labeled compound, such as a deuterium and/or tritium labeled compound that may be used in reaction kinetic studies.

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound that may not be specifically disclosed, but that converts to the specified compound in vivo after administration to the patient. Conventional procedures for the selection and preparation of suitable prodrug derivatives are described, for example, in “Design of Prodrugs”, Bundgaard, H. ed., Elsevier, 1985.

Reference to a compound herein stands for a reference to any one of: (a) the actually recited form of such compound, and (b) any of the forms of such compound in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent, such as water; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In this regard, if such entity is for example in an aqueous environment, it is understood that the compound R—COOH is in such same medium, and therefore the entity is being exposed to species such as R—COOH_{(aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

Embodiments of this invention are made according to the synthetic methods outlined in Schemes A-K, have demonstrated LTA4H inhibitory activity, and are selected from the group consisting of:

| Ex. | Compound Name |
|---|---|
| 12 | Phenyl-carbamic acid 4-(3-dibutylamino-propyl)-phenyl ester |
| | hydrochloride; |
| 17 | Phenyl-carbamic acid 4-[2-(4-hydroxy-4-phenyl-piperidin-1-yl)- |
| | ethyl]-phenyl ester; |
| 36 | Phenyl-carbamic acid 4-[3-(4-hydroxy-4-phenyl-piperidin-1-yl)- |
| | propyl]-phenyl ester; |
| 37 | Phenyl-carbamic acid 4-(3-piperidin-1-yl-propyl)-phenyl ester; |
| 38 | Phenyl-carbamic acid 4-[3-(cyclopropylmethyl-propyl-amino)- |
| | propyl]-phenyl ester hydrochloride; |
| 52 | Phenyl-carbamic acid 4-(2-piperidin-1-yl-ethyl)-phenyl ester; |
| 58 | Phenyl-carbamic acid 4-[2-(4-hydroxy-piperidin-1-yl)-ethyl]- |
| | phenyl ester; |
| 59 | Phenyl-carbamic acid 4-[2-(cyclohexyl-ethyl-amino)-ethyl]- |
| | phenyl ester; |
| 60 | Phenyl-carbamic acid 4-(2-pyrrolidin-1-yl-ethyl)-phenyl ester; |
| 61 | Phenyl-carbamic acid 4-(2-azepan-1-yl-ethyl)-phenyl ester; |
| 62 | Phenyl-carbamic acid 4-[2-(cyclopropylmethyl-propyl-amino)- |
| | ethyl]-phenyl ester; |
| 63 | Phenyl-carbamic acid 4-(2-dibutylamino-ethyl)-phenyl ester; |
| 138 | Phenyl-carbamic acid 4-{2-[4-(2-hydroxy-acetylamino)- |
| | piperidin-1-yl]-ethyl}-phenyl ester; |
| 139 | Methyl-phenyl-carbamic acid 4-{2-[4-(2-hydroxy-acetylamino)- |
| | piperidin-1-yl]-ethyl}-phenyl ester; |
| 148 | Phenyl-carbamic acid 4-[2-(4-methanesulfonylamino-piperidin- |
| | 1-yl)-ethyl]-phenyl ester; |
| 149 | Methyl-phenyl-carbamic acid 4-[2-(4-methanesulfonylamino- |
| | piperidin-1-yl)-ethyl]-phenyl ester; and |
| 164 | Phenyl-carbamic acid 2-fluoro-4-(2-morpholin-4-yl-ethyl)-phenyl |
| | ester. |

Further embodiments of this invention are made according to the synthetic methods outlined in Schemes A-K, have demonstrated LTA4H inhibitory activity, and are selected from the group consisting of:

| Ex. | Compound Name |
|---|---|
| 8 | 1-(2-{4-[(3-Hydroxy-phenyl)-methyl-carbamoyloxy]- |
| | phenoxy}-ethyl)-piperidine-4-carboxylic acid ethyl ester; |
| 9 | 1-(2-{4-[(3-Hydroxy-phenyl)-methyl-carbamoyloxy]-phenoxy}- |
| | ethyl)-piperidine-4-carboxylic acid; |
| 10 | Dimethyl-carbamic acid 4-[2-(4-hydroxy-4-phenyl-piperidin-1-yl)- |
| | ethoxy]-phenyl ester; |
| 11 | (3-Hydroxy-phenyl)-carbamic acid 4-[2-(4-hydroxy-4-phenyl- |
| | piperidin-1-yl)-ethoxy]-phenyl ester; |
| 13 | N-(2-Hydroxy-phenyl)-2-{4-[2-(4-hydroxy-4-phenyl-piperidin- |
| | 1-yl)-ethoxy]-phenyl}-acetamide; |
| 14 | [4-(2-Piperidin-1-yl-ethoxy)-phenyl]-carbamic acid phenyl ester |
| | hydrochloride; |
| 15 | Phenyl-carbamic acid 4-(2-piperidin-1-yl-ethoxy)-phenyl ester; |
| 16 | Phenyl-carbamic acid 4-(2-piperidin-1-yl-ethoxy)-benzyl ester; |
| 39 | (4-Hydroxy-phenyl)-carbamic acid 4-[2-(4-hydroxy-4-phenyl- |
| | piperidin-1-yl)-ethoxy]-phenyl ester; |
| 40 | Methyl-phenyl-carbamic acid 4-[2-(4-hydroxy-4-phenyl- |
| | piperidin-1-yl)-ethoxy]-phenyl ester; |
| 41 | Phenyl-carbamic acid 4-[2-(4-propyl-piperidin-1-yl)-ethoxy]- |
| | phenyl ester; |
| 42 | Phenyl-carbamic acid 4-[3-(4-hydroxy-4-phenyl-piperidin-1-yl)- |
| | propoxy]-phenyl ester; |
| 43 | (2-Fluoro-phenyl)-carbamic acid 4-(2-piperidin-1-yl-ethoxy)- |
| | phenyl ester; |
| 44 | N-(2-Hydroxy-phenyl)-2-[4-(2-piperidin-1-yl-ethoxy)-phenyl]- |
| | acetamide; |
| 45 | (3-Chloro-phenyl)-carbamic acid 4-(2-piperidin-1-yl-ethoxy)- |
| | phenyl ester; |
| 46 | Phenyl-carbamic acid 4-(2-diethylamino-ethoxy)-phenyl ester; |
| 47 | Phenyl-carbamic acid 4-[2-(4-hydroxy-4-phenyl-piperidin-1-yl)- |
| | ethoxy]-phenyl ester; |
| 48 | Phenyl-carbamic acid 4-(2-dibutylamino-ethoxy)-phenyl ester; |
| 49 | Phenyl-carbamic acid 4-[2-(cyclopropylmethyl-propyl-amino)- |
| | ethoxy]-phenyl ester; |
| 50 | Phenyl-carbamic acid 4-[2-(4-benzyl-piperidin-1-yl)-ethoxy]- |
| | phenyl ester; |
| 51 | Phenyl-carbamic acid 4-[2-(4-hydroxymethyl-piperidin-1-yl)- |
| | ethoxy]-phenyl ester; |
| 53 | Phenyl-carbamic acid 4-[2-(4-hydroxy-piperidin-1-yl)-ethoxy]- |
| | phenyl ester; |
| 54 | Phenyl-carbamic acid 4-{2-[4-(4-chloro-3-trifluoromethyl- |
| | phenyl)-4-hydroxy-piperidin-1-yl]-ethoxy}-phenyl ester; |
| 55 | Phenyl-carbamic acid 4-(2-azepan-1-yl-ethoxy)-phenyl ester; |
| 56 | Phenyl-carbamic acid 4-{2-[4-(4-bromo-phenyl)-4-hydroxy- |
| | piperidin-1-yl]-ethoxy}-phenyl ester; |
| 57 | Phenyl-carbamic acid 4-{2-[4-(4-chloro-phenyl)-4-hydroxy- |
| | piperidin-1-yl]-ethoxy}-phenyl ester; |
| 64 | Thiophen-3-yl-carbamic acid 4-(2-piperidin-1-yl-ethoxy)- |
| | phenyl ester; |
| 65 | Thiophen-2-yl-carbamic acid 4-(2-piperidin-1-yl-ethoxy)- |
| | phenyl ester; |
| 134 | Phenyl-carbamic acid 4-{2-[4-(2-hydroxy-acetylamino)- |
| | piperidin-1-yl]-ethoxy}-phenyl ester; |
| 135 | Methyl-phenyl-carbamic acid 4-{2-[4-(2-hydroxy-acetylamino)- |
| | piperidin-1-yl]-ethoxy}-phenyl ester; |
| 144 | Phenyl-carbamic acid 4-[2-(4-methanesulfonylamino- |
| | piperidin-1-yl)-ethoxy]-phenyl ester; |
| 145 | Methyl-phenyl-carbamic acid 4-[2-(4-methanesulfonylamino- |
| | piperidin-1-yl)-ethoxy]-phenyl ester; |
| 150 | Phenyl-carbamic acid 5-{2-[4-(2-hydroxy-acetylamino)- |
| | piperidin-1-yl]-ethoxy}-pyridin-2-yl ester; and |
| 151 | Phenyl-carbamic acid 5-[2-(4-acetylamino-piperidin-1-yl)-ethoxy]- |
| | pyridin-2-yl ester. |

Further embodiments of this invention are made according to the synthetic methods outlined in Schemes A-K, have demonstrated LTA4H inhibitory activity, and are selected from the group consisting of:

| Ex. | Compound Name |
|---|---|
| 93 | N-[1-(4-Phenethyloxy-benzyl)-piperidin-4-yl]methanesulfonamide; |
| 94 | 1-(6-Phenethyloxy-pyridin-3-ylmethyl)-piperidine-4-carboxylic |
| | acid; |
| 95 | 1-(4-Phenethyloxy-benzyl)-piperidine; |
| 96 | 1-(4-Phenethyloxy-benzyl)-piperidine-4-carboxylic acid; |
| 97 | 1-[4-(3-Phenyl-propoxy)-benzyl]-piperidine; |
| 98 | 1-[4-(4-Phenyl-butoxy)-benzyl]-piperidine; |
| 99 | 1-[1-(4-Phenethyloxy-benzyl)-piperidin-4-yl]-pyrrolidin-2-one; |
| 100 | 8-(4-Phenethyloxy-benzyl)-2,8-diaza-spiro[4.5]decan-1-one; |
| 101 | 1-(4-Phenethyloxy-benzyl)-piperidine-4-carboxylic acid amide; |
| 102 | 1-(4-Phenethyloxy-benzyl)-piperidine-3-carboxylic acid amide; |
| 103 | 1-(4-Phenethyloxy-benzyl)-piperidin-4-ol; |
| 104 | 1-(4-Phenethyloxy-benzyl)-4-(1H-tetrazol-5-yl)-piperidine; |
| 105 | 1-(4-Phenethyloxy-benzyl)-piperidin-4-ylamine; |
| 106 | 1-[4-(3-Phenyl-propoxy)-benzyl]-piperidine-4-carboxylic acid ethyl |
| | ester; |
| 107 | 1-{1-[4-(3-Phenyl-propoxy)-benzyl]-piperidin-4-yl}-pyrrolidin- |
| | 2-one; |
| 108 | 1-[4-(3-Phenyl-propoxy)-benzyl]-piperidin-4-ol; |
| 109 | 1-[4-(3-Phenyl-propoxy)-benzyl]-piperidin-3-ol; |
| 110 | 1-[4-(3-Phenyl-propoxy)-benzyl]-piperidine-4-carboxylic acid |
| | amide; |
| 111 | 1-[4-(3-Phenyl-propoxy)-benzyl]-piperidine-3-carboxylic acid |
| | amide; |
| 112 | 1-[4-(3-Phenyl-propoxy)-benzyl]-piperidine-4-carboxylic acid; |
| 113 | N-[1-(4-Phenethyloxy-benzyl)-piperidin-4-yl]-acetamide; |
| 114 | [1-(4-Phenethyloxy-benzyl)-piperidin-4-yl]-urea; |
| 115 | [1-(4-Phenethyloxy-benzyl)-piperidin-4-yl]-carbamic acid methyl |
| | ester; |
| 118 | 1-[4-(3-Phenyl-propoxy)-benzyl]-piperidin-4-ylamine; |
| 119 | N-{1-[4-(3-Phenyl-propoxy)-benzyl]-piperidin-4-yl}- |
| | methanesulfonamide; |
| 120 | N-{1-[4-(3-Phenyl-propoxy)-benzyl]-piperidin-4-yl}-acetamide; |
| 121 | {1-[4-(3-Phenyl-propoxy)-benzyl]-piperidin-4-yl}-carbamic acid |
| | methyl ester; |
| 122 | {1-[4-(3-Phenyl-propoxy)-benzyl]-piperidin-4-yl}-urea; |
| 140 | 2-Hydroxy-N-{1-[4-(3-phenyl-propoxy)-benzyl]-piperidin-4-yl}- |
| | acetamide; |
| 141 | 2-Hydroxy-N-[1-(4-phenethyloxy-benzyl)-piperidin-4-yl]- |
| | acetamide; |
| 157 | N-{1-[6-(3-Phenyl-propoxy)-pyridin-3-ylmethyl]-piperidin-4-yl}- |
| | acetamide; |
| 158 | N-{1-[6-(3-Phenyl-propoxy)-pyridin-3-ylmethyl]-piperidin-4-yl}- |
| | methanesulfonamide; |
| 159 | 1-{1-[6-(3-Phenyl-propoxy)-pyridin-3-ylmethyl]-piperidin-4-yl}- |
| | pyrrolidin-2-one; and |
| 163 | N-[1-(3-Fluoro-4-phenethyloxy-benzyl)-piperidin-4-yl]-acetamide. |

Further embodiments of this invention are made according to the synthetic methods outlined in Schemes A-K, have demonstrated LTA4H inhibitory activity, and are selected from the group consisting of:

| Ex. | Compound Name |
|---|---|
| 18 | 1′-[2-(4-Phenethyloxy-phenyl)-ethyl]-[1,4′]bipiperidinyl-3- |
| | carboxylic acid ethyl ester; |
| 19 | 1′-[2-(4-Phenethyloxy-phenyl)-ethyl]-[1,4′]bipiperidinyl-3- |
| | carboxylic acid; |
| 26 | 1′-{2-[4-(2-Oxo-2-phenyl-ethoxy)-phenyl]-ethyl}- |
| | [1,4′]bipiperidinyl-2-one; |
| 27 | 1′-{2-[4-(2-Hydroxy-2-phenyl-ethoxy)-phenyl]-ethyl}- |
| | [1,4′]bipiperidinyl-2-one; |
| 29 | 1-[2-(4-Phenethyloxy-phenyl)-ethyl]-piperidine-4-carbonitrile; |
| 30 | 1-[2-(4-Phenethyloxy-phenyl)-ethyl]-4-(1H-tetrazol-5-yl)- |
| | piperidine; |
| 31 | 1-[2-(4-Phenethyloxy-phenyl)-ethyl]-4-(1H-[1,2,3]triazol-4-yl)- |
| | piperidine; |
| 32 | Cyclopropyl-[2-(4-phenethyloxy-phenyl)-ethyl]-amine; |
| 33 | 4-{Cyclopropyl-[2-(4-phenethyloxy-phenyl)-ethyl]-amino}- |
| | butyronitrile; |
| 34 | 3-{Cyclopropyl-[2-(4-phenethyloxy-phenyl)-ethyl]-amino}- |
| | propionic acid ethyl ester; |
| 35 | 3-{Cyclopropyl-[2-(4-phenethyloxy-phenyl)-ethyl]-amino}- |
| | propionic acid trifluoroacetic acid salt; |
| 66 | 1′-[2-(4-Phenethyloxy-phenyl)-ethyl]-[1,4′]bipiperidinyl-2- |
| | carboxylic acid ethyl ester; |
| 67 | 1-[2-(4-Phenethyloxy-phenoxy)-ethyl]-piperidine; |
| 72 | 2-{4-[2-(Cyclohexyl-ethyl-amino)-ethyl]-phenoxy}-1- |
| | phenyl-ethanone; |
| 73 | 2-{4-[2-(Cyclohexyl-ethyl-amino)-ethyl]-phenoxy}-1- |
| | phenyl-ethanol; |
| 75 | 1-{2-[4-(2-Oxo-2-phenyl-ethoxy)-phenyl]-ethyl}-piperidine-4- |
| | carboxylic acid methyl ester; |
| 76 | 1-{2-[4-(2-Hydroxy-2-phenyl-ethoxy)-phenyl]-ethyl}-piperidine-4- |
| | carboxylic acid methyl ester; |
| 77 | 1-[2-(4-Phenethyloxy-phenyl)-ethyl]-piperidine-4-carboxylic acid |
| | methyl ester; |
| 78 | 1-{2-[4-(2-Hydroxy-2-phenyl-ethoxy)-phenyl]-ethyl}-piperidine-4- |
| | carboxylic acid amide; |
| 79 | 1-[2-(4-Phenethyloxy-phenyl)-ethyl]-piperidine-4-carboxylic acid |
| | amide; |
| 80 | 1′-[2-(4-Phenethyloxy-phenyl)-ethyl]-[1,4′]bipiperidinyl-2-one; |
| 81 | 1-[2-(4-Phenethyloxy-phenyl)-ethyl]-piperidine-4-carboxylic acid; |
| 83 | 4-[2-(4-Phenethyloxy-phenyl)-ethyl]-piperazin-2-one; |
| 84 | 3-[2-(4-Phenethyloxy-phenyl)-ethylamino]-propionic acid ethyl |
| | ester; |
| 85 | 3-{Methyl-[2-(4-phenethyloxy-phenyl)-ethyl]-amino}- |
| | propionic acid ethyl ester; |
| 87 | 3-{Cyclohexyl-[2-(4-phenethyloxy-phenyl)-ethyl]-amino}- |
| | propionic acid ethyl ester; |
| 88 | 3-{Methyl-[2-(4-phenethyloxy-phenyl)-ethyl]-amino}-propionic |
| | acid; |
| 89 | 3-{Cyclohexyl-[2-(4-phenethyloxy-phenyl)-ethyl]-amino}- |
| | propionic acid; |
| 90 | 3-{(1-Acetyl-piperidin-4-yl)-[2-(4-phenethyloxy-phenyl)-ethyl]- |
| | amino}-propionic acid ethyl ester; |
| 91 | 3-{(1-Acetyl-piperidin-4-yl)-[2-(4-phenethyloxy-phenyl)-ethyl]- |
| | amino}-propionic acid; |
| 92 | 1-{2-[4-(2-Hydroxy-2-phenyl-ethoxy)-phenyl]-ethyl}-piperidine-4- |
| | carboxylic acid; |
| 127 | 2-[4-(2-Piperidin-1-yl-ethyl)-phenoxy]-indan-1-one; |
| 128 | Chloro-phenyl-acetic acid 4-(2-piperidin-1-yl-ethyl)-phenyl ester; |
| 129 | Indan-2-carboxylic acid 4-(2-piperidin-1-yl-ethyl)-phenyl ester; |
| 130 | 2-[4-(2-Piperidin-1-yl-ethyl)-phenoxy]-indan-1-ol; |
| 131 | 1-{2-[4-(3-Phenyl-propoxy)-phenyl]-ethyl}-piperidine-4-carboxylic |
| | acid; |
| 133 | 2-Hydroxy-N-{1-[2-(4-phenethyloxy-phenoxy)-ethyl]-piperidin-4- |
| | yl}-acetamide; |
| 136 | 2-Hydroxy-N-(1-{2-[4-(3-phenyl-propoxy)-phenyl]-ethyl}- |
| | piperidin-4-yl)-acetamide; |
| 137 | 2-Hydroxy-N-{1-[2-(4-phenethyloxy-phenyl)-ethyl]-piperidin- |
| | 4-yl}-acetamide; |
| 143 | N-{1-[2-(4-Phenethyloxy-phenoxy)-ethyl]-piperidin-4-yl}- |
| | methanesulfonamide; |
| 146 | N-(1-{2-[4-(3-Phenyl-propoxy)-phenyl]-ethyl}-piperidin-4-yl)- |
| | methanesulfonamide; |
| 147 | N-{1-[2-(4-Phenethyloxy-phenyl)-ethyl]-piperidin-4-yl}- |
| | methanesulfonamide; |
| 154 | N-{1-[2-(6-Phenethyloxy-pyridin-3-yl)-ethyl]-piperidin-4-yl}- |
| | methanesulfonamide; |
| 155 | 2-Hydroxy-N-{1-[2-(6-phenethyloxy-pyridin-3-yl)-ethyl]- |
| | piperidin-4-yl}-acetamide; and |
| 156 | 1-{1-[2-(6-Phenethyloxy-pyridin-3-yl)-ethyl]-piperidin-4-yl}- |
| | pyrrolidin-2-one. |

Further embodiments of this invention are made according to the synthetic methods outlined in Schemes A-K, have demonstrated LTA4H inhibitory activity, and are selected from the group consisting of:

| Ex. | Compound Name |
|---|---|
| 20 | Carbonic acid phenyl ester 4-(2-piperidin-1-yl-ethoxy)-phenyl |
| | ester; |
| 21 | Phenyl-acetic acid 4-(2-piperidin-1-yl-ethoxy)-phenyl ester; |
| 22 | 2-Phenyl-propionic acid 4-(2-piperidin-1-yl-ethoxy)-phenyl ester; |
| 23 | 1H-Indole-2-carboxylic acid 4-(2-piperidin-1-yl-ethoxy)-phenyl |
| | ester; |
| 24 | 1-Phenyl-2-[4-(2-piperidin-1-yl-ethoxy)-phenoxy]-ethanone; |
| 28 | 3-{2-[4-(2-Piperidin-1-yl-ethoxy)-phenoxy]-ethyl}-phenol; |
| 68 | 1H-Indole-3-carboxylic acid 4-(2-piperidin-1-yl-ethoxy)-phenyl |
| | ester; |
| 69 | 1-{2-[4-(Indan-2-yloxy)-phenoxy]-ethyl}-piperidine; |
| 70 | 1-(2-{4-[2-(2-Fluoro-phenyl)-ethoxy]-phenoxy}-ethyl)-piperidine; |
| 71 | 1-Phenyl-2-[4-(2-piperidin-1-yl-ethoxy)-phenoxy]-ethanol; |
| 74 | 4-{2-[4-(2-Piperidin-1-yl-ethoxy)-phenoxy]-ethyl}-phenol; |
| 82 | 1-{1-[2-(4-Phenethyloxy-phenoxy)-ethyl]-piperidin-4-yl}- |
| | pyrrolidin-2-one; |
| 86 | 1-[2-(4-Phenethyloxy-phenoxy)-ethyl]-piperidine-4-carboxylic |
| | acid; |
| 116 | 1-{2-[4-(3-Phenyl-propoxy)-phenoxy]-ethyl}-piperidine-4- |
| | carboxylic acid ethyl ester; |
| 117 | 1-{2-[4-(3-Phenyl-propoxy)-phenoxy]-ethyl}-piperidine-4- |
| | carboxylic acid; |
| 123 | Chloro-phenyl-acetic acid 4-(2-piperidin-1-yl-ethoxy)-phenyl ester; |
| 124 | Indan-2-carboxylic acid 4-(2-piperidin-1-yl-ethoxy)-phenyl ester; |
| 125 | 2-[4-(2-Piperidin-1-yl-ethoxy)-phenoxy]-indan-1-ol; |
| 126 | 2-[4-(2-Piperidin-1-yl-ethoxy)-phenoxy]-indan-1-one; |
| 132 | 2-Hydroxy-N-(1-{2-[4-(3-phenyl-propoxy)-phenoxy]-ethyl}- |
| | piperidin-4-yl)-acetamide; |
| 142 | N-(1-{2-[4-(3-Phenyl-propoxy)-phenoxy]-ethyl}-piperidin-4-yl)- |
| | methanesulfonamide; |
| 152 | N-{1-[2-(6-Phenethyloxy-pyridin-3-yloxy)-ethyl]-piperidin-4-yl}- |
| | methanesulfonamide; |
| 153 | 1-{2-[6-(3-Phenyl-propoxy)-pyridin-3-yloxy]-ethyl}-piperidine-4- |
| | carboxylic acid; |
| 160 | 1-(4-Phenethyloxy-phenoxy)-3-piperidin-1-yl-propan-2-ol; |
| 161 | 2-Hydroxy-N-(1-{2-hydroxy-3-[4-(3-phenyl-propoxy)-phenoxy]- |
| | propyl}-piperidin-4-yl)-acetamide; |
| 162 | N-{1-[2-(3-Fluoro-4-phenethyloxy-phenoxy)-ethyl]-piperidin-4- |
| | yl}-2-hydroxy-acetamide; |
| 165 | 1-(2-{4-[2-(3-Fluoro-phenyl)-ethoxy]-phenoxy}-ethyl)- |
| | piperidine; and |
| 166 | 1-(2-{4-[2-(4-Fluoro-phenyl)-ethoxy]-phenoxy}-ethyl)- |
| | piperidine. |

Compounds according to the present invention may be made according to processes within the skill of the art and/or according to processes of this invention, such as those described in the schemes and examples that follow and by matrix or combinatorial methods. To obtain the various compounds herein, starting materials may be employed that carry the ultimately desired substituents though the reaction scheme with or without protection as appropriate. Starting materials may be obtained from commercial sources or synthesized by methods known to one skilled in the art. Alternatively, it may be necessary to employ, in the place of the ultimately desired substituent, a suitable group, which may be carried through the reaction scheme and replaced as appropriate with the desired substituent. Those of ordinary skill in the art will be able to modify and adapt the guidance provided herein to make compounds according to the present invention.

Embodiments of processes illustrated herein include, when chemically meaningful, one or more steps such as hydrolysis, halogenation, protection, and deprotection. These steps can be implemented in light of the teachings provided herein and the ordinary skill in the art.

During any of the processes for preparation of the compounds of the present invention, it may be necessary and/or desirable to protect sensitive or reactive groups on any of the molecules concerned. In addition, compounds of this invention may be modified by using protecting groups; such compounds, precursors, or prodrugs are also within the scope of the invention. This modification may be achieved by means of conventional protecting groups, such as those described in “Protective Groups in Organic Chemistry”, J. F. W. McOmie, ed., Plenum Press, 1973; and T. W. Greene & P. G. M. Wuts, “Protective Groups in Organic Synthesis”, 3rd ed., John Wiley & Sons, 1999. The protecting groups may be removed at a convenient subsequent stage using methods known in the art.

Table of Acronyms

| Term | Acronym |
|---|---|
| Tetrahydrofuran | THF |
| N,N-Dimethylformamide | DMF |
| N,N-Dimethylacetamide | DMA |
| Dimethyl sulfoxide | DMSO |
| tert-Butylcarbamoyl | BOC |
| Bovine serum albumin | BSA |
| High-pressure liquid chromatography | HPLC |
| Thin layer chromatography | TLC |
| N,N-diisopropylethylamine | DIEA |
| Triethylamine | TEA |
| 1,8-Diazabicyclo[5.4.0]undec-7-ene | DBU |
| 1-(3-Dimethylaminopropyl)-3- | EDC |
| ethylcarbodiimide hydrochloride | |

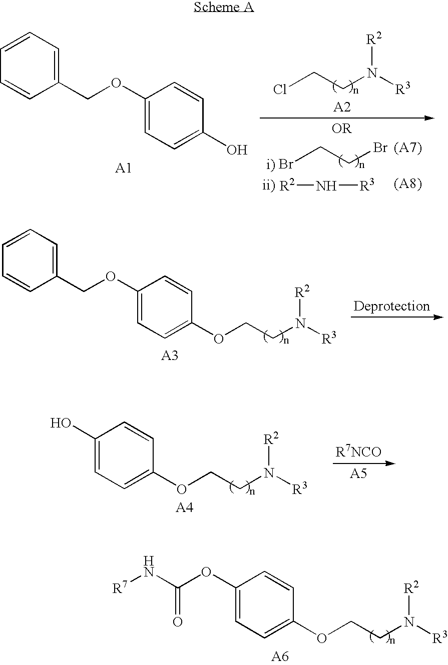

Referring to Scheme A, commercially available 4-benzyloxyphenol, A1, is alkylated with amino alkyl halides A2 in which several amino alkyl chlorides are commercially available. The reactions can be run under a wide range of temperatures, including room temperature and more elevated temperatures, in the presence of an inorganic base known to facilitate O-alkylation, such as, but not limited to, K₂CO₃, Cs₂CO₃ and mixtures thereof (Palkowitz, A. D., et al., J. Med. Chem. 1997, 40(10):1407-1416). Suitable solvents include but are not limited to DMF.

Alternatively, A1 is alkylated with dihaloalkanes A7, preferably dibromoalkanes such as 1,2-dibromoethane and 1,3-dibromopropane, both of which are commercially available, under a wide range of temperatures with elevated temperatures preferred (Zhou, Z.-L., et al., J. Med. Chem. 1999, 42(15):2993-3000). The reactions are conducted in the presence of an inorganic base known to facilitate O-alkylation such as, but not limited to, K₂CO₃, Cs₂CO₃ and mixtures thereof. Suitable solvents include, but are not limited to, CH₃CN and DMF. Resulting intermediate bromides are treated with amines A8, either in the presence or absence of a suitable base under a wide range of temperatures with elevated temperatures preferred. Suitable amine bases include, but are not limited to, TEA, DIEA, DBU, resin-bound amine bases, and mixtures thereof. Suitable inorganic bases include, but are not limited to, K₂CO₃, Cs₂CO₃ and mixtures thereof. Suitable solvents include, but are not limited to, CH₃CN, CH₂Cl₂ and DMF.

Removal of the benzyl group on A3 may be accomplished using catalytic hydrogenation conditions well known to those skilled in the art (Greene, T. W.; Wuts, P. G. M., 1999.). Suitable catalysts include, but are not limited to, Pd on carbon (Pd/C), in solvents such as, but not limited to, ethyl acetate, alcohols and mixtures thereof. Examples of alcohols include, but are not limited to, CH₃OH, EtOH, and i-PrOH. These reactions are typically run at room temperature. Removal of the benzyl group on A3 may be accomplished in some embodiments by using dissolving metal reductions or transfer hydrogenation conditions at suitable temperatures. For example, dissolving metal reductions are typically performed at temperatures below room temperature (−33° C.). Reaction of A4 with isocyanates A5 may be accomplished within a range of temperatures including room temperature and lower temperatures in the presence of a suitable base including, but not limited to, an amine or inorganic base as defined above. Suitable amine bases include, but are not limited to, TEA, DIEA, DBU, resin-bound amine bases, and mixtures thereof. Suitable solvents include, but are not limited to, CH₂Cl₂ and THF.

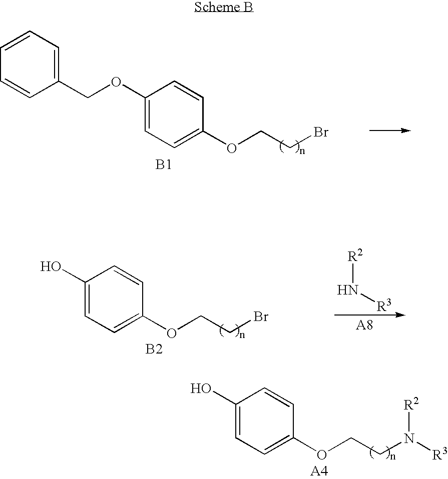

Referring to Scheme B, the benzyl group of compounds of structure B1, intermediate bromides prepared as described in Scheme A, are removed using conditions as described for A3 in Scheme A. Compounds of general structure B2 are also prepared from commercially available 4-(2-hydroxyethyl)phenol or 4-(2-hydroxypropyl)phenol using typical brominating conditions. These conditions include, but are not limited to, treatment with 48% HBr solutions at elevated temperatures. Compounds B2 are then treated with amines A8, either in the presence or absence of a base under a wide range of temperatures with elevated temperatures preferred. Suitable amine bases include, but are not limited to, TEA, DIEA, DBU, resin-bound amine bases, and mixtures thereof. Suitable inorganic bases include, but are not limited to, K₂CO₃, Cs₂CO₃ and mixtures thereof. Suitable solvents include, but are not limited to, CH₃CN, CH₂Cl₂ and DMF. Further conversion of the resulting products A4 to compounds A6 is as detailed above for Scheme A.

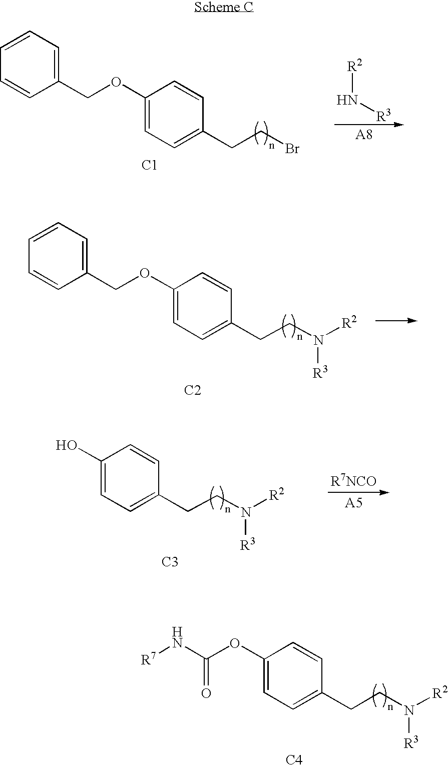

Referring to Scheme C, Cl, n=2, is a commercially available material, and C1, n=1, is available using standard alkylation and bromination conditions starting from 4-(2-hydroxyethyl)phenol and benzyl bromide followed by treatment with 48% HBr at elevated temperatures. Compounds with the general structure C2 can be obtained by treatment of C3 with amines A8, either in the presence or absence of a suitable base under a wide range of temperatures. Suitable amine bases include, but are not limited to, TEA, DIEA, DBU, resin-bound amine bases, and mixtures thereof. Suitable inorganic bases include, but are not limited to, K₂CO₃, Cs₂CO₃ and mixtures thereof. Suitable solvents include, but are not limited to, CH₃CN and DMF. Removal of the benzyl group is accomplished using catalytic hydrogenation conditions well known to those skilled in the art. Suitable catalysts include, but are not limited to palladium on carbon (Pd/C) in solvents such as, but not limited to, ethyl acetate, alcohols and mixtures thereof. Examples of alcohols include, but are not limited to, CH₃OH, EtOH, and i-PrOH. These reactions are typically run at room temperature. Removal of the benzyl group on C2 may be accomplished in some embodiments using transfer-hydrogenation conditions at suitable temperatures. Further conversion of the resulting products C3 to the final target compounds C4 is as detailed above for Scheme A.

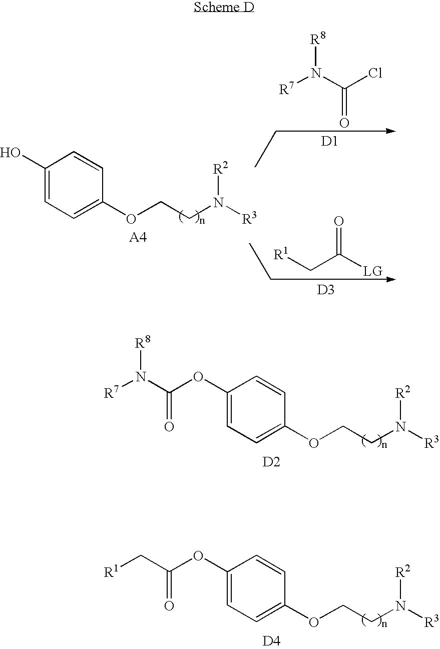

Referring to Scheme D, commercially available carbamoyl chlorides D1 are reacted with phenols A4, prepared as described in Scheme A, to form carbamates D2. Reactions are run within a range of temperatures including room temperature, lower, or elevated temperatures in the presence of a suitable base. Suitable bases include, but are not limited to, t-BuOK, NaH, CH₃ONa, EtONa, K₂CO₃, Cs₂CO₃, TEA, DIEA, DBU, and mixtures thereof. Suitable solvents include, but are not limited to, THF and CH₃CN.

Alternatively, compounds of the structure A4 may be coupled with commercially available compounds D3 to give compounds of structure D4. When LG is Cl, reactions can be run at a wide range of temperatures, including room temperatures and low temperatures in the presence of an amine base. Suitable amine bases include, but are not limited to, TEA, DIEA, DBU, resin-bound amine bases, and mixtures thereof. Suitable solvents include, but are not limited to, CH₂Cl₂ and THF. When LG is OH, compounds of the structure D4 can be prepared using standard peptide coupling conditions well know to those skilled in the art such as, but not limited to, EDCI, DCC, HATU, HBTU, and mixtures thereof. Suitable solvents include, but are not limited to, CH₂Cl₂ and THF.

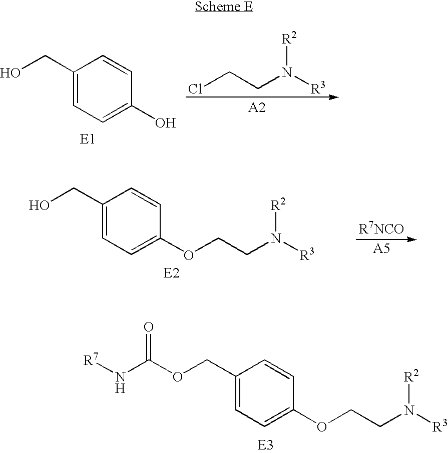

Referring to Scheme E, commercially available 4-hydroxybenzyl alcohol, E1, is alkylated with amino alkyl halides A2; in which several amino alkyl chlorides are commercially available. The reactions can be run under a wide range of temperatures, including room temperature, and more elevated temperatures, in the presence of an inorganic base known to facilitate O-alkylation, such as, but not limited to, K₂CO₃, Cs₂CO₃ and mixtures thereof. Suitable solvents include, but are not limited to, DMF and CH₃CN. Coupling of the alcohols E2 with aromatic isocyanates A5 to form carbamates E3 may be accomplished within a range of temperatures including, room temperature, and elevated temperatures in the presence of a suitable base including, but not limited to, an amine or inorganic base. Suitable inorganic bases include, but are not limited to, K₂CO₃, Cs₂CO₃ and mixtures thereof. Suitable amine bases include, but are not limited to, TEA, DIEA, DBU, resin-bound amine bases, and mixtures thereof. Suitable solvents include, but are not limited to, CH₂Cl₂ and THF.

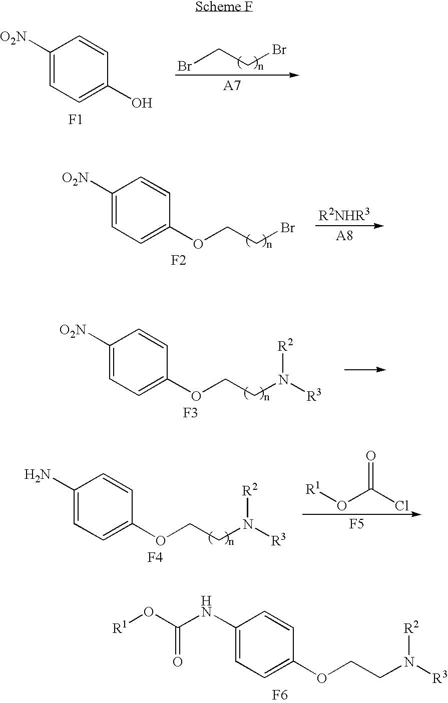

Referring to Scheme F, commercially available 4-nitrophenol, F1, is alkylated with dihaloalkanes, preferably dibromoalkanes such as 1,2-dibromoethane and 1,3-dibromopropane, A7, as described in Scheme A. Compounds of structure F2 are treated with amines A8 as described in Scheme A. Reduction of the nitro group on F3 may be accomplished using catalytic hydrogenation conditions well known to those skilled in the art. Suitable catalysts include, but are not limited to palladium on carbon (Pd/C), in solvents such as, but not limited to, ethyl acetate, alcohols and mixtures thereof. Examples of alcohols include, but are not limited to, CH₃0H, EtOH, and i-PrOH. These reactions are typically run at room temperature. Reaction of the products, F4, with chloroformates, F5, to form carbamates F6 may be accomplished within a range of temperatures, including room temperature, and lower temperatures in the presence of a suitable base including, but not limited to, an amine or inorganic base. Suitable inorganic bases include, but are not limited to, K₂CO₃, Cs₂CO₃ and mixtures thereof. Suitable amine bases include, but are not limited to, TEA, DIEA, DBU, resin-bound amine bases, and mixtures thereof. Suitable solvents include, but are not limited to, CH₂Cl₂ and THF.

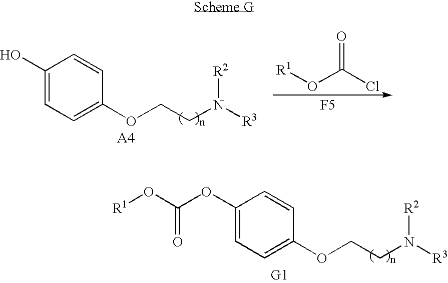

Referring to Scheme G, carbonates G1 may be prepared by coupling of phenols, A4, prepared as described in Scheme A, and chloroformates, F5, within a range of temperatures, including room temperature and lower temperatures, in the presence of a suitable base including, but not limited to, an amine or inorganic base. Suitable inorganic bases include, but are not limited to, K₂CO₃, Cs₂CO₃ and mixtures thereof. Suitable amine bases include, but are not limited to, TEA, DIEA, DBU, resin-bound amine bases, and mixtures thereof. Suitable solvents include, but are not limited to, CH₂Cl₂ and THF.

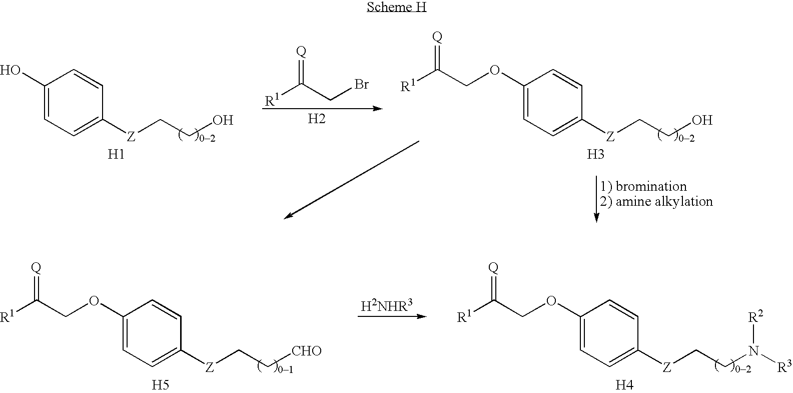

Referring to Scheme H, phenols H1, some of which are commercially available, are alkylated with alkyl halides H2 (Q=O or Q=H,H), under a wide range of temperatures, including room temperature and more elevated temperatures, in the presence of an inorganic base known to facilitate O-alkylation, such as, but not limited to, K₂CO₃, Cs₂CO₃ and mixtures thereof. Suitable solvents include, but are not limited to, acetone, CH₃CN, and DMF. The alcohols H3 are converted to amines H4 according to procedures described in Scheme B. Alternatively, alcohols H4 can be oxidized to give structures of the type H5 using oxidative conditions such as, but not limited to, Dess-Martin periodinane (1,1,1-tris(acetyloxy)-1,1-dihydro-1,2-beniodoxol-3-(1H)-one). Aldehydes H5 are converted to amines H4 using reduction amination conditions well known to those skilled in the art, including but not limited to NaBH(OAc)₃ in an appropriate solvent such as CH₂Cl₂, ClCH₂CH₂Cl or CF₃CH₂OH (J. Org. Chem. 1996, 61, 3849-3862).

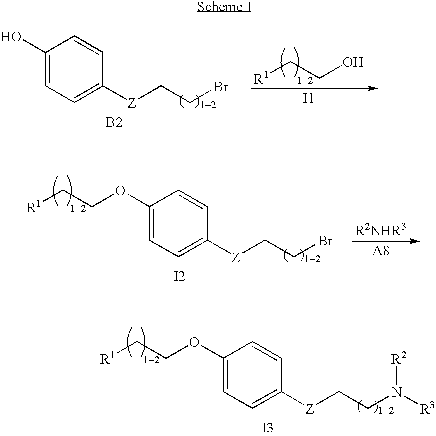

Referring to Scheme I, phenols of the structure B2, described in Scheme B (Z═O) or available from compounds C2 (Z=bond), can be coupled with commercially available alcohols, I1, to give structures of the type I2 under Mitsunobu conditions, well known to those skilled in the art, including but not limited to diisopropyl azodicarboxylate and triphenyl phosphine in solvents such as, but not limited to, CH₂Cl₂, and THF (Organic Reactions, 1992, 42, 335-656). Compounds of structure 12 are then treated with amines, A8, as described in Scheme A.

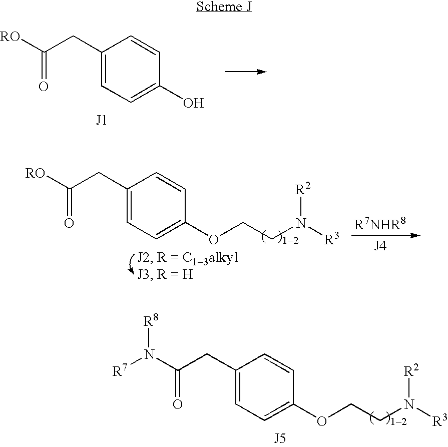

Referring to Scheme J, commercially available esters, J1, are converted to amines, J2, according to procedures outlined in Scheme A. Compounds of structure J3 can be obtained by hydrolysis of J2 using methods well known to those skilled in the art such as, but not limited to, the use of aqueous solutions of LiOH, KOH or NaOH, or aqueous solutions of HCl or CH₃CO₂H, or the use of (CH₃)₃SiOK. Furthermore, persons skilled in the art will recognize that certain compounds are more advantageously produced by one method as compared to another and that salts of the desired compounds may initially result. The compounds of structure J5 can be prepared using standard peptide coupling conditions well know to those skilled in the art such as, but not limited to, 1-(3-dimethylaminopropyl)-3-ethylcarbodiimide hydrochloride (EDCI), 1,3-dicyclohexylcarbodiimide (DCC), O-(7-azabenzotriazol-1-yl)-N,N,N′,N′-tetramethyluronium hexafluorophoshate (HATU), O-benzotriazol-1-N,N,N′,N′-tetramethyluronium hexafluorophosphate (HBTU), and mixtures thereof. Suitable solvents include, but are not limited to, CH₂Cl₂ and THF.

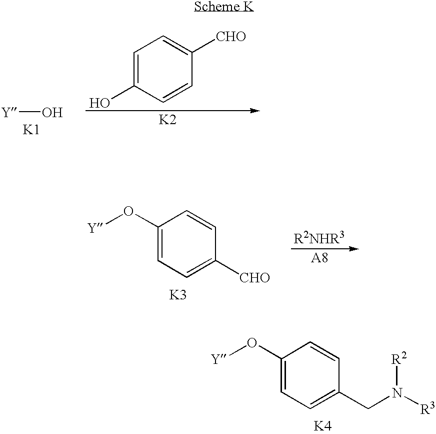

Referring to Scheme K, alcohols of the structure K1, where Y″═R¹(CH₂)₂₋₃—, R¹C(O)—, R¹CH(R⁹)C(O)—, or suitably protected R¹C(O)CH₂—, can be coupled with commercially available 4-hydroxybenzaldehyde, K2, to give structures of the type K3 under Mitsunobu conditions or peptide coupling conditions as described in the preceeding Schemes I and J. Compounds of structure K3 are treated with amines, A8, under standard reductive amination conditions as described in Scheme H to give compounds of the structure K4.

It is understood in light of the nomenclature for R², R³, R^{2′}, and R^{3′}, that the synthetic methods described herein and equivalents thereof apply not only to the structures that comprise groups R² and R³, but also to the structures that comprise R^{2′} and R^{3′}. Analogously, the synthetic methods described herein and equivalents thereof are applicable whether the structures comprise Y or Y′.

Where the processes for the preparation of the compounds according to the invention give rise to mixture of stereoisomers, these isomers may be separated by conventional techniques such as resolution, for example by formation of diastereomeric salts, kinetic resolution including variants thereof, such as dynamic resolution, preferential crystallization, biotransformation, enzymatic transformation, and preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric amines, esters, or amides, followed by chromatographic separation and removal of the chiral auxiliary. Alternatively, the compounds may be separated using a chiral HPLC column. Regioisomeric mixtures may also be separated into their constituent regioisomers by conventional techniques.

For therapeutic use, salts of the compounds of the present invention are those that are pharmaceutically acceptable. However, salts of acids and bases which are non-pharmaceutically acceptable may also find use, for example, in the preparation or purification of a pharmaceutically acceptable compound. All salts, whether pharmaceutically acceptable or not are included within the ambit of the present invention.

Pharmaceutically acceptable salts, esters, and amides of compounds according to the present invention refer to those salt, ester, and amide forms of the compounds of the present invention which would be apparent to the pharmaceutical chemist, i.e., those which are non-toxic and which would favorably affect the pharmacokinetic properties of said compounds of the present invention. Those compounds having favorable pharmacokinetic properties would be apparent to the pharmaceutical chemist, i.e., those which are non-toxic and which possess such pharmacokinetic properties to provide sufficient palatability, absorption, distribution, metabolism and excretion. Other factors, more practical in nature, which are also important in the selection, are cost of raw materials, ease of crystallization, yield, stability, hygroscopicity and flowability of the resulting bulk drug.

Examples of acids that may be used in the preparation of pharmaceutically acceptable salts include the following: acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid and undecylenic acid.

Compounds of the present invention containing acidic protons may be converted into their therapeutically active non-toxic metal or amine addition salt forms by treatment with appropriate organic and inorganic bases. Appropriate base salt forms comprise, for example, the ammonium salts; the alkali and earth alkaline metal salts (e.g. lithium, sodium, potassium, magnesium, calcium salts, which may be prepared by treatment with, for example, magnesium hydroxide, calcium hydroxide, potassium hydroxide, zinc hydroxide, or sodium hydroxide); and amine salts made with organic bases (e.g. primary, secondary and tertiary aliphatic and aromatic amines such as L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine). See, e.g., S. M. Berge, et al., “Pharmaceutical Salts”, J. Pharm. Sci., 1977, 66:1-19, which is incorporated herein by reference.

“Salt” also comprises the hydrates and solvent addition forms that compounds of the present invention are able to form. Examples of such forms are hydrates, alcoholates, and generally solvates.

Examples of suitable esters include C₁₋₇alkyl, C₅₋₇cycloalkyl, phenyl, substituted phenyl, and phenylC₁₋₆alkyl-esters. Preferred esters include methyl esters. Furthermore, examples of suitable esters include such esters where one or more carboxyl substituents is replaced with p-methoxybenzyloxy-carbonyl, 2,4,6-trimethylbenzyloxycarbonyl, 9-anthryloxycarbonyl, CH₃SCH₂COO—, tetrahydrofur-2-yloxycarbonyl, tetrahydropyran-2-yloxy-carbonyl, fur-2-yloxycarbonyl, benzoylmethoxycarbonyl, p-nitrobenzyloxy-carbonyl, 4-pyridylmethoxycarbonyl, 2,2,2-trichloroethoxycarbonyl, 2,2,2-tribromoethoxycarbonyl, t-butyloxycarbonyl, t-amyloxycarbonyl, diphenylmethoxycarbonyl, triphenylmethoxycarbonyl, adamantyloxycarbonyl, 2-benzyloxyphenyloxycarbonyl, 4-methylthiophenyloxycarbonyl, or tetrahydropyran-2-yloxycarbonyl.

Whether referred to herein explicitly or not, each of the terms “pharmaceutically acceptable salts,” “pharmaceutically acceptable esters,” and “pharmaceutically acceptable amides” include those salts, esters and amides, respectively that do not change the intrinsic properties of the active ingredient. See, for example, Remington, The Science and Practice of Pharmacy, 704 (20^{th} ed., 2000).

“Subject” or “patient” includes mammals such as human beings and animals (e.g., dogs, cats, horses, rats, rabbits, mice, non-human primates) in need of observation, experiment, treatment or prevention in connection with the relevant disease or condition. Preferably, the patient or subject is a human being.

“Composition” includes a product comprising the specified ingredients in the specified amounts, including in the effective amounts, as well as any product that results directly or indirectly from combinations of the specified ingredients in the specified amounts.

“Therapeutically effective amount” or “effective amount” and grammatically related terms mean that amount of active compound or pharmaceutical agent that elicits the biological or medicinal response in an in vitro system, a tissue system, an animal or human being, that is being sought by a researcher, veterinarian, medical doctor, or other clinician, where the medicinal response includes, but is not limited to, alleviation of the symptoms of the disease or disorder being treated. Analogously, terms such as “inhibitory amount,” “anti-inflammatory amount,” and grammatically related terms refer to the amount of active compound or pharmaceutical agent that elicits the response being referred to, such as inhibition and anti-inflammatory effect, respectively, in the system being studied, whether an in vitro system, a tissue system, an animal or a human being that is sought by a researcher, veterinarian, medical doctor, or other clinician, where the medicinal response includes, but is not limited to, alleviation of the symptoms of the disease or disorder being treated.

As used herein, “treating” a disorder, and grammatically related terms, mean eliminating or otherwise ameliorating the cause and/or effects thereof. Terms such as to “inhibit”, and grammatically related terms, the onset of a disorder or event, and to “prevent” a disorder or condition, and grammatically related terms, mean preventing, delaying or reducing the likelihood of such onset.

The terms “unit dose” and their grammatical equivalent forms are used herein to refer to physically discrete units suitable as unitary dosages for human patients and other animals, each unit containing a predetermined effective, pharmacologic amount of the active ingredient calculated to produce the desired pharmacological effect. The specifications for the novel unit dosage forms of this invention are determined by, and are directly dependent on, the characteristics of the active ingredient, and on the limitations inherent in the art of compounding such an active ingredient for therapeutic use in humans and other animals.

Compounds of the present invention may be used in pharmaceutical compositions to treat patients (humans and other mammals) with disorders involving the action of the LTA4H enzyme. In particular, compounds of the present invention may be used in pharmaceutical compositions to treat inflammation. More particularly, compounds of the present invention may be used in pharmaceutical compositions to treat inflammatory conditions such as inflammatory bowel disease (IBD) (such as Crohn's disease and ulcerative colitis), chronic obstructive pulmonary disease (COPD), arthritis, psoriasis, asthma, cystic fibrosis, atherosclerosis, rheumatoid arthritis, and multiple sclerosis. Compounds of the present invention may also be used in pharmaceutical compositions to treat, prevent, or inhibit inflammatory conditions such as cardiovascular disease, myocardial infarction, aortic aneurysm, or stroke.

The present invention features pharmaceutical compositions containing such compounds and methods of using such compositions in the treatment or prevention of conditions that are mediated by LTA4H enzyme activity. Accordingly, the present invention also contemplates a pharmaceutical composition that comprises at least one compound according to this invention, preferably in a pharmaceutically acceptable carrier. The at least one compound according to this invention is present in such composition in an amount sufficient to inhibit LTA4H enzyme activity. More particularly, the at least one compound according to this invention is present in such composition in an anti-inflammatory amount.

Accordingly, a pharmaceutical composition that comprises an anti-inflammatory amount of at least one compound according to the present invention in a pharmaceutically acceptable carrier is also contemplated herein. The composition comprises a unit dosage of the at least one compound according to this invention. In preferred practice, the at least one compound according to the present invention that is comprised in the pharmaceutical composition is capable of inhibiting LTA4H enzyme activity in the amount at which that compound is present in the pharmaceutical composition, when that pharmaceutical composition is introduced as a unit dose into an appropriate patient or subject.

The pharmaceutical compositions can be prepared using conventional pharmaceutical excipients and compounding techniques. Examples of suitable unit dosage forms are tablets, capsules, pills, powder packets, granules, wafers, and the like, segregated multiples of any unit dosage form, as well as liquid solutions, and suspensions. Oral dosage forms may be elixirs, syrups, capsules, tablets, and the like. Examples of solid carriers include those materials usually employed in the manufacture of pills or tablets, such as lactose, starch, glucose, methylcellulose, magnesium stearate, dicalcium phosphate, mannitol, and the like, thickeners such as tragacanth and methylcellulose USP, finely divided SiO₂, polyvinylpyrrolidone, magnesium stearate, and the like. Typical liquid oral excipients include ethanol, glycerol, water, and the like. All excipients may be mixed as needed with inert diluents (for example, sodium and calcium carbonates, sodium and calcium phosphates, and lactose), disintegrants (for example, cornstarch and alginic acid), diluents, granulating agents, lubricants (for example, magnesium stearate, stearic acid, and talc), binders (for example, starch and gelatin), thickeners (for example, paraffin, waxes, and petrolatum), flavoring agents, coloring agents, preservatives, and the like by conventional techniques known to those of ordinary skill in the art of preparing dosage forms. Coatings can be present and include, for example, glyceryl monostearate and/or glyceryl distearate. Capsules for oral use include hard gelatin capsules in which the active ingredient is mixed with a solid diluent, and soft gelatin capsules, in which the active ingredient is mixed with water or oil, such as peanut oil, liquid paraffin, or olive oil.

Parenteral dosage forms may be prepared using water or another sterile carrier. For intramuscular, intraperitoneal, subcutaneous, and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions may include suspending agents such as cellulose derivatives, sodium alginate, polyvinyl-pyrrolidone, and gum tragacanth, and a wetting agent, such as lecithin. Suitable preservatives for aqueous suspensions include ethyl and n-propyl p-hydroxybenzoate. Parenteral formulations include pharmaceutically acceptable aqueous or nonaqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Carriers for solid dosage forms include (a) fillers or extenders, (b) binders, (c) humectants, (d) disintegrating agents, (e) solution retarders, (f) absorption accelerators, (g) adsorbants, (h) lubricants, (i) buffering agents, and (j) propellants.

To aid solubility, suitable ingredients, such as cyclodextrins, may be included in the compositions. Appropriate cyclodextrins (CD) are α-, β-, γ-cyclodextrins or ethers and mixed ethers thereof wherein one or more of the hydroxy groups of the anhydroglucose units of the cyclodextrin are substituted with C₁₋₆alkyl, particularly methyl, ethyl or isopropyl, for example randomly methylated β-CD; hydroxyC₁₋₆alkyl, particularly hydroxyethyl, hydroxy-propyl or hydroxybutyl; carboxyC₁₋₆alkyl, particularly carboxymethyl or carboxy-ethyl; C₁₋₆alkylcarbonyl, particularly acetyl. Especially noteworthy as complexants and/or solubilizers are β-CD, randomly methylated β-CD, 2,6-dimethyl-β-CD, 2-hydroxyethyl-β-CD, 2-hydroxyethyl-β-CD, 2-hydroxypropyl-β-CD and (2-carboxymethoxy)propyl-β-CD, and in particular 2-hydroxypropyl-β-CD (2—HP-β-CD). The term mixed ether denotes cyclodextrin derivatives wherein at least two cyclodextrin hydroxy groups are etherified with different groups such as, for example, hydroxy-propyl and hydroxyethyl.

Compositions may also contain adjuvants such as preserving, wetting, emulsifying, and dispensing agents; antimicrobial agents such as parabens, chlorobutanol, phenol, and sorbic acid; isotonic agents such as a sugar or sodium chloride; absorption-prolonging agents such as aluminum monostearate and gelatin; and absorption-enhancing agents.

Physiologically acceptable carriers are well known in the art. Examples of liquid carriers are solutions in which compounds according to the present invention form solutions, emulsions, and dispersions. Compatible antioxidants, such as methlyparaben and propylparaben, can be present in solid and liquid compositions, as can sweeteners.

Pharmaceutical compositions according to the present invention may include suitable emulsifiers typically used in emulsion compositions. Such emulsifiers are described in standard publications such as H. P. Fiedler, 1989, Lexikon der Hilfsstoffe für Pharmazie, Kosmetic und agrenzende Gebiete, Cantor ed., Aulendorf, Germany, and in Handbook of Pharmaceutical Excipients, 1986, American Pharmaceutical Association, Washington, D.C., and the Pharmaceutical Society of Great Britain, London, UK, which are incorporated herein by reference. Examples of emulsifiers are given in U.S. Pat. No. 6,352,998, cols. 4-5. Gelling agents may also be added to compositions according to this invention. Polyacrylic acid derivatives, such as carbomers, are examples of gelling agents, and more particularly, various types of carbopol, which are typically used in amounts from about 0.2% to about 2%. Suspensions may be prepared as a cream, an ointment, including a water-free ointment, a water-in-oil emulsion, an oil-in-water emulsion, an emulsion gel, or a gel.

It is anticipated that the compounds of the invention can be administered by oral or parenteral routes, including intravenous, intramuscular, intraperitoneal, subcutaneous, rectal, and topical administration, and inhalation. For oral administration, the compounds of the invention will generally be provided in the form of tablets, capsules, or as a solution or suspension. Other methods of administration include controlled release formulations, such as subcutaneous implants and dermal patches.

Compounds according to the present invention and mixtures thereof provide embodiments of active substance in pharmaceutical compositions that can be made with excipients and ingredients and with ordinary skill in the art. Lists of excipients and ingredients for pharmaceutical compositions are available in standard references. For example, a standard text such as The Science and Practice of Pharmacy, A. R. Gennaro, ed., provides 20 chapters in part 5, pp. 669-1050, on pharmaceutical manufacturing, including lists of ingredients to manufacture pharmaceutical compositions such as solutions (including aromatic waters, aqueous acids, douches, enemas, gargles, mouthwashes, juices, nasal solutions, optic solutions, irrigation solutions, syrups, honeys, mucilages, jellies, collodions, elixirs, glycerins, inhalants, liniments, oleopreparations, spirits, and drops), emulsions (including multiple emulsions and microemulsions), suspensions, (including gels, lotions, tablet-formulated suspensions, magmas and milks, mixtures, and official suspensions), extracts, parenteral preparations, intravenous preparations, ophthalmic preparations, topical preparations, oral solid dosage forms, coatings, controlled-release drug delivery systems, aerosols, packaging materials, antioxidants, preservatives, coloring agents, flavoring agents, diluting agents, vehicles, emulsifying agents, suspending agents, ointment bases; pharmaceutical solvents, and miscellaneous pharmaceutical necessities, including the techniques and devices for manufacturing such preparations.

Effective doses of the compounds of the present invention may be ascertained by conventional methods. The specific dosage level required for any particular patient will depend on a number of factors, including severity of the condition, type of symptoms needing treatment, the route of administration, the weight, age, and general condition of the patient, and the administration of other medicaments.

In general, it is anticipated that the daily dose (whether administered as a single dose or as divided doses) will be in the range from about 0.01 mg to about 1000 mg per day, more usually from about 1 mg to about 500 mg per day, and most usually form about 10 mg to about 200 mg per day. Expressed as dosage per unit body weight, a typical dose will be expected to be between about 0.0001 mg/kg and about 15 mg/kg, especially between about 0.01 mg/kg and about 7 mg/kg, and most especially between about 0.15 mg/kg and 2.5 mg/kg.

Anticipated oral dose ranges include from about 0.01 to 500 mg/kg, daily, more preferably from about 0.05 to about 100 mg/kg, taken in 1-4 separate doses. Some compounds of the invention may be orally dosed in the range of about 0.05 to about 50 mg/kg daily, while others may be dosed at 0.05 to about 20 mg/kg daily. Infusion doses can range from about 1.0 to about 1.0×10⁴ μg/(kg.min) of inhibitor, admixed with a pharmaceutical carrier over a period ranging from several minutes to several days. For topical administration, compounds of the present invention may be mixed with a pharmaceutical carrier at a concentration from about 0.1 to about 10% of drug to vehicle. Capsules, tablets or other formulations (such as liquids and film-coated tablets) may be of between 0.5 and 200 mg, such as 1, 3, 5, 10,15, 25, 35, 50 mg, 60 mg, and 100 mg and can be administered according to the disclosed methods. Daily dosages are envisaged to be, for example, between 10 mg and 5000 mg for an adult human being of normal weight.

A method for treating inflammation in a patient exhibiting or susceptible to an inflammatory condition is also contemplated. A method for treating an LTA4H-mediated condition is also contemplated. The methods comprise administering to that patient an effective amount of a pharmaceutical composition that includes a unit dose of an active ingredient that is at least one of the compounds according to this invention dispersed in a pharmaceutically acceptable carrier.

### EXAMPLES

In order to illustrate the invention, the following examples are provided. These examples do not limit the invention. They are meant to illustrate embodiments of the invention. Those skilled in the art may find additional embodiments in light of the teachings and examples provided herein, additional embodiments that are deemed to be within the scope of this invention.

General Experimental Procedures:

NMR spectra were obtained on either a Bruker model DPX400 (400 MHz) or DPX500 (500 MHz) spectrometer. The format of the ¹H NMR data below is: chemical shift in ppm down field of the tetramethylsilane reference (multiplicity, coupling constant J in Hz, integration).

Mass spectra were obtained on an Agilent series 1100 MSD using electrospray ionization (ESI) in either positive or negative mode as indicated. The “mass calculated” for a molecular formula is the monoisotopic mass of the compound.

Reversed-Phase HPLC retention times are reported in minutes, using the methods and conditions reported below.
Instrument: Gilson 215
Solvent: CH₃CN (0.05% trifluoroacetic acid, TFA)/H₂O (0.05% TFA)
Flow rate: 25 mL/min
Gradient: 0 min at 10% CH₃CN; 20 min linear ramp to 99% CH₃CN;
Column: YMC-Pack ODS-A M 12505-1530WT SH-362-5 (S-5 um, 12 nM, 150×30 mm)
Temperature: 25° C.
Wavelength: Dual detection at 220 and 254 nM

Flash column chromatography was accomplished using ISCO Foxy 200 or ISCO OPTIX 10× systems employing one of the following commercially available prepacked columns: Biotage 40S (SiO₂ 40 g), Biotage 40M (SiO₂ 90 g), Biotage 40L (SiO₂ 120 g), Biotage 65M (SiO₂ 300 g) or ISCO Redisep (SiO₂, 10 g, 12 g, 35 g, 40 g, or 120 g).

### Example 1

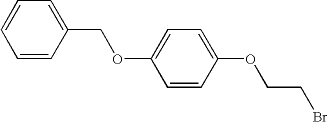

### 2-(4-Benzyloxy-phenoxy)-ethyl bromide

To a stirred solution of 4-benzyloxyphenol (72 g, 359.6 mmol) in CH₃CN (600 mL) was added dibromoethane (155 mL, 1.80 mol) and K₂CO₃ (105 g, 759.9 mmol). This brown suspension was heated at reflux and allowed to stir for 96 h. The resulting suspension was cooled to room temperature (rt), diluted with acetone (250 mL), and filtered through diatomaceous earth, which was then rinsed with additional acetone. The filtrate was concentrated. The resulting oil was dissolved in CH₃OH (500 mL), and the solution was stirred for 2 h. The title compound was obtained by filtration and air-dried to give 70 g (228 mmol, 63%) as a tan solid. ¹H NMR (400 MHz, CDCl₃): 7.60-7.30 (m, 5H), 6.88 (d, J=8.4, 2H), 6.80 (d, J=8.4, 2H), 4.70 (s, 2H), 3.79 (t, J=5.8, 2H), 3.07 (t, J=5.8, 2H).

### Example 2

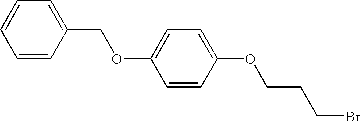

### 1-[3-(4-Benzyloxy-phenoxy)-propyl]-bromide

To a stirred solution of 4-benzyloxyphenol (25 g, 124.9 mmol) in CH₃CN (125 mL) was added dibromopropane (63 mL, 624 mmol) and K₂CO₃ (34.5 g, 250 mmol). This brown suspension was heated at reflux and stirred for 66 h. The suspension was then cooled to rt and filtered twice through diatomaceous earth pads. The pads were rinsed with CH₃CN, and the combined filtrates were concentrated. The resultant oil was purified on SiO₂ (300 g; 33% CH₂Cl₂/hexanes). The desired fractions were combined and concentrated to give 35.4 g (110 mmol, 88%) of a brown solid. ¹H NMR (400 MHz, CDCl₃): 7.46-7.29 (m, 5H), 6.85 and 6.82 (q, J=8.0 and 7.2, 4H), 5.03 (s, 2H), 4.06 (t, J=5.8, 2H), 3.61 (t, J=6.5, 2H), 2.39 (m, J=6.2, 2H).

### Example 3

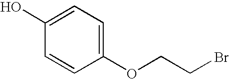

### 4-(2-Bromo-ethoxy)-phenol

2-(4-Benzyloxy-phenoxy)-ethyl bromide (EXAMPLE 1; 70 g, 227 mmol) was dissolved in THF (500 mL). To this solution was added 10% Pd/C (7 g) as a suspension in ethanol (50 mL). The resulting suspension was placed on a Parr hydrogenator at 40 psi of H₂ and shaken overnight. The reaction mixture was filtered through a pad of diatomaceous earth, and the filtrate was concentrated to give 48.5 g (224 mmol, 99%) of a tan solid. ¹H NMR (400 MHz, CDCl₃): 6.83 (d, J=9.1, 2H), 6.77 (d, J=9.1, 2H), 4.51 (s, 1H), 4.24 (t, J=6.3, 2H), 3.62 (t, J=6.3, 2H).

### Example 4

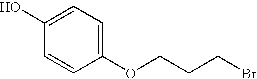

### 4-(3-Bromo-propoxy)-phenol

[3-(4-Benzyloxy-phenoxy)-propyl]-bromide (10 g, 31.1 mmol) was dissolved in THF (100 mL). To this solution was added 10% Pd/C (1 g) as a suspension in THF (20 mL). The resulting suspension was placed on a Parr hydrogenator at 40 psi of H₂, and shaken overnight. The reaction mixture was filtered through a pad of diatomaceous earth, and the filtrate was concentrated to give 7 g (30.5 mmol, 98%) of a tan solid. ¹H NMR (400 MHz, CDCl₃): 6.76 (d, J=9.1, 2H), 6.69 (d, 9.1, 2H), 4.00 (t, J=5.9, 2H), 3.60 (t, J=6.6, 2H), 2.23 (m, J=6.1, 2H).

### Example 5

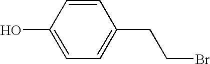

### 4-(2-Bromo-ethyl)-phenol

4-(2-Hydroxy-ethyl)-phenol (50 g, 362 mmol) was dissolved in 48 wt % HBr (250 mL). This light yellow solution was heated to 80° C. and stirred for 16 h. The reaction mixture was allowed to cool to rt and was then extracted with CH₂Cl₂ (3×50 mL). The combined extracts were dried, filtered, and concentrated to afford 72 g (100% crude) of a tan solid. ¹H NMR (400 MHz, CDCl₃): 9.25 (s, 1H), 7.04 (d, J=8.4, 2H), 6.67 (d, J=8.4, 2H), 3.62 (t, J=7.4, 2H), 2.97 (t, J=7.4, 2H).

### Example 6

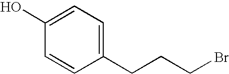

### 4-(3-bromo-propyl)-phenol

A mixture of 4-(3-hydroxy-propyl)-phenol (52.7 9, 346.3 mmol) in 48 wt % HBr (265 mL) was stirred at 80° C. for 20 h and then cooled to rt. Water (400 mL) was added, and the product was extracted with CH₂Cl₂ (500 mL). The extract was dried (MgSO₄) and concentrated to give the desired product as a beige solid (69 g, 92%). TLC (SiO₂, CH₂Cl₂): R_{f}=0.37. ¹H NMR (400 MHz, DMSO-d₆): 9.18 (s, 1H), 6.99 (d, J=8.3, 2H), 6.67 (d, J=8.4, 2H), 3.47 (t, J=6.6, 2H), 2.58 (t, J=7.2, 2H), 2.05-1.95 (m, 2H).

### Example 7

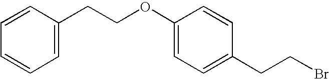

### 1-(4-Phenethyloxy-phenyl)-ethyl bromide

To a stirred solution of 4-(2-bromo-ethoxy)-phenol (2.01 g, 10 mmol) in CH₂Cl₂ (200 mL), was added 2-phenylethanol (1.79 mL, 15 mmol), followed by polymer-supported triphenylphosphine (5 g, 15 mmol) and di-tert-butyl azodicarboxylate (4.6 g, 20 mmol). The mixture was stirred for 2 h at rt. The resulting suspension was filtered, and the filtrate was concentrated. The resultant oil was purified on SiO₂ (110 g; 10-100% EtOAc/hexanes). The desired fractions were combined and concentrated to give 2.58 g (85%) of a brown oil. ¹H NMR (400 MHz, CDCl₃): 7.36-7.19 (m, 5H), 7.09 (d, J=8.8, 2H), 6.83 (d, J=8.8, 2H), 4.14 (t, J=7.1, 2H), 3.50 (t, J=7.6, 2H), 3.11-3.04 (m, 4H).

### Example 8

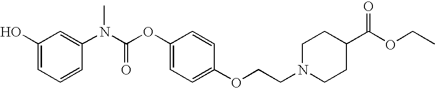

### 1-(2-{4-[(3-Hydroxy-phenyl)-methyl-carbamoyloxy]-phenoxy}-ethyl)-piperidine-4-carboxylic acid ethyl ester

A. 1-[2-(4-Hydroxy-phenoxy)-ethyl-piperidine-4-carboxylic acid ethyl ester. To a stirred solution of 4-(2-bromo-ethoxy)-phenol (5 g, 23.1 mmol) in CH₃CN (200 mL) was added ethyl isonipecotate (5.3 mL, 34.7 mmol). The reaction mixture was heated to reflux and stirred for 16 h, then cooled to rt and concentrated. The resultant oil was dissolved in CH₂Cl₂ and purified on SiO₂ (300 g; 0-25% acetone/CH₂Cl₂). The desired fractions were collected and concentrated, giving a white solid (6.3 g, 93%). MS (ESI): mass calculated for C₁₆H₂₃NO₄, 293.16; m/z found, 294.3 [M+H]⁺. ¹H NMR (400 MHz, CDCl₃): 6.74-6.56 (m, 4H), 4.07 (q, J=7.2, 2H), 3.96 (t, J=5.7, 2H), 3.08-2.87 (m, 2H), 2.74 (t, J=5.6, 2H), 2.26-2.23 (m, 3H), 1.88-1.77 (m, 5H), 1.17 (t, J=7.2, 3H).

B. N-(3-Benzyloxy-phenyl)-formamide. A stirred mixture of ethyl formate (10 mL, 124 mmol) and 3-benzyloxyaniline (7.0 g, 35 mmol) was heated to reflux and stirred for 20 h, then cooled to rt and concentrated. The resultant oil was dried under high vacuum and a white solid formed. The solid was dissolved in CH₂Cl₂ and purified on SiO₂ (110 g, 0-5% acetone/CH₂Cl₂). The desired fractions were collected and concentrated to give a white solid (7.0 g, 88%). TLC (SiO₂, 5% acetone/CH₂Cl₂): R_{f}=0.28. MS (ESI): mass calculated for C₁₄H₁₃NO₂, 227.09; m/z found, 228.2 [M+H]⁺. ¹H NMR (400 MHz, DMSO-d₆): 10.17 (s, 1H), 8.25 (d, J=1.8, 1H), 7.51-7.28 (m, 6H), 7.23-7.18 (m, 1H), 7.11 (d, J=8.1, 1H), 6.74 (d, J=8.2, 1H), 5.06 (s, 2H).

C. (3-Benzyloxy-phenyl)-methyl-amine. To a stirred solution of N-(3-benzyloxy-phenyl)-formamide (7.0 g, 31 mmol) in THF (100 mL) at 5° C. was added 2.0 M BH₃—Me₂NH in THF (46 mL, 92 mmol). The reaction mixture was stirred and slowly warmed to rt for 24 h, then quenched by the slow addition of satd. aq. NH₄Cl (400 mL). To the mixture was added CH₂Cl₂ (200 mL) and the organic layer was separated, dried (MgSO₄) and concentrated to give a dark brown oil (5.3 g, 80%). MS (ESI): mass calculated for C₁₄H₁₅NO, 213.12; m/z found, 214.3 [M+H]⁺. ¹H NMR (400 MHz, DMSO-d₆): 7.48-7.25 (m, 5H), 6.97 (t, J=8.3, 1H), 6.20 (d, J=8.9, 1H), 6.18-6.12 (m, 2H), 5.63 (s, 1H), 5.02 (s, 2H), 2.64 (s, 3H).

D. (3-Benzyloxy-phenyl)-methyl-carbamoyl chloride. To a stirred solution of (3-benzyloxy-phenyl)-methyl-amine (5.3 g, 25 mmol) in CH₂Cl₂ (50 mL) at 5° C. was added 20% phosgene in toluene (20 mL, 37.8 mmol) followed by DIEA (5.0 mL, 29 mmol). The reaction mixture was stirred and slowly warmed to rt over 24 h, then H₂O (150 mL) was added and the organic layer was separated. The organic solution was dried (MgSO₄) and concentrated to give a clear golden oil. The oil was dissolved in 2:1 hexanes/CH₂Cl₂ and purified on SiO₂ (120 g, 60-0% hexanes/CH₂Cl₂). The desired fractions were collected and concentrated to give a brown solid (5.1 g, 74%). MS (ESI): mass calculated for C₁₅H₁₄ClNO₂, 275.07; m/z found, 276.3 [M+H]⁺. ¹H NMR (400 MHz, DMSO-d₆): 7.52-7.25 (m, 6H), 7.13 (s, 1H), 7.05 (d, J=8.9, 1H), 6.97 (d, J=8.1, 1H), 5.12 (s, 2H), 3.30 (s, 3H).

E. 1-(2-[4-[(3-Benzyloxy-phenyl)-methyl-carbamoyloxy-phenoxy}-ethyl)-piperidine-4-carboxylic acid ethyl ester. To a stirred solution of 1-[2-(4-hydroxy-phenoxy)-ethyl]-piperidine-4-carboxylic acid ethyl ester (993 mg, 3.39 mmol) in THF (15 mL) at 5° C. was added potassium tert-butoxide (411 mg, 3.48 mmol). After 15 min, (3-benzyloxy-phenyl)-methyl-carbamoyl chloride (944 mg, 3.42 mmol) was added in one portion and the mixture was stirred and warmed to rt over 72 h, then concentrated. The residue was diluted with EtOAc, washed with brine (50 mL), dried (MgSO₄) and concentrated to give a clear light golden oil. The oil was dissolved in CH₂Cl₂ and purified on SiO₂ (40 g, 0-50% acetone/CH₂Cl₂) to give a clear and colorless oil (1.26 g, 70%). TLC (SiO₂, 50% acetone/CH₂Cl₂): R_{f}=0.62.). MS (ESI): mass calculated for C₃₁H₃₆N₂O₆, 532.26; m/z found, 533.4 [M+H]⁺. ¹H NMR (400 MHz, DMSO-d₆): 7.46 (d, J=7.05, 2H), 7.38 (t, J=7.6, 2H), 7.36-7.28 (m, 2H), 7.14 (t, J=2.1, 1H), 7.08-6.98 (m, 3H), 5.12 (s, 2H), 4.10-4.00 (m, 4H), 3.35 (s, 3H), 3.31 (s, 3H), 2.86 (d, J=11.6, 2H), 2.66 (t, J=5.80, 2H), 2.32-2.20 (m, 1H), 2.09 (dt, J=11.4, 2.1, 2H), 1.78 (d, J=13.3, 2H), 1.57 (q, J=7.86, 2H), 1.18 (t, J=7.09, 3H).

F. 1-(2-{4-[(3-Hydroxy-phenyl)-methyl-carbamoyloxy]-phenoxy}-ethyl)-piperidine-4-carboxylic acid ethyl ester. To a solution of 1-(2-{4-[(3-benzyloxy-phenyl)-methyl-carbamoyloxy]-phenoxy}-ethyl)-piperidine-4-carboxylic acid ethyl ester (1.13 g, 2.12 mmol) in THF (15 mL) was added Pd on carbon (10 wt %, 102 mg). The mixture was placed on a Parr hydrogenator at 40 psi of H₂ for 20 h. The resultant mixture was filtered through diatomaceous earth, and the filtrate was concentrated to give a clear and colorless oil(1.06 g, 100%). TLC (SiO₂, 50% acetone/CH₂Cl₂): R_{f}=0.35. MS (ESI): mass calculated for C₂₄H₃₀N₂O₆, 442.21; m/z found, 443.4 [M+H]⁺. ¹H NMR (400 MHz, DMSO-d₆): 9.62 (s, 1H), 7.19 (t, J=8.03, 1H), 7.02 (d, J=8.89, 2H), 6.91 (d, J=7.00, 2H), 6.88-6.80 (m, 2H), 6.67 (dd, J=8.14, 2.13, 1H), 4.10-4.00 (m, 4H), 3.35 (s, 3H), 2.86 (d, J=11.6, 2H), 2.66 (t, J=5.80, 2H), 2.32-2.20 (m, 1H), 2.09 (dt, J=11.4, 2.1, 2H), 1.78 (d, J=13.3, 2H), 1.57 (q, J=7.86, 2H), 1.18 (t, J=7.09, 3H).

### Example 9

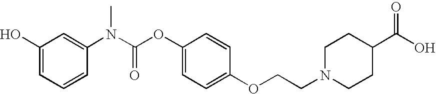

### 1-(2-{4-[(3—hydroxy-phenyl)-methyl-carbamoyloxy]-phenoxy}-ethyl)-piperidine-4-carboxylic acid

To a stirred solution of 1-(2-{4-[(3-hydroxy-phenyl)-methyl-carbamoyloxy]-phenoxy}-ethyl)-piperidine-4-carboxylic acid ethyl ester (987 mg, 2.23 mmol) in 25% i-PrOH/CHCl₃ (20 mL) was added KOH (438 mg, 7.81 mmol). After 20 h, 1 M HCl was added to the mixture until the pH was adjusted to 5. The mixture was extracted with CHCl₃ (2×50 mL). The organic layers were combined, dried (MgSO₄) and concentrated to give a light beige solid (612 mg, 66%). MS (ESI): mass calculated for C₂₂H₂₆N₂O₆, 414.18; m/z found, 415.4 [M+H]⁺. ¹H NMR (400 MHz, DMSO-d₆): 9.62 (s, 1H), 7.19 (t, J=8.0, 1H), 7.02 (d, J=8.9, 2H), 6.91 (d, J=9.0, 2H), 6.88-6.80 (m, 2H), 6.67 (dd, J=8.1, 1.8, 1H), 4.04 (t, J=5.8, 2H), 3.28 (s, 3H), 2.86 (d, J=11.4, 2H), 2.66 (t, J=5.7, 2H), 2.20-2.13 (m, 1H), 2.07 (t, J=10.9, 2H), 1.78 (d, J=13.1, 2H), 1.53 (d, J=9.6, 2H).

### Example 10

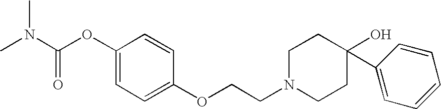

### Dimethyl-carbamic acid 4-[2-(4-hydroxy-4-phenyl-piperidin-1-yl)-ethoxy]-phenyl ester

A. 1-[2-(4-Hydroxy-phenoxy)-ethyl]-4-phenyl-piperidin-4-ol. To a solution of 4-(2-bromo-ethoxy)-phenol (8.0 g, 36.8 mmol) and 4-hydroxy-4-phenylpiperidine (8.2 g, 46.3 mmol) in CH₃CN (150 mL) was added DIEA (7.0 mL, 40.2 mmol). The mixture was stirred for 20 h at rt and for an additional 4 h at 65° C. The mixture was then concentrated to give a brown solid. The solid was dissolved in EtOAc (250 mL), and the solution was washed with H₂O (250 mL), dried (MgSO₄), and concentrated to give a brown solid. The solid was purified on SiO₂ (120 g; 0-100% acetone/CH₂Cl₂). The desired fractions were combined and concentrated to give 8.9 g (77%) of the desired product as a tan solid. TLC (SiO₂, acetone): R_{f}=0.42. MS (ESI): mass calculated for C₁₉H₂₃NO₃, 313.17; m/z found, 314.2 [M+H]⁺. ¹H NMR (400 MHz, CDCl₃): 7.52 (d, J=8.6, 2H), 7.37 (t, J=7.3, 2H), 7.27 (m, 1H), 6.75 (s, 4H), 4.08 (t, J=5.8, 2H), 3.05-2.90 (m, 2H), 2.88 (t, J=5.8, 2H), 2.80-2.62 (m, 2H), 2.31-2.18 (m, 2H), 1.81 (d, J=11.8, 2H).

B. Dimethyl-carbamic acid 4-[2-(4-hydroxy-4-phenyl-piperidin-1-yl)-ethoxyl-phenyl ester. To a stirred solution of 1-[2-(4-hydroxy-phenoxy)-ethyl]-4-phenyl-piperidin-4-ol (150 mg, 0.48 mmol) in CH₃CN (5 mL) containing K₂CO₃ (100 mg, 0.72 mmol) was added dimethylcarbamoyl chloride (66 μL, 0.72 mmol). The mixture was stirred and heated to reflux for 20 h then filtered and concentrated to give a clear golden oil. The oil was dissolved in CH₂Cl₂ and purified on SiO₂ (12 g, 100-0% CH₂Cl₂/acetone). The desired fractions were combined and concentrated to give a white solid (145 mg, 79%). TLC (SiO₂, acetone): R_{f}=0.31. MS (ESI): mass calculated for C₂₂H₂₈N₂O₄, 384.20; m/z found 385.4 [M+H]⁺. ¹H NMR (400 MHz, DMSO-d₆): 7.48 (d, J=8.5, 2H), 7.31 (t, J=7.4, 2H), 7.19 (t, J=7.4,1H), 7.00 (d, J=9.1, 2H), 6.93 (d, J=6.8, 2H), 4.79 (s, 1H), 4.08 (t, J=5.9, 2H), 3.02 (s, 3H), 2.89 (s, 3H), 2.72 (t, J=6.0, 4H), 2.54-2.45 (m, 2H), 1.93 (dt, J=11.4, 2.1, 2H), 1.57 (d, J=12.0, 2H).

### Example 11

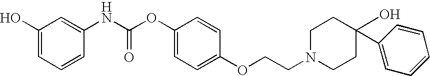

### (3-hydroxy-phenyl)-carbamic acid 4-[2-(4-hydroxy-4-phenyl-piperidin-1-yl)-ethoxy]-phenyl ester

A. 1-[2-(4-Hydroxy-phenoxy)-ethyl]-4-phenyl-piperidin-4-ol. To a solution of 4-(2-bromo-ethoxy)-phenol (8.0 g, 37 mmol) and 4-hydroxy-4-phenylpiperidine (8.2 g, 46 mmol) in CH₃CN (150 mL) was added DIEA (7.0 mL, 40.2 mmol). The mixture was stirred for 20 h at rt and for an additional 4 h at 65° C., then was concentrated to give a brown solid. The solid was dissolved in EtOAc (250 mL), and the solution was washed with H₂O (250 mL), dried (MgSO₄), and concentrated to give a brown solid. The solid was purified on SiO₂ (120 g; 0-100% acetone/CH₂Cl₂). The desired fractions were combined and concentrated to give 8.9 g (77%) of the desired product as a tan solid. TLC (SiO₂, acetone): R_{f}=0.42. MS (ESI): mass calculated for C₁₉H₂₃NO₃, 313.17; m/z found 314.2 [M+H]⁺. ¹H NMR (400 MHz, CDCl₃): 7.52 (d, J=8.6, 2H), 7.37 (t, J=7.3, 2H), 7.27 (m, 1H), 6.75 (s, 4H), 4.08 (t, J=5.8, 2H), 3.05-2.90 (m, 2H), 2.88 (t, J=5.8, 2H), 2.80-2.62 (m, 2H), 2.31-2.18 (m, 2H), 1.81 (d, J=11.8, 2H).

B. 3-Benzyloxyphenyl isocyante. To a stirred solution of 3-benzyloxyaniline (507 mg, 2.54 mmol) in toluene (5 mL) containing TEA (740 μL, 5.34 mmol) was added 20% phosgene in toluene (1.5 mL, 2.83 mmol). The reaction was stirred for 20 h at rt. The organic layer was washed with H₂O (20 mL), dried (MgSO₄) and concentrated to give a clear brown oil (497 mg, 87%). The material was used in subsequent steps without charaterization.

C. (3-Benzyloxy-phenyl)-carbamic acid 4-[2-(4-hydroxy-4-phenyl-piperidin-1-yl)-ethoxy]-phenyl ester. To a stirred solution of 1-[2-(4-hydroxy-phenoxy)-ethyl]-4-phenyl-piperidin-4-ol (504 mg, 1.61 mmol) and TEA (383 μL, 2.8 mmol) in CH₂Cl₂ was added 3-benzyloxyphenyl isocyante (497 mg, 2.21 mmol). The mixture was stirred for 20 h and concentrated to give a clear golden oil. The oil was dissolved in CH₂Cl₂ and purified on SiO₂ (12 g, 100-0% CH₂Cl₂/acetone). The desired fractions were combined and concentrated to give a clear and colorless oil (460 mg, 53%). TLC (SiO₂, acetone): R_{f}=0.52. MS (ESI): mass calculated for C₃₃H₃₄N₂O₅, 538.25; m/z found, 539.5 [M+H]⁺. ¹H NMR (400 MHz, DMSO-d₆): 10.18 (s, 1H), 7.55-7.36 (m, 10H), 7.35-7.06 (m, 6H), 6.97 (d, J=7.0, 2H), 6.65 (d, J=7.3, 1H), 5.06 (s, 2H), 4.79 (s, 1H), 4.08 (t, J=5.8, 2H), 2.75 (s, 4H), 2.52 (m, 2H), 1.93 (dt, J=11.3, 1.9, 2H), 1.57 (d, J=12.1, 2H).

### Example 12

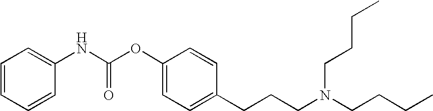

### Phenyl-carbamic acid 4-(3-dibutylamino-propyl)-phenyl ester hydrochloride

A. [3-(4-Benzyloxy-phenyl)-propyl]-dibutyl-amine. To a stirred a solution of 3-(4-benzyloxy-phenyl)-propyl-1-bromide (985 mg, 3.23 mmol) and K₂CO₃ (1.4 g, 10.1 mmol) in CH₃CN (20 mL) was added dibutylamine (1.1 mL, 6.5 mmol). The mixture was heated to reflux for 20 h, cooled to room temperature, filtered, and concentrated to give a light golden oil. The oil was dissolved in CH₂Cl₂ and purified on SiO₂ (40 g, 100-50% CH₂Cl₂/acetone). The desired fractions were combined and concentrated to give a clear light yellow liquid (1.0 g, 88%). TLC (SiO₂, 50% CH₂Cl₂/acetone): R_{f}=0.34. MS (ESI): mass calculated for C₂₄H₃₅NO, 353.27; m/z found, 354.4 [M+H]⁺. ¹H NMR (400 MHz, DMSO-d₆): 7.50-7.30 (m, 5H), 7.08 (d, J=8.6, 2H), 6.89 (d, J=8.6, 2H), 5.05 (s, 2H), 2.52-2.48 (m, 2H), 2.37-2.25 (m, 6H), 1.65-1.58 (m, 2H), 1.35-1.22 (m, 8H), 0.85 (t, J=7.1, 6H).

B. 4-(3-Dibutylamino-propyl)-phenol. To a solution of [3-(4-benzyloxy-phenyl)-propyl]-dibutyl-amine (962 mg, 2.72 mmol) in 1:1 EtOH/EtOAc (25 mL) was added 10% Pd/C (104 mg). The mixture was placed on a Parr hydrogenator at 40 psi of H₂ for 20 h. The resultant mixture was filtered through diatomaceous earth, and the filtrate was concentrated to give a clear yellow oil (700 mg, 98%). TLC (SiO₂, acetone): R_{f}=0.22. MS (ESI): mass calculated for C₁₇H₂₉NO, 263.22; m/z found, 264.3 [M+H]⁺. ¹H NMR (400 MHz, DMSO-d₆): 9.09 (s, 1H), 6.93 (d, J=8.4, 2H), 6.64 (d, J=6.6, 2H), 2.49 (t, J=3.5, 2H), 2.31 (t, J=7.0, 6H), 1.63-1.52 (m, 2H), 1.35-1.21 (m, 8H), 0.85 (t, J=7.1, 6H).

C. {3-[4-(Benzothiazol-2-yloxy)-phenyl-propy}-dibutyl-amine hydrochloride. To a stirred solution of 4-(3-dibutylamino-propyl)-phenol (116 mg, 0.44 mmol) and DIEA (85 μL, 0.49 mmol) in CH₂Cl₂ (8 mL) was added phenylisocyanate (53 μL, 0.49 mmol). The mixture was stirred at rt for 20 h and concentrated to give a clear golden oil. The oil was dissolved in CH₂Cl₂ and purified on SiO₂ (12 g, 100-0% CH₂Cl₂/acetone). The desired fractions were combined and concentrated to give the free base as a clear light golden oil (86 mg, 51%). TLC (SiO₂, acetone): R_{f}=0.22. The oil was dissolved in CH₃OH (3 mL) and 1 M HCl in Et₂O (0.5 mL, 0.5 mmol) was added. A white solid formed, which was filtered and air-dried to give a light beige solid (92 mg, 50%). MS (ESI): mass calculated for C₂₄H₃₄N₂O₂, 382.26; m/z found, 383.2 [M+H]⁺. ¹H NMR (400 MHz, DMSO-d₆): 10.31 (s, 1H), 9.97 (s, 1H), 7.50 (d, J=7.9, 2H), 7.37-7.24 (m, 4H), 7.15 (d, J=8.4, 2H), 7.11-6.98 (m, 1H), 3.08-2.95 (m, 6H), 2.68-2.57 (m, 2H), 1.99-1.87 (m, 2H), 1.62-1.55 (m, 4H), 1.38-1.24 (m, 4H), 0.90 (t, J=7.3, 6H).

### Example 13

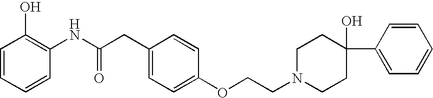

### N-(2—hydroxy-phenyl)-2-{4-[2-(4-hydroxy-4-phenyl-piperidin-1-yl)-ethoxy]-phenyl}-acetamide

A. [4-(2-Bromo-ethoxy)-phenyl]-acetic acid methyl ester. To a stirred suspension of Cs₂CO₃ (226 g, 693 mmol) and 4-hydroxyphenylacetate methyl ester (90 g, 542 mmol) in CH₃CN (270 mL) was added 1,2-dibromoethane (270 mL, 3.1 mol), and the resulting suspension was heated to 78° C. and stirred for 18 h. The suspension was then cooled, and Et₂O (1.35 L) was added, and the suspension was filtered and concentrated. The resultant oil was dissolved in CH₂Cl₂ (65 mL) and purified on SiO₂ (1 L, CH₂Cl₂). The desired fractions were collected and concentrated, and then vacuum distilled (155° C., ˜2 torr) to give 57.9 g (39%) of a clear oil. ¹H NMR (400 MHz, CDCl₃): 7.24 (d, J=8.6, 2H), 6.91 (d, J=8.6, 2H), 4.31 (t, J=6.3, 2H), 3.72 (s, 3H), 3.67 (t, J=6.3, 2H), 3.61 (s, 2H).

B. {4-[2-(4-Hydroxy-4-phenyl-piperidin-1-yl)-ethoxy]-phenyl}-acetic acid methyl ester. To a stirred solution of [4-(2-bromo-ethoxy)-phenyl]-acetic acid methyl ester (5 g, 18.3 mmol) in CH₃CN (92 mL) was added 4-hydroxy-4-phenylpiperidine (4.8 g, 28 mmol). The resulting suspension was warmed to 60° C., and TEA (2.54 mL, 18.3 mmol) was added. The resulting solution was stirred for 90 min, cooled, and stirred at rt overnight. The suspension was then filtered and concentrated. The resultant oil was purified on SiO₂ (110 g, 25-100% acetone/CH₂Cl₂) to give 2.6 g (39%) of a white solid. MS (ESI): exact mass calculated for C₂₂H₂₇NO₄, 369.19; m/z found, 370.2 [M+H]⁺. ¹H NMR (400 MHz, CD₃OD): 7.52 (d, J=7.6, 2H), 7.38 (t, J=7.8, 2H), 7.32-7.27 (m, 1H), 7.20 (d, J=8.6, 2H), 6.91 (d, J=8.6, 2H) 4.57 (t, J=4.4, 2H), 3.68 (s, 3H), 3.57-3.43 (m, 8H), 2.90 (dt, J=14.5, 4.6, 2H), 1.96 (d, J=13.9, 2H).

C. {4-[2-(4—hydroxy-4-phenyl-piperidin-1-yl)-ethoxy]-phenyl}-acetic acid. To a stirred solution of {4-[2-(4-hydroxy-4-phenyl-piperidin-1-yl)-ethoxy]-phenyl}-acetic acid methyl ester (2.6 g, 7 mmol) in THF (17 mL) and H₂O (17 mL) was added LiOH (552 mg, 23.1 mmol). The resulting solution was stirred overnight, and then concentrated to give 3.1 g (>100%) of a white solid. MS (ESI): exact mass calculated for C₂₁H₂₅NO₄, 355.18; m/z found, 356.4 [M+H]⁺. ¹H NMR (400 MHz, CD₃OD): 7.47 (d, J=7.4, 2H), 7.30 (t, J=7.5, 2H), 7.19 (t, J=7.3, 1H), 7.12 (d, J=8.5, 2H), 6.78 (d, J=8.5, 2H), 4.04 (t, J=5.8, 2H), 3.12 (s, 2H), 2.72 (t, J=5.8, 4H), 2.50 (m, 4H), 1.94 (dt, J=12.7, 3.8, 2H), 1.56 (d, J=12.4, 2H).

D. N-(2-hydroxy-phenyl)-2-[4-[2-(4-hydroxy-4-phenyl-piperidin-1-yl)-ethoxy]-phenyl}-acetamide. A stirred solution of {4-[2-(4-hydroxy-4-phenyl-piperidin-1-yl)-ethoxy]-phenyl}-acetic acid (2.2 g, 6.2 mmol) in DMF (30 mL) was heated to 50° C. To this solution was added O-benzotriazol-1-N,N,N′,N′-tetramethyluronium hexafluorophosphate (HBTU, 3.5 g, 9.3 mmol). The reaction mixture was stirred for 30 min, then 2-aminophenol (1.35 g, 12.4 mmol) was added. The solution was then heated to 70° C. and stirred overnight. The reaction was then cooled to rt, concentrated, and partitioned between EtOAc (150 mL) and satd. aq. NaHCO₃ (150 mL). The organic layer was dried (Na₂SO₄), concentrated, and purified on SiO₂ (110 g, 0-5% 2 M NH₃ in CH₃OH)/CH₂Cl₂) to give 370 mg (13%) of a white solid. MS (ESI): exact mass calculated for C₂₇H₃₀N₂O₄, 446.22; m/z found, 447.2 [M+H]⁺. ¹H NMR (400 MHz, CD₃OD): 7.70 (dd, J=8.0, 1.5, 1H), 7.49 (dd, J=8.1, 1.3, 2H), 7.33-7.28 (m, 4H), 7.20 (t, J=7.3, 1H), 6.97-6.93 (m, 3H), 6.82-6.75 (m, 2H), 4.18 (t, J=5.5, 2H), 3.68 (s, 2H), 2.90 (q, J=5.2, 4H), 2.69 (t, J=10.7, 2H), 2.17 (m, 2H), 1.74 (d, J=12.3, 2H).

### Example 14

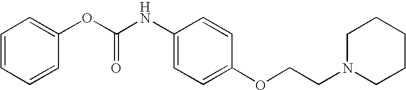

### [4-(2-Piperidin-1-yl-ethoxy)-phenyl]-carbamic acid phenyl ester hydrochloride

A. 1-(2-Bromo-ethoxy)-4-nitro-benzene. A solution of 4-nitrophenol (13.6 g, 97.8 mmol) and 1,2-dibromoethane (42.1 mL, 489 mmol) in CH₃CN (100 mL) was treated with finely powdered K₂CO₃ (27 g, 196 mmol) and the resulting suspension was stirred for 24 h at 85° C. The reaction mixture was filtered through diatomaceous earth and concentrated to a crude solid that was triturated with Et₂O and filtered to yield 20 g of crude solid. Recrystallization from hexanes gave 11.6 g (48%) of a solid. H NMR (400 MHz, CDCl₃): 8.25-8.18 (m, 2H), 7.06-6.98 (m, 2H), 4.43, (t, J=6.9, 2H), 3.72 (t, J=6.9, 2H).

B. 1-[2-(4-Nitro-phenoxy)-ethyl-piperidine. A solution of 1-(2-bromo-ethoxy)-4-nitro-benzene (5.0 g, 20.3 mmol) in CH₃CN (100 mL) was treated with piperidine (3.0 mL, 30.4 mmol) and DIEA (8.8 mL, 50.8 mmol). The resulting solution was stirred at rt for 16 h, then heated to 60° C. for 2 h. The reaction mixture was cooled and concentrated. The resulting crude oil was dissolved in EtOAc (250 mL) and the solution washed successively with H₂O (3×30 mL) and brine (30 mL), dried, and concentrated to yield 4.15 g (82%) of a brown oil, which was used without purification. ¹H NMR (400 MHz, CDCl₃): 8.25-8.18 (m, 2H), 7.06-6.98 (m, 2H), 4.28 (t, J=5.8, 2H), 2.89 (t, J=5.8, 2H), 2.65-2.50 (m, 4H), 1.72-1.63 (m, 4H), 1.58-1.44 (m, 2H).

C. 4-(2-Piperidin-1-yl-ethoxy)-phenylamine. To a solution of 1-[2-(4-nitro-phenoxy)-ethyl]-piperidine (300 mg, 1.20 mmol) in EtOH (50 mL) was added 10% Pd/C (50 mg). The mixture was placed on a Parr hydrogenator at 40 psi of H₂ for 30 min. The resultant mixture was filtered through diatomaceous earth, and the filtrate was concentrated to give a clear and colorless oil (264 mg, 100%). ¹H NMR (400 MHz, DMSO-d₆): 6.64 (d, J=8.7, 2H), 6.50 (d, J=8.0, 2H), 4.58 (s, 2H), 3.90 (t, J=6.0, 2H), 2.57 (t, J=6.0, 2H), 2.39 (s, 4H), 1.52-1.44 (m, 4H), 1.41-1.34 (m, 2H).

D. [4-(2-Piperidin-1-yl-ethoxy)-phenyl]-carbamic acid phenyl ester hydrochloride. To a solution of 4-(2-piperidin-1-yl-ethoxy)-phenylamine (142 mg, 0.64 mmol) in CH₂Cl₂ (3 mL) was added phenylchloroformate (100 mg, 0.64 mmol) and the resulting mixture was stirred for 30 min. The reaction mixture was concentrated and the resulting solid was triturated with Et₂O and filtered to yield 120 mg (50%) of a white solid. MS (ESI): mass calculated for C₂₀H₂₄N₂O₃, 340.18; m/z found, 341.4 [M+H]⁺. H NMR (400 MHz, DMSO-d₆): 10.12 (s, 1H), 7.58-7.35 (m, 4H), 7.26 (t, J=7.4, 1H), 7.22 (d, J=7.5, 2H), 6.98 (d, J=9.1, 2H), 4.34 (t, J=4.9, 2H), 3.42 (s, 2H), 3.37 (t, J=4.9, 2H), 2.99 (s, 2H), 1.85-1.55 (m, 5H), 1.40 (s, 1H).

### Example 15

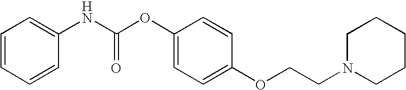

### Phenyl-carbamic acid 4-(2-piperidin-1-yl-ethoxy)-phenyl ester

A. 1-[2-(4-Benzyloxy-phenoxy)-ethyl]-piperidine. To a mixture of 4-(benzyloxy)phenol (24.6 g, 123 mmol) and 1-(2-chloroethyl)piperidine hydrochloride (20.6 g, 112 mmol) in DMF (175 mL) was added K₂CO₃ (25 g, 181 mmol) and Cs₂CO₃ (40 g, 123 mmol). The reaction mixture was stirred for 3 d at rt. To the mixture was added H₂O (300 mL) and CH₂Cl₂. The organic layer was separated and washed sequentially with 10% aq. NaOH and brine, dried (MgSO₄), filtered, and concentrated to give 33 g of a clear, dark purple liquid. The liquid was purified on SiO₂ (300 g; 0-50% EtOAc/hexanes) to give 23.4 g (67%) of a light yellow solid. TLC (SiO₂, 50% hexanes/EtOAc): R_{f}=0.11. MS (ESI): mass calculated for C₂₀H₂₅NO₂, 311.19; m/z found, 312.2 [M+H]⁺. ¹H NMR (400 MHz, CDCl₃): 7.50-7.26 (m, 5H), 6.91 (d, J=9.2, 2H), 6.85 (d, J=9.2, 2H), 5.02 (s, 2H), 4.06 (t, J=6.1, 2H), 2.76 (t, J=6.1, 2H), 2.51 (br s, 4H), 1.65-1.55 (m, 4H), 1.45 (br s, 2H).

B. 4-(2-Piperidin-1-yl-ethoxy)-phenol. To a solution of 1-[2-(4-benzyloxy-phenoxy)-ethyl]-piperidine (15.0 g, 48.2 mmol) in 1:1 EtOH/EtOAc (400 mL) was added 10% Pd/C (1.5 g). The mixture was placed on a Parr hydrogenator at 40 psi of H₂ for 20 h. The reaction mixture was filtered through diatomaceous earth, and the filtrate was concentrated to give 9.4 g (88%) of the desired product as a light gray solid. TLC (SiO₂, 50% acetone/CH₂Cl₂): R_{f}=0.16. MS (ESI): mass calculated for C₁₃H₁₉NO₂, 221.14; m/z found, 222.1 [M+H]⁺. ¹H NMR (400 MHz, DMSO-d₆): 8.88 (s, 1H), 6.73 (d, J=6.6, 2H), 6.65 (d, J=6.6, 2H), 3.93 (t, J=6.0, 2H), 2.58 (t, J=6.0, 2H), 2.40 (s, 4H), 1.51-1.45 (m, 4H), 1.35 (br s, 2H).

C. Phenyl-carbamic acid 4-(2-piperidin-1-yl-ethoxy)-phenyl ester. A solution of 4-(2-piperidin-1-yl-ethoxy)-phenol (1.0 g, 4.5 mmol), phenyl isocyanate (588 μL, 4.97 mmol), and TEA (865 μL, 6.21 mmol) in CH₂Cl₂ (20 mL) was stirred at rt for 18 h. The reaction mixture was diluted with CH₂Cl₂ (50 mL) and washed with 1 N NaOH (3×10 mL) and H₂O (1×10 mL). The organic layer was dried (Na₂SO₄), filtered and concentrated to yield the crude product as a white solid. The solid was triturated with Et₂O (100 mL) and filtered. Flash column chromatography (0-100% acetone/CH₂Cl₂) gave 1.15 g (75%) of the desired product as a white solid. MS (ESI): exact mass calculated for C₂₀H₂₄N₂O₃, 340.18; m/z found, 341.2 [M+H]⁺. ¹H NMR (400 MHz, CDCl₃): 7.47-7.45 (m, 2H), 7.38-7.34 (m, 2H), 7.12-7.08 (m, 3H), 6.95-6.91 (m, 3H), 4.11 (t, J=6.1, 2H), 2.79 (t, J=6.1, 2H), 2.56-2.47 (br t, 2H), 1.66-1.60 (m, 6H), 1.49-1.47 (m, 2H).

### Example 16

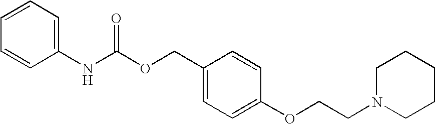

### Phenyl-carbamic acid 4-(2-piperidin-1-yl-ethoxy)-benzyl ester

A. [4-(2-Piperidin-1-yl-ethoxy)-phenyl]-methanol. To a mixture of 4-hydroxybenzyl alcohol (30 g, 241 mmol) and 1-(2-chloroethyl)piperidine hydrochloride (53 g, 289 mmol) in CH₃CN (600 mL) was added K₂CO₃ (40 g, 289 mmol) and Cs₂CO₃ (79 g, 241 mmol). The reaction mixture was stirred for 24 h at 90° C. The resulting mixture was diluted with CH₂Cl₂ (300 mL) and filtered through diatomaceous earth. The organic layer was concentrated to the crude product as a brown oil. The oil was purified on SiO₂ (300 g; 0-100% acetone/CH₂Cl₂) to give a brown oil. The oil was treated with charcoal, filtered and concentrated to provide the desired product (25.1 g, 44%) as a brown oil. MS (ESI): mass calculated for C₁₄H₂₁NO₂, 235.16; m/z found, 236.2 [M+H]⁺. ¹H NMR (400 MHz, CDCl₃): 7.29-7.27 (m, 2H), 6.90-6.88 (m, 2H), 5.31 (s, 2H), 4.10 (t, J=6.1, 2H), 2.77 (t, J=6.1, 2H), 2.53-2.46 (br t, 4H), 1.64-1.54 (m, 5H), 1.48-1.43 (m, 2H).

B. Phenyl-carbamic acid 4-(2-piperidin-1-yl-ethoxy)-benzyl ester. A solution of [4-(2-piperidin-1-yl-ethoxy)-phenyl]-methanol (750 mg, 3.2 mmol), phenyl isocyanate (380 μL, 3.5 mmol), and TEA (480 μL, 3.4 mmol) in CH₂Cl₂ (20 mL) was stirred at rt for 18 h. The reaction mixture was diluted with CH₂Cl₂ (50 mL) and washed with 1 N NaOH (3×10 mL) and H₂O (1×10 mL). The organic layer was dried (Na₂SO₄), filtered and concentrated to yield the crude product as a pale oil. The oil was purified on SiO₂ (35 g; 0-100% acetone/CH₂Cl₂) to provide the desired product (534 mg, 47%) as a white solid. MS (ESI): exact mass calculated for C₂₁H₂₆N₂O₃, 354.19; m/z found, 355.3.2 [M+H]⁺. ¹H NMR (400 MHz, CDCl₃): 7.40-7.27 (m, 6H), 7.12-7.03 (m, 1H), 6.94-6.91 (m, 2H), 6.65 (br s, 1H), 5.14 (s, 2H), 4.14 (t, J=6.1, 2H), 2.79 (t, J=6.1, 2H), 2.52 (br s, 3H), 1.66-1.59 (m, 5H), 1.50-1.42 (m, 2H).

### Example 17

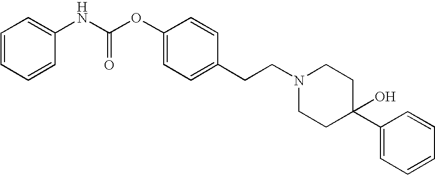

### Phenyl-carbamic acid 4-[2-(4-hydroxy-4-phenyl-piperidin-1-yl)-ethyl]-phenyl ester

A. 1-[2-(4-Hydroxy-phenyl)-ethyl]-4-phenyl-piperidin4-ol. A solution of 4-(2-bromo-ethyl)-phenol (3.6 g, 18.1 mmol), 4-phenyl-piperidin-4-ol (4.8 g, 27.1 mmol), and DIEA (4.7 mL, 27.1 mmol) in CH₃CN (75 mL) was stirred at 60° C. for 18 h. The resulting solution was cooled to rt and concentrated to yield a pale orange solid. Diethyl ether (100 mL) was added, and the desired compound was collected by filtration as a pale solid (5.4 g, 100% crude). TLC (SiO₂, 5% 2 M NH₃ in CH₃OH/CH₂Cl₂): R_{f}=0.19. MS (ESI): mass calculated for C₁₉H₂₃NO₂, 297.17; m/z found, 298.1 M+H]⁺. ¹H NMR (400 MHz, CD₃OD): 7.51-7.48 (m, 3H), 7.38-7.34 (m, 3H), 7.28-7.24 (m, 1H), 7.12 (d, J=8.4, 1H), 6.75 (d, J=8.4, 1H), 3.48-3.24 (m, 5H), 3.00-2.96 (m, 1H), 2.36-2.18 (m, 2H), 1.97-1.91 (m, 2H).

B. Phenyl-carbamic acid 4-[2-(4-hydroxy-4-phenyl-piperidin-1-yl)-ethyl]-phenyl ester. A solution of 1-[2-(4-hydroxy-phenyl)-ethyl]4-phenyl-piperidin4-ol (800 mg, 2.69 mmol), phenyl isocyanate (350 μL, 2.95 mmol), and TEA (412 μL, 2.95 mmol) in CH₂Cl₂ (5 mL) was stirred at rt for 18 h. The reaction mixture was concentrated to yield the crude product as a pale solid. The solid was purified on SiO₂ (90 g; 0-100% acetone/CH₂Cl₂) to provide a white solid, which was further purified by trituration with Et₂O. The desired product was collected by filtration as a pale solid (458 mg, 41%). MS (ESI): exact mass calculated for C₂₆H₂₈N₂O₃, 416.21; m/z found, 417.2 [M+H]⁺. ¹H NMR (400 MHz, CD₃OD): 7.52-7.46 (m, 4H), 7.36-7.22 (m, 7H), 7.16-7.11 (m, 2H), 7.06-7.03 (m, 1H), 3.21-3.14 (m, 2H), 3.02-2.69 (m, 6H), 2.26-2.18 (m, 2H), 1.88-1.84 (m, 2H).

### Example 18

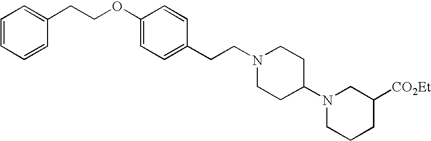

### 1′-[2-(4-Phenethyloxy-phenyl)-ethyl]-[1,4]′bipiperidinyl-3-carboxylic acid ethyl ester

A. 2-(4-Phenethyloxy-phenyl)-ethanol. To a solution of 4-(2-hydroxy-ethyl)-phenol (10 g, 72.3 mmol), and 2-bromoethyl-benzene (14.7 mL, 79.6. mmol) in CH₃CN (150 mL) was added K₂CO₃ (10 g, 72.3 mmol). The resulting mixture was stirred at rt for 72 h, followed by heating at reflux for 48 h. The resulting solution was cooled to rt, filtered to remove solids and concentrated to yield a yellow oil. The oil was purified on SiO₂ (300 g; 0-100% CH₂Cl₂/hexane) to provide 6.9 g (39%) of the desired product as a white solid. MS (ESI): mass calculated for C₁₆H₁₈O₂, 242.12; m/z found, 243.4 M+H]⁺. H NMR (400 MHz, CDCl₃): 7.35-7.25 (m, 5H), 7.15-7.13 (m, 2H), 6.88-6.86 (m, 2H), 4.17 (t, J=7.1, 2H), 3.83 (t, J=6.4, 2H), 3.11 (t, J=7.1, 2H), 2.81 (t, J=6.5, 2H), 1.37 (t, J=6.0, 1H).

B. (4-Phenethyloxy-phenyl)-acetaldehyde. A solution of 2-(4-phenethyloxy-phenyl)-ethanol (200 mg, 0.83 mmol) and Dess-Martin periodinane (650 mg, 1.53 mmol) in CH₂Cl₂ (5 mL) was stirred at rt for 1 h. Satd. aq. NaHCO₃ (5 mL) and 1.0 g Na₂S₂O₃ were added and the resulting mixture was stirred overnight at rt. The organic layer was separated and the aqueous layer further extracted with CH₂Cl₂ (3×5 mL). The combined organic layers were dried (Na₂SO₄), filtered and concentrated to yield the crude product as a pale oil. This material was used without further purification. ¹H NMR (400 MHz, CDCl₃): 9.73 (t, J=2.4, 1H), 7.34-7.25 (m, 5H), 7.14-7.11 (m, 2H), 6.92-6.89 (m, 2H), 4.18 (t, J=7.1, 2H), 3.63 (t, J=2.4, 2H), 3.11 (t, J=7.1, 2H).

C. [1,4′]Bipiperidinyl-3,1′-dicarboxylic acid 1′-tert-butyl ester 3-ethyl ester. A mixture of 4-oxo-piperidine-1-carboxylic acid tert-butyl ester (5 g, 25.1 mmol), ethyl nipecotate (4.67 mL, 30.1 mmol) and ground molecular sieves (4 Å, 5 g) in CF₃CH₂OH (20 mL) was stirred for 1 h. To this mixture was added NaBH(OAc)₃ (9.56 g, 45.1 mmol) and the resulting mixture was stirred at rt for 5 d. The reaction mixture was diluted with CH₂Cl₂, filtered through diatomaceous earth and concentrated to yield a brown oil. The brown oil was purified using SiO₂ (120 g; 0-100% acetone/CH₂Cl₂) to provide 6.9 g (81%) of the desired product as an orange oil. MS (ESI): mass calculated for C₁₈H₃₂N₂O₄, 340.2; m/z found, 341.2 [M+H]⁺. ¹H NMR (400 MHz, CDCl₃): 4.13 (q, J=7.1, 2H), 3.25-3.21 (m, 1H), 3.08-2.99 (m, 1H), 2.86-2.69 (m, 4H), 2.50-2.44 (m, 1H), 2.37-2.31 (m, 1H), 2.08-2.05 (m, 1H), 1.87-1.74 (m, 4H), 1.55-1.37 (m, 14H), 1.25 (t, J=7.1, 3H).

D. [1,4′]Bipiperidinyl-3-carboxylic acid ethyl ester. To a solution of [1,4′]bipiperidinyl-3,1′-dicarboxylic acid 1′-tert-butyl ester 3-ethyl ester (6.9 g, 20.3 mmol) in CH₂Cl₂ (100 mL) was added 25.4 mL 4 N HCl (101 mmol). The resulting solution was stirred for 18 h. The reaction mixture was concentrated to yield a solid. The solid was tritrated with Et₂O and collected by filtration to yield 4.6 g of the desired product (97%). MS (ESI): mass calculated for C₁₆H₂₈N₂O₄, 240.2; m/z found, 241.2 [M+H]⁺. ¹H NMR (400 MHz, CDCl₃): 4.18 (q, J=7.1, 2H), 3.77-3.58 (m, 6H), 3.37-3.00 (m, 2H), 2.45-2.42 (m, 4H), 2.23-1.90 (m, 6H), 1.76-1.58 (m, 1H), 1.27 (t, J=7.1, 3H).

E. 1′-[2-(4-Phenethyloxy-phenyl)-ethyl-[1,4′]bipiperidinyl-3-carboxylic acid ethyl ester. A mixture of (4-phenethyloxy-phenyl)-acetaldehyde (500 mg, 2.1 mmol) and [1,4′]bipiperidinyl-3-carboxylic acid ethyl ester (2.5 mmol) in dichloroethane (5 mL) was stirred at rt for 1 h. To the resulting mixture was added NaBH(OAc)₃ (668 mg, 3.2 mmol) and the mixture was stirred at rt for 6 d. The reaction mixture was diluted with CH₂Cl₂ (20 mL) and washed with satd. aq. NaHCO₃ (1×10 mL). The organic layer was dried (Na₂SO₄), filtered and concentrated to yield a brown oil. The oil was purified on SiO₂ (40 g; 0-10% 2 M NH₃ in CH₃OH/CH₂Cl₂) to provide 390 mg (41%) of the desired product as a yellow oil. MS (ESI): mass calculated for C₂₉H₄₀N₂O₃, 464.3; m/z found, 465.4 [M+H]⁺. ¹H NMR (400 MHz, CDCl₃): 7.34-7.22 (m, 5H), 7.11-7.08 (m, 2H), 6.84-6.81 (m, 2H), 4.17-4.11 (m, 4H), 3.10-3.01 (m, 5H), 2.81-2.72 (m, 3H), 2.54-2.51 (m, 3H), 2.42-2.33 (m, 1H), 2.26-2.21 (m, 1H), 2.01-1.92 (m, 3H) 1.76-1.43 (m, 8H), 1.26 (t, J=7.1, 2H).

### Example 19

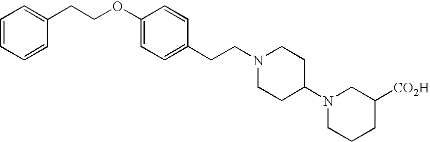

### 1′-[2-(4-Phenethyloxy-phenyl)-ethyl]-[1,4′]bipiperidinyl-3-carboxylic acid

To a stirring solution of 1′-[2-(4-phenethyloxy-phenyl)-ethyl]-[1,4′]bipiperidinyl-3-carboxylic acid ethyl ester (350 mg, 0.75 mmol) in THF (10 mL) was added potassium trimethylsilanoate (386 mg, 3.01 mmol). The reaction mixture was stirred at rt for 6 h, then stored overnight at 5° C. The reaction mixture was concentrated. The resultant semi-solid was dissolved in H₂O (3 mL), and the solution was adjusted to pH 5 with 1 M HCl. The resulting solution was extracted with 1:3 i-PrOH/CHCl₃ (3×25 mL). The combined extracts were concentrated to yield a white solid that was triturated with Et₂O and filtered to afford a white solid (232 mg, 71%). MS (ESI): mass calculated for C₂₇H₃₆N₂O₃, 436.6; m/z found, 437.4 [M+H]⁺. ¹H NMR (400 MHz, CDCl₃): 7.27 (d, J=4.3, 4H), 7.22-7.16 (m, 3H), 7.27 (d, J=8.5, 2H), 4.15 (t, J=4.3, 2H), 3.65-3.54 (br d, 2H), 3.30-3.12 (m, 6H), 3.04 (t, J=6.8, 2H), 2.96-2.87 (m, 5H), 2.74-2.68 (m, 1H), 2.26-2.18 (m, 2H), 2.08-1.91 (m, 4H) 1.82-1.72 (m, 2H).

### Example 20

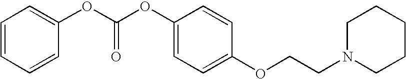

### Carbonic acid phenyl ester 4-(2-piperidin-1-yl-ethoxy)-phenyl ester

To a mixture of 4-(2-piperidin-1-yl-ethoxy)-phenol (221 mg, 1.0 mmol) and phenyl chloroformate (151 μL, 1.2 mmol) in CH₂Cl₂ (10 mL) was added TEA (279 μL, 2 mmol). The reaction mixture was stirred at rt for 16 h. To the mixture was added CH₂Cl₂ (100 mL). The organic layer was washed with H₂O, dried (MgSO₄), filtered, and concentrated to give a clear liquid, which was purified on SiO₂ (10 g; 0-10% CH₃OH/CH₂Cl₂) to give a light brown solid (340 mg, 99%). TLC (SiO₂, 10% CH₃OH/CH₂Cl₂): R_{f}=0.60. MS (ESI): mass calculated for C₂₀H₂₃NO₄, 341.41; m/z found, 342.4 [M+H]⁺. ¹H NMR (400 MHz, CDCl₃): 7.42-7.34 (m, 2H), 7.28-7.22 (m, 3H), 7.19-7.12 (m, 2H), 6.93-6.86 (m, 2H), 4.07 (t, J=5.9, 2H), 2.74 (t, J=5.9, 2H), 2.47 (br s, 4H), 1.63-1.55 (m, 4H), 1.46-1.38 (m, 2H).

### Example 21

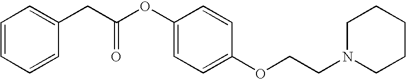

### Phenyl-acetic acid 4-(2-piperidin-1-yl-ethoxy)-phenyl ester

To a mixture of 4-(2-piperidin-1-yl-ethoxy)-phenol (221 mg, 1.0 mmol) and phenyl-acetyl chloride (159 μL, 1.2 mmol) in CH₂Cl₂ (10 mL) was added TEA (279 μL, 2 mmol). The reaction mixture was stirred at rt for 16 h. To the mixture was added CH₂Cl₂ (100 mL). The organic layer was washed with H₂O, dried (MgSO₄), filtered, and concentrated to give a clear liquid, which was purified on SiO₂ (10 g; 0-10% CH₃OH/CH₂Cl₂) to give a light yellow oil (303 mg, 89%). TLC (SiO₂, 10% CH₃OH/CH₂Cl₂): R_{f}=0.60. MS (ESI): mass calculated for C₂₁H₂₅NO₃, 339.44; m/z found, 340.4 [M+H]⁺. ¹H NMR (400 MHz, CD₃OD): 7.39-7.32 (m, 4H), 7.32-7.25 (m, 1H), 7.00 (dd, J=13.1, 9.2, 4H), 4.26 (t, J=4.5, 2H), 3.87 (s, 2H), 3.54 (d, J=12.3, 2H), 3.45 (t, J=4.5, 2H), 2.96 (t, J=11.7, 2H), 1.92-1.71 (m, 5H), 1.54-1.40 (m, 1H).

### Example 22

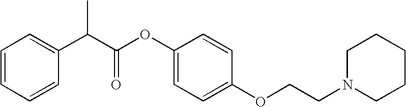

### 2-Phenyl-propionic acid 4-(2-piperidin-1-yl-ethoxy)-phenyl ester

To a mixture of 4-(2-piperidin-1-yl-ethoxy)-phenol (221 mg, 1.0 mmol) and 2-phenyl-propionic acid (164 μL, 1.2 mmol) in CH₂Cl₂ (10 mL) was added 1-(3-dimethylaminopropyl)-3-ethylcarbodiimide hydrochloride (EDCl, 288 mg, 1.5 mmol). The reaction mixture was stirred at rt for 16 h. To the mixture was added CH₂Cl₂ (100 mL). The organic layer was washed with H₂O, dried (MgSO₄), filtered, and concentrated to give a clear liquid, which was purified on SiO₂ (10 g; 0-10% CH₃OH/CH₂Cl₂) to give a light yellow oil (208 mg, 59%). TLC (SiO₂, 10% CH₃OH/CH₂Cl₂): R_{f}=0.65. MS (ESI): mass calculated for C₂₂H₂₇NO₃, 353.47; m/z found, 354.4 [M+H]⁺. ¹H NMR (400 MHz, CDCl₃): 7.41-7.32 (m, 4H), 7.31-7.23 (m, 1H), 6.86 (dd, J=10.8, 9.4, 4H), 4.05 (t, J=6.1, 2H), 3.93 (dd, J=7.2, 7.0, 1H), 2.73 (t, J=6.1, 2H), 2.47 (br s, 4H), 1.62-1.55 (m, 7H), 1.46-1.38 (m, 2H).

### Example 23

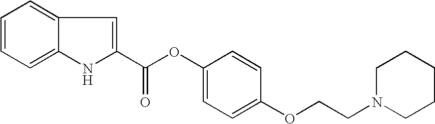

### 1H-Indole-2-carboxylic acid 4-(2-piperidin-1-yl-ethoxy)-phenyl ester

To a mixture of 4-(2-piperidin-1-yl-ethoxy)-phenol (221 mg, 1.0 mmol) and 1H-indole-2-carboxylic acid (240 mg, 1.5 mmol) in CH₂Cl₂ (10 mL) was added EDCl (288 mg, 1.5 mmol). The reaction mixture was stirred at rt for 16 h. To the mixture was added CH₂Cl₂ (100 mL). The organic layer was washed with H₂O, dried (MgSO₄), filtered, and concentrated to give a clear liquid, which was purified on SiO₂ (10 g; 0-10% CH₃OH/CH₂Cl₂) to give a white solid (160 mg, 44%). TLC (SiO₂, 10% CH₃OH/CH₂Cl₂): R_{f}=0.55. MS (ESI): mass calculated for C₂₂H₂₄N₂O₃, 364.45; m/z found, 365.4 [M+H]⁺. ¹H NMR (400 MHz, CDCl₃): 9.56 (s, 1H), 7.71 (d, J=8.2, 1H), 7.41 (s, 1H), 7.39-7.28 (m, 2H), 7.16 (t, J=7.2, 1H), 7.11 (d, J=9.0, 2H), 6.90 (d, J=9.0, 2H), 4.15 (t, J=6.1, 2H), 2.79 (t, J=5.9, 2H), 2.53 (br s, 4H), 1.66-1.56 (m, 4H), 1.49-1.40 (m, 2H).

### Example 24

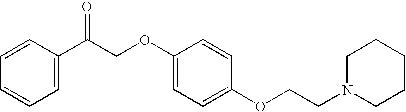

### 1-Phenyl-2-[4-(2-piperidin-1-yl-ethoxy)-phenoxy]-ethanone

To a mixture of 2-bromo-1-phenyl-ethanone (119 mg, 1.0 mmol) and 4-(2-piperidin-1-yl-ethoxy)-phenol (332 mg, 1.5 mmol) in acetone (10 mL) was added Cs₂CO₃ (652 mg, 2 mmol). The reaction mixture was stirred at rt for 16 h. To the mixture was added CH₂Cl₂ (100 mL). The organic layer was washed with H₂O, dried (MgSO₄), filtered, and concentrated to give a brown liquid, which was purified on SiO₂ (10 g; 0-10% CH₃OH/CH₂Cl₂) to give a light yellow solid (175 mg, 51%). TLC (SiO₂, 10% CH₃OH/CH₂Cl₂): R_{f}=0.50. MS (ESI): mass calculated for C₂₁H₂₅NO₃, 339.44; m/z found, 340.4 [M+H]⁺. ¹H NMR (400 MHz, CDCl₃): 8.00 (d, J=7.2, 2H), 7.61 (t, J=7.4, 1H), 7.49 (t, J=7.6, 2H), 6.85 (dd, J=12.1, 9.0, 4H), 5.22 (s, 2H), 4.04 (t, J=6.1, 2H), 2.74 (t, J=6.1, 2H), 2.49 (br s, 4H), 1.63-1.55 (m, 4H), 1.47-1.39 (m, 2H).

### Example 25

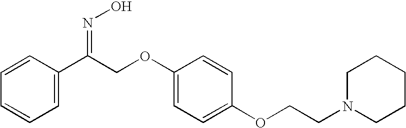

### 1-Phenyl-2-[4-(2-piperidin-1-yl-ethoxy)-phenoxy]-ethanone oxime

To a stirred solution of 1-phenyl-2-[4-(2-piperidin-1-yl-ethoxy)-phenoxy]-ethanone (340 mg, 1.0 mmol) in pyridine (10 mL) was added hydroxylamine hydrochloride (104 mg, 1.5 mmol)., The reaction mixture was stirred at rt for 16 h. To the mixture was added CH₂Cl₂ (100 mL). The organic layer was washed with H₂O and satd. aq. NaHCO₃, dried (MgSO₄), filtered, and concentrated to give a clear liquid, which was purified on SiO₂ (10 g; 0-10% CH₃OH/CH₂Cl₂) to give a white solid (173 mg, 49%). TLC (SiO₂, 10% CH₃OH/CH₂Cl₂): R_{f}=0.50. MS (ESI): mass calculated for C₂₁H₂₆N₂O₃, 354.45; m/z found, 355.4 [M+H]⁺.

¹H NMR (400 MHz, CDCl₃): 7.68-7.61 (m, 2H), 7.33-7.27 (m, 3H), 6.80 (dd, J=11.0, 9.2, 4H), 5.19 (s, 2H), 4.09 (t, J=5.9, 2H), 2.79 (t, J=5.9, 2H), 2.59 (br s, 4H), 1.72-1.64 (m, 4H), 1.51-1.42 (m, 2H).

### Example 26

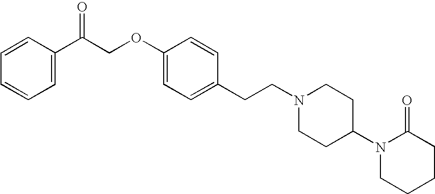

### 1′-{2-[4-(2-Oxo-2-phenyl-ethoxy)-phenyl]-ethyl}-[1,4′]bipiperidinyl-2-one

A. 1′-[2-(4-Hydroxy-phenyl)-ethyl-[1,4′]bipiperidinyl-2-one. To a stirred solution of 4-(2-bromo-ethyl)-phenol (7.3 g, 36.2 mmol) in CH₃CN (150 mL) was added [1,4′]bipiperidinyl-2-one (5.28 g, 24.1 mmol), followed by DIEA (10.5 mL, 60.3 mmol). The resulting solution was stirred overnight at 60° C., yielding a suspension. The suspension was filtered, and the filtrate was concentrated. To the resultant oil was added Et₂O, and the mixture was warmed to reflux for 2 min, forming a white precipitate. This suspension was stirred at rt for 2 h, then filtered, giving 6.54 g (90%) of an off-white solid. MS (ESI): mass calculated for C₁₈H₂₆N₂O₂, 302.42; m/z found, 303.4 [M+H]⁺. ¹H NMR (400 MHz, CD₃OD): 6.99 (d, J=8.4, 2H), 6.76 (d, J=8.4, 2H), 4.60-4.51 (m, 1H), 3.21-3.14 (m, 2H), 3.05 (d, J=11.5, 2H), 2.70 (dd, J=6.6, 5.3, 2H), 2.53 (dd, J=4.9, 4.9, 2H), 2.42 (t, J=6.1, 2H), 2.07 (t, J=11.4, 2H), 1.85-1.69 (m, 6H), 1.61 (d, J=11.9, 2H).

B. 1′-{2-[4-(2-Oxo-2-phenyl-ethoxy)-phenyl]-ethyl}-[1,4′]bipiperidinyl-2-one.

To a mixture of 2-bromo-1-phenyl-ethanone (1.32 g, 6.6 mmol) and 1′-[2-(4-hydroxy-phenyl)-ethyl]-[1,4′]bipiperidinyl-2-one (1.0 mg, 3.3 mmol) in acetone (26 mL) was added Cs₂CO₃ (2.15, 6.6 mmol). The reaction mixture was stirred at rt for 16 h. To the mixture was added CH₂Cl₂ (200 mL). The organic layer was washed with H₂O, dried (MgSO₄), filtered, and concentrated to give a brown liquid, which was purified on SiO₂ (40 g; 0-10% CH₃OH/CH₂Cl₂) to give a yellow oil (2.42 g, 87%). TLC (SiO₂, 10% CH₃OH/CH₂Cl₂): R_{f}=0.33. MS (ESI): mass calculated for C₂₆H₃₂N₂O₃, 420.56; m/z found, 421.2 [M+H]⁺. ¹H NMR (400 MHz, CDCl₃): 7.97 (d, J=7.1, 2H), 7.58 (t, J=7.3, 1H), 7.46 (t, J=7.3, 2H), 7.09 (d, J=9.1, 2H), 6.85 (d, J=9.1, 2H), 5.22 (s, 2H), 4.60-4.49 (m, 1H), 3.17 (t, J=5.3, 2H), 3.02 (d, J=11.4, 2H), 2.75-2.67 (m, 2H), 2.58-2.48 (m, 2H), 2.37 (t, J=6.6, 2H), 2.12 (dd, J=11.1, 4.8, 2H), 1.79-1.67 (m, 6H), 1.60 (d, J=11.1, 2H).

### Example 27

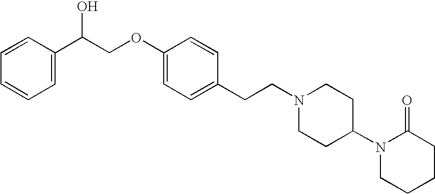

### 1′-{2-[4-(2-Hydroxy-2-phenyl-ethoxy)-phenyl]-ethyl}-[1,4′]bipiperidinyl-2-one

To a stirred solution of 1′-{2-[4-(2-oxo-2-phenyl-ethoxy)-phenyl]-ethyl}-[1,4′]bipiperidinyl-2-one (700 mg, 1.66 mmol) in EtOH (33 mL) was added sodium borohydride (126 mg, 3.33 mmol). The resulting solution was stirred overnight at rt. To the mixture was added CH₂Cl₂ (100 mL). The organic layer was washed with H₂O, dried (MgSO₄), filtered, and concentrated to give a brown liquid, which was purified on SiO₂ (40 g; 0-10% CH₃OH/CH₂Cl₂) to give a white solid (421 mg, 60%). TLC (SiO₂, 10% CH₃OH/CH₂Cl₂): R_{f}=0.35. MS (ESI): mass calculated for C₂₆H34N₂O₃, 422.57; m/z found, 423.2 [M+H]⁺. ¹H NMR (400 MHz, CDCl₃): 7.44 (d, J=7.1, 2H), 7.33 (t, J=7.1, 2H), 7.28 (dd, J=7.3, 6.6, 1H), 7.03 (d, J=8.6, 2H), 6.80 (d, J=8.6, 2H), 5.05 (t, J=6.1, 1H), 4.92 (br s, 1H), 4.56-4.45 (m, 1H), 4.06-3.98 (m, 2H), 3.09 (br s, 2H), 2.98 (d, J=11.1, 2H), 2.69 (dd, J=7.3,4.29, 2H), 2.49 (dd, J=5.3,5.0, 2H), 2.33 (t, J=5.3, 2H), 2.09 (t, J=11.1, 2H), 1.79-1.63 (m, 6H), 1.55 (d, J=10.6, 2H).

### Example 28

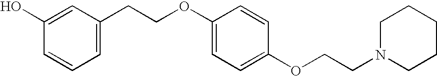

### 3-{2-[4-(2-Piperidin-1-yl-ethoxy)-phenoxy]-ethyl}-phenol

To a mixture of 3-(2-hydroxy-ethyl)-phenol (276 mg, 2.0 mmol) and 4-(2-piperidin-1-yl-ethoxy)-phenol (221 mg, 1.0 mmol) in toluene (20 mL) was added triphenylphosphine (534 mg, 2 mmol) and diethyl azodicarboxylate (364 μL, 2 mmol). The reaction mixture was stirred at rt for 2 h. To the mixture was added CH₂Cl₂ (100 mL). The organic layer was washed with H₂O, dried (MgSO₄), filtered, and concentrated to give a brown liquid, which was purified on SiO₂ (40 g; 0-10% CH₃OH/CH₂Cl₂) to give a light yellow oil (220 mg, 64%). MS (ESI): mass calculated for C₂₁H₂₇NO₃, 341.45; m/z found, 342.4 [M+H]⁺. ¹H NMR (400 MHz, CDCl₃): 7.13-6.99 (m, 1H), 6.88 (dd, J=13.7, 9.0, 4H), 6.75-6.68 (m, 2H), 6.64-6.59 (m, 1H), 4.26 (t, J=5.1, 2H), 4.09 (t, J=6.8, 2H), 3.59 (d, J=12.1, 2H), 3.50 (t, J=4.9, 2H), 3.03 (t, J=11.5, 2H), 2.94 (t, J=7.0, 2H), 1.99-1.89 (m, 2H), 1.87-1.72 (m, 3H), 1.59-1.47 (m, 1H).

### Example 29

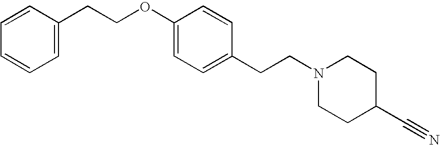

### 1-[2-(4-Phenethyloxy-phenyl)-ethyl]-piperidine-4-carbonitrile

To a stirred solution of 1-(4-phenethyloxy-phenyl)-ethyl bromide (1.83 g, 6 mmol) in CH₃CN (24 mL) was piperidine-4-carbonitrile. (881 mg, 8 mmol) and DIEA (2.09 mL, 12 mmol). The resulting solution was stirred overnight at 60° C. The mixture was cooled to rt. To the mixture was added CH₂Cl₂ (200 mL). The organic layer was washed with H₂O, dried (MgSO₄), filtered, and concentrated to give a clear liquid, which was purified on SiO₂ (110 g; 0-10% CH₃OH/CH₂Cl₂) to give a white solid (1.76 g, 88%). TLC (SiO₂, 10% CH₃OH/CH₂Cl₂): R_{f}=0.75. MS (ESI): mass calculated for C₂₂H₂₆N₂O, 334.45; m/z found, 335.4 [M+H]⁺. ¹H NMR (400 MHz, CDCl₃): 7.23-7.08 (m, 5H), 6.98 (d, J=8.6, 2H), 6.71 (d, J=8.6, 2H), 4.02 (t, J=7.1, 2H), 2.96 (t, J=7.1, 2H), 2.64-2.54 (m, 4H), 2.52-2.40 (m, 2H), 2.31-2.19 (m, 2H), 1.86-1.69 (m, 4H).

### Example 30

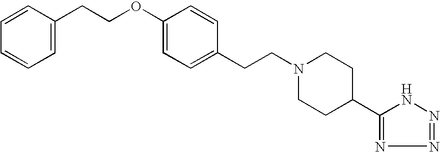

### 1-[2-(4-Phenethyloxy-phenyl)-ethyl]-4-(1H-tetrazol-5-yl)-piperidine

To a stirred solution of 1-[2-(4-phenethyloxy-phenyl)-ethyl]-piperidine-4-carbonitrile (0.50 g, 1.49 mmol) in toluene (10 mL), was added trimethylaluminum (2.0 M in hexanes, 3.7 mL, 7.47 mmol) and trimethylsilyl azide (982 μL, 7.47 mmol). The resulting solution was stirred overnight at 80° C. The mixture was cooled to rt. To the mixture was added CH₂Cl₂ (100 mL). The organic layer was washed with H₂O and satd. aq. NaHCO₃, dried (MgSO₄), filtered, and concentrated to give a light yellow solid, which was purified on SiO₂ (40 g; 0-10% CH₃OH/CH₂Cl₂) to give a white solid (503 mg, 89%). TLC (SiO₂, 15% CH₃OH/CH₂Cl₂): R_{f}=0.4. MS (ESI): mass calculated for C₂₂H₂₇N₅O, 377.48; m/z found, 378.4 [M+H]⁺. ¹H NMR (400 MHz, CDCl₃): 7.25-7.10 (m, 5H), 7.00 (d, J=8.8, 2H), 6.73 (d, J=8.8, 2H), 4.21 (br s, 1H), 4.05 (t, J=7.1, 2H), 3.02-2.09 (m, 4H), 2.83-2.73 (m, 1H), 2.70-2.62 (m, 2H), 2.52-2.44 (m, 2H), 2.07 (t, J=10.6, 2H), 1.93-1.85 (m, 2H), 1.83-1.71 (m, 2H).

### Example 31

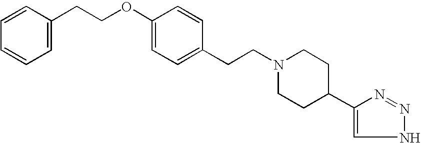

### 1-[2-(4-Phenethyloxy-phenyl)-ethyl]-4-(1H-[1,2,3]triazol-4-yl)-piperidine

A. 1-[2-(4-Phenethyloxy-phenyl)-ethyl]-4-(5-trimethylsilanyl-1H-[1,2,3]triazol-4-yl)-piperidine. n-Butyllithium (2.5 M in hexane, 3.0 mL, 7.5 mmol) was added dropwise to a solution of trimethylsilyldiazomethane (3.6 mL, 7.2 mmol) in Et₂O (30 mL) at 0° C. under nitrogen and the mixture was stirred for 20 min at 0° C. To the resulting solution was added dropwise a solution of 1-[2-(4-phenethyloxy-phenyl)-ethyl]-piperidine-4-carbonitrile (1.0 g, 3 mmol) in THF (10 mL) at 0° C., then the mixture was stirred for 3 h at 0° C. The mixture was treated with satd. aq. NH₄Cl and extracted with CH₂Cl₂. The organic extracts was washed with H₂O and satd. aq. NaHCO₃, dried (MgSO₄), filtered, and concentrated to give a light yellow solid, which was purified on SiO₂ (40 g; 0-10% CH₃OH/CH₂Cl₂) to give a white solid (765 mg, 57%). TLC (SiO₂, 10% CH₃OH/CH₂Cl₂): R_{f}=0.60. MS (ESI): mass calculated for C₂₆H₃₆N₄OSi, 448.68; m/z found, 449.4 [M+H]⁺. ¹H NMR (400 MHz, CDCl₃): 7.18-7.04 (m, 5H), 6.95 (d, J=8.3, 2H), 6.66 (d, J=8.3, 2H), 3.99 (t, J=7.3, 2H), 3.03 (d, J=9.4, 2H), 2.92 (t, J=7.1, 2H), 2.69-2.60 (m, 3H), 2.53-2.45 (m, 2H), 2.09-1.91 (m, 4H), 1.77-1.67 (m, 2H), 0.21 (s, 9H).

B. 1-[2-(4-Phenethyloxy-phenyl)-ethyl-4-(1H-[1,2,3]triazol-4-yl)-piperidine. To a stirred solution of 1-[2-(4-phenethyloxy-phenyl)-ethyl]-4-(5-trimethylsilanyl-1H-[1,2,3]triazol-4-yl)-piperidine (755 mg, 1.68 mmol) in CH₃OH (17 mL) was added ammonium fluoride (0.5 M in CH₃OH, 17 mL, 8.4 mmol). The reaction mixture was heated to 50° C. for 18 h, then was cooled to rt and concentrated. The residue was dissolved in CH₂Cl₂ and washed with H₂O, dried (MgSO₄), filtered, and concentrated to give a light yellow solid, which was purified on SiO₂ (10 g; 0-15% CH₃OH/CH₂Cl₂) to provide a white solid (493 mg, 78%). TLC (SiO₂, 10% CH₃OH/CH₂Cl₂): R_{f}=0.40. MS (ESI): mass calculated for C₂₃H₂₈N₄O, 376.49; m/z found, 377.4 [M+H]⁺. ¹H NMR (400 MHz, CDCl₃): 7.58 (s, 1H), 7.39-7.26 (m, 5H), 7.16 (d, J=8.8, 2H), 6.88 (d, J=8.8, 2H), 4.19 (t, J=7.3, 2H), 3.21 (d, J=11.6, 2H), 3.13 (t, J=7.1, 2H), 2.95-2.85 (m, 3H), 2.76-2.68 (m, 2H), 2.30 (t, J=10.6, 2H), 2.17-2.08 (m, 2H), 2.05-1.91 (m, 2H).

### Example 32

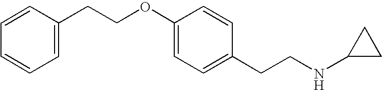

### Cyclopropyl-[2-(4-phenethyloxy-phenyl)-ethyl]-amine

To a stirred solution of 1-(4-phenethyloxy-phenyl)-ethyl bromide (6.4 g, 21 mmol) in CH₃CN (100 mL) was added cyclopropylamine (12 g, 210 mmol) and DIEA (11 mL, 63 mmol). The resulting solution was stirred overnight at 60° C. The mixture was cooled to rt. To the mixture was added CH₂Cl₂ (400 mL). The organic layer was washed with H₂O, dried (MgSO₄), filtered, and concentrated to give a clear liquid, which was purified on SiO₂ (330 g; 0-10% CH₃OH/CH₂Cl₂) to give a white solid (5.2 g, 88%). TLC (SiO₂, 10% CH₃OH/CH₂Cl₂): R_{f}=0.5. MS (ESI): mass calculated for C₁₉H₂₃NO, 281.39; m/z found, 282.3 [M+H]⁺. ¹H NMR (400 MHz, CDCl₃): 7.19-7.03 (m, 5H), 6.96 (d, J=8.6, 2H), 6.69 (d, J=8.6, 2H), 3.99 (t, J=7.1, 2H), 2.93 (t, J=7.1, 2H), 2.77 (t, J=7.1, 2H), 2.58 (t, J=7.1, 2H), 2.00-1.92 (m, 1H), 0.31-0.24 (m, 2H), 0.23-0.16 (m, 2H).

### Example 33

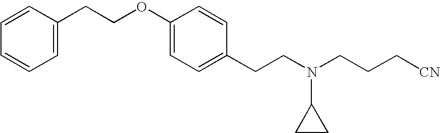

### 4-{Cyclopropyl-[2-(4-phenethyloxy-phenyl)-ethyl]-amino}-butyronitrile

To a stirred solution of cyclopropyl-[2-(4-phenethyloxy-phenyl)-ethyl]-amine (800 mg, 2.84 mmol) in CH₃CN (30 mL) was added 4-bromobutyronitrile (842 mg, 5.69 mmol) and DIEA (0.99 mL, 5.69 mmol). The resulting solution was stirred overnight at 60° C. The mixture was cooled to rt. To the mixture was added CH₂Cl₂ (100 mL). The organic layer was washed with H₂O, dried (MgSO₄), filtered, and concentrated to give a clear liquid, which was purified on SiO₂ (40 g; 10-100% EtOAc/hexanes) to give a clear oil (874 mg, 88%). TLC (SiO₂, 50% EtOAc/hexanes): R_{f}=0.70. MS (ESI): mass calculated for C₂₃H₂₈N₂O, 348.48; m/z found, 349.3 [M+H]⁺. ¹H NMR (400 MHz, CDCl₃): 7.40-7.26 (m, 5H), 7.13 (d, J=8.6, 2H), 6.88 (d, J=8.6, 2H), 4.20 (t, J=7.1, 2H), 3.14 (t, J=7.1, 2H), 2.84-2.75 (m, 6H), 2.33 (t, J=7.1, 2H), 1.90-1.82 (m, 3H), 0.59-0.52 (m, 2H), 0.47-0.40 (m, 2H).

### Example 34

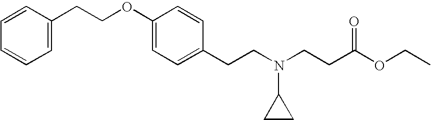

### 3-{Cyclopropyl-[2-(4-phenethyloxy-phenyl)-ethyl]-amino}-propionic acid ethyl ester

To a stirred solution of cyclopropyl-[2-(4-phenethyloxy-phenyl)-ethyl]-amine (800 mg, 2.84 mmol) in CH₃CN (30 mL) was added 4-ethyl 3-bromopropionate (1.03 mg, 5.69 mmol) and DIEA (0.99 mL, 5.69 mmol). The resulting solution was stirred overnight at 60° C. The mixture was cooled to rt. To the mixture was added CH₂Cl₂ (100 mL). The organic layer was washed with H₂O, dried (MgSO₄), filtered, and concentrated to give a clear liquid, which was purified on SiO₂ (40 g; 10-100% EtOAc/hexanes) to give a clear oil (958 mg, 82%). TLC (SiO₂, 10% CH₃OH/CH₂Cl₂): R_{f}=0.75. MS (ESI): mass calculated for C₂₄H₃₁NO₃, 381.51; m/z found, 382.2 [M+H]⁺. ¹H NMR (400 MHz, CDCl₃): 7.33-7.19 (m, 5H), 7.07 (d, J=8.6, 2H), 6.80 (d, J=8.6, 2H), 4.16-4.07 (m, 4H), 3.07 (t, J=7.1, 2H), 3.00 (t, J=7.1, 2H), 2.82-2.68 (m, 4H), 2.53 (t, J=7.1, 2H), 1.81-1.73 (m,1H), 1.24 (t, J=7.1, 3H), 0.50-0.43 (m, 2H), 0.42-0.35 (m, 2H).

### Example 35

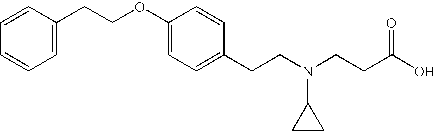

### 3-{Cyclopropyl-[2-(4-phenethyloxy-phenyl)-ethyl]-amino}-propionic acid trifluoroacetic acid salt

To a solution of 3-{cyclopropyl-[2-(4-phenethyloxy-phenyl)-ethyl]-amino}-propionic acid ethyl ester (330 mg, 0.8 mmol) in 3:1 THF/CH₃OH (20 mL), was added LiOH (77 mg, 3.2 mmol) in H₂O (10 mL). This light yellow solution was stirred at rt for 16 h and then concentrated. The residue was dissolved in CH₃OH and purified by reversed-phase HPLC to give the TFA salt of the desired product as a clear oil (334 mg, 84%). MS (ESI): mass calculated for C₂₂H₂₇NO₃, 353.45; m/z found, 354.2 [M+H]⁺. ¹H NMR (400 MHz, CD₃OD): 7.30-7.22 (m, 5H), 7.18 (d, J=8.6, 2H), 6.85 (d, J=8.6, 2H), 4.10 (t, J=7.1, 2H), 3.56 (t, J=7.1, 2H), 3.42-3.34 (m, 2H), 3.07-2.96 (m, 4H), 2.81-2.74 (m, 3H), 1.10-1.03 (m, 2H), 0.98-0.91 (m, 2H).

### Example 36

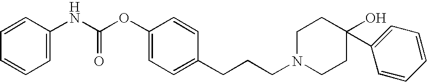

### Phenyl-carbamic acid 4-[3-(4-hydroxy-4-phenyl-piperidin-1-yl)-propyl]-phenyl ester

The title compound was prepared according to the procedure for EXAMPLE 12 using phenyl isocyanate and 4-phenyl-4-hydroxypiperidine. MS (ESI): mass calculated for C₂₇H₃₀N₂O₃, 430.23; m/z found, 431.2 [M+H]⁺. ¹H NMR (400 MHz, DMSO-d₆): 10.18 (s, 1H), 7.50 (t, J=8.3, 4H), 7.38-7.23 (m, 5H), 7.20 (t, J=7.2, 2H), 7.12 (d, J=8.4, 2H), 7.05 (t, J=7.4,1H), 4.76 (s, 2H), 2.69-2.58 (m, 4H), 2.41-2.28 (m, 4H), 1.93 (dt, J=12.9, 3.9, 2H), 1.85-1.70 (m, 2H), 1.57 (d, J=12.2, 2H).

### Example 37

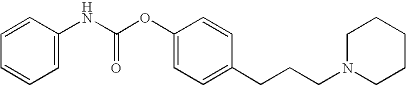

### Phenyl-carbamic acid 4-(3-piperidin-1-yl-propyl)-phenyl ester

The title compound was prepared according to the procedure for EXAMPLE 12 using phenyl isocyanate and piperidine. MS (ESI): mass calculated for C₂₁H₂₆N₂O₂, 338.20; m/z found, 339.1 [M+H]⁺. ¹H NMR (400 MHz, DMSO-d₆): 10.19 (s, 1H), 7.52 (d, J=7.9, 2H), 7.32 (t, J=7.6, 2H), 7.24 (d, J=8.4, 2H), 7.11 (d, J=8.4, 2H), 7.05 (t, J=7.4, 1H), 2.59 (t, J=7.6, 2H), 2.31 (s, 4H), 2.25 (t, J=7.2, 2H), 1.82-1.65 (m, 2H), 1.55-1.42 (m, 4H), 1.41-1.30 (m, 2H).

### Example 38

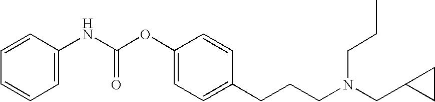

### Phenyl-carbamic acid 4-[3-(cyclopropylmethyl-propyl-amino)-propyl]-phenyl ester hydrochloride

The title compound was prepared according to the procedure for EXAMPLE 12 using phenyl isocyanate and N-propylcyclopropanemethylamine. MS (ESI): mass calculated for C₂₃H₃₀N₂O₂, 366.23; m/z found, 367.2 [M+H]⁺. ¹H NMR (400 MHz, DMSO-d₆): 10.21 (s,1H), 9.76 (s, 12H), 7.51 (d, J=7.9, 2H), 7.40-7.22 (m, 4H), 7.16 (d, J=8.5, 2H), 7.05 (t, J=7.4, 1H), 3.21-2.93 (m, 7H), 2.66 (t, J=7.5, 2H), 2.05-1.90 (m, 2H), 1.72-1.59 (m, 2H), 0.91 (t, J=7.3, 3H), 0.60 (d, J=6.4, 2H), 0.36 (d, J=4.5, 2H).

### Example 39

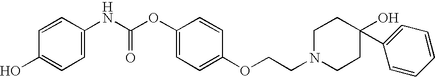

### (4—hydroxy-phenyl)-carbamic acid 4-[2-(4-hydroxy-4-phenyl-piperidin-1-yl)-ethoxy]-phenyl ester

The title compound was prepared according to the procedure for EXAMPLE 11 using 4-benzyloxyphenylamine and 4-phenyl-4-hydroxypiperidine. MS (ESI): mass calculated for C₂₆H₂₈N₂O₅, 448.20; m/z found, 449.4 [M+H]⁺. ¹H NMR (400 MHz, DMSO-d₆): 9.18 (s, 1H), 9.76 (s, 12H), 7.49 (d, J=7.4, 2H), 7.40-7.22 (m, 4H), 7.20 (t, J=7.2,1H), 7.09 (d, J=9.0, 2H), 6.96 (d, J=9.0, 2H), 6.70 (d, J=8.8, 2H), 4.80 (s, 1H), 4.10 (t, J=5.7, 2H), 2.80-2.70 (m, 4H), 2.60-2.48 (m, 2H), 1.95 (t, J=12.4, 2H), 1.58 (d, J=12.3, 2H).

### Example 40

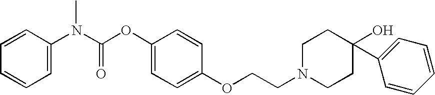

### Methyl-phenyl-carbamic acid 4-[2-(4-hydroxy-4-phenyl-piperidin-1-yl)-ethoxy]-phenyl ester

The title compound was prepared according to the procedure for EXAMPLE 8 using N-methyl-N-phenylcarbamoyl chloride and N-phenyl4-hydroxypiperidine. MS (ESI): mass calculated for C₂₇H₃₀N₂O₄, 446.22; m/z found, 447.2 [M+H]⁺. ¹H NMR (400 MHz, DMSO-d₆): 7.55-7.35 (m, 6H), 7.34-7.23 (m, 3H), 7.20 (t, J=7.3,1H), 7.05 (d, J=8.8, 2H), 6.93 (d, J=9.0, 2H), 4.79 (s, 1H), 4.08 (t, J=5.8, 2H), 2.78-2.68 (m, 4H), 2.55-2.48 (m, 2H), 1.95 (dt, J=12.8, 4.0, 2H), 1.58 (d, J=12.3, 2H).

### Example 41

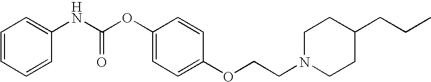

### Phenyl-carbamic acid 4-[2-(4-propyl-piperidin-1-yl)-ethoxy]-phenyl ester

The title compound was prepared according to the procedure for EXAMPLE 11 using phenyl isocyanate and 4-propylpiperidine. MS (ESI): exact mass calculated for C₂₂H₃₀N₂O₃, 382.23; m/z found, 383.4 [M+H]⁺. ¹H NMR (400 MHz, CD₃OD): 7.52 (d, J=7.9, 2H), 7.34 (t, J=8.4, 2H), 7.14 (d, J=9.0, 2H), 7.10 (t, J=6.8,1H), 7.00 (d, J=9.1, 2H), 4.18 (t, J=5.6, 2H), 3.09 (d, J=11.8, 2H), 2,85 (t, J=5.6, 2H), 2.19 (t, J=11.0, 2H), 1.76 (d, J=9.8, 2H), 1.41-1.27 (m, 7H), 0.96 (t, J=7.1, 3H).

### Example 42

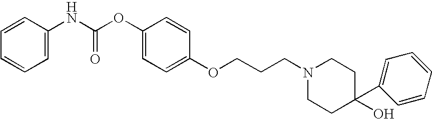

### Phenyl-carbamic acid 4-[3-(4-hydroxy-4-phenyl-piperidin-1-yl)-propoxy]-phenyl ester

The title compound was prepared according to the procedure for EXAMPLE 15 using 4-(3-bromo-propoxy)-phenol in step A, phenyl isocyanate and N-phenyl-4-hydroxypiperidine. MS (ESI): exact mass calculated for C₂₇H₃₀N₂O₄, 446.22; m/z found, 447.1 [M+H]⁺. ¹H NMR (400 MHz, CD₃OD): 7.49 (t, J=8.4, 4H), 7.31 (q, J=10.8 and 7.6, 4H), 7.21 (t, J=7.1, 1H), 7.10-7.03 (m, 3H), 6.95 (d, J=8.0, 2H), 4.06 (t, J=5.9, 2H), 2.85 (d, J=10.8, 2H), 2.67-2.55 (m, 5H), 2.17-2.02 (m, 4H), 1.74 (d, J=13.0, 2H).

### Example 43

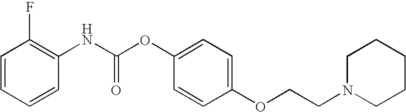

### (2-Fluoro-phenyl)-carbamic acid 4-(2-piperidin-1-yl-ethoxy)-phenyl ester

The title compound was prepared according to the procedure for EXAMPLE 15 using 2-fluorophenyl isocyanate and piperidine. MS (ESI): exact mass calculated for C₂₀H₂₃FN₂O₃, 358.17; m/z found, 359.1 [M+H]⁺. ¹H NMR (400 MHz, CD₃OD): 7.89 (brs, 1H), 7.21-7.14 (m, 5H), 7.00 (d, J=9.1, 2H), 4.17 (t, J=5.6, 2H), 2.83 (d, J=5.6, 2H), 2.61 (br s, 4H), 1.67 (quint, J=5.6, 4H), 1.53 (m, 2H).

### Example 44

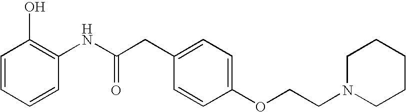

### N-(2—hydroxy-phenyl)-2-[4-(2-piperidin-1-yl-ethoxy)-phenyl]-acetamide

The title compound was prepared according to the procedure for EXAMPLE 13 using 2-hydroxyaniline and piperidine. MS (ESI): exact mass calculated for C₂₁H₂₆N₂O₃, 354.19; m/z found, 355.3 [M+H]⁺. ¹H NMR (400 MHz, DMSO-d₆): 9.78 (s, 1H), 9.30 (s, 1H), 7.75 (d, J=7.8, 1H), 7.30 (d, J=8.5, 2H), 6.96 (d, J=8.5, 2H), 6.91 (d, J=8.0, 1H), 6.84 (d, J=8.0, 1H), 6.74 (t, J=6.8, 1H), 4.29 (m, 2H), 3.67 (s, 2H), 3.46-3.37 (m, 2H), 3.14-2.98 (m, 2H), 1.79-1.68 (m, 4H), 1.24 (m, 2H).

### Example 45

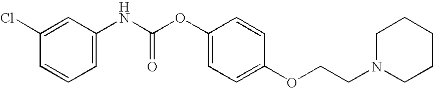

### (3-Chloro-phenyl)-carbamic acid 4-(2-piperidin-1-yl-ethoxy)-phenyl ester

The title compound was prepared according to the procedure for EXAMPLE 15 using 3-chlorophenylisocyanate and piperidine. MS (ESI): exact mass calculated for C₂₀H₂₃ClN₂O₃, 374.14; m/z found, 375.2 [M+H]⁺. ¹H NMR (400 MHz, CDCl₃): 7.49 (br s,1H), 7.23-7.15 (m, 2H), 7.03-6.99 (m, 2H), 7.00 (d, J=9.0, 2H), 6.82 (d, J=9.1, 2H), 4.17 (brs, 2H), 2.89 (brs, 2H), 2.65 (brs, 4H), 1.69 (br s, 4H), 1.44 (br s, 2H).

### Example 46

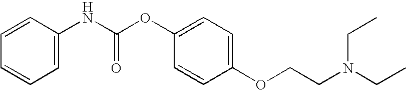

### Phenyl-carbamic acid 4-(2-diethylamino-ethoxy)-phenyl ester

The title compound was prepared according to the procedure for EXAMPLE 15 using phenyl isocyanate and diethylamine. MS (ESI): exact mass calculated for C₁₉H₂₄N₂O₃, 328.18 m/z found, 329.1 M+H]⁺. ¹H NMR (400 MHz, C₆D₆): 7.03 (d, J=7.8, 2H), 6.81 (t, J=8.9, 4H), 6.58 (t, J=7.4, 1H), 6.53 (d, J=9.0, 2H), 5.95 (br s, 1H), 3.52 (t, J=6.4, 2H), 2.45 (d, J=6.4, 2H), 2.18 (q, J=7.1, 4H), 0.69 (t, J=7.1, 6H), 1.58-1.47 (m, 2H).

### Example 47

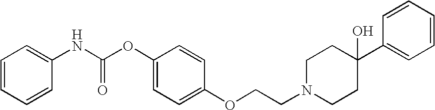

### Phenyl-carbamic acid 4-[2-(4-hydroxy-4-phenyl-piperidin-1-yl)-ethoxy]-phenyl ester

The title compound was prepared according to the procedure for EXAMPLE 11 using phenyl isocyanate and 1-[2-(4-hydroxy-phenoxy)-ethyl]-4-phenyl-piperidin-4-ol. MS (ESI): mass calculated for C₂₆H₂₈N₂O₄, 432.20; m/z found, 433.3 [M+H]⁺. H NMR (400 MHz, CDCl₃): 7.56-7.53 (m, 2H), 7.48-7.46 (m, 2H), 7.40-7.36 (m, 4H), 7.14-7.10 (m, 3H), 6.97-6.92 (m, 3H), 4.17 (t, J=5.8, 2H), 2.95-2.90 (m, 4H), 2.69-2.64 (m, 2H), 2.23-2.19 (m, 2H), 1.83-1.77 (m, 2H), 1.59 (br s, 1H).

### Example 48

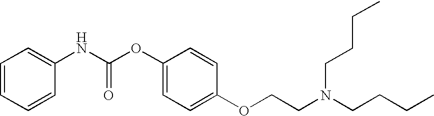

### Phenyl-carbamic acid 4-(2-dibutylamino-ethoxy)-phenyl ester

The title compound was prepared according to the procedure for EXAMPLE 11 using phenyl isocyanate and dibutylamine. MS (ESI): mass calculated for C₂₃H₃₂N₂O₃, 384.24; m/z found, 385.3 [M+H]⁺. ¹H NMR (400 MHz, CDCl₃): 7.46-7.28 (m, 4H), 7.15-7.05 (m, 3H), 6.92-6.85 (m, 2H), 4.054.02 (m, 2H), 2.94-2.87 (m, 2H), 2.57-2.53 (m, 4H), 1.48-1.31 (m, 8H), 0.97-0.93 (m, 6H).

### Example 49

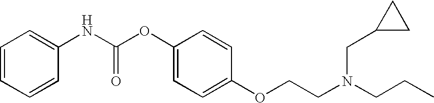

### Phenyl-carbamic acid 4-[2-(cyclopropylmethyl-propyl-amino)-ethoxy]-phenyl ester

The title compound was prepared according to the procedure for EXAMPLE 11 using phenyl isocyanate and N-propylcyclopropanemethylamine. MS (ESI): mass calculated for C₂₂H₂₈N₂O₃, 368.21; m/z found, 369.3 [M+H]⁺. ¹H NMR (400 MHz, CDCl₃): 7.47-7.44 (m, 2H), 7.37-7.32 (m, 2H), 7.13-7.05 (m, 4H), 6.94-6.90 (m, 2H), 4.08 (t, J=6.2, 2H), 3.00 (t, J=6.2, 2H), 2.61 (t, J=7.6, 2H), 2.48 (d, J=6.4, 2H), 1.56-1.51 (m, 2H), 0.93 (t, J=7.3, 4H), 0.55-0.52 (m, 2H), 0.16-0.13 (m, 2H).

### Example 50

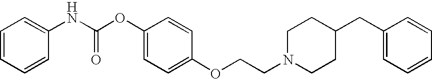

### Phenyl-carbamic acid 4-[2-(4-benzyl-piperidin-1-yl)-ethoxy]-phenyl ester

The title compound was prepared according to the procedure for EXAMPLE 11 using phenyl isocyanate and 4-benzylpiperidine. MS (ESI): mass calculated for C₂₇H₃₀N₂O₃, 430.23; m/z found, 431.2 [M+H]⁺. ¹H NMR (400 MHz, CDCl₃): 7.47-7.44 (m, 2H), 7.37-7.28 (m, 3H), 7.22-7.08 (m, 6H), 6.94-6.89 (m, 3), 4.10 (t, J=6.0, 2H), 3.05-2.90 (m, 2H), 2.79 (t, J=6.0, 2H), 2.55 (d, J=7.1, 2H), 2.08-2.03 (m, 2H), 1.67-1.53 (m, 3H), 1.41-1.34 (m, 2H).

### Example 51

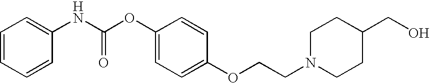

### Phenyl-carbamic acid 4-[2-(4-hydroxymethyl-piperidin-1-yl)-ethoxy]-phenyl ester

The title compound was prepared according to the procedure for EXAMPLE 11 using pheny isocyanate and 4-hydroxymethylpiperidine. MS (ESI): mass calculated for C₂₁H₂₆N₂O₄, 370.19; m/z found, 371.1 [M+H]⁺. ¹H NMR (400 MHz, CDCl₃): 7.47-7.45 (m, 2H), 7.37-7.31 (m, 2H), 7.14-7.09 (m, 3H), 6.94-6.91 (m, 3H), 4.12 (t, J=6.0, 2H), 3.52 (t, J=6.4, 2H), 3.08-3.00 (m, 2H), 2.84-2.81 (m, 2H), 2.17-2.10 (m, 2H), 1.79-1.74 (m, 2H), 1.54-1.49 (m, 1H), 1.36-1.27 (m, 2H).

### Example 52

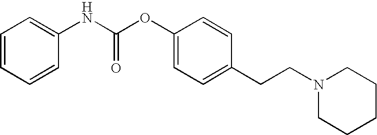

### Phenyl-carbamic acid 4-(2-piperidin-1-yl-ethyl)-phenyl ester

The title compound was prepared according to the procedure for EXAMPLE 17 using phenyl isocyanate and 4-(2-piperidin-1-yl-ethyl)-phenolpiperidine. MS (ESI): mass calculated for C₂₀H₂₄N₂O₂, 324.18; m/z found, 325.1 [M+H]⁺. ¹H NMR (400 MHz, CDCl₃): 7.47-7.45 (m, 2H), 7.37-7.34 (m, 2H), 7.27-7.23 (m, 3H), 7.16-7.09 (m, 3H), 2.85-2.811 (m, 2H), 2.59-2.55 (m, 2H), 2.48 (br s, 3H), 1.67-1.62 (m, 6H), 1.51-1.45 (m, 2H).

### Example 53

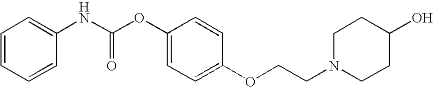

### Phenyl-carbamic acid 4-[2-(4-hydroxy-piperidin-1-yl)-ethoxy]-phenyl ester

The title compound was prepared according to the procedure for EXAMPLE 11 using phenyl isocyanate and 4-hydroxypiperidine. MS (ESI): mass calculated for C₂₀H₂₄N₂O₄, 356.17; m/z found, 357.1 [M+H]⁺. ¹H NMR (400 MHz, CDCl₃): 7.47-7.45 (m, 2H), 7.37-7.34 (m, 2H), 7.14-7.09 (m, 3H), 6.95-6.91 (m, 3H), 4.11 (t, J=5.9, 2H), 3.73 (brs, 1H), 2.91-2.87 (m, 2H), 2.83 (t, J=5.9, 2H), 2.34-2.29 (m, 2H), 1.94-1.92 (m, 2H), 1.68-1.63 (m, 3H).

### Example 54

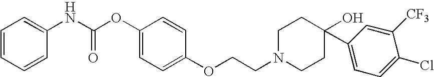

### Phenyl-carbamic acid 4-{2-[4-(4-chloro-3-trifluoromethyl-phenyl)-4-hydroxy-piperidin-1-yl]-ethoxy}-phenyl ester

The title compound was prepared according to the procedure for EXAMPLE 11 using phenyl isocyanate and 4-[4-chloro-3-(trifluoromethyl )phenyl]-4-piperidinol. MS (ESI): mass calculated for C₂₀H₂₄N₂O₄, 356.17; m/z found, 357.1 [M+H]⁺. ¹H NMR (400 MHz, CD₃OD): 7.94 (s, 1H), 7.75-7.71 (m, 1H), 7.65-7.63 (m, 1H), 7.48-7.45 (m, 2H), 7.32-7.27 (m, 2H), 7.17-7.14 (m, 2H), 7.09-7.03 (m, 3H), 4.44-4.42 (m, 2H), 3.69-3.56 (m, 6H), 3.51-3.45 (m, 2H), 2.41-2.34 (m, 2H), 2.02-1.99 (m, 2H).

### Example 55

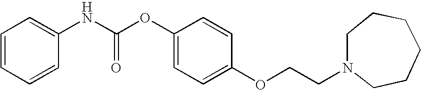

### Phenyl-carbamic acid 4-(2-azepan-1-yl-ethoxy)-phenyl ester

The title compound was prepared according to the procedure for EXAMPLE 11 using phenyl isocyanate and homopiperidine. MS (ESI): mass calculated for C₂₁H₂₆N₂O₃, 354.19; m/z found, 355.4 [M+H]⁺. ¹H NMR (400 MHz, CD₃OD): 7.47-7.45 (m, 2H), 7.30-7.26 (m, 2H), 7.15-7.12 (m, 2H), 7.06-7.02 (m, 3H), 4.35 (t, J=5.0, 2H), 3.63-3.54 (m, 4H), 3.35-3.30 (m, 2H), 1.94 (br s, 4H), 1.75 (br s, 4H).

### Example 56

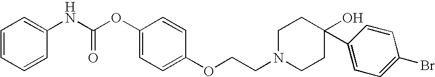

### Phenyl-carbamic acid 4-{2-[4-(4-bromo-phenyl)-4-hydroxy-piperidin-1-yl]-ethoxy}-phenyl ester

The title compound was prepared according to the procedure for EXAMPLE 11 using phenyl isocyanate and 4-(4′-bromophenyl)-4-hydroxypiperidine. MS (ESI): mass calculated for C₂₆H₂₇BrN₂O₄, 510.12; m/z found, 511.4 [M+H]⁺. ¹H NMR (400 MHz, CD₃OD): 7.54-7.43 (m, 6H), 7.32-7.27 (m, 2H), 7.17-7.14 (m, 2H), 7.09-7.04 (m, 3H), 4.44-4.41 (m, 2H), 3.69-3.54 (m, 6H), 2.39-2.31 (m, 2H), 2.02-1.98 (m, 2H).

### Example 57

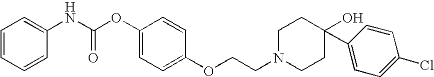

### Phenyl-carbamic acid 4-{2-[4-(4-chloro-phenyl )-4-hydroxy-piperidin-1-yl]-ethoxy}-phenyl ester

The title compound was prepared according to the procedure for EXAMPLE 11 using pheny isocyanate and 4-(4′-chlorophenyl)-4-hydroxypiperidine. MS (ESI): mass calculated for C₂₆H₂₇ClN₂O₄, 466.17; m/z found, 467.4 [M+H]⁺. ¹H NMR (400 MHz, CD₃OD): 9.73 (br s,1H), 7.52-7.46 (m, 4H), 7.39-7.37 (m, 2H), 7.31-7.27 (m, 2H), 7.17-7.14 (m, 2H), 7.09-7.06 (m, 3H), 4.44-4.42 (m, 2H), 3.69-3.54 (m, 6H), 2.39-2.32 (m, 2H), 2.03-1.97 (m, 2H).

### Example 58

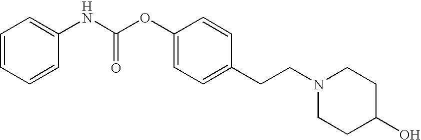

### Phenyl-carbamic acid 4-[2-(4-hydroxy-piperidin-1-yl)-ethyl]-phenyl ester

The title compound was prepared according to the procedure for EXAMPLE 17 using phenyl isocyanate and 4-hydroxypiperidine. MS (ESI): mass calculated for C₂₀H₂₄N₂O₃, 340.18; m/z found, 341.4 [M+H]⁺. ¹H NMR (400 MHz, CDCl₃): 7.44 (d, J=8.0, 2H), 7.34 (d, J=7.4, 2H), 7.23 (d, J=8.4, 2H), 7.13-7.06 (m, 3H), 3.72 (s, 1H), 2.90-2.75 (m, 4H), 2.62-2.53 (m, 2H), 2.22 (t, J=9.8, 2H), 1.98-1.88 (m, 2H), 1.68-1.52 (m, 4H).

### Example 59

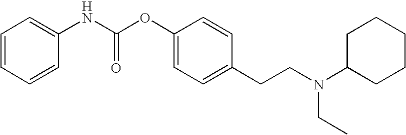

### Phenyl-carbamic acid 4-[2-(cyclohexyl-ethyl-amino)-ethyl]-phenyl ester

The title compound was prepared according to the procedure for EXAMPLE 17 using phenyl isocyanate and N-cyclohexyl-N-ethylamine. MS (ESI): mass calculated for C₂₃H₃₀N₂O₂, 366.23; m/z found, 367.2 [M+H]⁺. ¹H NMR (400 MHz, CDCl₃): 8.13 (s, 1H), 7.55 (d, J=8.4, 2H), 7.27 (t, J=8.4, 2H), 7.18 (d, J=8.6, 2H), 7.08-6.99 (m, 3H), 3.29-3.08 (m, 7H), 2.22 (d, J=11.0, 2H), 1.87 (d, J=12.9, 2H), 1.67 (d, J=12.5,1H), 1.53-1.41 (m, 5H), 1.34-1.21 (m, 2H), 1.18-1.04 (m,1H).

### Example 60

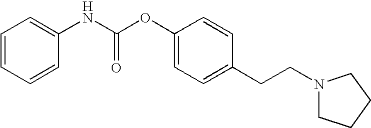

### Phenyl-carbamic acid 4-(2-pyrrolidin-1-yl-ethyl)-phenyl ester

The title compound was prepared according to the procedure for EXAMPLE 17 using phenyl isocyanate and pyrrolidine. MS (ESI): mass calculated for C₁₉H₂₂N₂O₂, 310.17; m/z found, 311.1 [M+H]⁺. ¹H NMR (400 MHz, CDCl₃): 7.47 (d, J=7.8, 2H), 7.35-7.25 (m, 4H), 7.16-7.05 (m, 3H), 3.93 (br s, 1H), 3.73 (t, J=6.5, 1H), 3.40-3.31 (m, 2H), 3.21-3.08 (m, 4H), 1.37 (t, J=7.2, 4H).

### Example 61

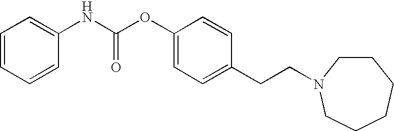

### Phenyl-carbamic acid 4-(2-azepan-1-yl-ethyl)-phenyl ester

The title compound was prepared according to the procedure for EXAMPLE 17 using phenyl isocyanate and homopiperidine. MS (ESI): mass calculated for C₂₁H₂₆N₂O₂, 338.20; m/z found, 339.1 [M+H]⁺. H NMR (400 MHz, CD₃OD): 7.47 (d, J=8.2, 2H), 7.33-7.21 (m, 4H), 7.12-7.02 (m, 3H), 2.87-2.72 (m, 8H), 1.77-1.06 (m, 8H).

### Example 62

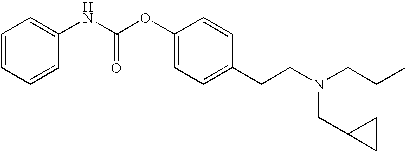

### Phenyl-carbamic acid 4-[2-(cyclopropylmethyl-propyl-amino)-ethyl]-phenyl ester

The title compound was prepared according to the procedure for EXAMPLE 17 using phenyl isocyanate and N-propylcyclopropanemethylamine. MS (ESI): mass calculated for C₂₂H₂₈N₂O₂, 352.22; m/z found, 353.2 [M+H]⁺. ¹H NMR (400 MHz, CDCl₃): 7.46 (d, J=8.2, 2H), 7.30 (t, J=7.8, 2H), 7.19 (d, J=8.8, 2H), 7.11-7.04 (m, 3H), 2.98-2.86 (m, 4H), 2.75 (t, J=17.4, 2H), 2.62 (d, J=6.6, 2H), 1.68-1.56 (m, 2H), 1.04-0.95 (m, 1H), 0.92 (t, J=7.4, 4H), 0.59 (dd, J=7.2, 5.7, 2H), 0.22 (dd, J=6.3, 5.7, 2H).

### Example 63

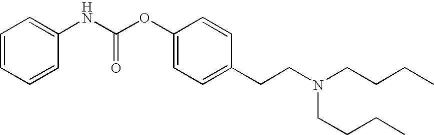

### Phenyl-carbamic acid 4-(2-dibutylamino-ethyl)-phenyl ester

The title compound was prepared according to the procedure for EXAMPLE 17 using phenyl isocyanate and dibutylamine. MS (ESI): mass calculated for C₂₃H₃₂N₂O₂, 368.25; m/z found, 369.2 [M+H]⁺. H NMR (400 MHz, CDCl₃): 7.41 (d, J=8.0, 2H), 7.28 (t, J=8.2, 2H), 7.17 (d, J=8.4, 2H), 7.09-7.03 (m, 3H), 2.77-2.65 (m, 4H), 2.50 (t, J=7.6, 4H), 1.50-1.40 (m, 4H), 1.35-1.25 (m, 4H), 0.91 (t, J=7.4, 6H).

### Example 64

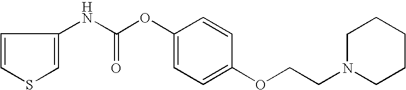

### Thiophen-3-yl-carbamic acid 4-(2-piperidin-1-yl-ethoxy)-phenyl ester

The title compound was prepared according to the procedure for EXAMPLE 11 using 3-isocyanato-thiophene and piperidine. MS (ESI): mass calculated for C₁₈H₂₂N₂O₃S, 346.14; m/z found, 347.4 [M+H]⁺. ¹H NMR (400 MHz, CD₃OD): 7.32-7.26 (m, 1H), 7.23 (s, 1H), 7.10 (d, J=9.1, 2H), 7.06-7.02 (m, 1H), 6.98 (d, J=9.1, 2H), 4.29 (t, J=5.0, 2H), 3.56 (d, J=5.0, 2H), 3.48 (t, J=5.0, 2H), 3.29-3.26 (m, 1H), 2.99 (t, J=12.1, 2H), 1.94-1.70 (m, 5H), 1.54-1.41 (m, 1H).

### Example 65

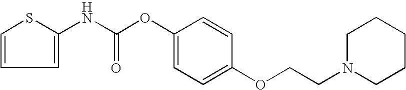

### Thiophen-2-yl-carbamic acid 4-(2-piperidin-1-yl-ethoxy)-phenyl ester

The title compound was prepared according to the procedure for EXAMPLE 11 using 2-isocyanato-thiophene and piperidine. MS (ESI): mass calculated for C₁₈H₂₂N₂O₃S, 346.14; m/z found, 347.4 [M+H]⁺. ¹H NMR (400 MHz, CD₃OD): 7.13 (d, J=9.1, 2H), 7.01 (d, J=9.1, 2H), 6.90-6.85 (m, 1H), 6.84-6.79 (m, 1H), 6.69-6.63 (m, 1H), 4.33 (t, J=5.0, 2H), 3.60 (d, J=11.6, 2H), 3.53 (t, J=5.0, 2H), 3.30-3.26 (m,1H), 3.04 (t, J=12.6, 2H), 1.99-1.89 (m, 2H), 1.87-1.73 (m, 3H), 1.58-1.45 (m, 1H).

### Example 66

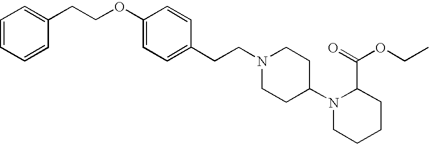

### 1′-[2-(4-Phenethyloxy-phenyl)-ethyl]-[1,4′]bipiperidinyl-2-carboxylic acid ethyl ester

The title compound was prepared according to the procedure for EXAMPLE 18 using 2-bromoethyl-benzene and [1,4′]bipiperidinyl-2-carboxylic acid ethyl ester. MS (ESI): mass calculated for C₂₉H₄₀N₂O₃, 464.30; m/z found, 465.5 [M+H]⁺. ¹H NMR (400 MHz, CDCl₃): 7.34-7.22 (m, 5H), 7.11-7.07 (m, 2H), 6.84-6.81 (m, 2H), 4.23-4.13 (m, 4H), 3.41-3.37 (m, 1H), 3.11-2.99 (m, 5H), 2.75-2.71 (m, 2H), 2.54-2.34 (m, 4H), 2.00-1.85 (m, 2H), 1.84-1.70 (m, 5H), 1.68-1.56 (m, 4H), 1.36-1.26 (m, 4H).

### Example 67

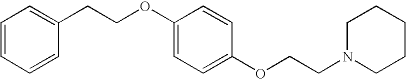

### 1-[2-(4-Phenethyloxy-phenoxy)-ethyl]-piperidine

The title compound was prepared according to the procedure for EXAMPLE 28 using phenethyl alcohol. MS (ESI): mass calculated for C₂₁H₂₇NO₂, 325.20; m/z found, 326.4 [M+H]⁺. ¹H NMR (400 MHz, CDCl₃): 7.33-7.20 (m, 5H), 6.81 (s, 4H), 4.11 (t, J=7.2, 2H), 4.05 (t, J=6.3, 2H), 3.06 (t, J=6.3, 2H), 2.51 (br s, 4), 1.65-1.57 (m, 4H), 1.48-1.40 (m, 2H).

### Example 68

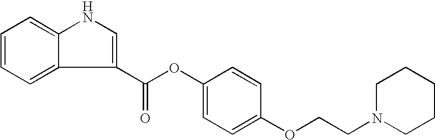

### 1H-Indole-3-carboxylic acid 4-(2-piperidin-1-yl-ethoxy)-phenyl ester

The title compound was prepared according to the procedure for EXAMPLE 23 using 1H-indole-3-carboxylic acid. MS (ESI): mass calculated for C₂₂H₂₄N₂O₃, 364.18; m/z found, 365.4 [M+H]⁺. ¹H NMR (400 MHz, CDCl₃): 9.89 (br s,1H), 8.25-8.19 (m, 1H), 7.98 (s, 1H), 7.43-7.37 (m, 1H), 7.30-7.23 (m, 2H), 7.11 (d, J=9.0, 2H), 6.84 (d, J=9.0, 2H), 4.09 (t, J=5.9, 2H), 2.82 (t, J=5.9, 2H), 2.58 (br s, 4H), 1.68-1.59 (m, 4H), 1.51-1.41 (m, 2H).

### Example 69

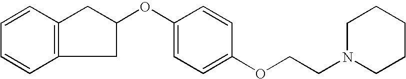

### 1-{2-[4-(Indan-2-yloxy)-phenoxy]-ethyl}-piperidine

The title compound was prepared according to the procedure for EXAMPLE 28 using indane-2-ol. MS (ESI): mass calculated for C₂₂H₂₇NO₂, 337.20; m/z found, 338.4 [M+H]⁺. ¹H NMR (400 MHz, CDCl₃): 7.25-7.20 (m, 2H), 7.19-7.14 (m, 2H), 6.82 (br s, 4H), 5.10-5.03 (m, 1H), 4.05 (t, J=6.3, 2H), 3.35-3.27 (m, 2H), 3.18-3.11 (m, 2H), 2.74 (t, J=6.3, 2H), 2.50 (br s, 4H), 1.64-1.56 (m, 4H), 1.48-1.39 (m, 2H).

### Example 70

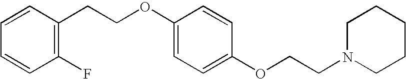

### 1-(2-{4-[2-(2-Fluoro-phenyl )-ethoxy]-phenoxy}-ethyl)-piperidine

The title compound was prepared according to the procedure for EXAMPLE 28 using 2-fluorophenethyl alcohol. MS (ESI): mass calculated for C₂₁H₂₆FNO₂, 343.19; m/z found, 344.4 [M+H]⁺. ¹H NMR (400 MHz, CDCl₃): 7.32-7.17 (m, 2H), 7.10-6.99 (m, 2H), 6.81 (s, 4H), 4.12 (t, J=7.0, 2H), 4.04 (t, J=6.3, 2H), 3.10 (t, J=7.0, 2H), 2.74 (t, J=6.3, 2H), 2.49 (br s, 4H), 1.64-1.56 (m, 4H), 1.47-1.39 (m, 2H).

### Example 71

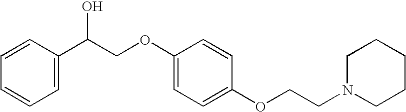

### 1-Phenyl-2-[4-(2-piperidin-1-yl-ethoxy)-phenoxy]-ethanol

The title compound was prepared according to the procedure for EXAMPLE 28 using 1-phenylethane-1,2-diol. MS (ESI): mass calculated for C₂₁H₂₇NO₃, 341.20; m/z found, 342.4 [M+H]⁺. ¹H NMR (400 MHz, CDCl₃): 7.46-7.39 (m, 2H), 7.38-7.27 (m, 3H), 6.81 (s, 4H), 5.09-5.03 (m,1H), 4.05-3.98 (m, 3H), 3.95 (t, J=8.6,1H), 3.46 (br s,1H), 2.71 (t, J=6.3, 2H), 2.47 (br s, 4H), 1.63-1.54 (m, 4H), 1.46-1.38 (m, 2H).

### Example 72

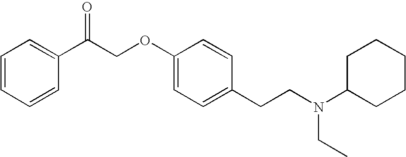

### 2-{4-[2-(Cyclohexyl-ethyl-amino)-ethyl]-phenoxy}-1-phenyl-ethanone

The title compound was prepared according to the procedure for EXAMPLE 26 using 2-bromo-1-phenyl-ethanone and N-cyclohexylethylamine. MS (ESI): mass calculated for C₂₄H₃₁NO₂, 365.24; m/z found, 366.4 [M+H]⁺. ¹H NMR (400 MHz, CDCl₃): 7.99 (d, J=7.2, 2H), 7.60 (t, J=7.2,1H), 7.48 (d, J=7.2, 2H), 7.10 (d, J=8.4, 2H), 6.86 (d, J=8.8, 2H), 5.23 (s, 2H), 2.96-2.57 (m, 6H), 2.56-2.46 (m, 1H), 1.78 (t, J=10.6, 4H), 1.61 (d, J=11.7, 1H), 1.28-1.13 (m, 4H), 1.05 (t, J=7.2, 4H).

### Example 73

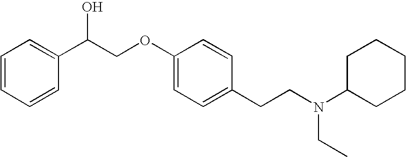

### 2-{4-[2-(Cyclohexyl-ethyl-amino)-ethyl]-phenoxy}-1-phenyl-ethanol

The title compound was prepared according to the procedure for EXAMPLE 27 using 1-phenylethane-1,2-diol and N-cyclohexylethylamine. MS (ESI): mass calculated for C₂₄H₃₃NO₂, 367.25; m/z found, 368.4 [M+H]⁺. ¹H NMR (400 MHz, CDCl₃): 7.42 (d, J=7.2, 2H), 7.35 (t, J=7.2, 1H), 7.32-7.26 (m, 2H), 7.07 (d, J=8.4, 2H), 6.81 (d, J=8.4, 2H), 5.07 (dd, J=5.3, 3.3, 1H), 4.04 (dd, J=6.1, 3.3, 1H), 3.97 (t, J=9.6, 1H), 3.41 (br s, 1H), 2.67-2.54 (m, 6H), 2.53-2.44 (m, 1H), 1.77 (t, J=10.8, 4H), 1.60 (d, J=12.5, 1H), 1.24-1.13 (m, 4H), 1.04 (t, J=7.2, 4H).

### Example 74

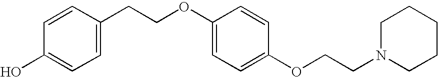

### 4-{2-[4-(2-Piperidin-1-yl-ethoxy)-phenoxy]-ethyl}-phenol

The title compound was prepared according to the procedure for EXAMPLE 28 using 4-(2-hydroxy-ethyl)-phenol. MS (ESI): mass calculated for C₂₁H₂₇NO₃, 341.20; m/z found, 342.4 [M+H]⁺. ¹H NMR (400 MHz, CD₃OD): 7.06 (d, J=8.2, 2H), 6.86 (dd, J=15.3, 9.2, 4H), 6.69 (d, J=8.2, 2H), 4.25 (t, J=5.1, 2H), 4.04 (t, J=6.8, 2H), 3.58 (d, J=12.1, 2H), 3.48 (t, J=5.1, 2H), 3.28 (s, 1H), 3.01 (t, J=12.1, 2H), 2.90 (t, J=6.6, 2H), 1.97-1.88 (m, 2H), 1.85-1.72 (m, 4H).

### Example 75

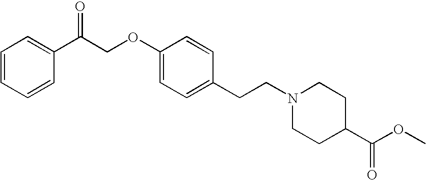

### 1-{2-[4-(2-Oxo-2-phenyl-ethoxy)-phenyl]-ethyl}-piperidine-4-carboxylic acid methyl ester

The title compound was prepared according to the procedure for EXAMPLE 26 using 2-bromo-1-phenyl-ethanone and methyl-4-piperidinecarboxylate. MS (ESI): mass calculated for C₂₃H₂₇NO₄, 381.19; m/z found, 382.4 [M+H]⁺. ¹H NMR (400 MHz, CDCl₃): 7.99 (d, J=7.3, 2H), 7.61 (t, J=7.3, 1H), 7.49 (t, J=7.6, 2H), 7.10 (d, J=8.6, 2H), 6.86 (d, J=8.6, 2H), 5.24 (s, 2H), 3.67 (s, 3H), 2.98-2.89 (m, 2H), 2.72 (dd, J=7.3, 3.8, 2H), 2.53 (dd, J=7.8, 3.5, 2H), 2.35-2.25 (m, 1H), 2.08 (t, J=11.1, 2H), 1.95-1.87 (m, 2H), 1.84-1.72 (m, 2H).

### Example 76

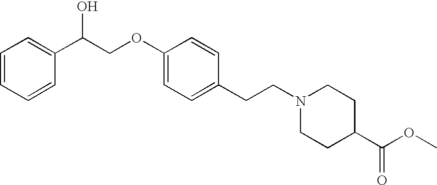

### 1-{2-[4-(2—hydroxy-2-phenyl-ethoxy)-phenyl]-ethyl}-piperidine-4-carboxylic acid methyl ester

The title compound was prepared according to the procedure for EXAMPLE 27 using 1-phenylethane-1,2-diol and methyl-4-piperidinecarboxylate. MS (ESI): mass calculated for C₂₃H₂₉NO₄, 383.21; m/z found, 384.4 [M+H]⁺. ¹H NMR (400 MHz, CDCl₃): 7.42 (d, J=7.1, 2H), 7.34 (t, J=7.1, 2H), 7.30-7.25 (m, 1H), 7.04 (d, J=8.3, 2H), 6.80 (d, J=8.3, 2H), 5.05 (dd, J=4.0, 3.8, 1H), 4.05-3.95 (m, 2H), 3.64 (s, 3H), 2.91-2.84 (m, 2H), 2.69 (dd, J=7.6, 3.3, 2H), 2.38 (dd, J=7.6, 3.3, 2H), 2.32-2.22 (m, 1H), 2.03 (t, J=10.6, 2H), 1.92-1.82 (m, 2H), 1.82-1.69 (m, 2H).

### Example 77

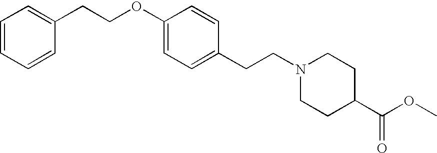

### 1-[2-(4-Phenethyloxy-phenyl)-ethyl]-piperidine-4-carboxylic acid methyl ester

The title compound was prepared according to the procedure for EXAMPLE 29 using methyl-4-piperidinecarboxylate. MS (ESI): mass calculated for C₂₃H₂₉NO₃, 367.21; m/z found, 368.4 [M+H⁺. ¹H NMR (400 MHz, CDCl₃): 7.32-7.17 (m, 5H), 7.08 (d, J=8.6, 2H), 6.80 (d, J=8.6, 2H), 4.12 (t, J=7.1, 2H), 3.66 (s, 3H), 3.06 (t, J=7.1, 2H), 2.96-2.89 (m, 2H), 2.72 (dd, J=7.3, 4.0, 2H), 2.52 (dd, J=7.8, 4.0, 2H), 2.33-2.25 (m,1 H), 2.05 (t, J=10.1, 2H), 1.94-1.87 (m, 2H), 1.84-1.71 (m, 2H).

### Example 78

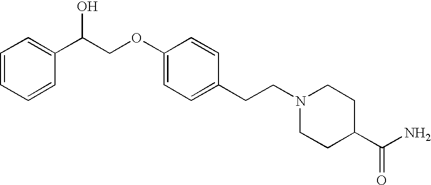

### 1-{2-[4-(2-Hydroxy-2-phenyl-ethoxy)-phenyl]-ethyl}-piperidine-4-carboxylic acid amide

The title compound was prepared according to the procedure for EXAMPLE 27 using 1-phenylethane-1,2-diol and piperidine-4-carboxylic acid amide. MS (ESI): mass calculated for C₂₂H₂₈N₂O₃, 368.21; m/z found, 369.4 [M+H⁺. ¹H NMR (400 MHz, CDCl₃): 7.57-7.22 (m, 5H), 7.10-7.02 (m, 2H), 6.86-6.76 (m, 2H), 5.99 (s, 1 H), 5.69 (s,1 H), 5.08 (dd, J=5.3, 3.3, 1H), 4.28-3.95 (m, 2H), 3.82-3.49 (m, 1 H), 2.99 (d, J=11.7, 2H), 2.70 (dd, J=7.0, 3.9, 2H), 2.51 (dd, J=7.0, 3.9, 2H), 2.19-2.06 (m,1 H), 1.99 (t, J=11.4, 2H), 1.90-1.80 (m, 2H), 1.79-1.66 (m, 2H).

### Example 79

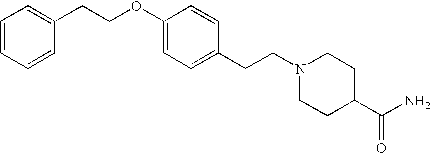

### 1-[2-(4-Phenethyloxy-phenyl)-ethyl]-piperidine-4-carboxylic acid amide

The title compound was prepared according to the procedure for EXAMPLE 29 using piperidine-4-carboxylic acid amide. MS (ESI): mass calculated for C₂₂H₂₈N₂O₂, 352.22; m/z found, 353.4 [M+H⁺. ¹H NMR (400 MHz, CDCl₃): 7.34-7.18 (m, 5H), 7.07 (d, J=8.6, 2H), 6.80 (d, J=8.6, 2H), 6.30 (s, 1H), 6.08 (s, 1H), 4.12 (t, J=7.3, 2H), 3.10-2.98 (m, 4H), 2.72 (dd, J=7.3,4.6, 2H), 2.54 (dd, J=7.3, 4.6, 2H), 2.20-2.10 (m, 1H), 2.04 (t, J=10.4, 2H), 1.91-1.83 (m, 2H), 1.82-1.70 (m, 2H).

### Example 80

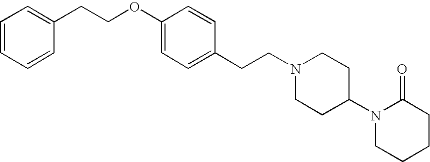

### 1′-[2-(4-Phenethyloxy-phenyl )-ethyl]-[1,4′]bipiperidinyl-2-one

The title compound was prepared according to the procedure for EXAMPLE 29 using [1,4′]bipiperidinyl-2-one. MS (ESI): mass calculated for C₂₆H₃₄N₂O₂, 406.26; m/z found, 407.3 [M+H⁺. ¹H NMR (400 MHz, CDCl₃): 7.32-7.18 (m, 5H), 7.08 (d, J=8.6, 2H), 6.80 (d, J=8.6, 2H), 4.61-4.50 (m,1H), 4.12 (t, J=7.1, 2H), 3.17 (t, J=5.6, 2H), 3.09-2.99 (m, 4H), 2.71 (dd, J=7.3, 4.0, 2H), 2.54 (dd, J=7.3, 4.0, 2H), 2.38 (t, J=6.1, 2H), 2.14 (t, J=11.6, 2H), 1.81-1.68 (m, 6H), 1.64-1.57 (m, 2H).

### Example 81

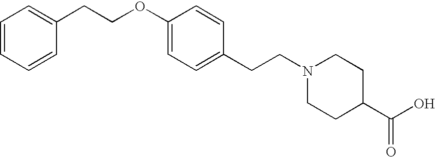

### 1-[2-(4-Phenethyloxy-phenyl)-ethyl]-piperidine-4-carboxylic acid

The title compound was prepared according to the procedure for EXAMPLE 35. MS (ESI): mass calculated for C₂₂H₂₇NO₃, 353.20; m/z found, 354.2 [M+H⁺. ¹H NMR (400 MHz, CD₃OD): 7.27 (d, J=4.3, 4H), 7.21-7.14 (m, 3H), 6.87 (d, J=8.8, 2H), 4.14 (t, J=6.8, 2H), 3.66 (t, J=12.9, 2H), 3.32-3.22 (m, 2H), 3.17-2.93 (m, 6H), 2.67-2.56 (m, 1H), 2.32-2.18 (m, 2H), 1.95-1.81 (m, 2H).

### Example 82

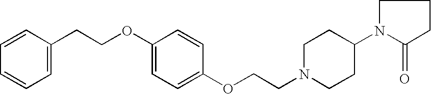

### 1-{1-[2-(4-Phenethyloxy-phenoxy)-ethyl]-piperidin-4-yl}-pyrrolidin-2-one

The title compound was prepared according to the procedure for EXAMPLE 28 using 2-phenyl-ethanol and 1-{1-[2-(4-hydroxy-phenoxy)-ethyl]-piperidin-4-yl}-pyrrolidin-2-one. MS (ESI): mass calculated for C₂₅H₃₂N₂O₃, 408.24; m/z found, 409.2 [M+H⁺. ¹H NMR (400 MHz, CDCl₃): 7.33-7.17 (m, 5H), 6.81 (s, 4H), 4.09 (t, J=7.3, 2H), 4.04-3.94 (m, 3H), 3.31 (t, J=7.1, 2H), 3.08-3.00 (m, 4H), 2.75 (t, J=5.8, 2H), 2.36 (t, J=7.8, 2H), 2.20 (t, J=11.6, 2H), 1.80-1.69 (m, 2H), 1.67-1.60 (m, 2H).

### Example 83

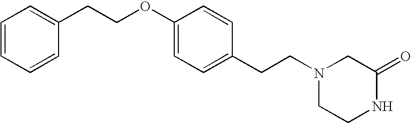

### 4-[2-(4-Phenethyloxy-phenyl)-ethyl]-piperazin-2-one

The title compound was prepared according to the procedure for EXAMPLE 29 using piperazin-2-one. MS (ESI): mass calculated for C₂₀H₂₄N₂O₂, 324.18; m/z found, 325.2 [M+H⁺. ¹H NMR (400 MHz, CDCl₃): 7.77 (s, 1H), 7.32-7.16 (m, 5H), 7.07 (d, J=8.6, 2H), 6.80 (d, J=8.6, 2H), 4.10 (t, J=7.1, 2H), 3.31-3.25 (m, 2H), 3.15 (s, 2H), 3.04 (t, J=7.1, 2H), 2.74-2.67 (m, 2H), 2.63-2.55 (m, 2H).

### Example 84

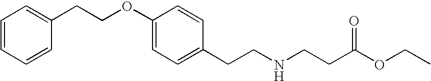

### 3-[2-(4-Phenethyloxy-phenyl)-ethylamino]-propionic acid ethyl ester

The title compound was prepared according to the procedure for EXAMPLE 32 using 3-amino-propionic acid ethyl ester. MS (ESI): mass calculated for C₂₁H₂₇NO₃, 341.20; m/z found, 342.2 [M+H⁺. ¹H NMR (400 MHz, CDCl₃): 7.34-7.19 (m, 5H), 7.09 (d, J=8.6, 2H), 6.82 (d, J=8.6, 2H), 4.18-4.06 (m, 4H), 3.08 (t, J=7.1, 2H), 2.89 (t, J=7.1, 2H), 2.83 (t, J=7.1, 2H), 2.72 (t, J=7.1, 2H), 2.48 (t, J=6.6, 2H), 1.63 (br s, 1 H), 1.21 (t, J=7.1, 3H).

### Example 85

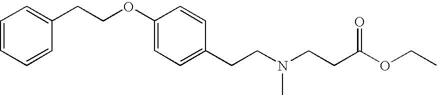

### 3-{Methyl-[2-(4-phenethyloxy-phenyl)-ethyl]-amino}-propionic acid ethyl ester

The title compound was prepared according to the procedure for EXAMPLE 34 using methyl iodide. MS (ESI): mass calculated for C₂₂H₂₉NO₃, 355.21; m/z found, 356.2 [M+H⁺. ¹H NMR (400 MHz, CDCl₃): 7.32-7.18 (m, 5H), 7.08 (d, J=8.6, 2H), 6.80 (d, J=8.6, 2H), 4.15-4.08 (m, 4H), 3.05 (t, J=7.1, 2H), 2.90 (t, J=7.1, 2H), 2.80-2.66 (m, 4H), 2.56 (t, J=7.1, 2H), 2.38 (s, 3H), 1.23 (t, J=7.1, 3H).

### Example 86

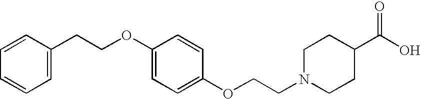

### 1-[2-(4-Phenethyloxy-phenoxy)-ethyl]-piperidine-4-carboxylic acid

The title compound was prepared according to the procedure for EXAMPLE 35. MS (ESI): mass calculated for C₂₂H₂₇NO₄, 369.19; m/z found, 370.2 [M+H⁺. ¹H NMR (400 MHz, CD₃OD): 7.28 (d, J=4.3, 4H), 7.22-7.15 (m, 1H), 6.85-6.78 (m, 4H), 4.11 (t, J=7.1, 2H), 4.05 (t, J=6.1, 2H), 3.06-2.97 (m, 4H), 2.75 (t, J=6.1, 2H), 2.24-2.05 (m, 3H), 1.92-1.84 (m, 2H), 1.81-1.69 (m, 2H).

### Example 87

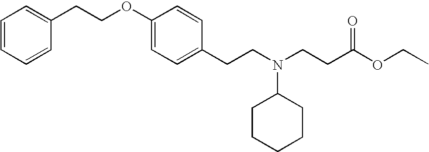

### 3-{Cyclohexyl-[2-(4-phenethyloxy-phenyl)-ethyl]-amino}-propionic acid ethyl ester

The title compound was prepared according to the procedure for EXAMPLE 32 using cycohexylamine and EXAMPLE 34 using 4-ethyl-3-bromopropionate. MS (ESI): mass calculated for C₂₇H₃₇NO₃, 423.28; m/z found, 424.2 [M+H⁺. ¹H NMR (400 MHz, CDCl₃): 7.33-7.18 (m, 5H), 7.09 (d, J=8.6, 2H), 6.81 (d, J=8.6, 2H), 4.16-4.08 (m, 4H), 3.07 (t, J=7.1, 2H), 2.95 (t, J=7.1, 2H), 2.73 (br s, 4H), 2.68-2.58 (m, 1 H), 2.55 (t, J=7.1, 2H), 1.87-1.74 (m, 4H), 1.66-1.57 (m, 1H), 1.27-1.18 (m, 7H), 1.14-1.00 (m, 1H).

### Example 88

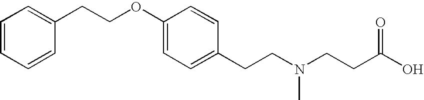

### 3-{Methyl-[2-(4-phenethyloxy-phenyl)-ethyl]-amino}-propionic acid

The title compound was prepared according to the procedure for EXAMPLE 35. MS (ESI): mass calculated for C₂₀H₂₅NO₃, 327.18; m/z found, 328.1 [M+H⁺. ¹H NMR (400 MHz, CD₃OD): 7.30-7.24 (m, 4H), 7.22-7.17 (m, 3H), 6.86 (d, J=8.8, 2H), 4.13 (t, J=6.8, 2H), 3.49-3.40 (m, 2H), 3.36-3.28 (m, 2H), 3.05-2.95 (m, 4H), 2.89 (s, 3H), 2.83 (t, J=7.1, 3H).

### Example 89

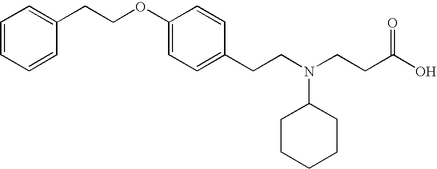

### 3-{Cyclohexyl-[2-(4-phenethyloxy-phenyl)-ethyl]-amino}-propionic acid

The title compound was prepared according to the procedure for EXAMPLE 35. MS (ESI): mass calculated for C₂₅H₃₃NO₃, 395.25; m/z found, 396.2 [M+H⁺. ¹H NMR (400 MHz, CDCl₃): 7.30-7.15 (m, 7H), 6.87 (d, J=8.6, 2H), 4.13 (t, J=7.1, 2H), 3.52-3.27 (m, 5H), 3.06-2.95 (m, 4H), 2.83 (t, J=7.1, 2H), 2.00 (d, J=10.6, 2H), 1.88 (d, J=13.1, 2H), 1.67 (d, J=12.6, 1H), 1.52 (dd, J=11.6, 10.9, 2H), 1.35 (dd, J=12.8, 12.8, 2H), 1.26-1.12 (m, 1H).

### Example 90

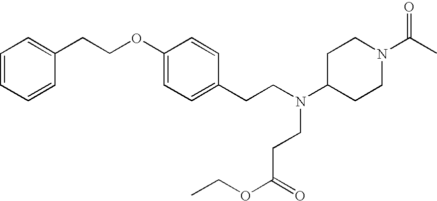

### 3-{(1-Acetyl-piperidin-4-yl)-[2-(4-phenethyloxy-phenyl)-ethyl]-amino}-propionic acid ethyl ester

The title compound was prepared according to the procedure for EXAMPLE 32 using 1-(4-amino-piperidin-1-yl)-ethanone and EXAMPLE 34 using 4-ethyl-3-bromopropionate. MS (ESI): mass calculated for C₂₈H₃₈N₂O₄, 466.28; m/z found, 467.2 [M+H⁺. ¹H NMR (400 MHz, CDCl₃): 7.34-7.18 (m, 5H), 7.06 (d, J=8.6, 2H), 6.80 (d, J=8.6, 2H), 4.63 (d, J=12.1, 1H), 4.16-4.07 (m, 2H), 3.86 (t, J=6.6, 2H), 3.74 (t, J=6.1, 1H), 3.69 (t, J=12.1, 1H), 3.48 (t, J=6.6, 2H), 3.35 (t, J=8.3, 2H), 3.15 (t, J=12.1, 1H), 3.07-2.96 (m, 4H), 2.85 (t, J=6.6, 2H), 2.62 (t, J=12.1, 1H), 2.15-2.02 (m, 5H), 1.87-1.73 (m, 1H), 1.70-1.56 (m, 1H), 1.25 (t, J=7.3, 3H).

### Example 91

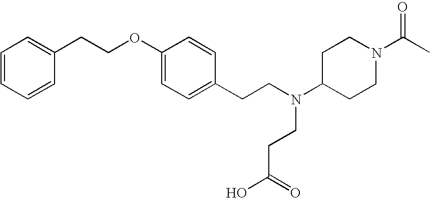

### 3-{(1-Acetyl-piperidin-4-yl)-[2-(4-phenethyloxy-phenyl)-ethyl]-amino}-propionic acid

The title compound was prepared according to the procedure for EXAMPLE 35. MS (ESI): mass calculated for C₂₆H₃₄N₂O₄, 438.25; m/z found, 439.2 [M+H⁺. ¹H NMR (400 MHz, CD₃OD): 7.28-7.15 (m, 7H), 6.86 (d, J=8.3, 2H), 4.67 (d, J=13.4, 1H), 4.12 (t, J=7.1, 2H), 4.03 (d, J=13.6, 1H), 3.69 (t, J=12.1, 1H), 3.48 (t, J=6.6, 2H), 3.35 (t, J=8.3, 2H), 3.15 (t, J=12.1, 1H), 3.07-2.96 (m, 4H), 2.85 (t, J=6.6, 2H), 2.62 (t, J=12.1, 1H), 2.15-2.02 (m, 5H), 1.87-1.73 (m, 1H), 1.70-1.56 (m, 1H).

### Example 92

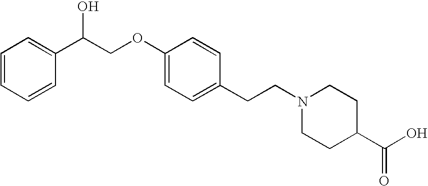

### 1-2-[4-(2-Hydroxy-2-phenyl-ethoxy)-phenyl]-ethyl}-piperidine-4-carboxylic acid

The title compound was prepared according to the procedure for EXAMPLE 35. MS (ESI): mass calculated for C₂₂H₂₇NO₄, 369.19; m/z found, 370.4 [M+H⁺. ¹H NMR (400 MHz, CD₃OD): 7.44 (d, J=7.1, 2H), 7.34 (t, J=7.1, 2H), 7.30-7.24 (m, 1H), 7.17 (d, J=8.6, 2H), 6.89 (d, J=8.6, 2H), 5.00 (dd, J=4.0, 3.8, 1H), 4.03 (d, J=5.8, 2H), 3.65 (d, J=12.6, 2H), 3.29-3.20 (m, 2H), 3.02-2.92 m, 3H), 2.66-2.56 (m, 1H), 2.31-2.15 (m, 2H), 2.13-1.98 (m, 2H), 1.97-1.83 (m, 2H).

### Example 93

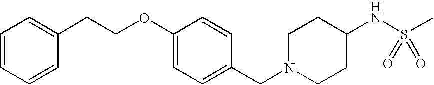

### N-[1-(4-Phenethyloxy-benzyl)-piperidin-4-yl]methanesulfonamide

A. 4-Phenethyloxy-benzaldehyde. To a stirred solution of 4-hydroxy-benzaldehyde (6.1 g, 50 mmol) in CH₂Cl₂ (500 mL), was added 2-phenylethanol (6.1 g, 50 mmol), followed by polymer-supported triphenylphosphine (16.7 g, 50 mmol) and di-tert-butyl azodicarboxylate (11.5 g, 50 mmol). The mixture was stirred for 2 h at rt. The resulting suspension was filtered, and the filtrate was concentrated. The resultant oil was purified on SiO₂ (330 g; 10-30% EtOAc/hexanes) to give 8.68 g (77%) of a clear oil. MS (ESI): mass calculated for C₁₅H₁₄O₂, 226.10; m/z found, 227.4 [M+H⁺. ¹H NMR (400 MHz, CDCl₃): 9.86 (s, 1H), 7.80 (d, J=8.5, 2H), 7.35-7.22 (m, 5H), 6.97 (d, J=8.5, 2H), 4.24 (t, J=7.1, 2H), 3.12 (t, J=7.1, 2H).

B. [1-(4-Phenethyloxy-benzyl)-piperidin-4-yl]-carbamic acid tert-butyl ester. A mixture of 4-phenethyloxy-benzaldehyde (5.1 g, 22.5 mmol) and piperidin-4-yl-carbamic acid tert-butyl ester (5.4 g, 27.0 mmol) in CH₂Cl₂ (225 mL) was stirred at rt for 40 min. To the resulting reaction mixture was added NaBH(OAc)₃ (7.15 g, 33.8 mmol) portion wise over 1.5 h. The resulting mixture was stirred at rt for 24 h, filtered through diatomaceous earth and rinsed with CH₂Cl₂ (300 mL). The filtrate was washed with satd. aq. NaHCO₃ (1×50 mL), dried (Na₂SO₄) and concentrated to yield the crude product as a white solid. The crude product was purified on SiO₂ (330 g; 0-100% EtOAc/hexanes) to give a white solid (7.56 g, 82%). MS (ESI): mass calculated for C₂₅H₃₄N₂O₃, 410.2; m/z found, 411.5 [M+H⁺. ¹H NMR (400 MHz, CDCl₃): 9.00 (br s, 1H), 7.33-7.15 (m, 5H), 7.18 (d, J=8.5, 2H), 6.83 (d, J=8.5, 2H), 4.53 (d, J=8.5, 1H), 4.15 (t, J=7.3, 2H), 3.48 (s, 2H), 3.08 (t, J=7.1, 2H), 2.85 (d, J=10.6, 2H), 2.10 (t, J=11.1, 2H), 1.89 (d, J=11.6, 2H), 1.50-1.44 (m, 2H), 1.42 (s, 9H).

C. 1-(4-Phenethyloxy-benzyl)-piperidin-4-ylamine. To a solution of [1-(4-phenethyloxy-benzyl)-piperidin-4-yl]-carbamic acid tert-butyl ester (7.5 g, 18.3 mmol) in CH₂Cl₂ (90 mL) at 0° C. was added 4 N HCl in dioxane (18.3 mL, 73.0 mmol) dropwise. The resulting mixture was stirred at rt for 2 h. The desired product was isolated by filtration and was washed with Et₂O (300 mL) to yield a white powder (5.3 g, 69%). MS (ESI): mass calculated for C₂₀H₂₆N₂O, 310.2; m/z found, 311.5 [M+H⁺.

D. N-[1-(4-Phenethyloxy-benzyl)-piperidin-4-yl]methanesulfonamide. To a solution of 1-(4-phenethyloxy-benzyl)-piperidin-4-ylamine dihydrochloride (420 mg, 1.0 mmol) in CH₂Cl₂ (20 mL) at rt was added triethylamine (0.70 mL, 5.0 mmol), followed by methanesulfonyl chloride (0.12 mL, 1.5 mmol). The resulting mixture was stirred at rt overnight. The mixture was dissolved in CH₂Cl₂ (100 mL), washed with satd. aq. NaHCO₃ (1×25 mL), dried (Na₂SO₄) and concentrated to yield the crude product as a white solid. The crude product was purified on SiO₂ (40 g; 0-10% CH₃OH/CH₂Cl₂) to give a white solid (299 mg, 77%). MS (ESI): mass calculated for C₂₁H₂₈N₂O₃S, 388.1; m/z found, 389.4 [M+H⁺. ¹H NMR (400 MHz, CDCl₃): 7.33-7.19 (m, 5H), 7.17 (d, J=8.5, 2H), 6.83 (d, J=8.5, 2H), 4.66 (d, J=7.3, 1H), 4.14 (t, J=7.1, 2H), 3.39 (s, 2H), 3.34-3.24 (m, 1H), 3.07 (t, J=7.1, 2H), 2.94 (s, 3H), 2.77 (d, J=11.6, 2H), 2.05 (t, J=11.1, 2H), 1.92 (d, J=11.6, 2H), 1.60-1.49 (m, 2H).

### Example 94

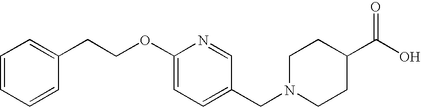

### 1-(6-Phenethyloxy-pyridin-3-ylmethyl)-piperidine-4-carboxylic acid

The title compound was prepared using procedures analogous to Step B for EXAMPLE 93 (using ethyl 4-piperidinecarboxylic acid) followed by EXAMPLE 35. MS (ESI): mass calculated for C₂₀H₂₄N₂O₃, 340.18; m/z found, 341.4 [M+H⁺.

### Example 95

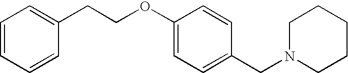

### 1-(4-Phenethyloxy-benzyl)-piperidine

The title compound was prepared according to Step B for EXAMPLE 93 using piperidine. MS (ESI): mass calculated for C₂₀H₂₅NO, 295.19; m/z found, 296.4 [M+H⁺].

### Example 96

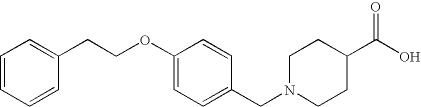

### 1-(4-Phenethyloxy-benzyl)-piperidine-4-carboxylic acid

The title compound was prepared using procedures analogous to Step B for EXAMPLE 93 (using ethyl 4-piperidinecarboxylic acid) followed by EXAMPLE 35. MS (ESI): mass calculated for C₂₀H₂₅NO₃, 339.18; m/z found, 340.3 [M+H⁺. ¹H NMR (400 MHz, CDCl₃): 12.60 (brs, 1H), 7.32-7.18 (m, 7H), 6.81 (d, J=8.3 Hz, 2H), 4.11 (t, J=7.3 Hz, 2H), 3.76 (s, 2H), 3.05 (t, J=6.6 Hz, 4H), 2.45-2.30 (m, 2H), 2.18-2.28 (m, 1H), 2.00-1.82 (m, 4H).

### Example 97

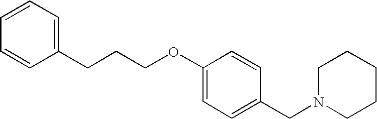

### 1-[4-(3-Phenyl-propoxy)-benzyl]-piperidine

The title compound was prepared according to Step A for EXAMPLE 93 using 3-phenpropanol followed by Step B for EXAMPLE 93 using piperidine. MS (ESI): mass calculated for C₂₁H₂₇NO, 309.21; m/z found, 310.4 [M+H⁺.

### Example 98

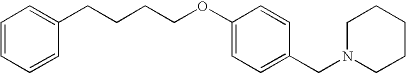

### 1-[4-(4-Phenyl-butoxy)-benzyl]-piperidine

The title compound was prepared according to Step A for EXAMPLE 93 using 3-phenbutanol followed by Step B for EXAMPLE 93 suing piperidine. MS (ESI): mass calculated for C₂₂H₂₉NO, 323.22; m/z found, 324.4 [M+H⁺.

### Example 99

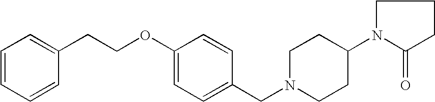

### 1-[1-(4-Phenethyloxy-benzyl)-piperidin-4-yl]-pyrrolidin-2-one

The title compound was prepared according to Step B for EXAMPLE 93 using 1-piperidin-4-yl-pyrrolidin-2-one. MS (ESI): mass calculated for C₂₄H₃₀N₂O₂, 378.23; m/z found, 379.5 [M+H⁺. ¹H NMR (400 MHz, CDCl₃): 7.32-7.23 (m, 4H), 7.17-7.22 (m, 3H), 6.83 (d, J=8.5, 2H), 4.13 (t, J=7.3, 2H), 4.02-3.92 (m, 1H), 3.52 (s, 2H), 3.30 (t, J=7.3, 2H), 3.06 (t, J=7.0, 2H), 3.01 (d, J=11.8, 2H), 2.34 (t, J=8.1, 2H), 2.16-2.08 (m, 2H), 1.98-1.89 (m, 2H), 1.85-1.74 (m, 2H), 1.64-1.57 (m, 2H).

### Example 100

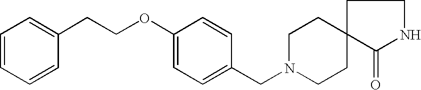

### 8-(4-Phenethyloxy-benzyl)-2,8-diaza-spiro[4.5]decan-1-one

The title compound was prepared according to Step B for EXAMPLE 93 using 2,8-diaza-spiro[4.5]decan-1-one. MS (ESI): mass calculated for C₂₃H₂₈N₂O₂, 364.22; m/z found, 365.5 [M+H⁺.

### Example 101

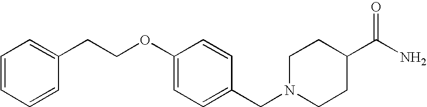

### 1-(4-Phenethyloxy-benzyl)-piperidine-4-carboxylic acid amide

The title compound was prepared according to Step B for EXAMPLE 93 using isonipecotamide. MS (ESI): mass calculated for C₂₁H₂₆N₂O₂, 338.2; m/z found, 339.5 [M+H⁺.

### Example 102

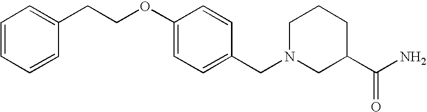

### 1-(4-Phenethyloxy-benzyl)-piperidine-3-carboxylic acid amide

The title compound was prepared according to Step B for EXAMPLE 93 using nipecotamide. MS (ESI): mass calculated for C₂₁H₂₆N₂O₂, 338.2; m/z found, 339.5 [M+H⁺.

### Example 103

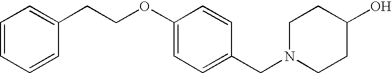

### 1-(4-Phenethyloxy-benzyl)-piperidin-4-ol

The title compound was prepared according to Step B for EXAMPLE 93 using 4-hydroxypiperidine. MS (ESI): mass calculated for C₂₀H₂₅NO₂, 311.19; m/z found, 312.4 [M+H⁺.

### Example 104

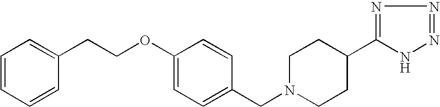

### 1-(4-Phenethyloxy-benzyl)-4-( 1H-tetrazol-5-yl)-piperidine

The title compound was prepared according to Step B for EXAMPLE 93 using 4-(1H-tetrazol-5-yl)-piperidine. MS (ESI): mass calculated for C₂₁H₂₅N₅O, 363.21; m/z found, 364.5 [M+H⁺.

### Example 105

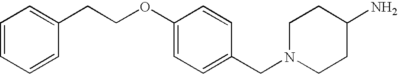

### 1-(4-Phenethyloxy-benzyl)-piperidin-4-ylamine

The title compound was prepared according to Steps A, B, and C for EXAMPLE 93. MS (ESI): mass calculated for C₂₀H₂₆N₂O, 310.2; m/z found, 311.5 [M+H⁺.

### Example 106

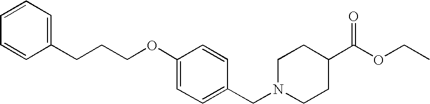

### 1-[4-(3-Phenyl-propoxy)-benzyl]-piperidine-4-carboxylic acid ethyl ester

The title compound was prepared according to Step A for EXAMPLE 93 using 3-phenpropanol followed by Step B for EXAMPLE 93 using ethyl 4-piperidinecarboxylic acid. MS (ESI): mass calculated for C₂₄H₃₁NO₃, 381.23; m/z found, 382.5 [M+H⁺.

### Example 107

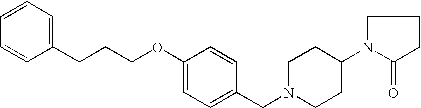

### 1-{1-[4-(3-Phenyl-propoxy)-benzyl]-piperidin-4-yl}-pyrrolidin-2-one

The title compound was prepared according to Step A for EXAMPLE 93 using 3-phenpropanol followed by Step B for EXAMPLE 93 using 1-piperidin-4-yl-pyrrolidin-2-one. MS (ESI): mass calculated for C₂₅H₃₅N₂O₂, 392.25; m/z found, 393.5 [M+H⁺. ¹H NMR (400 MHz, CDCl₃): 7.33-7.11 (m, 7H), 6.84 (d, J=8.5, 2H), 4.04-3.96 (m, 1H), 3.93 (t, J=6.3, 2H), 3.55 (s, 2H), 3.31 (t, J=6.8, 2H), 3.05 (d, J=11.6, 2H), 2.79 (t, J=7.3, 2H), 2.35 (t, J=7.8, 2H), 2.20-2.05 (m, 5H), 2.03 (s, 2H), 2.00-1.91 (m, 2H), 1.88-1.77 (m, 2H), 1.66-1.59 (m, 2H).

### Example 108

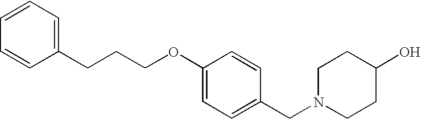

### 1-[4-(3-Phenyl-propoxy)-benzyl]-piperidin-4-ol

The title compound was prepared according to Step A for EXAMPLE 93 using 3-phenpropanol followed by Step B for EXAMPLE 93 using 4-hydroxypiperidine. MS (ESI): mass calculated for C₂₁H₂₇NO₂, 325.2; m/z found, 326.5 [M+H⁺.

### Example 109

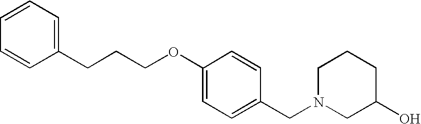

### 1-[4-(3-Phenyl-propoxy)-benzyl]-piperidin-3-ol

The title compound was prepared according to Step A for EXAMPLE 93 using 3-phenpropanol followed by Step B for EXAMPLE 93 using 3-hydroxypiperidine. MS (ESI): mass calculated for C₂₁H₂₇NO₂, 325.2; m/z found, 326.5 [M+H⁺.

### Example 110

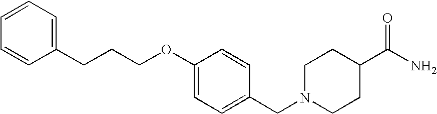

### 1-[4-(3-Phenyl-propoxy)-benzyl]-piperidine-4-carboxylic acid amide

The title compound was prepared according to Step A for EXAMPLE 93 using 3-phenpropanol followed by Step B for EXAMPLE 93 using isonipecotamide. MS (ESI): mass calculated for C₂₂H₂₈N₂O₂, 352.22; m/z found, 353.4 [M+H⁺.

### Example 111

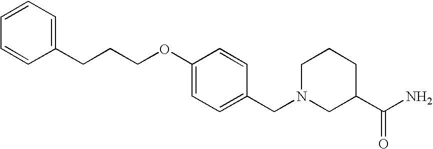

### 1-[4-(3-Phenyl-propoxy)-benzyl]-piperidine-3-carboxylic acid amide

The title compound was prepared according to Step A for EXAMPLE 93 using 3-phenpropanol followed by Step B for EXAMPLE 93 using nipecotamide. MS (ESI): mass calculated for C₂₂H₂₈N₂O₂, 352.22; m/z found, 353.4 [M+H⁺.

### Example 112

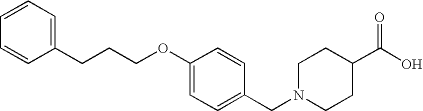

### 1-[4-(3-Phenyl-propoxy)-benzyl]-piperidine-4-carboxylic acid

The title compound was prepared from EXAMPLE 106 according to the procedure for EXAMPLE 35. MS (ESI): mass calculated for C₂₂H₂₇NO₃, 353.20; m/z found, 354.4 [M+H⁺. ¹H NMR (400 MHz, CDCl₃): 7.32-7.25 (m, 4H), 7.24-7.16 (m, 3H), 6.86 (d, J=8.8, 2H), 3.95 (t, J=6.3, 2H), 3.88 (s, 2H), 3.24 (d, J=11.6, 2H), 2.80 (t, J=7.5, 2H), 2.40-2.28 (m, 2H), 2.25-2.15 (m, 1H), 2.14-2.07 (m, 2H), 2.06-1.98 (m, 2H), 1.93-1.81 (m, 2H).

### Example 113

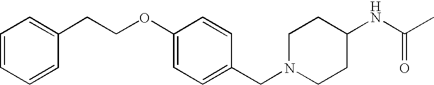

### N-[1-(4-Phenethyloxy-benzyl)-piperidin-4-yl]-acetamide

The title compound was prepared from EXAMPLE 105 according to Step C for EXAMPLE 93 using acetyl chloride. MS (ESI): mass calculated for C₂₂H₂₈N₂O₂, 352.22; m/z found, 353.4 [M+H⁺.

### Example 114

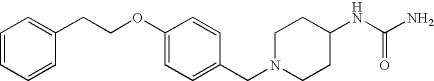

### [1-(4-Phenethyloxy-benzyl)-piperidin-4-yl]-urea

The title compound was prepared from EXAMPLE 105 according to Step C for EXAMPLE 93 using trimethylsilyl isocyanate. MS (ESI): mass calculated for C₂₁H₂₇N₃O₂, 353.21; m/z found, 354.4 [M+H⁺.

### Example 115

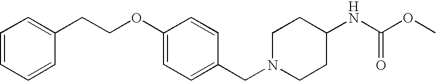

### [1-(4-Phenethyloxy-benzyl)-piperidin-4-yl]-carbamic acid methyl ester

The title compound was prepared from EXAMPLE 105 according to Step C for EXAMPLE 93 using methyl chloroformate. MS (ESI): mass calculated for C₂₂H₂₈N₂O₃, 368.21; m/z found, 369.4 [M+H⁺.

### Example 116

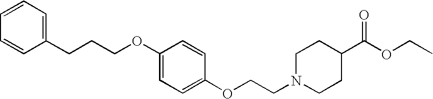

### 1-{2-[4-(3-Phenyl-propoxy)-phenoxy]-ethyl}-piperidine-4-carboxylic acid ethyl ester

The title compound was prepared using 1-[2-(4-hydroxy-phenoxy)-ethyl]-piperidine-4-carboxylic acid ethyl ester from Step A for EXAMPLE 35 and Step A for EXAMPLE 93 using 3-phenpropanol. MS (ESI): mass calculated for C₂₅H₃₃NO₄, 411.24; m/z found, 412.5 [M+H⁺.

### Example 117

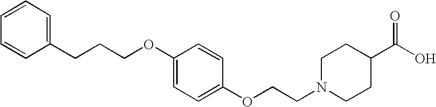

### 1-{2-[4-(3-Phenyl-propoxy)-phenoxy]-ethyl}-piperidine-4-carboxylic acid

The title compound was prepared using EXAMPLE 116 according to the procedure for EXAMPLE 35. MS (ESI): mass calculated for C₂₃H₂₉NO₄, 383.21; m/z found, 384.4 [M+H⁺. ¹H NMR (400 MHz, CDCl₃): 7.32-7.24 (m, 2H), 7.23-7.15 (m, 3H), 6.87-6.78 (m, 4H), 4.40 (br s, 3H), 3.89 (t, J=6.3, 2H), 3.78-3.22 (m, 5H), 2.78 (t, J=7.5, 2H), 2.77-2.73 (m, 1H), 2.32-2.19 (m, 4H), 2.10-2.02 (m, 2H).

### Example 118

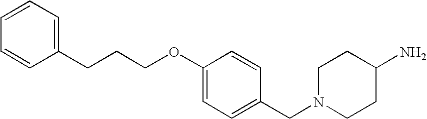

### 1-[4-(3-Phenyl-propoxy)-benzyl]-piperidin-4-ylamine

The title compound was prepared according to Step A for EXAMPLE 93 using 3-phenpropanol, followed by Step B for EXAMPLE 93 using piperidin-4-yl-carbamic acid tert-butyl ester, followed by Step C for EXAMPLE 93. MS (ESI): mass calculated for C₂₁H₂₈N₂O, 324.22; m/z found, 325.4 [M+H⁺.

### Example 119

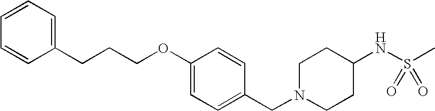

### N-{1-[4-(3-Phenyl-propoxy)-benzyl]-piperidin-4-yl}-methanesulfonamide

The title compound was prepared from EXAMPLE 118 following Step C for EXAMPLE 93. MS (ESI): mass calculated for C₂₂H₃₀N₂O₃S, 402.20; m/z found, 403.4 [M+H⁺. ¹H NMR (400 MHz, CDCl₃): 7.32-7.14 (m, 7H), 6.83 (d, J=8.5, 2H), 4.73 (d, J=7.5, 1H), 3.94 (t, J=6.8, 2H), 3.41 (s, 2H), 3.35-3.23 (m, 1H), 2.94 (s, 3H), 2.79 (t, J=7.8, 4H), 2.13-2.00 (m, 4H), 1.97-1.88 (m, 2H), 1.62-1.49 (m, 2H).

### Example 120

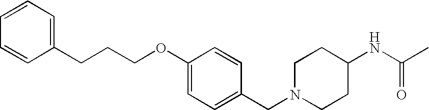

### N-{1-[4-(3-Phenyl-propoxy)-benzyl]-piperidin-4-yl}-acetamide

The title compound was prepared from EXAMPLE 118 following Step C for EXAMPLE 93 using acetyl chloride. MS (ESI): mass calculated for C₂₃H₃₀N₂O₂, 366.23; m/z found, 367.5 [M+H⁺.

### Example 121

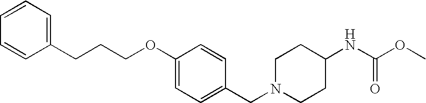

### {1-[4-(3-Phenyl-propoxy)-benzyl]-piperidin-4-yl}-carbamic acid methyl ester

The title compound was prepared from EXAMPLE 118 following Step C for EXAMPLE 93 using methyl chloroformate. MS (ESI): mass calculated for C₂₃H₃₀N₂O₃, 382.23; m/z found, 383.4 [M+H⁺.

### Example 122

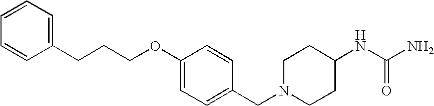

### {1-[4-(3-Phenyl-propoxy)-benzyl]-piperidin-4-yl}-urea

The title compound was prepared from EXAMPLE 118 following Step C for EXAMPLE 93 using trimethylsilyl isocyanate. MS (ESI): mass calculated for C₂₂H₂₉N₃O₂, 367.23; m/z found, 368.5 [M+H⁺.

The following Examples 123-126 were prepared according to the methods described in the preceeding examples.

### Example 123

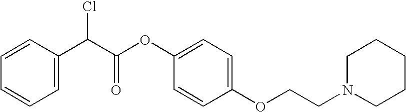

### Chloro-phenyl-acetic acid 4-(2-piperidin-1-yl-ethoxy)-phenyl ester

MS (ESI): mass calculated for C₂₁H₂₄ClNO₃, 373.14; m/z found, 374.3 [M+H⁺.

### Example 124

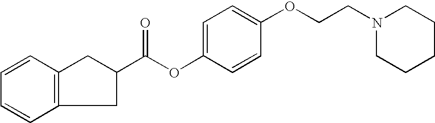

### Indan-2-carboxylic acid 4-(2-piperidin-1-yl-ethoxy)-phenyl ester

MS (ESI): mass calculated for C₂₃H₂₇NO₃, 365.20; m/z found, 366.4 [M+H⁺.

### Example 125

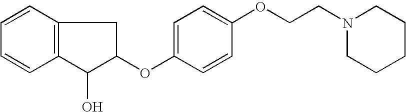

### 2-[4-(2-Piperidin-1-yl-ethoxy)-phenoxy]-indan-1-ol

MS (ESI): mass calculated for C₂₂H₂₇NO₃, 353.20; m/z found, 354.4 [M+H⁺.

### Example 126

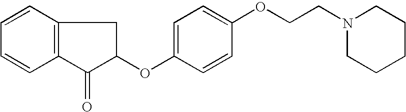

### 2-[4-(2-Piperidin-1-yl-ethoxy)-phenoxy]-indan-1-one

MS (ESI): mass calculated for C₂₂H₂₅NO₃, 351.18; m/z found, 352.4 [M+H⁺.

The following Examples 127-164 are prepared according to the procedures described in the preceeding examples.

### Example 127

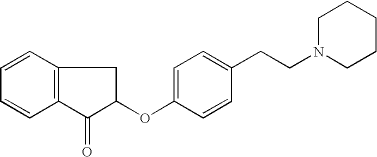

### 2-[4-(2-Piperidin-1-yl-ethyl)-phenoxy]-indan-1-one

### Example 128

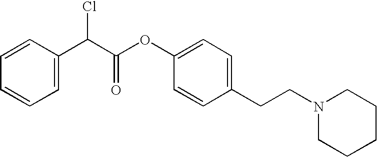

### Chloro-phenyl-acetic acid 4-(2-piperidin-1-yl-ethyl)-phenyl ester

### Example 129

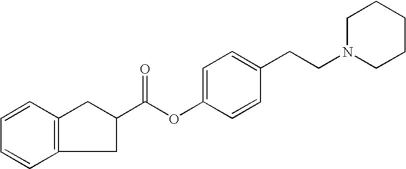

### Indan-2-carboxylic acid 4-(2-piperidin-1-yl-ethyl)-phenyl ester

### Example 130

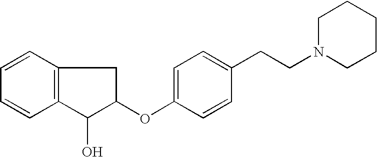

### 2-[4-(2-Piperidin-1-yl-ethyl)-phenoxy]-indan-1-ol

### Example 131

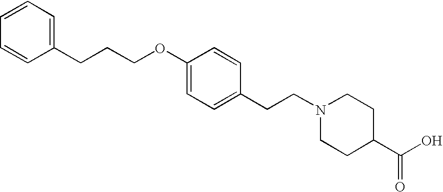

### 1-{2-[4-(3-Phenyl-propoxy)-phenyl]-ethyl}-piperidine-4-carboxylic acid

### Example 132

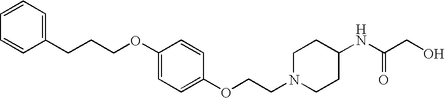

### 2-Hydroxy-N-(1-{2-[4-(3-phenyl-propoxy)-phenoxy]-ethyl}-piperidin-4-yl)-acetamide

### Example 133

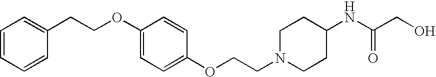

### 2-Hydroxy-N-{1-[2-(4-phenethyloxy-phenoxy)-ethyl]-piperidin-4-yl}-acetamide

### Example 134

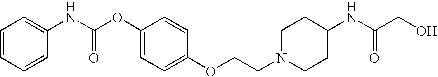

### Phenyl-carbamic acid 4-{2-[4-(2-hydroxy-acetylamino)-piperidin-1-yl]-ethoxy}-phenyl ester

### Example 135

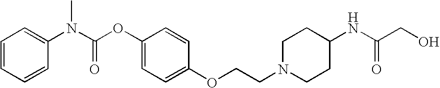

### Methyl-phenyl-carbamic acid 4-{2-[4-(2-hydroxy-acetylamino)-piperidin-1-yl]-ethoxy}-phenyl ester

### Example 136

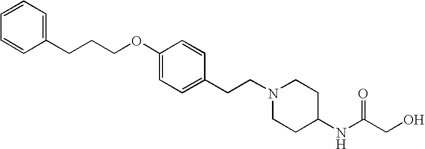

### 2-Hydroxy-N-(1-{2-[4-(3-phenyl-propoxy)-phenyl]-ethyl}-piperidin-4-yl )-acetamide

### Example 137

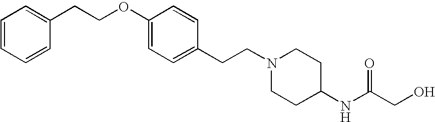

### 2-Hydroxy-N-{1-[2-(4-phenethyloxy-phenyl)-ethyl]-piperidin-4-yl}-acetamide

### Example 138

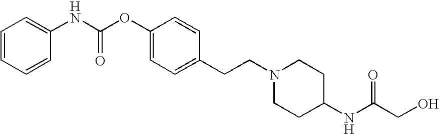

### Phenyl-carbamic acid 4-{2-[4-(2-hydroxy-acetylamino)-piperidin-1-yl]-ethyl}-phenyl ester

### Example 139

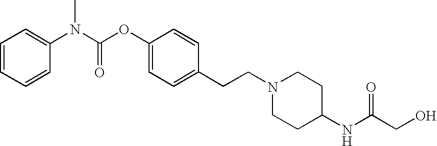

### Methyl-phenyl-carbamic acid 4-{2-[4-(2-hydroxy-acetylamino)-piperidin-1-yl]-ethyl}-phenyl ester

### Example 140

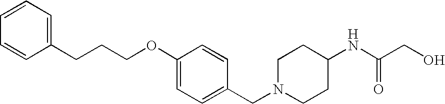

### 2-Hydroxy-N-{1-[4-(3-phenyl-propoxy)-benzyl]-piperidin-4-yl}-acetamide

### Example 141

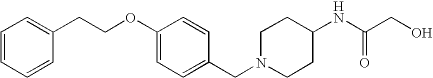

### 2-Hydroxy-N-[1-(4-phenethyloxy-benzyl)-piperidin-4-yl]-acetamide

### Example 142

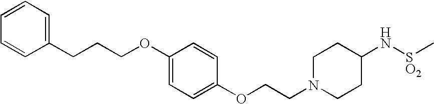

### N-(1-{2-[4-(3-Phenyl-propoxy)-phenoxy]-ethyl}-piperidin-4-yl)-methanesulfonamide

### Example 143

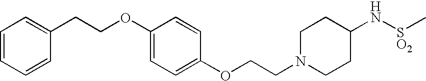

### N-{1-[2-(4-Phenethyloxy-phenoxy)-ethyl]-piperidin-4-yl}-methanesulfonamide

### Example 144

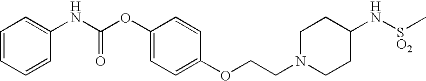

### Phenyl-carbamic acid 4-[2-(4-methanesulfonylamino-piperidin-1-yl)-ethoxy]-phenyl ester

### Example 145

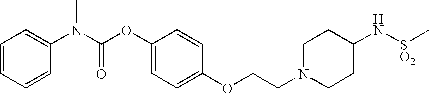

### Methyl-phenyl-carbamic acid 4-[2-(4-methanesulfonylamino-piperidin-1-yl )-ethoxy]-phenyl ester

### Example 146

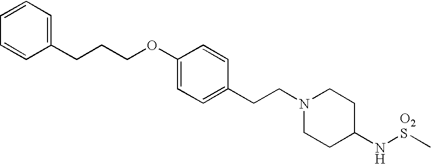

### N-(1-{2-[4-(3-Phenyl-propoxy)-phenyl]-ethyl}-piperidin-4-yl)-methanesulfonamide

### Example 147

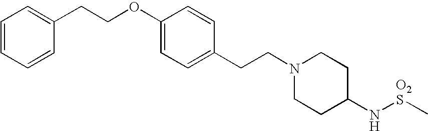

### N-{1-[2-(4-Phenethyloxy-phenyl)-ethyl]-piperidin-4-yl}-methanesulfonamide

### Example 148

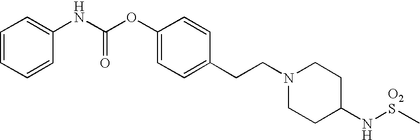

### Phenyl-carbamic acid 4-[2-(4-methanesulfonylamino-piperidin-1-yl)-ethyl]-phenyl ester

### Example 149

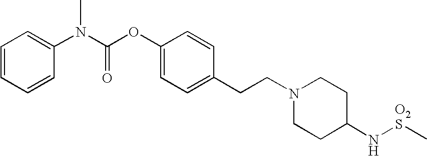

### Methyl-phenyl-carbamic acid 4-[2-(4-methanesulfonylamino-piperidin-1-yl)-ethyl]-phenyl ester

### Example 150

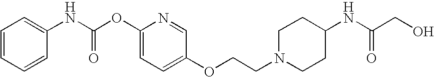

### Phenyl-carbamic acid 5-{2-[4-(2-hydroxy-acetylamino)-piperidin-1-yl]-ethoxy}-pyridin-2-yl ester

### Example 151

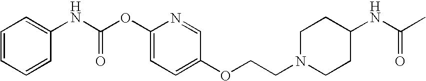

### Phenyl-carbamic acid 5-[2-(4-acetylamino-piperidin-1-yl)-ethoxy]-pyridin-2-yl ester

### Example 152

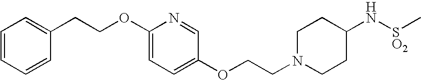

### N-{1-[2-(6-Phenethyloxy-pyridin-3-yloxy)-ethyl]-piperidin-4-yl}-methanesulfonamide

### Example 153

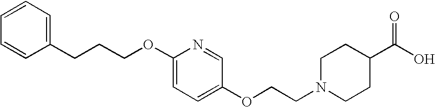

### 1-{2-[6-(3-Phenyl-propoxy)-pyridin-3-yloxy]-ethyl}-piperidine-4-carboxylic acid

### Example 154

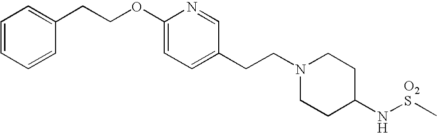

### N-{1-[2-(6-Phenethyloxy-pyridin-3-yl)-ethyl]-piperidin-4-yl}-methanesulfonamide

### Example 155

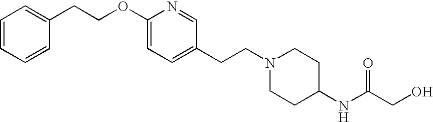

### 2-Hydroxy-N-{1-[2-(6-phenethyloxy-pyridin-3-yl)-ethyl]-piperidin-4-yl}-acetamide

### Example 156

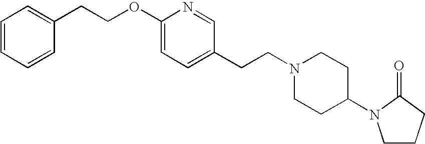

### 1-{1-[2-(6-Phenethyloxy-pyridin-3-yl)-ethyl]-piperidin-4-yl}-pyrrolidin-2-one

### Example 157

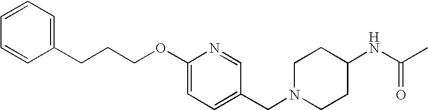

### N-{1-[6-(3-Phenyl-propoxy)-pyridin-3-ylmethyl]-piperidin-4-yl}-acetamide

### Example 158

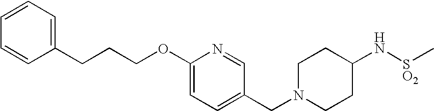

### N-{1-[6-(3-Phenyl-propoxy)-pyridin-3-ylmethyl]-piperidin-4-yl}-methanesulfonamide

### Example 159

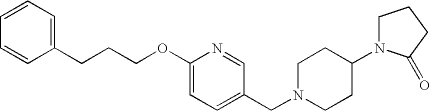

### 1-{1-[6-(3-Phenyl-propoxy)-pyridin-3-ylmethyl]-piperidin-4-yl}-pyrrolidin-2-one

### Example 160

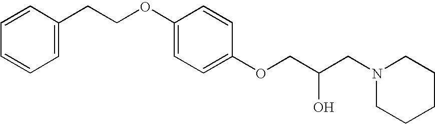

### 1-(4-Phenethyloxy-phenoxy)-3-piperidin-1-yl-propan-2-ol

### Example 161

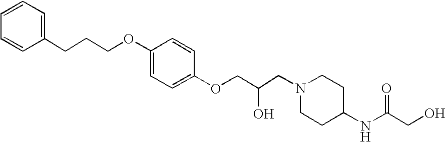

### 2-Hydroxy-N-(1-{2-hydroxy-3-[4-(3-phenyl-propoxy)-phenoxy]-propyl}-piperidin-4-yl)-acetamide

### Example 162

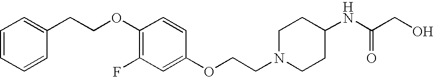

### N-{1-[2-(3-Fluoro-4-phenethyloxy-phenoxy)-ethyl]-piperidin-4-yl}-2-hydroxy-acetamide

### Example 163

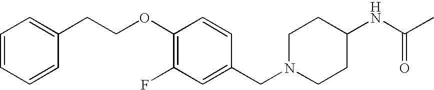

### N-[1-(3-Fluoro-4-phenethyloxy-benzyl)-piperidin-4-yl]-acetamide

### Example 164

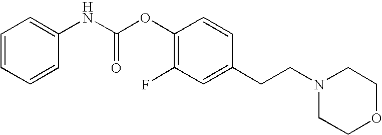

### Phenyl-carbamic acid 2-fluoro-4-(2-morpholin-4-yl-ethyl)-phenyl ester

### Example 165

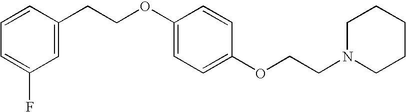

### 1-(2-{4-[2-(3-Fluoro-phenyl)-ethoxy]-phenoxy}-ethyl)-piperidine

The title compound was prepared from 3-fluorophenethyl alcohol according to the procedure for EXAMPLE 28. MS (ESI): mass calculated for C₂₁H₂₆FNO₂, 343.19; m/z found, 344.4 [M+H⁺.

### Example 166

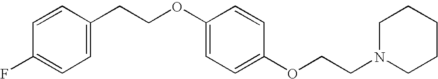

### 1-(2-{4-[2-(4-Fluoro-phenyl)-ethoxy]-phenoxy}-ethyl)-piperidine

The title compound was prepared from 4-fluorophenethyl alcohol according to the procedure for EXAMPLE 28. MS (ESI): mass calculated for C₂₁H₂₆FNO₂, 343.19; m/z found, 344.4 [M+H⁺.

Further examples of embodiments of this invention are provided by salt, ester and amide forms of compounds exemplified herein and equivalents thereof. By way of illustration, the carboxylic group in compounds such as Example 117 can form salts and esters, preferably pharmaceutically acceptable salts and esters; the basic nitrogen member in compounds such as Examples 1-166 can form salts, preferably pharmaceutically acceptable salts; and the carboxylic acid group in compounds such as Example 117 can form amides, wherein such salts, esters and amides are formed by methods known in the art.

### Assay Methods

Assay results provided herein are illustrative results of the assays that were performed for compounds of this invention.

Recombinant Human LTA4 Hydrolase Assay for LTA4 Hydrolase Inhibitor Activity

Compounds of the present invention were tested for LTA4 hydrolase inhibitor activity against recombinant human LTA4 hydrolase (rhLTA4H). Vectors were prepared and used to express rhLTA4H essentially as follows: LTA4 hydrolase encoding DNA was amplified by polymerase chain reaction (PCR) using a human placental cDNA library as a template. Oligonucleotide primers for the PCR reaction were based on the 5′-end, and the complement of the 3′-end, of the published nucleotide sequence for the coding region of the human LTA4 hydrolase gene (C. D. Funk et al., Proc. Natl. Acad. Sci. USA 1987, 84:6677-6681). The amplified 1.9 kD DNA fragment encoding LTA4 hydrolase was isolated and cloned into the pFastBac1 vector (Invitrogen). Recombinant baculovirus was generated as described by the manufacturer, and used to infect Spodoptera frugiperda (Sf-9) cells. Recombinant LTA4 hydrolase enzyme was purified from the infected Sf-9 cells essentially as described by J. K. Gierse et al. (Protein Expr. Purif. 1993, 4(5):358-366). The purified enzyme solution was adjusted to contain 0.29 mg/mL LTA4 hydrolase, 50 mM Tris (pH 8.0),150 mM NaCl, 5 mM dithiothreitol, 50% glycerol, and EDTA-free Complete protease inhibitor cocktail (Roche). The specific activity of the enzyme was about 3.8 μmol/min/mg.

LTA4 substrate was prepared from the methyl ester of LTA4 (Cayman Chemical) by treatment with 67 equiv. of NaOH under nitrogen at rt for 40 min. The LTA4 substrate in its free acid form was kept frozen at −80° C. until needed. Each compound was diluted to different concentrations in assay buffer (0.1 M potassium phosphate (pH 7.4), 5 mg/mL fatty acid free BSA) containing 10% DMSO. A 25-μL aliquot of each compound dilution was incubated for 10 min at rt with an equal volume of assay buffer containing 36 ng of recombinant human LTA4H. The solution was then adjusted to 200 μL with assay buffer. LTA4 (free acid) was thawed and diluted in assay buffer to a concentration of 357 ng/mL, and 25 μL (9 ng) of LTA4 substrate was added to the reaction mixture (total volume=225 μL) at time zero. Each reaction was carried out at rt for 10 min. The reaction was stopped by diluting 10 μL of the reaction mixture with 200 μL of assay buffer. LTB4 was quantifiedin the diluted sample by a commercially available enzyme-linked immunoassay (Cayman Chemical Co.), as recommended by the manufacturer. Positive controls, under essentially identical conditions but without addition of an inhibitor compound, and negative controls, containing all assay components except enzyme, were routinely run in each experiment. IC₅₀ values were determined by fitting the activity data at different compound concentrations to a 4-parameter equation using the Grafit program (Erithacus software).

The IC₅₀ values presented in the table below should be expected to fall within the typical three-fold variability of assays of this type. The values presented here are, in general, an average of one to three determinations.

TABLE 1

| Example | IC₅₀ (nM) |
|---|---|
| 8 | 100 |
| 9 | 389 |
| 10 | 52 |
| 11 | 0.3 |
| 12 | 3 |
| 13 | 10 |
| 14 | 1786 |
| 15 | 13 |
| 16 | 352 |
| 17 | 33 |
| 18 | 100 |
| 19 | 223 |
| 20 | 1 |
| 21 | 1 |
| 22 | 21 |
| 23 | 75 |
| 24 | 58 |
| 26 | 343 |
| 27 | 4 |
| 28 | 22 |
| 29 | 55 |
| 30 | 27 |
| 31 | 33 |
| 32 | 177 |
| 33 | 49 |
| 35 | 0.5 |
| 36 | 5 |
| 37 | 15 |
| 38 | 6 |
| 39 | 0.4 |
| 40 | 73 |
| 41 | 1 |
| 42 | 1 |
| 43 | 7 |
| 44 | 64 |
| 45 | 64 |
| 46 | 7 |
| 47 | 1 |
| 48 | 9 |
| 49 | 4 |
| 50 | 21 |
| 51 | 4 |
| 52 | 3 |
| 53 | 3 |
| 54 | 7 |
| 55 | 0.5 |
| 56 | 3 |
| 57 | 0.3 |
| 58 | 2 |
| 59 | 0.6 |
| 60 | 4 |
| 61 | 2 |
| 62 | 4 |
| 63 | 0.6 |
| 64 | 3 |
| 65 | 7 |
| 66 | 88 |
| 67 | 23 |
| 68 | 86 |
| 69 | 20 |
| 70 | 100 |
| 71 | 5 |
| 72 | 10 |
| 73 | 10 |
| 74 | 50 |
| 75 | 42 |
| 76 | 3 |
| 77 | 6 |
| 78 | 6 |
| 79 | 50 |
| 80 | 11 |
| 81 | 19 |
| 82 | 6 |
| 83 | 100 |
| 84 | 10 |
| 85 | 43 |
| 86 | 17 |
| 87 | 5 |
| 88 | 4 |
| 89 | 7 |
| 90 | 21 |
| 91 | 0.7 |
| 92 | 81 |
| 93 | 18 |
| 94 | 254 |
| 95 | 36 |
| 96 | 10 |
| 97 | 31 |
| 98 | 167 |
| 99 | 17 |
| 100 | 121 |
| 101 | 28 |
| 102 | 36 |
| 103 | 70 |
| 104 | 61 |
| 105 | 25 |
| 106 | 154 |
| 107 | 5 |
| 108 | 16 |
| 109 | 23 |
| 110 | 9 |
| 111 | 95 |
| 112 | 4 |
| 113 | 13 |
| 114 | 17 |
| 115 | 78 |
| 116 | 195 |
| 117 | 51 |
| 118 | 40 |
| 119 | 19 |
| 120 | 25 |
| 121 | 8 |
| 122 | 35 |
| 123 | 150 |
| 124 | 184 |
| 125 | 192 |
| 126 | 64 |
| 165 | 900 |
| 166 | 248 |

LTB4 Production by Calcium Ionophore-Stimulated Murine Blood for LTA4H Inhibitor Activity

CD-1 mice were sacrificed, and blood was collected in heparin-containing syringes by cardiac puncture. The blood was diluted 1:15 with RPMI-1640 medium, and 200-μL aliquots of the diluted blood were added to wells of a 96-well microtiter plate. LTA4H inhibitor test compounds were prepared at different concentrations in RPMI-1640 medium containing 1 % DMSO, and 20 μL of each test solution was added to a well containing diluted whole blood (final DMSO concentration of 0.1%). After the microtiter plate contents were incubated for 15 min at 37° C. in a humidified incubator, calcium ionophore A23187 (Sigma Chemical Co., St. Louis, Mo.) was added to each sample well (final concentration=20 ng/mL). The incubation was continued under the same conditions for an additional 10 min to allow LTB4 formation. The reaction was terminated by centrifugation (833×g, 10 min at 4° C.), and supernatants were analyzed for LTB4 by a commercially available enzyme-linked immunoassay (Cayman Chemical Co.) according to the manufacturer's instructions. Positive controls, under essentially identical conditions but without addition of an inhibitor compound, and negative unstimulated controls, containing all assay components except calcium ionophore, were routinely run in each experiment. IC₅₀ values were determined by fitting the activity data at different compound concentrations to a 4-parameter equation using the Grafit program (Erithacus software). Data are shown in Table 2.

TABLE 2

| Example | IC₅₀ (nM) |
|---|---|
| 11 | 12 |
| 12 | 88 |
| 13 | 47 |
| 24 | 202 |
| 30 | 169 |
| 40 | 150 |
| 51 | 12 |
| 64 | 48 |
| 67 | 175 |
| 69 | 215 |
| 79 | 192 |
| 81 | 181 |
| 93 | 16 |
| 96 | 7 |
| 99 | 123 |
| 104 | 284 |
| 107 | 33 |
| 112 | 10 |
| 113 | 56 |
| 117 | 79 |
| 119 | 93 |
| 120 | 126 |

Murine Arachidonic Acid-Induced Inflammation Model

LTA4H inhibitor compounds of the present invention were dissolved in 20% cyclodextran/H₂O at a concentration of 3 mg/mL. The solutions were administered by oral gavage to female Balb/c mice weighing approximately 20 grams each (0.2 mL per mouse, 30 mg of LTA4H inhibitor compound per kg). Sixty minutes after being administered an LTA4 inhibitor, each mouse received topical application of 20 μL of arachidonic acid (100 mg/mL in acetone) to the left ear and 20 μL of acetone only to the right ear. After 3 h, the mice were sacrificed, blood was withdrawn in heparinized syringes, and 8 mm ear biopsies were taken. Ear biopsies were weighed to determine edema and then frozen at −80° C. until needed for determination of neutrophil influx.

One hundred-microliter aliquots of heparinized blood were added to wells of a microtiter plate, along with equal volumes of RPMI-1640 medium, and calcium ionophore A23187 was added to each sample well (final concentration=20 ng/μL). The microtiter plate contents were incubated for 10 min at 37° C. in a humidified incubator. The reaction was terminated by centrifugation (833×g, 10 min at 4° C.). Supernatants were analyzed for LTB4 by a commercially available enzyme-linked immunoassay (Cayman Chemical Co.) in accordance with the manufacturer's instructions. The percent inhibition of ex vivo stimulated LTB4 production (% Inh. LTB4) was determined by comparison to animals treated identically except that the solution admininstered by oral gavage was devoid of inhibitor compound.

Neutrophil influx was quantified by measuring the activity of myeloperoxidase (MPO), a neutrophil-specific enzyme. The ear biopsies were homogenized in 0.5 mL extraction buffer (0.3 M sucrose, 0.22% (w/v) hexadecyl trimethyl ammonium bromide (CTAB), and 2.5 mM citrate prepared from 0.5 M citrate stock solution (pH 5.0)). Debris was removed by centrifugation at 14000×g for 10 min. Aliquots of 10 μL of the resulting supernatant were added to wells of a microtiter plate, along with 90-μL aliquots of dilution buffer (10 mM citrate, 0.22% CTAB), followed by addition of 20 μL TMB liquid substrate system (Sigma Chemical Co.) to each sample well. The microtiter plate contents were held at rt for 1 h. The reaction was stopped by addition of 100 μL 1 M H₂SO₄ to each sample well, and the myeloperoxidase activity in each sample was determined from the absorbance at 405 nm. The background value from the right ear, treated only with acetone, was subtracted from that for the left ear, treated with arachidonic acid in acetone, for each animal. The percent inhibition of neutrophil influx (% lnh. MPO) by compounds of the invention was determined by comparison to animals treated identically, except that the solution administered by oral gavage was devoid of inhibitor compound. Data are shown in Table 3.

TABLE 3

| | % Inh. | % Inh. |
|---|---|---|
| Example | LTB4 | MPO |
| 15 | 78 | 92 |
| 35 | 84 | 92 |
| 40 | 79 | 75 |
| 47 | 80 | 95 |
| 48 | 69 | 66 |
| 52 | 80 | 87 |
| 81 | 78 | 64 |
| 89 | 36 | 41 |
| 96 | 80 | 95 |
| 99 | 68 | 17 |
| 112 | 91 | 79 |
| 117 | 74 | 62 |

References cited in the specification are incorporated herein by reference. Having described the invention in specific detail and exemplified the manner in which it may be carried into practice, it will be apparent to those skilled in the art that innumerable variations, applications, modifications, and extensions of the basic principles involved may be made without departing from its spirit or scope. It is to be understood that the foregoing is merely exemplary and the present invention is not to be limited to the specific form or arrangements of parts herein described and shown.

What is claimed is:

1. A method for inhibiting LTA4H enzyme activity, comprising exposing LTA4H enzyme to an inhibitory amount of at least one compound of formula (I): 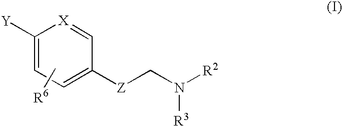
wherein
X is selected from the group consisting of CH and N;
Y is selected from the group consisting of R¹(CH₂)₂₋₃O—, R⁷N(R⁸)CO₂—, R⁷N(R⁸)C(O)N(R⁸)—, R⁷N(R⁸)CO₂CH₂—, R⁷N(R⁸)C(O)CH₂—, R¹OC(O)N(R⁸)—, R¹OCO₂—, R¹CO₂—, R¹CH(R⁹)CO₂—, R¹C(O)CH(R¹⁰)O—, and R¹CH(R⁹)CH(R¹⁰)O—, provided that when one of R⁹ and R¹⁰ in R¹CH(R⁹)CH(R¹⁰)O— is —H, then the other is not —H;
R¹ is a moiety selected from the group consisting of phenyl, thienyl, pyrrolyl, furanyl, oxazolyl, imidazolyl, thiazolyl, indolyl, indanyl, and tetrahydronaphthyl, wherein R¹ is substituted with 0, 1, or 2 substituents R⁴;
R⁴ is selected from the group consisting of —H, —OCH₃, —Cl, —F, —Br, —I, —OH, —NH₂, —CN, —CF₃, and —CH₃;
R⁷ is —C₁₄alkyl or is selected from the group consisting of phenyl, thienyl, pyrrolyl, furanyl, oxazolyl, imidazolyl, thiazolyl, indolyl, indanyl, and tetrahydronaphthyl, wherein R⁷ is substituted with 0, 1, or 2 substituents R⁴;
R⁸ is —H or —C₁₋₄alkyl;
or, R and R8 are taken together with the nitrogen member to which they are attached to form pyrrolidinyl, piperidinyl, morpholinyl, or thiomorpholinyl;
R⁹ is —H, —C₁₋₄alkyl, —Cl, or —OH;
R¹⁰ is —H, —C₁₋₄alkyl or is taken together with one of R⁴ to form a 5- or 6-membered carbocyclic ring;
Z is selected from the group consisting of bond, —CH₂—, —OCH₂—, —OCH₂CH(R¹¹)—, and —CH₂CH(R¹¹)—;
R¹¹ is —H or —OH;
provided that when Z is bond, then Y is one of R¹(CH₂)₂₋₃O—, R¹CO₂—, R¹CH(R⁹)CO₂—, R¹C(O)CH(R¹⁰)O—, and R¹CH(OH)CH(R¹⁰)O—;
R⁶ is —H or —F; and
R² and R³ are each independently selected from the group consisting of
A) —H, —C₁₋₇alkyl, —C₃₋₇alkenyl, wherein the carbon in said alkenyl that is attached to the nitrogen member has only single bonds, —C₃₋₇alkynyl, wherein the carbon in said alkynyl that is attached to the nitrogen member has only single bonds, —C₃₋₇cycloalkyl optionally benzofused, —C₅₋₇cycloalkenyl, —C₃₋₇cycloalkylC₁₋₇alkyl, —C₁₋₇alkylC₃₋₇cycloalkyl and phenyl, wherein each of the substituents A) is independently substituted with 0, 1, or 2 substituents R^{Q}, and each of said R^{Q} is a substituent at a carbon member that is at least one carbon member removed from the nitrogen member;
B) a 4-7 membered saturated heterocyclic ring HetR^{a}, said 4-7 membered saturated heterocyclic ring HetR^{a}, having 0 or 1 double bonds, having a carbon member point of attachment and containing a member >NR^{M} as a heteroatom member, and said heteroatom member being separated from said carbon member point of attachment by at least one additional carbon member;
C) —C₁₋₇alkylC(O)R^{x}, optionally substituted with CH₂R^{Ar} or CH₂R^{Ar′};
D) —C₂₋₅alkylC(O)R^{x}, wherein two valence allowed carbon members in the C₂₋₅alkyl of said —C₂₋₅alkylC(O)R^{x} are part of a saturated C₃₋₆carbocycle;
E) —C₂₋₅alkylOH wherein two valence allowed carbon members in the C₂₋₅alkyl of said —C₂₋₅alkylOH are part of a saturated C₃₋₆carbocycle;
F) —C₀₋₄alkylphenyl, wherein the phenyl in said —C₀₋₄alkylphenyl is fused at two adjacent carbon members in said phenyl to R^{f}, or is benzofused;
G) —C₀₋₄alkylAr⁶, where Ar⁶ is a 6-membered heteroaryl having a carbon member point of attachment and having 1 or 2 —N═ heteroatom members, and benzofused;
H) —C₀₋₄alkylAr⁵, where Ar⁵ is a 5-membered heteroaryl, having one heteroatom member selected from the group consisting of O, S, and >NR^{Y}, and having 0 or 1 —N═ additional heteroatom member, optionally containing 1 or 2 carbonyl groups, and optionally benzofused;
I) —C₁₋₄alkylAr^{5′}, where Ar^{5′} is a 5-membered heteroaryl containing 3 or 4 nitrogen members, optionally substituted with R^{Y}, and having a valence allowed site as a point of attachment;
J) —C₀₋₄alkylAr⁶⁻⁶, where Ar⁶⁻⁶ is a C₀₋₄alkyl-attached phenyl fused at valence allowed sites to a 6-membered heteroaryl, wherein said 6-membered heteroaryl has 1 or 2 —N═ heteroatom members;
K) —C₀₋₄alkylAr⁶⁻⁵, where Ar⁶⁻⁵ is a C₀₋₄alkyl-attached phenyl fused at valence allowed sites to a 5-membered heteroaryl, said 5-membered heteroaryl having one heteroatom member selected from the group consisting of O, S, and >NR^{Y}, and said 5-membered heteroaryl having 0 or 1 additional heteroatom member which is —N═;
L) one of 2-(4-ethyl-phenoxy)-benzothiazole, 2-(4-ethyl-phenoxy)-benzooxazole, and 2-(4-ethyl-phenoxy)-1H-benzoimidazole; and
M) —SO₂C₁₋₄alkyl;
alternatively R² and R³ are taken together with the nitrogen to which they are attached to form a heterocyclic ring that contains at least one heteroatom member that is said attachment nitrogen, said heterocyclic ring being selected from the group consisting of
i) a 4-7 membered saturated heterocyclic ring HetR^{b}, said 4-7 membered saturated heterocyclic ring HetR^{b} having one heteroatom member that is said attachment nitrogen, and being substituted with 0, 1, or 2 substituents at the same or at different ring members, said substituents being selected from the group consisting of —R^{Y}, —CN, —C(O)R^{Y}, —C₀₋₄alkylCO₂R^{Y}, —C₀₋₄alkylC(O)CO₂R^{Y}, —C₀₋₄alkylOR^{Y},—C₀₋₄alkylC(O)NR^{Y}R^{Z}, —C₀₋₄alkylNR^{Y}C(O)R^{Z}, —C(O)NR^{Z}OR^{Y}, —C₀₋₄alkylNR^{Y}C(O)CH₂OR^{Y}, —C₀₋₄alkylNR^{Y}C(O)CH₂C(O)R^{Y}, —C₀₋₄alkylNR^{Y}CO₂R^{Y}, —C₀₋₄alkylNR^{Y}C(O)NR^{Y}R^{Z}, —C₀₋₄alkylNR^{Y}C(S)NR^{Y}R^{Z}, —NR^{Y}C(O)CO₂R^{Y}, —NR^{Y}R^{Z}, —C₀₋₄alkylNR^{W}SO₂R^{Y}, 1,3-dihydro-indol-2-one-1-yl, 1,3-dihydro-benzoimidazol-2-one-1-yl, tetrazol-5-yl, 1-R^{Y}-1H-tetrazol-5-yl, R^{Y}-triazolyl, 2-R^{Y}-2H-tetrazol-5-yl, pyrrolidine-2-thion-1-yl, piperidine-2-thion-1-yl, —C₀₋₄alkylC(O)N(R^{Y})(SO₂R^{Y}), —C₀₋₄alkylN(R^{Y})(SO₂)NR^{Y}R^{Y}, —C₀₋₄alkylN(R^{Y})(SO₂)NR^{Y}CO₂R^{Y}, halo, 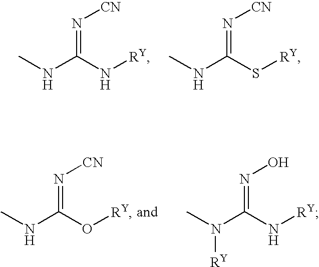
ii) a 5-7 membered saturated heterocyclic ring HetR^{c}, said 5-7 membered saturated heterocyclic ring HetR^{c} having one additional heteroatom member separated from said attachment nitrogen by at least one carbon member, said additional heteroatom member being selected from the group consisting of O, S(═O)₀₋₂, and >NR^{M}, said 5-7 membered saturated heterocyclic ring HetR^{c} having 0 or 1 carbonyl members, and being substituted with 0, 1, or 2 substituents at the same or at different carbon ring members, said substituents being selected from the group consisting of —C(O)R^{Y}, —CO₂R^{Y}, —C₃₋₄alkylCO₂R^{Y} and R^{Z};
iii) one of imidazolidin-1-yl, 2-imidazolin-1-yl, pyrazol-1-yl, imidazol-1-yl, 2H-tetrazol-2-yl, 1H-tetrazol-1-yl, pyrrol-1-yl, 2-pyrrolin-1-yl, and 3-pyrrolin-1-yl, wherein each of said 2H-tetrazol-2-yl and 1H-tetrazol-1-yl is substituted at the carbon member with 0 or 1 of —C₀₋₄alkylR^{Z}, —C₀₋₄alkylSR^{Y}, —C₀₋₄alkylC₀₋₂R^{Y}, and substituent HetR^{a}; and
iv) one of 1,2,3,4-tetrahydro-quinolin-1-yl, 1,2,3,4-tetrahydro-isoquinolin-2-yl, indol-1-yl, isoindol-2-yl, indolin-1-yl, benzimidazol-1-yl, 2,8-diaza-spiro[4.5]decan-1-one-8-yl, 4-{[(2-tert-butoxycarbonylamino-cyclobutanecarbonyl)-amino]-methyl}-piperidin-1-yl, 4-{[(2-amino-cyclobutanecarbonyl)-amino]-methyl}-piperidin-1-yl, 3,9-diaza-spiro[5.5]undecane-3-carboxylic acid-9-yl tert-butyl ester, 4-oxo-1-phenyl-1,3,8-triaza-spiro[4.5]dec-8-yl, and 4-oxo-1,3,8-triaza-spiro[4.5]dec-8-yl;
wherein
R^{K} is selected from the group consisting of —H, —C₁₋₄alkyl and —C₀₋₄alkylR^{Ar}, each of said —C₁₋₄alkyl and —C₀₋₄alkylR^{Ar} being optionally substituted with 1, 2, or 3 substituents R^{N};
R^{L} is selected from the group consisting of —CO₂R^{S} and —C(O)NR^{S}R^{S′};
R^{M} is selected from the group consisting of R^{Z}, indol-7-yl, —SO₂R^{Y}, —C₃₋₄alkylC₀₋₂R^{Y}, —CO₂R^{Y}, —C(O)NR^{Z}OR^{Y}, —C(O)R^{Y}, —C(O)C₁₋₄alkylOR^{Y}, —C₀₋₄alkylC(O)NR^{S}R^{S}′, C₀₋₄alkylC(O)CO₂R^{Y}, 1,3-dihydro-indol-2-one-1-yl, 1,3-dihydro-benzoimidazol-2-one-1-yl, tetrazol-5-yl, 1-R^{Y}-1H-tetrazol-5-yl, R^{Y}-triazolyl, 2-R^{Y}-2H-tetrazol-5-yl and —C₀₋₄alkylC(O)N(R^{Y})(SO₂R^{Y}), each of said R^{M} that is not —H being optionally substituted with 1, 2, or 3 substituents R^{N};
R^{N} is selected from the group consisting of —OCH₃, —Cl, —F, —Br, —I, —OH, —NH₂, —CN, —CF₃, —CH₃, —OC(O)CH₃, and —NO₂;
R^{Q} is selected from the group consisting of —Cl, —F, —Br, —I, —CF₃, —CCl₃, —CN, —C₁₋₄alkyl, —CO₄alkylR^{Ar}, —C₀₋₄alkylR^{Ar′}, —C₀₋₄alkylOR^{Y}, —C₀₋₄alkylCO₂R^{Y}, —C₀₋₄alkylNR^{Y}R^{Z}, —C₀₋₄alkylNR^{Y}COR^{Y}, —C₀₋₄alkylNR^{Y}CONR^{Y}R^{Z}, —C₀₋₄alkylNR^{Y}SO₂R^{Y}, and —C₀₋₄alkylSR^{Y};
R^{S} and R^{S′} are independently selected from the group consisting of —H, —C₁₋₄alkyl, and —C₀₋₄alkylphenyl; alternatively, R^{S} and R^{S′} are taken together with the nitrogen member to which said R^{S} and R^{S′} are attached to form a 4-7 membered heterocyclic ring having 0 or 1 additional heteroatom member selected from the group consisting of O, S, and >NR^{Y}, provided that said additional heteroatom member is separated by at least two carbon members from said nitrogen member to which said R^{S} and R^{S′} are attached, and provided that where R^{Y} is C₀₋₄alkylR^{Ar}, then R^{Ar} is not substituted with R^{L};
R^{W} is selected from the group consisting of R^{Y}, and —C₃₋₇cycloalkyl;
R^{X} is selected from the group consisting of —OR^{Y}, —NR^{Y}R^{Z}, —C₁₋₄alkyl, and —C₀₋₄alkylR^{Ar};
R^{Y} is selected from the group consisting of —H, —C₁₋₄alkyl, —C₀₋₄alkylR^{Ar} and —C₀₋₄alkylR^{Ar′}, each of said R^{Y} that is not —H being optionally substituted with 1, 2, or 3 substituents R^{N};
R^{Z} is selected from the group consisting of R^{Y}, —C₂₋₄alkylOR^{Y}, —C₁₋₂alkylCO₂R^{Y}, —C₁₋₂alkylC(O)NR^{S}R^{S′}, and —C₂₋₄alkylNR^{S}R^{S′};
provided that when R^{Y} and R^{Z} are attached to a nitrogen member, then R^{Y} and R^{Z} are selected as defined above, or R^{Y} and R^{Z} are taken together with the R^{Y}— and R^{Z}— attached nitrogen member to form a 4-7 membered heterocyclic ring HetR^{d} having 0 or 1 additional heteroatom members selected from the group consisting of O, S, and >NR^{M}, said 4-7 membered heterocyclic ring HetR^{d} having 0 or 1 carbonyl members, and said 4-7 membered heterocyclic ring HetR^{d} having 0 or 1 valence allowed carbon members substituted with at least one of R^{M}, —CO₂H, and —C₀₋₁alkylOR^{Y};
R^{Ar} is a moiety with a carbon member attachment point and said R^{Ar} is selected from the group consisting of phenyl, pyridyl, pyrimidyl, and pyrazinyl, wherein each valence allowed carbon member in each of said R^{Ar} is independently substituted with at least one of 0, 1, 2, or 3 substituents R^{N}, and 0 or 1 substituent R^{L};
R^{Ar′} is a 3-8 membered ring having 0, 1, or 2 heteroatom members selected from the group consisting of O, S, N, and >NR^{Y}, said R^{Ar′} having 0, 1, or 2 unsaturated bonds and having 0 or 1 carbonyl members, wherein each valence allowed member in each of said R^{Ar′} ring is independently substituted with 0, 1, or 2 substituents R^{K}; and
R^{f} is a linear 3- to 5-membered hydrocarbon moiety having 0 or 1 unsaturated carbon-carbon bonds and having 0 or 1 carbonyl members;
or an enantiomer, diasteromer, racemate, tautomer, hydrate, solvate, or a pharmaceutically acceptable salt, ester, or amide thereof.

2. The method of claim 1, wherein said at least one compound of formula (I) is at least one compound of formula (II): 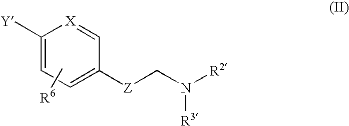
wherein
X is selected from the group consisting of CH and N;
Y′ is selected from the group consisting of R¹(CH₂)₂₋₃O—, R⁷N(R⁸)CO₂—, R⁷N(R⁸)C(O)N(R⁸)—, R⁷N(R⁸)CO₂CH₂—, R⁷N(R⁸)C(O)CH₂—, R¹OC(O)N(R⁸)—, R¹OCO₂—, R¹CO₂—, R¹CH(R⁹)CO₂—, R¹C(O)CH(R¹⁰)O—, and R¹CH(R⁹)CH(R¹⁰)O—, provided that when one of R⁹ and R¹⁰ in R¹CH(R⁹)CH(R¹⁰)O— is —H, then the other is not —H;
R¹ is a moiety selected from the group consisting of phenyl, thienyl, pyrrolyl, furanyl, oxazolyl, imidazolyl, thiazolyl, indolyl, indanyl, and tetrahydronaphthyl, wherein R¹ is substituted with 0, 1, or 2 substituents R⁴;
R⁴ is selected from the group consisting of —H, —OCH₃, —Cl, —F, —Br, —I, —OH, —NH₂, —CN, —CF₃, and —CH₃;
R⁷ is —C₁₋₄-alkyl or is selected from the group consisting of phenyl, thienyl, pyrrolyl, furanyl, oxazolyl, imidazolyl, thiazolyl, indolyl, indanyl, and tetrahydronaphthyl, wherein R⁷ is substituted with 0, 1, or 2 substituents R⁴;
R⁸ is —H or —C₁₋₄alkyl;
or, R⁷ and R⁸ are taken together with the nitrogen member to which they are attached to form pyrrolidinyl, piperidinyl, morpholinyl, or thiomorpholinyl;
R⁹ is —H, —C₁₋₄alkyl, —Cl, or —OH;
R¹⁰ is —H, —C₁₋₄alkyl or is taken together with one of R⁴ to form a 5- or 6-membered carbocyclic ring;
R¹¹ is —H or —OH;
Z is selected from the group consisting of bond, —CH₂—, —OCH₂—, —OCH₂CH(R¹¹)—, and —CH₂CH(R¹¹)—;
provided that when Z is bond, then Y′ is one of R¹(CH₂)₂₋₃O—, R¹CO₂—, R¹CH(R⁹)CO₂—, R¹C(O)CH(R¹⁰)O—, and R¹CH(OH)CH(R¹⁰)O—;
R⁶ is —H or —F; and
R^{2′} and R^{3′} are each independently selected from the group consisting of
A) H, C₁₋₇alkyl, C₃₋₇alkenyl, wherein the carbon in said alkenyl that is attached to the nitrogen member has only single bonds, C₃₋₇alkynyl, wherein the carbon in said alkynyl that is attached to the nitrogen member has only single bonds, C₃₋₇cycloalkyl optionally benzofused, C₅₋₇cycloalkenyl, C₃₋₇cycloalkylC₁₋₇alkyl, C₁₋₇alkylC₃₋₇cycloalkyl and phenyl, wherein each of the substituents A) is independently substituted with 0, 1, or 2 substituents R^{Q}, and each of said R^{Q} is a substituent at a carbon member that is at least one carbon member removed from the nitrogen member;
B) a 4-7 membered saturated heterocyclic ring HetR^{a}, said 4-7 membered saturated heterocyclic ring HetR^{a}, having 0 or 1 double bonds, having a carbon member point of attachment and containing a member >NR^{M} as a heteroatom member, and said heteroatom member being separated from said carbon member point of attachment by at least one additional carbon member;
C) —C₁₋₇alkylC(O)R^{x}, optionally substituted with CH₂R^{Ar} or CH₂R^{Ar′};
D) —C₂₋₅alkylC(O)R^{x}, wherein two valence allowed carbon members in the C₂₋₅alkyl of said —C₂₋₅alkylC(O)R^{x} are part of a saturated C₃₋₆carbocycle;
E) —C₂₋₅alkylOH wherein two valence allowed carbon members in the C₂₋₅alkyl of said —C₂₋₅alkylOH are part of a saturated C₃₋₆carbocycle;
F) —C₀₋₄alkylphenyl, wherein the phenyl in said —C₀₋₄alkylphenyl is fused at two adjacent carbon members in said phenyl to R^{f}, or is benzofused;
G) —C₀₋₄alkylAr⁶, where Ar⁶ is a 6-membered heteroaryl having a carbon member point of attachment and having 1 or 2 —N═ heteroatom members, and benzofused;
H) —C₀₋₄alkylAr⁵, where Ar⁵ is a 5-membered heteroaryl, having one heteroatom member selected from the group consisting of O, S, and >NR^{Y}, and having 0 or 1 —N═ additional heteroatom member, optionally containing 1 or 2 carbonyl groups, and optionally benzofused;
I) —C₁₋₄alkylAr^{5′}, where Ar^{5′} is a 5-membered heteroaryl containing 3 or 4 nitrogen members, optionally substituted with R^{Y}, and having a valence allowed site as a point of attachment;
J) —C₀₋₄alkylAr⁶⁻⁶, where Ar⁶⁻⁶ is a C₀₋₄alkyl-attached phenyl fused at valence allowed sites to a 6-membered heteroaryl, wherein said 6-membered heteroaryl has 1 or 2 —N═ heteroatom members;
K) —C₀₋₄alkylAr⁶⁻⁵, where Ar⁶⁻⁵ is a C₀₋₄alkyl-attached phenyl fused at valence allowed sites to a 5-membered heteroaryl, said 5-membered heteroaryl having one heteroatom member selected from the group consisting of O, S, and >NR^{Y}, and said 5-membered heteroaryl having 0 or 1 additional heteroatom member which is —N═;
L) one of 2-(4-ethyl-phenoxy)-benzothiazole, 2-(4-ethyl-phenoxy)-benzooxazole, and 2-(4-ethyl-phenoxy)-1H-benzoimidazole; and
M) —SO₂C₁₋₄alkyl;
alternatively R^{2′} and R^{3′} are taken together with the nitrogen to which they are attached to form a heterocyclic ring that contains at least one heteroatom member that is said attachment nitrogen, said heterocyclic ring being selected from the group consisting of
i) a 4-7 membered saturated heterocyclic ring HetR^{b}, said 4-7 membered saturated heterocyclic ring HetR^{b} having one heteroatom member that is said attachment nitrogen, and being substituted with 0, 1, or 2 substituents at the same or at different ring members, said substituents being selected from the group consisting of —R^{Y}, —CN, —C(O)R^{Y}, —C₀₋₄alkylCO₂R^{Y}, —C₀₋₄alkylC(O)CO₂R^{Y}, —C₀₋₄alkylOR^{Y},—C₀₋₄alkylC(O)NR^{Y}R^{Z}, —C₀₋₄alkylNR^{Y}C(O)R^{Z}, —C(O)NR^{Z}OR^{Y}, —C₀₋₄alkylNR^{Y}C(O)CH₂OR^{Y}, —C₀₋₄alkylNR^{Y}C(O)CH₂C(O)R^{Y}, —C₀₋₄alkylNR^{Y}CO₂R^{Y}, —C₀₋₄alkylNR^{Y}C(O)NR^{Y}R^{Z}, —C₀₋₄alkylNR^{Y}C(S)NR^{Y}R^{Z}, —NR^{Y}C(O)CO₂R^{Y}, —NR^{Y}R^{Z}, —C₀₋₄alkylNR^{W}SO₂R^{Y}, 1,3-dihydro-indol-2-one-1-yl, 1,3-dihydro-benzoimidazol-2-one-1-yl, tetrazol-5-yl, 1-R^{Y}-1H-tetrazol-5-yl, R^{Y}-triazolyl, 2-R^{Y}-2H-tetrazol-5-yl, pyrrolidine-2-thion-1-yl, piperidine-2-thion-1-yl, —C₀₋₄alkylC(O)N(R^{Y})(SO₂R^{Y}), —C₀₋₄alkylN(R^{Y})(SO₂)NR^{Y}R^{Y}, —C₀₋₄alkylN(R^{Y})(SO₂)NR^{Y}CO₂R^{Y}, halo, 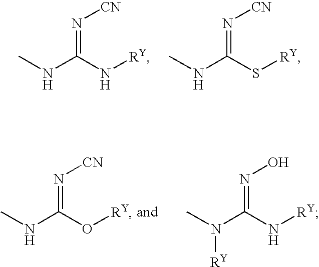
ii) a 5-7 membered saturated heterocyclic ring HetR^{c}, said 5-7 membered saturated heterocyclic ring HetR^{c} having one additional heteroatom member separated from said attachment nitrogen by at least one carbon member, said additional heteroatom member being selected from the group consisting of O, S(═O)₀₋₂, and >NR^{M}, said 5-7 membered saturated heterocyclic ring HetR^{c} having 0 or 1 carbonyl members, and being substituted with 0, 1, or 2 substituents at the same or at different carbon ring members, said substituents being selected from the group consisting of —C(O)R^{Y}, —CO₂R^{Y}, —C₃₋₄alkylCO₂R^{Y} and R^{Z};
iii) one of imidazolidin-1-yl, 2-imidazolin-1-yl, pyrazol-1-yl, imidazol-1-yl, 2H-tetrazol-2-yl, 1H-tetrazol-1-yl, pyrrol-1-yl, 2-pyrrolin-1-yl, and 3-pyrrolin-1-yl, wherein each of said 2H-tetrazol-2-yl and 1H-tetrazol-1-yl is substituted at the carbon member with 0 or 1 of —C₀₋₄alkylR^{Z}, —C₀₋₄alkylSR^{Y}, —C₀₋₄alkylC₀₋₂R^{Y}, and substituent HetR^{a}; and
iv) one of 1,2,3,4-tetrahydro-quinolin-1-yl, 1,2,3,4-tetrahydro-isoquinolin-2-yl, indol-1-yl, isoindol-2-yl, indolin-1-yl, benzimidazol-1-yl, 2,8-diaza-spiro[4.5]decan-1-one-8-yl, 4-{[(2-tert-butoxycarbonylamino-cyclobutanecarbonyl)-amino]-methyl}-piperidin-1-yl, 4-{[(2-amino-cyclobutanecarbonyl)-amino]-methyl}-piperidin-1-yl, 3,9-diaza-spiro[5.5]undecane-3-carboxylic acid-9-yl tert-butyl ester, 4-oxo-1-phenyl-1,3,8-triaza-spiro[4.5]dec-8-yl, and 4-oxo-1,3,8-triaza-spiro[4.5]dec-8-yl;
wherein
R^{K} is selected from the group consisting of —H, —C₁₋₄alkyl and —C₀₋₄alkylR^{Ar}, each of said —C₁₋₄alkyl and —C₀₋₄alkylR^{Ar} being optionally substituted with 1, 2, or 3 substituents R^{N};
R^{L} is selected from the group consisting of —CO₂R^{S} and —C(O)NR^{S}R^{S′};
R^{M} is selected from the group consisting of R^{Z}, indol-7-yl, —SO₂R^{Y}, —C₃₋₄alkylC₀₋₂R^{Y}, —CO₂R^{Y}, —C(O)NR^{Z}OR^{Y}, —C(O)R^{Y}, —C(O)C₁₋₄alkylOR^{Y}, —C₀₋₄alkylC(O)NR^{S}R^{S}′, C₀₋₄alkylC(O)CO₂R^{Y}, 1,3-dihydro-indol-2-one-1-yl, 1,3-dihydro-benzoimidazol-2-one-1-yl, tetrazol-5-yl, 1-R^{Y}-1H-tetrazol-5-yl, R^{Y}-triazolyl, 2-R^{Y}-2H-tetrazol-5-yl and —C₀₋₄alkylC(O)N(R^{Y})(SO₂R^{Y}), each of said R^{M} that is not —H being optionally substituted with 1, 2, or 3 substituents R^{N};
R^{N} is selected from the group consisting of —OCH₃, —Cl, —F, —Br, —I, —OH, —NH₂, —CN, —CF₃, —CH₃, —OC(O)CH₃, and —NO₂;
R^{Q} is selected from the group consisting of —Cl, —F, —Br, —I, —CF₃, —CCl₃, —CN, —C₁₋₄alkyl, —C₀₋₄alkylR^{Ar}, —C₀₋₄alkylR^{Ar′}, —C₀₋₄alkylOR^{Y}, —C₀₋₄alkylCO₂R^{Y}, —C₀₋₄alkylNR^{Y}R^{Z}, —C₀₋₄alkylNR^{Y}COR^{Y}, —C₀₋₄alkylNR^{Y}CONR^{Y}R^{Z}, —C₀₋₄alkylNR^{Y}SO₂R^{Y}, and —C₀₋₄alkylSR^{Y};
R^{S} and R^{S′} are independently selected from the group consisting of —H, —C₁₋₄alkyl, and —C₀₋₄alkylphenyl; alternatively, R^{S} and R^{S′} are taken together with the nitrogen member to which said R^{S} and R^{S′} are attached to form a 4-7 membered heterocyclic ring having 0 or 1 additional heteroatom member selected from the group consisting of O, S, and >NR^{Y}, provided that said additional heteroatom member is separated by at least two carbon members from said nitrogen member to which said R^{S} and R^{S′} are attached, and provided that where R^{Y} is C₀₋₄alkylR^{Ar}, then R^{Ar} is not substituted with R^{L};
R^{W} is selected from the group consisting of R^{Y}, and —C₃₋₇cycloalkyl;
R^{X} is selected from the group consisting of —OR^{Y}, —NR^{Y}R^{Z}, —C₁₋₄alkyl, and —C₀₋₄alkylR^{Ar};
R^{Y} is selected from the group consisting of —H, —C₁₋₄alkyl, —C₀₋₄alkylR^{Ar} and —C₀₋₄alkylR^{Ar′}, each of said R^{Y} that is not —H being optionally substituted with 1, 2, or 3 substituents R^{N};
R^{Z} is selected from the group consisting of R^{Y}, —C₂₋₄alkylOR^{Y}, —C₁₋₂alkylCO₂R^{Y}, —C₁₋₂alkylC(O)NR^{S}R^{S′}, and —C₂₋₄alkylNR^{S}R^{S′};
provided that when R^{Y} and R^{Z} are attached to a nitrogen member, then R^{Y} and R^{Z} are selected as defined above, or R^{Y} and R^{Z} are taken together with the R^{Y}— and R^{Z}— attached nitrogen member to form a 4-7 membered heterocyclic ring HetR^{d} having 0 or 1 additional heteroatom members selected from the group consisting of O, S, and >NR^{M}, said 4-7 membered heterocyclic ring HetR^{d} having 0 or 1 carbonyl members, and said 4-7 membered heterocyclic ring HetR^{d} having 0 or 1 valence allowed carbon members substituted with at least one of R^{M}, —CO₂H, and —C₀₋₁alkylOR^{Y};
R^{Ar} is a moiety with a carbon member attachment point and said R^{Ar} is selected from the group consisting of phenyl, pyridyl, pyrimidyl, and pyrazinyl, wherein each valence allowed carbon member in each of said R^{Ar} is independently substituted with at least one of 0, 1, 2, or 3 substituents R^{N}, and 0 or 1 substituent R^{L};
R^{Ar′} is a 3-8 membered ring having 0, 1, or 2 heteroatom members selected from the group consisting of O, S, N, and >NR^{Y}, said R^{Ar′} having 0, 1, or 2 unsaturated bonds and having 0 or 1 carbonyl members, wherein each valence allowed member in each of said R^{Ar′} ring is independently substituted with 0, 1, or 2 substituents R^{K}; and
R^{f} is a linear 3- to 5-membered hydrocarbon moiety having 0 or 1
unsaturated carbon-carbon bonds and having 0 or 1 carbonyl members;
or an enantiomer, diasteromer, racemate, tautomer, hydrate, solvate, or a pharmaceutically acceptable salt, ester, or amide thereof.
provided that when
(c1) Y′ is R¹(CH₂)₂₋₃O—,
(c2) Z is —CH₂—, and
(c3) X is CH,
then R^{2′} and R^{3′} independently are not —H, —C₁₋₇alkyl, or unsubstituted —C₁₋₇alkylC(O)R^{x}; or R^{2′} and R^{3′} taken together with the nitrogen member to which they are attached do not form HetR^{b} or HetR^{c} where R^{Y} or R^{M} are phenyl, pyridyl, or pyrimidyl.

3. The method of claim 1, wherein said at least one compound of formula (I) is selected from the group consisting of:
Phenyl-carbamic acid 4-(3-dibutylamino-propyl)-phenyl ester hydrochloride;
Phenyl-carbamic acid 4-[2-(4-hydroxy-4-phenyl-piperidin-1-yl)-ethyl]-phenyl ester;
Phenyl-carbamic acid 4-[3-(4-hydroxy-4-phenyl-piperidin-1-yl)-propyl]-phenyl ester;
Phenyl-carbamic acid 4-(3-piperidin-1-yl-propyl)-phenyl ester;
Phenyl-carbamic acid 4-[3-(cyclopropylmethyl-propyl-amino)-propyl]-phenyl ester hydrochloride;
Phenyl-carbamic acid 4-(2-piperidin-1-yl-ethyl)-phenyl ester;
Phenyl-carbamic acid 4-[2-(4-hydroxy-piperidin-1-yl)-ethyl]-phenyl ester;
Phenyl-carbamic acid 4-[2-(cyclohexyl-ethyl-amino)-ethyl]-phenyl ester;
Phenyl-carbamic acid 4-(2-pyrrolidin-1-yl-ethyl)-phenyl ester;
Phenyl-carbamic acid 4-(2-azepan-1-yl-ethyl)-phenyl ester;
Phenyl-carbamic acid 4-[2-(cyclopropylmethyl-propyl-amino)-ethyl]-phenyl ester;
Phenyl-carbamic acid 4-(2-dibutylamino-ethyl)-phenyl ester;
Phenyl-carbamic acid 4-{2-[4-(2-hydroxy-acetylamino)-piperidin-1-yl]-ethyl}-phenyl ester;
Methyl-phenyl-carbamic acid 4-{2-[4-(2-hydroxy-acetylamino)-piperidin-1-yl]-ethyl}-phenyl ester;
Phenyl-carbamic acid 4-[2-(4-methanesulfonylamino-piperidin-1-yl)-ethyl]-phenyl ester;
Methyl-phenyl-carbamic acid 4-[2-(4-methanesulfonylamino-piperidin-1-yl)-ethyl]-phenyl ester; and
Phenyl-carbamic acid 2-fluoro-4-(2-morpholin-4-yl-ethyl)-phenyl ester.

4. The method of claim 1, wherein said at least one compound of formula (I) is selected from the group consisting of:
1-(2-{4-[(3-Hydroxy-phenyl)-methyl-carbamoyloxy]-phenoxy}-ethyl)-piperidine-4-carboxylic acid ethyl ester;
1-(2-{4-[(3-Hydroxy-phenyl)-methyl-carbamoyloxy]-phenoxy}-ethyl)-piperidine-4-carboxylic acid;
Dimethyl-carbamic acid 4-[2-(4-hydroxy-4-phenyl-piperidin-1-yl)-ethoxy]-phenyl ester;
(3-Hydroxy-phenyl)-carbamic acid 4-[2-(4-hydroxy-4-phenyl-piperidin-1-yl)-ethoxy]-phenyl ester;
N-(2-Hydroxy-phenyl)-2-{4-[2-(4-hydroxy-4-phenyl-piperidin-1-yl)-ethoxy]-phenyl}-acetamide;
[4-(2-Piperidin-1-yl-ethoxy)-phenyl]-carbamic acid phenyl ester hydrochloride;
Phenyl-carbamic acid 4-(2-piperidin-1-yl-ethoxy)-phenyl ester;
Phenyl-carbamic acid 4-(2-piperidin-1-yl-ethoxy)-benzyl ester;
(4-Hydroxy-phenyl)-carbamic acid 4-[2-(4-hydroxy-4-phenyl-piperidin-1-yl )-ethoxy]-phenyl ester;
Methyl-phenyl-carbamic acid 4-[2-(4-hydroxy-4-phenyl-piperidin-1-yl)-ethoxy]-phenyl ester;
Phenyl-carbamic acid 4-[2-(4-propyl-piperidin-1-yl)-ethoxy]-phenyl ester;
Phenyl-carbamic acid 4-[3-(4-hydroxy-4-phenyl-piperidin-1-yl)-propoxy]-phenyl ester;
(2-Fluoro-phenyl)-carbamic acid 4-(2-piperidin-1-yl-ethoxy)-phenyl ester;
N-(2-Hydroxy-phenyl)-2-[4-(2-piperidin-1-yl-ethoxy)-phenyl]-acetamide;
(3-Chloro-phenyl)-carbamic acid 4-(2-piperidin-1-yl-ethoxy)-phenyl ester;
Phenyl-carbamic acid 4-(2-diethylamino-ethoxy)-phenyl ester;
Phenyl-carbamic acid 4-[2-(4-hydroxy-4-phenyl-piperidin-1-yl)-ethoxy]-phenyl ester;
Phenyl-carbamic acid 4-(2-dibutylamino-ethoxy)-phenyl ester;
Phenyl-carbamic acid 4-[2-(cyclopropylmethyl-propyl-amino)-ethoxy]-phenyl ester;
Phenyl-carbamic acid 4-[2-(4-benzyl-piperidin-1-yl)-ethoxy]-phenyl ester;
Phenyl-carbamic acid 4-[2-(4-hydroxymethyl-piperidin-1-yl)-ethoxy]-phenyl ester;
Phenyl-carbamic acid 4-[2-(4-hydroxy-piperidin-1-yl)-ethoxy]-phenyl ester;
Phenyl-carbamic acid 4-{2-[4-(4-chloro-3-trifluoromethyl-phenyl)-4-hydroxy-piperidin-1-yl]-ethoxy}-phenyl ester;
Phenyl-carbamic acid 4-(2-azepan-1-yl-ethoxy)-phenyl ester;
Phenyl-carbamic acid 4-{2-[4-(4-bromo-phenyl)-4-hydroxy-piperidin-1-yl]-ethoxy}-phenyl ester;
Phenyl-carbamic acid 4-{2-[4-(4-chloro-phenyl)-4-hydroxy-piperidin-1-yl]-ethoxy}-phenyl ester;
Thiophen-3-yl-carbamic acid 4-(2-piperidin-1-yl-ethoxy)-phenyl ester;
Thiophen-2-yl-carbamic acid 4-(2-piperidin-1-yl-ethoxy)-phenyl ester;
Phenyl-carbamic acid 4-{2-[4-(2-hydroxy-acetylamino)-piperidin-1-yl]-ethoxy}-phenyl ester;
Methyl-phenyl-carbamic acid 4-{2-[4-(2-hydroxy-acetylamino)-piperidin-1-yl]-ethoxy}-phenyl ester;
Phenyl-carbamic acid 4-[2-(4-methanesulfonylamino-piperidin-1-yl)-ethoxy]-phenyl ester;
Methyl-phenyl-carbamic acid 4-[2-(4-methanesulfonylamino-piperidin-1-yl)-ethoxy]-phenyl ester;
Phenyl-carbamic acid 5-{2-[4-(2-hydroxy-acetylamino)-piperidin-1-yl]-ethoxy}-pyridin-2-yl ester; and
Phenyl-carbamic acid 5-[2-(4-acetylamino-piperidin-1-yl)-ethoxy]-pyridin-2-yl ester.

5. The method of claim 1, wherein said at least one compound of formula (I) is selected from the group consisting of:
N-[1-(4-Phenethyloxy-benzyl)-piperidin-4-yl]methanesulfonamide;
1-(6-Phenethyloxy-pyridin-3-ylmethyl)-piperidine-4-carboxylic acid;
1-(4-Phenethyloxy-benzyl)-piperidine;
1-(4-Phenethyloxy-benzyl )-piperidine-4-carboxylic acid;
1-[4-(3-Phenyl-propoxy)-benzyl]-piperidine;
1-[4-(4-Phenyl-butoxy)-benzyl]-piperidine;
1-[1-(4-Phenethyloxy-benzyl)-piperidin-4-yl]-pyrrolidin-2-one;
8-(4-Phenethyloxy-benzyl)-2,8-diaza-spiro[4.5]decan-1-one;
1-(4-Phenethyloxy-benzyl)-piperidine-4-carboxylic acid amide;
1-(4-Phenethyloxy-benzyl)-piperidine-3-carboxylic acid amide;
1-(4-Phenethyloxy-benzyl )-piperidin-4-ol;
1-(4-Phenethyloxy-benzyl)-4-(1H-tetrazol-5-yl)-piperidine;
1-(4-Phenethyloxy-benzyl)-piperidin-4-ylamine;
1-[4-(3-Phenyl-propoxy)-benzyl]-piperidine-4-carboxylic acid ethyl ester;
1-{1-[4-(3-Phenyl-propoxy)-benzyl]-piperidin-4-yl}-pyrrolidin-2-one;
1-[4-(3-Phenyl-propoxy)-benzyl]-piperidin-4-ol;
1-[4-(3-Phenyl-propoxy)-benzyl]-piperidin-3-ol;
1-[4-(3-Phenyl-propoxy)-benzyl]-piperidine-4-carboxylic acid amide;
1-[4-(3-Phenyl-propoxy)-benzyl]-piperidine-3-carboxylic acid amide;
1-[4-(3-Phenyl-propoxy)-benzyl]-piperidine-4-carboxylic acid;
N-[1-(4-Phenethyloxy-benzyl)-piperidin-4-yl]-acetamide;
[1-(4-Phenethyloxy-benzyl)-piperidin-4-yl]-urea;
[1-(4-Phenethyloxy-benzyl )-piperidin-4-yl]-carbamic acid methyl ester;
1-[4-(3-Phenyl-propoxy)-benzyl]-piperidin-4-ylamine;
N-{1-[4-(3-Phenyl-propoxy)-benzyl]-piperidin-4-yl}-methanesulfonamide;
N-{1-[4-(3-Phenyl-propoxy)-benzyl]-piperidin-4-yl}-acetamide;
{1-[4-(3-Phenyl-propoxy)-benzyl]-piperidin-4-yl}-carbamic acid methyl ester;
{1-[4-(3-Phenyl-propoxy)-benzyl]-piperidin-4-yl}-urea;
2-Hydroxy-N-{1-[4-(3-phenyl-propoxy)-benzyl]-piperidin-4-yl}-acetamide;
2-Hydroxy-N-[1-(4-phenethyloxy-benzyl)-piperidin-4-yl]-acetamide;
N-{1-[6-(3-Phenyl-propoxy)-pyridin-3-ylmethyl]-piperidin-4-yl}-acetamide;
N-{1-[6-(3-Phenyl-propoxy)-pyridin-3-ylmethyl]-piperidin-4-yl}-methanesulfonamide;
{1-1-[6-(3-Phenyl-propoxy)-pyridin-3-ylmethyl]-piperidin-4-yl}-pyrrolidin-2-one;
N-[1-(3-Fluoro4-phenethyloxy-benzyl)-piperidin-4-yl]-acetamide.

6. The method of claim 1, wherein said at least one compound of formula (I) is selected from the group consisting of:
1′-[2-(4-Phenethyloxy-phenyl)-ethyl]-[1,4′]bipiperidinyl-3-carboxylic acid ethyl ester;
1′-[2-(4-Phenethyloxy-phenyl)-ethyl]-[1,4′]bipiperidinyl-3-carboxylic acid;
1′-{2-[4-(2-Oxo-2-phenyl-ethoxy)-phenyl]-ethyl}-[1,4′]bipiperidinyl-2-one;
1′-{2-[4-(2-Hydroxy-2-phenyl-ethoxy)-phenyl]-ethyl}-[1,4′]bipiperidinyl-2-one;
1-[2-(4-Phenethyloxy-phenyl)-ethyl]-piperidine-4-carbonitrile;
1-[2-(4-Phenethyloxy-phenyl)-ethyl]-4-(1H-tetrazol-5-yl)-piperidine;
1-[2-(4-Phenethyloxy-phenyl)-ethyl]-4-(1H-[1,2,3]triazol-4-yl)-piperidine;
Cyclopropyl-[2-(4-phenethyloxy-phenyl)-ethyl]-amine;
4-{Cyclopropyl-[2-(4-phenethyloxy-phenyl)-ethyl]-amino}-butyronitrile;
3-{Cyclopropyl-[2-(4-phenethyloxy-phenyl)-ethyl]-amino}-propionic acid ethyl ester;
3-{Cyclopropyl-[2-(4-phenethyloxy-phenyl)-ethyl]-amino}-propionic acid trifluoroacetic acid salt;
1′-[2-(4-Phenethyloxy-phenyl)-ethyl]-[1,4′]bipiperidinyl-2-carboxylic acid ethyl ester;
1-[2-(4-Phenethyloxy-phenoxy)-ethyl]-piperidine;
2-{4-[2-(Cyclohexyl-ethyl-amino)-ethyl]-phenoxy}-1-phenyl-ethanone;
2-{4-[2-(Cyclohexyl-ethyl-amino)-ethyl]-phenoxy}-1-phenyl-ethanol;
1-{2-[4-(2-Oxo-2-phenyl-ethoxy)-phenyl]-ethyl}-piperidine-4-carboxylic acid methyl ester;
1-{2-[4-(2-Hydroxy-2-phenyl-ethoxy}-phenyl]-ethyl-piperidine-4-carboxylic acid methyl ester;
1-[2-(4-Phenethyloxy-phenyl)-ethyl]-piperidine-4-carboxylic acid methyl ester;
1-{2-[4-(2-Hydroxy-2-phenyl-ethoxy)-phenyl]-ethyl}-piperidine-4-carboxylic acid amide;
1-[2-(4-Phenethyloxy-phenyl)-ethyl]-piperidine-4-carboxylic acid amide;
1′-[2-(4-Phenethyloxy-phenyl)-ethyl]-[1,4′]bipiperidinyl-2-one;
1-[2-(4-Phenethyloxy-phenyl)-ethyl]-piperidine-4-carboxylic acid;
4-[2-(4-Phenethyloxy-phenyl)-ethyl]-piperazin-2-one;
3-[2-(4-Phenethyloxy-phenyl)-ethylamino]-propionic acid ethyl ester;
3-{Methyl-[2-(4-phenethyloxy-phenyl)-ethyl]-amino}-propionic acid ethyl ester;
3-{Cyclohexyl-[2-(4-phenethyloxy-phenyl)-ethyl]-amino}-propionic acid ethyl ester;
3-{Methyl-[2-(4-phenethyloxy-phenyl)-ethyl]-amino}-propionic acid;
3-{Cyclohexyl-[2-(4-phenethyloxy-phenyl)-ethyl]-amino}-propionic acid;
3-{(1-Acetyl-piperidin-4-yl)-[2-(4-phenethyloxy-phenyl)-ethyl]-amino}-propionic acid ethyl ester;
3-{(1-Acetyl-piperidin-4-yl)-[2-(4-phenethyloxy-phenyl)-ethyl]-amino}-propionic acid;
1-{2-[4-(2-Hydroxy-2-phenyl-ethoxy)-phenyl]-ethyl}-piperidine-4-carboxylic acid;
2-[4-(2-Piperidin-1-yl-ethyl)-phenoxy]-indan-1-one;
Chloro-phenyl-acetic acid 4-(2-piperidin-1-yl-ethyl)-phenyl ester;
Indan-2-carboxylic acid 4-(2-piperidin-1-yl-ethyl)-phenyl ester;
2-[4-(2-Piperidin-1-yl-ethyl)-phenoxy]-indan-1-ol;
1-{2-[4-(3-Phenyl-propoxy)-phenyl]-ethyl}-piperidine-4-carboxylic acid;
2-Hydroxy-N-{1-[2-(4-phenethyloxy-phenoxy)-ethyl]-piperidin-4-yl}-acetamide;
2-Hydroxy-N-(1-{2-[4-(3-phenyl-propoxy)-phenyl]-ethyl}-piperidin-4-yl)-acetamide;
2-Hydroxy-N-{1-[2-(4-phenethyloxy-phenyl )-ethyl]-piperidin-4-yl}-acetamide;
N-{1-[2-(4-Phenethyloxy-phenoxy)-ethyl]-piperidin-4-yl}-methanesulfonamide;
N-(1-{2-[4-(3-Phenyl-propoxy)-phenyl]-ethyl}-piperidin-4-yl)-methanesulfonamide;
N-{1-[2-(4-Phenethyloxy-phenyl)-ethyl]-piperidin-4-yl}-methanesulfonamide;
N-{1-[2-(6-Phenethyloxy-pyridin-3-yl)-ethyl]-piperidin-4-yl}-methanesulfonamide;
2-Hydroxy-N-{1-[2-(6-phenethyloxy-pyridin-3-yl)-ethyl]-piperidin-4-yl}-acetamide; and
1-{1-[2-(6-Phenethyloxy-pyridin-3-yl)-ethyl]-piperidin-4-yl}-pyrrolidin-2-one.

7. The method of claim 1, wherein said at least one compound of formula
(I) is selected from the group consisting of:
Carbonic acid phenyl ester 4-(2-piperidin-1-yl-ethoxy)-phenyl ester;
Phenyl-acetic acid 4-(2-piperidin-1-yl-ethoxy)-phenyl ester;
2-Phenyl-propionic acid 4-(2-piperidin-1-yl-ethoxy)-phenyl ester;
1H-Indole-2-carboxylic acid 4-(2-piperidin-1-yl-ethoxy)-phenyl ester;
1-Phenyl-2-[4-(2-piperidin-1-yl-ethoxy)-phenoxy]-ethanone;
3-{2-[4-(2-Piperidin-1-yl-ethoxy)-phenoxy]-ethyl}-phenol;
1H-Indole-3-carboxylic acid 4-(2-piperidin-1-yl-ethoxy)-phenyl ester;
1-{2-[4-(Indan-2-yloxy)-phenoxy]-ethyl}-piperidine;
1-(2-{4-[2-(2-Fluoro-phenyl)-ethoxy]-phenoxy}-ethyl)-piperidine;
1-Phenyl-2-[4-(2-piperidin-1-yl-ethoxy)-phenoxy]-ethanol;
4-{2-[4-(2-Piperidin-1-yl-ethoxy)-phenoxy]-ethyl}-phenol;
1-{1-[2-(4-Phenethyloxy-phenoxy)-ethyl]-piperidin-4-yl}-pyrrolidin-2-one;
1-[2-(4-Phenethyloxy-phenoxy)-ethyl]-piperidine-4-carboxylic acid;
1-{2-[4-(3-Phenyl-propoxy)-phenoxy]-ethyl}-piperidine-4-carboxylic acid ethyl ester;
1-{2-[4-(3-Phenyl-propoxy)-phenoxy]-ethyl}-piperidine-4-carboxylic acid;
Chloro-phenyl-acetic acid 4-(2-piperidin-1-yl-ethoxy)-phenyl ester;
Indan-2-carboxylic acid 4-(2-piperidin-1-yl-ethoxy)-phenyl ester;
2-[4-(2-Piperidin-1-yl-ethoxy)-phenoxy]-indan-1-ol;
2-[4-(2-Piperidin-1-yl-ethoxy)-phenoxy]-indan-1-one;
2-Hydroxy-N-(1-{2-[4-(3-phenyl-propoxy)-phenoxy]-ethyl}-piperidin-4-yl)-acetamide;
N-(1-{2-[4-(3-Phenyl-propoxy)-phenoxy]-ethyl}-piperidin-4-yl)-methanesulfonamide;
N-{1-[2-(6-Phenethyloxy-pyridin-3-yloxy)-ethyl]-piperidin-4-yl}-methanesulfonamide;
1-{2-[6-(3-Phenyl-propoxy)-pyridin-3-yloxy]-ethyl}-piperidine-4-carboxylic acid;
1-(4-Phenethyloxy-phenoxy)-3-piperidin-1-yl-propan-2-ol;
2-Hydroxy-N-(1-{2-hydroxy-3-[4-(3-phenyl-propoxy)-phenoxy]-propyl}-piperidin-4-yl)-acetamide;
N-{1-[2-(3-Fluoro-4-phenethyloxy-phenoxy)-ethyl]-piperidin-4-yl}-2-hydroxy-acetamide;
1-(2-{4-[2-(3-Fluoro-phenyl)-ethoxy]-phenoxy}-ethyl)-piperidine; and
1-(2-{4-[2-(4-Fluoro-phenyl)-ethoxy]-phenoxy}-ethyl)-piperidine.

8. A method for preventing, inhibiting, or treating inflammation in a subject, comprising administering to the subject a therapeutically effective amount of a pharmaceutical composition comprising at least one compound of formula (I).

9. The method of claim 8, wherein said at least one compound of formula (I) is at least one compound of formula (II).

10. The method of claim 8, wherein inflammation is due to at least one of inflammatory bowel disease, chronic obstructive pulmonary disease, arthritis, psoriasis, asthma, cystic fibrosis, atherosclerosis, rheumatoid arthritis, and multiple sclerosis.

11. The method of claim 9, wherein inflammation is due to at least one of inflammatory bowel disease, chronic obstructive pulmonary disease, arthritis, psoriasis, asthma, cystic fibrosis, atherosclerosis, rheumatoid arthritis, and multiple sclerosis.

12. A compound of formula (II): 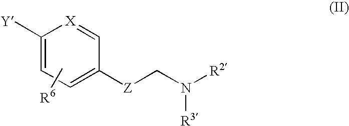
wherein
X is selected from the group consisting of CH and N;
Y′ is selected from the group consisting of R¹(CH₂)₂₋₃O—, R⁷N(R⁸)CO₂—, R⁷N(R⁸)C(O)N(R⁸)—, R⁷N(R⁸)CO₂CH₂—, R⁷N(R⁸)C(O)CH₂—, R¹OC(O)N(R⁸)—, R¹OCO₂—, R¹CO₂—, R¹CH(R⁹)CO₂—, R¹C(O)CH(R¹⁰)O—, and R¹CH(R⁹)CH(R¹⁰)O—, provided that when one of R⁹ and R¹⁰ in R¹CH(R⁹)CH(R¹⁰)O— is —H, then the other is not —H;
R¹ is a moiety selected from the group consisting of phenyl, thienyl, pyrrolyl, furanyl, oxazolyl, imidazolyl, thiazolyl, indolyl, indanyl, and tetrahydronaphthyl, wherein R¹ is substituted with 0, 1, or 2 substituents R⁴;
R⁴ is selected from the group consisting of —H, —OCH₃, —Cl, —F, —Br, —I, —OH, —NH₂, —CN, —CF₃, and —CH₃;
R⁷ is —C₁₋₄alkyl or is selected from the group consisting of phenyl, thienyl, pyrrolyl, furanyl, oxazolyl, imidazolyl, thiazolyl, indolyl, indanyl, and tetrahydronaphthyl, wherein R⁷ is substituted with 0, 1, or 2 substituents R⁴;
R⁸ is —H or —C₁₋₄alkyl;
or, R⁷ and R⁸ are taken together with the nitrogen member to which they are attached to form pyrrolidinyl, piperidinyl, morpholinyl, or thiomorpholinyl;
R⁹ is —H, —C₁₋₄alkyl, —Cl, or —OH;
R¹⁰ is —H, —C₁₋₄alkyl or is taken together with one of R⁴ to form a 5- or 6-membered carbocyclic ring;
R¹¹ is —H or —OH;
Z is selected from the group consisting of bond, —CH₂—, —OCH₂—, —OCH₂CH(R¹¹)—, and —CH₂CH(R¹¹)—;
provided that when Z is bond, then Y′ is one of R¹(CH₂)₂₋₃O—, R¹CO₂—, R¹CH(R⁹)CO₂—, R¹C(O)CH(R¹⁰)O—, and R¹CH(OH)CH(R¹⁰)O—;
R⁶ is —H or —F; and
R^{2′} and R^{3′} are each independently selected from the group consisting of
A) H, C₁₋₇alkyl, C₃₋₇alkenyl, wherein the carbon in said alkenyl that is attached to the nitrogen member has only single bonds, C₃₋₇alkynyl, wherein the carbon in said alkynyl that is attached to the nitrogen member has only single bonds, C₃₋₇cycloalkyl optionally benzofused, C₅₋₇cycloalkenyl, C₃₋₇cycloalkylC₁₋₇alkyl, C₁₋₇alkylC₃₋₇cycloalkyl and phenyl, wherein each of the substituents A) is independently substituted with 0, 1, or 2 substituents R^{Q}, and each of said R^{Q} is a substituent at a carbon member that is at least one carbon member removed from the nitrogen member;
B) a 4-7 membered saturated heterocyclic ring HetR^{a}, said 4-7 membered saturated heterocyclic ring HetR^{a}, having 0 or 1 double bonds, having a carbon member point of attachment and containing a member >NR^{M} as a heteroatom member, and said heteroatom member being separated from said carbon member point of attachment by at least one additional carbon member;
C) —C₁₋₇alkylC(O)R^{x}, optionally substituted with CH₂R^{Ar} or CH₂R^{Ar′};
D) —C₂₋₅alkylC(O)R^{x}, wherein two valence allowed carbon members in the C₂₋₅alkyl of said —C₂₋₅alkylC(O)R^{x} are part of a saturated C₃₋₆carbocycle;
E) —C₂₋₅alkylOH wherein two valence allowed carbon members in the C₂₋₅alkyl of said —C₂₋₅alkylOH are part of a saturated C₃₋₆carbocycle;
F) —C₀₋₄alkylphenyl, wherein the phenyl in said —C₀₋₄alkylphenyl is fused at two adjacent carbon members in said phenyl to R^{f}, or is benzofused;
G) —C₀₋₄alkylAr⁶, where Ar⁶ is a 6-membered heteroaryl having a carbon member point of attachment and having 1 or 2 —N═ heteroatom members, and benzofused;
H) —C₀₋₄alkylAr⁵, where Ar⁵ is a 5-membered heteroaryl, having one heteroatom member selected from the group consisting of O, S, and >NR^{Y}, and having 0 or 1 —N═ additional heteroatom member, optionally containing 1 or 2 carbonyl groups, and optionally benzofused;
I) —C₁₋₄alkylAr^{5′}, where Ar^{5′} is a 5-membered heteroaryl containing 3 or 4 nitrogen members, optionally substituted with R^{Y}, and having a valence allowed site as a point of attachment;
J) —C₀₋₄alkylAr⁶⁻⁶, where Ar⁶⁻⁶ is a C₀₋₄alkyl-attached phenyl fused at valence allowed sites to a 6-membered heteroaryl, wherein said 6-membered heteroaryl has 1 or 2 —N═ heteroatom members;
K) —C₀₋₄alkylAr⁶⁻⁵, where Ar⁶⁻⁵ is a C₀₋₄alkyl-attached phenyl fused at valence allowed sites to a 5-membered heteroaryl, said 5-membered heteroaryl having one heteroatom member selected from the group consisting of O, S, and >NR^{Y}, and said 5-membered heteroaryl having 0 or 1 additional heteroatom member which is —N═;
L) one of 2-(4-ethyl-phenoxy)-benzothiazole, 2-(4-ethyl-phenoxy)-benzooxazole, and 2-(4-ethyl-phenoxy)-1H-benzoimidazole; and
M) —SO₂C₁₋₄alkyl;
alternatively R^{2′} and R^{3′} are taken together with the nitrogen to which they are attached to form a heterocyclic ring that contains at least one heteroatom member that is said attachment nitrogen, said heterocyclic ring being selected from the group consisting of
i) a 4-7 membered saturated heterocyclic ring HetR^{b}, said 4-7 membered saturated heterocyclic ring HetR^{b} having one heteroatom member that is said attachment nitrogen, and being substituted with 0, 1, or 2 substituents at the same or at different ring members, said substituents being selected from the group consisting of —R^{Y}, —CN, —C(O)R^{Y}, —C₀₋₄alkylCO₂R^{Y}, —C₀₋₄alkylC(O)CO₂R^{Y}, —C₀₋₄alkylOR^{Y}, —C₀₋₄alkylC(O)NR^{Y}R^{Z}, —C₀₋₄alkylNR^{Y}C(O)R^{Z}, —C(O)NR^{Z}OR^{Y}, —C₀₋₄alkylNR^{Y}C(O)CH₂OR^{Y}, —C₀₋₄alkylNR^{Y}C(O)CH₂C(O)R^{Y}, —C₀₋₄alkylNR^{Y}CO₂R^{Y}, —C₀₋₄alkylNR^{Y}C(O)NR^{Y}R^{Z}, —C₀₋₄alkylNR^{Y}C(S)NR^{Y}R^{Z}, —NR^{Y}C(O)CO₂R^{Y}, —NR^{Y}R^{Z}, —C₀₋₄alkylNR^{W}SO₂R^{Y}, 1,3-dihydro-indol-2-one-1-yl, 1,3-dihydro-benzoimidazol-2-one-1-yl, tetrazol-5-yl, 1-R^{Y}-1H-tetrazol-5-yl, R^{Y}-triazolyl, 2-R^{Y}-2H-tetrazol-5-yl, pyrrolidine-2-thion-1-yl, piperidine-2-thion-1-yl, —C₀₋₄alkylC(O)N(R^{Y})(SO₂R^{Y}), —C₀₋₄alkylN(R^{Y})(SO₂)NR^{Y}R^{Y}, —C₀₋₄alkylN(R^{Y})(SO₂)NR^{Y}CO₂R^{Y}, halo, 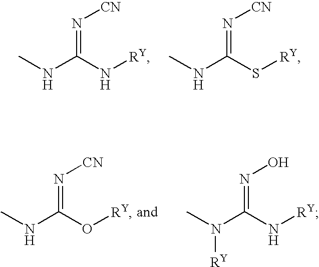
ii) a 5-7 membered saturated heterocyclic ring HetR^{c}, said 5-7 membered saturated heterocyclic ring HetR^{c} having one additional heteroatom member separated from said attachment nitrogen by at least one carbon member, said additional heteroatom member being selected from the group consisting of O, S(═O)₀₋₂, and >NR^{M}, said 5-7 membered saturated heterocyclic ring HetR^{c} having 0 or 1 carbonyl members, and being substituted with 0, 1, or 2 substituents at the same or at different carbon ring members, said substituents being selected from the group consisting of —C(O)R^{Y}, —CO₂R^{Y}, —C₃₋₄alkylCO₂R^{Y} and R^{Z};
iii) one of imidazolidin-1-yl, 2-imidazolin-1-yl, pyrazol-1-yl, imidazol-1-yl, 2H-tetrazol-2-yl, 1H-tetrazol-1-yl, pyrrol-1-yl, 2-pyrrolin-1-yl, and 3-pyrrolin-1-yl, wherein each of said 2H-tetrazol-2-yl and 1H-tetrazol-1-yl is substituted at the carbon member with 0 or 1 of —C₀₋₄alkylR^{Z}, —C₀₋₄alkylSR^{Y}, —C₀₋₄alkylC₀₋₂R^{Y}, and substituent HetR^{a}; and
iv) one of 1,2,3,4-tetrahydro-quinolin-1-yl, 1,2,3,4-tetrahydro-isoquinolin-2-yl, indol-1-yl, isoindol-2-yl, indolin-1-yl, benzimidazol-1-yl, 2,8-diaza-spiro[4.5]decan-1-one-8-yl, 4-{[(2-tert-butoxycarbonylamino-cyclobutanecarbonyl)-amino]-methyl}-piperidin-1-yl, 4-{[(2-amino-cyclobutanecarbonyl)-amino]-methyl}-piperidin-1-yl, 3,9-diaza-spiro[5.5]undecane-3-carboxylic acid-9-yl tert-butyl ester, 4-oxo-1-phenyl-1,3,8-triaza-spiro[4.5]dec-8-yl, and 4-oxo-1,3,8-triaza-spiro[4.5]dec-8-yl;
wherein
R^{K} is selected from the group consisting of —H, —C₁₋₄alkyl and —C₀₋₄alkylR^{Ar}, each of said —C₁₋₄alkyl and —C₀₋₄alkylR^{Ar} being optionally substituted with 1, 2, or 3 substituents R^{N};
R^{L} is selected from the group consisting of —CO₂R^{S} and —C(O)NR^{S}R^{S′};
R^{M} is selected from the group consisting of R^{Z}, indol-7-yl, —SO₂R^{Y}, —C₃₋₄alkylC₀₋₂R^{Y}, —CO₂R^{Y}, —C(O)NR^{Z}OR^{Y}, —C(O)R^{Y}, —C(O)C₁₋₄alkylOR^{Y}, —C₀₋₄alkylC(O)NR^{S}R^{S}′, C₀₋₄alkylC(O)CO₂R^{Y}, 1,3-dihydro-indol-2-one-1-yl, 1,3-dihydro-benzoimidazol-2-one-1-yl, tetrazol-5-yl, 1-R^{Y}-1H-tetrazol-5-yl, R^{Y}-triazolyl, 2-R^{Y}-2H-tetrazol-5-yl and —C₀₋₄alkylC(O)N(R^{Y})(SO₂R^{Y}), each of said R^{M} that is not —H being optionally substituted with 1, 2, or 3 substituents R^{N};
R^{N} is selected from the group consisting of —OCH₃, —Cl, —F, —Br, —I, —OH, —NH₂, —CN, —CF₃, —CH₃, —OC(O)CH₃, and —NO₂;
R^{Q} is selected from the group consisting of —Cl, —F, —Br, —I, —CF₃, —CCl₃, —CN, —C₁₋₄alkyl, —C₀₋₄alkylR^{Ar}, —C₀₋₄alkylR^{Ar′}, —C₀₋₄alkylOR^{Y}, —C₀₋₄alkylCO₂R^{Y}, —C₀₋₄alkylNR^{Y}R^{Z}, —C₀₋₄alkylNR^{Y}COR^{Y}, —C₀₋₄alkylNR^{Y}CONR^{Y}R^{Z}, —C₀₋₄alkylNR^{Y}SO₂R^{Y}, and —C₀₋₄alkylSR^{Y};
R^{S} and R^{S′} are independently selected from the group consisting of —H, —C₁₋₄alkyl, and —C₀₋₄alkylphenyl; alternatively, R^{S} and R^{S′} are taken together with the nitrogen member to which said R^{S} and R^{S′} are attached to form a 4-7 membered heterocyclic ring having 0 or 1 additional heteroatom member selected from the group consisting of O, S, and >NR^{Y}, provided that said additional heteroatom member is separated by at least two carbon members from said nitrogen member to which said R^{S} and R^{S′} are attached, and provided that where R^{Y} is C₀₋₄alkylR^{Ar}, then R^{Ar} is not substituted with R^{L};
R^{W} is selected from the group consisting of R^{Y}, and —C₃₋₇cycloalkyl;
R^{X} is selected from the group consisting of —OR^{Y}, —NR^{Y}R^{Z}, —C₁₋₄alkyl, and —C₀₋₄alkylR^{Ar};
R^{Y} is selected from the group consisting of —H, —C₁₋₄alkyl, —C₀₋₄alkylR^{Ar} and —C₀₋₄alkylR^{Ar′}, each of said R^{Y} that is not —H being optionally substituted with 1, 2, or 3 substituents R^{N};
R^{Z} is selected from the group consisting of R^{Y}, —C₂₋₄alkylOR^{Y}, —C₁₋₂alkylCO₂R^{Y}, —C₁₋₂alkylC(O)NR^{S}R^{S′}, and —C₂₋₄alkylNR^{S}R^{S′};
provided that when R^{Y} and R^{Z} are attached to a nitrogen member, then R^{Y} and R^{Z} are selected as defined above, or R^{Y} and R^{Z} are taken together with the R^{Y}— and R^{Z}— attached nitrogen member to form a 4-7 membered heterocyclic ring HetR^{d} having 0 or 1 additional heteroatom members selected from the group consisting of O, S, and >NR^{M}, said 4-7 membered heterocyclic ring HetR^{d} having 0 or 1 carbonyl members, and said 4-7 membered heterocyclic ring HetR^{d} having 0 or 1 valence allowed carbon members substituted with at least one of R^{M}, —CO₂H, and —C₀₋₁alkylOR^{Y};
R^{Ar} is a moiety with a carbon member attachment point and said R^{Ar} is selected from the group consisting of phenyl, pyridyl, pyrimidyl, and pyrazinyl, wherein each valence allowed carbon member in each of said R^{Ar} is independently substituted with at least one of 0, 1, 2, or 3 substituents R^{N}, and 0 or 1 substituent R^{L};
R^{Ar′} is a 3-8 membered ring having 0, 1, or 2 heteroatom members selected from the group consisting of O, S, N, and >NR^{Y}, said R^{Ar′} having 0, 1, or 2 unsaturated bonds and having 0 or 1 carbonyl members, wherein each valence allowed member in each of said R^{Ar′} ring is independently substituted with 0, 1, or 2 substituents R^{K}; and
R^{f} is a linear 3- to 5-membered hydrocarbon moiety having 0 or 1 unsaturated carbon-carbon bonds and having 0 or 1 carbonyl members;
or an enantiomer, diasteromer, racemate, tautomer, hydrate, solvate, or a pharmaceutically acceptable salt, ester, or amide thereof.
provided that when
(c1) Y′ is R¹(CH₂)₂₋₃₀—,
(c2) Z is CH₂, and
(c3) X is CH,
then R^{2′} and R^{3′} independently are not —H, —C₁₋₇alkyl, or unsubstituted —C₁₋₇alkylC(O)R^{x}; or R^{2′} and R^{3′} taken together with the nitrogen to which they are attached do not form HetR^{b} or HetR^{c} where R^{Y} or R^{M} are phenyl, pyridyl, or pyrimidyl.

13. The compound of claim 12, wherein said X is CH.

14. The compound of claim 12, wherein said Y′ is selected from the group consisting of R⁷N(R⁸)CO₂—, R⁷N(R⁸)C(O)N(R⁸)—, R⁷N(R⁸)CO₂CH₂—, R⁷N(R⁸)C(O)CH₂—, R¹OC(O)N(R⁸)—, R¹OCO₂—, R¹CO₂—, R¹CH(R⁹)CO₂—, R¹C(O)CH(R¹⁰)O—, and R¹CH(R⁹)CH(R¹⁰)O—, provided that when one of R⁹ and R¹⁰ in R¹CH(R⁹)CH(R¹⁰)O— is —H, then the other is not —H.

15. The compound of claim 12, wherein said Y′ is R¹(CH₂)₂₋₃O—.

16. The compound of claim 12, wherein said R¹ is selected from the group consisting of phenyl, thienyl, indolyl, and tetrahydronaphthyl, and said R¹ is substituted with 0, 1, or 2 substituents selected from the group consisting of —H, —OCH₃, —Cl, —F, —Br, —I, —OH, —NH₂, —CN, —CF₃ and —CH₃.

17. The compound of claim 12, wherein said R¹ is phenyl.

18. The compound of claim 12, wherein said R⁴ is selected from the group consisting of —H, —Cl, —F, and —OH.

19. The compound of claim 12, wherein said R⁴ is —H.

20. The compound of claim 12, wherein said R⁷ is —C₁₋₄alkyl.

21. The compound of claim 12, wherein said R⁷ is methyl or ethyl.

22. The compound of claim 12, wherein said R⁷ is selected from the group consisting of phenyl, thienyl, pyrrolyl, furanyl, oxazolyl, imidazolyl, thiazolyl, indolyl, indanyl, and tetrahydronaphthyl.

23. The compound of claim 12, wherein said R⁷ is selected from the group consisting of phenyl, thienyl, indolyl, indanyl, and tetrahydronaphthyl.

24. The compound of claim 12, wherein said R⁷ is phenyl.

25. The compound of claim 12, wherein said R⁸ is —C₁₋₄alkyl.

26. The compound of claim 12, wherein said R⁸ is methyl or ethyl.

27. The compound of claim 12, wherein said R⁷ and R⁸ are taken together with the nitrogen member to which they are attached to form pyrrolidinyl, piperidinyl, morpholinyl, or thiomorpholinyl.

28. The compound of claim 12, wherein said R⁷ and R⁸ are taken together with the nitrogen member to which they are attached to form piperidinyl.

29. The compound of claim 12, wherein said R⁹ is —H, —Cl, methyl, ethyl, or —OH.

30. The compound of claim 12, wherein said R⁹ is —H, methyl, or —OH.

31. The compound of claim 12, wherein said R⁹ is methyl.

32. The compound of claim 12, wherein said R¹⁰ is —H, methyl, ethyl, isopropyl, or butyl.

33. The compound of claim 12, wherein said R¹⁰ is —H.

34. The compound of claim 12, wherein said R¹¹ is —H.

35. The compound of claim 12, wherein said Z is selected from the group consisting of bond, —CH₂—, —OCH₂—, —OCH₂CH₂—, and —CH₂CH₂—.

36. The compound of claim 12, wherein said Z is bond, and said Y′ is one of R¹(CH₂)₂₋₃O—, R¹CO₂—, R¹CH(R⁹)CO₂—, R¹C(O)CH(R¹⁰)O—, or R¹CH(R⁹)CH(R¹⁰)O—, provided that when one of R⁹ and R¹⁰ in R¹CH(R⁹)CH(R¹⁰)O— is —H, then the other is not —H.

37. The compound of claim 12, wherein said Z is bond, and said Y′ is R¹(CH₂)₂₋₃O—.

38. The compound of claim 12, wherein said R⁶ is —H.

39. The compound of claim 12, wherein said R^{2′} and R^{3′} are each independently selected from the group consisting of —H, —C₁₋₇alkyl, —C₃₋₇alkenyl, —C₃₋₇alkynyl, —C₃₋₇cycloalkyl optionally benzofused,—C₅₋₇cycloalkenyl, —C₃₋₇cycloalkylC₁₋₇alkyl, —C₁₋₇alkylC₃₋₇cycloalkyl, and phenyl.

40. The compound of claim 12, wherein said Y′ is R¹(CH₂)₂₋₃O— and said R^{2′} and R^{3′} are each independently selected from the group consisting of —C₃₋₇alkenyl, —C₃₋₇alkynyl, —C₃₋₇cycloalkyl optionally benzofused, —C₅₋₇cycloalkenyl, —C₃₋₇cycloalkylC₁₋₇alkyl, —C₁₋₇alkylC₃₋₇cycloalkyl, and phenyl.

41. The compound of claim 12, wherein said R^{2′} and R^{3′} are each independently selected from the group consisting of a 4-7 membered saturated heterocyclic ring HetR^{a}, said 4-7 membered saturated heterocyclic ring HetR^{a}, having 0 or 1 double bonds, having a carbon member point of attachment and containing a member >NR^{M} as a heteroatom member, and said heteroatom member being separated from said carbon member point of attachment by at least one additional carbon member.

42. The compound of claim 12, wherein said R^{2′} and R^{3′} are each independently selected from the group consisting of —C₁₋₇alkylC(O)R^{x}, optionally substituted with CH₂R^{Ar} or CH₂R^{Ar′}.

43. The compound of claim 12, wherein said Y′ is R¹(CH₂)₂₋₃O— and said R^{2′} and R^{3′} are each independently selected from the group consisting of —C₁₋₇alkylC(O)R^{x}, substituted with CH₂R^{Ar} or CH₂R^{Ar′}.

44. The compound of claim 12, wherein said R^{2′} and R^{3′} are each independently selected from the group consisting of —C₂₋₅alkylC(O)R^{x}, wherein two valence allowed carbon members in the C₂₋₅alkyl of said —C₂₋₅alkylC(O)R^{x} are part of a saturated C₃₋₆carbocycle.

45. The compound of claim 12, wherein said R^{2′} and R^{3′} are each independently selected from the group consisting of —C₂₋₅alkylOH, wherein two valence allowed carbon members in the C₂₋₅alkyl of said —C₂₋₅alkylOH are part of a saturated C₃₋₆carbocycle.

46. The compound of claim 12, wherein said R^{2′} and R^{3′} are each independently —C₁₋₄alkylAr^{5′}, where Ar^{5′} is a 5-membered heteroaryl containing 3 or 4 nitrogen members, optionally substituted with R^{Y}, and having a valence allowed site as a point of attachment.

47. The compound of claim 12, wherein said R^{2′} and R^{3′} are taken together with the nitrogen member to which they are attached to form azetidinyl, pyrrolidinyl, piperidinyl, or homopiperidinyl.

48. The compound of claim 12, wherein said R^{2′} and R^{3′} are taken together with the nitrogen member to which they are attached to form piperidinyl.

49. The compound of claim 12, wherein said Y′ is R¹(CH₂)₂₋₃O—, and said R^{2′} and R^{3′} are taken together with the nitrogen member to which they are attached to form piperidinyl, said piperidinyl being substituted with 1 or 2 substituents at the same or at different substitution members, said substituents being selected from the group consisting of —R^{Y}, —CN, —C(O)R^{Y}, —C₀₋₄alkylCO₂R^{Y}, —C₀₋₄alkylC(O)CO₂R^{Y}, —C₀₋₄alkylOR^{Y},—C₀₋₄alkylC(O)NR^{Y}R^{Z},—C₀₋₄alkylNR^{Y}C(O)R^{Z}, —C(O)NR^{Z}OR^{Y}, —C₀₋₄alkylNR^{Y}C(O)CH₂OR^{Y}, —C₀₋₄alkylNR^{Y}C(O)CH₂C(O)R^{Y}, —C₀₋₄alkylNR^{Y}CO₂R^{Y}, —C₀₋₄alkylNR^{Y}C(O)NR^{Y}R^{Z}, —C₀₋₄alkylNR^{Y}C(S)NR^{Y}R^{Z}, —NR^{Y}C(O)CO₂R^{Y}, —NR^{Y}R^{Z}, —C₀₋₄alkylNR^{W}SO₂R^{Y}, 1,3-dihydro-indol-2-one-1-yl, 1,3-dihydro-benzoimidazol-2-one-1-yl, tetrazol-5-yl, 1-R^{Y}-1H-tetrazol-5-yl, R^{Y}-triazolyl, 2-R^{Y}-2H-tetrazol-5-yl, pyrrolidine-2-thion-1-yl, piperidine-2-thion-1-yl, —C₀₋₄alkylC(O)N(R^{Y})(SO₂R^{Y}), —C₀₋₄alkylN(R^{Y})(SO₂)NR^{Y}R^{Y}, —C₀₋₄alkylN(R^{Y})(SO₂)NR^{Y}CO₂R^{Y}, halo,

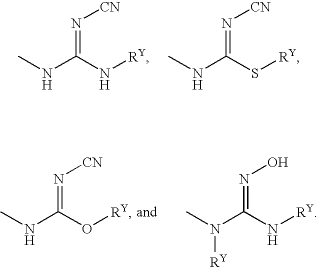

50. The compound of claim 12, wherein said R^{2′} and R^{3′} are taken together with the nitrogen member to which they are attached to form piperazinyl or piperazinonyl.

51. A compound selected from:
Phenyl-carbamic acid 4-(3-dibutylamino-propyl)-phenyl ester hydrochloride;
Phenyl-carbamic acid 4-[2-(4-hydroxy-4-phenyl-piperidin-1-yl)-ethyl]-phenyl ester;
Phenyl-carbamic acid 4-[3-(4-hydroxy-4-phenyl-piperidin-1-yl)-propyl]-phenyl ester;
Phenyl-carbamic acid 4-(3-piperidin-1-yl-propyl)-phenyl ester;
Phenyl-carbamic acid 4-[3-(cyclopropylmethyl-propyl-amino)-propyl]-phenyl ester hydrochloride;
Phenyl-carbamic acid 4-(2-piperidin-1-yl-ethyl)-phenyl ester;
Phenyl-carbamic acid 4-[2-(4-hydroxy-piperidin-1-yl)-ethyl]-phenyl ester;
Phenyl-carbamic acid 4-[2-(cyclohexyl-ethyl-amino)-ethyl]-phenyl ester;
Phenyl-carbamic acid 4-(2-pyrrolidin-1-yl-ethyl)-phenyl ester;
Phenyl-carbamic acid 4-(2-azepan-1-yl-ethyl)-phenyl ester;
Phenyl-carbamic acid 4-[2-(cyclopropylmethyl-propyl-amino)-ethyl]-phenyl ester;
Phenyl-carbamic acid 4-(2-dibutylamino-ethyl)-phenyl ester;
Phenyl-carbamic acid 4-{2-[4-(2-hydroxy-acetylamino)-piperidin-1-yl]-ethyl}-phenyl ester;
Methyl-phenyl-carbamic acid 4-{2-[4-(2-hydroxy-acetylamino)-piperidin-1-yl]-ethyl}-phenyl ester;
Phenyl-carbamic acid 4-[2-(4-methanesulfonylamino-piperidin-1-yl)-ethyl]-phenyl ester;
Methyl-phenyl-carbamic acid 4-[2-(4-methanesulfonylamino-piperidin-1-yl)-ethyl]-phenyl ester;
Phenyl-carbamic acid 2-fluoro-4-(2-morpholin-4-yl-ethyl)-phenyl ester; and mixtures thereof.

52. A compound selected from:
1-(2-{4-[(3-Hydroxy-phenyl)-methyl-carbamoyloxy]-phenoxy}-ethyl)-piperidine-4-carboxylic acid ethyl ester;
1-(2-{4-[(3-Hydroxy-phenyl)-methyl-carbamoyloxy]-phenoxy}-ethyl)-piperidine-4-carboxylic acid;
Dimethyl-carbamic acid 4-[2-(4-hydroxy-4-phenyl-piperidin-1-yl)-ethoxy]-phenyl ester;
(3-Hydroxy-phenyl)-carbamic acid 4-[2-(4-hydroxy-4-phenyl-piperidin-1-yl)-ethoxy]-phenyl ester;
N-(2-Hydroxy-phenyl)-2-{4-[2-(4-hydroxy-4-phenyl-piperidin-1-yl)-ethoxy]-phenyl}-acetamide;
[4-(2-Piperidin-1-yl-ethoxy)-phenyl]-carbamic acid phenyl ester hydrochloride;
Phenyl-carbamic acid 4-(2-piperidin-1-yl-ethoxy)-phenyl ester;
Phenyl-carbamic acid 4-(2-piperidin-1-yl-ethoxy)-benzyl ester;
(4-Hydroxy-phenyl)-carbamic acid 4-[2-(4-hydroxy-4-phenyl-piperidin-1-yl)-ethoxy]-phenyl ester;
Methyl-phenyl-carbamic acid 4-[2-(4-hydroxy-4-phenyl-piperidin-1-yl)-ethoxy]-phenyl ester;
Phenyl-carbamic acid 4-[2-(4-propyl-piperidin-1-yl)-ethoxy]-phenyl ester;
Phenyl-carbamic acid 4-[3-(4-hydroxy-4-phenyl-piperidin-1-yl)-propoxy]-phenyl ester;
(2-Fluoro-phenyl)-carbamic acid 4-(2-piperidin-1-yl-ethoxy)-phenyl ester;
N-(2-Hydroxy-phenyl)-2-[4-(2-piperidin-1-yl-ethoxy)-phenyl]-acetamide;
(3-Chloro-phenyl)-carbamic acid 4-(2-piperidin-1-yl-ethoxy)-phenyl ester;
Phenyl-carbamic acid 4-(2-diethylamino-ethoxy)-phenyl ester;
Phenyl-carbamic acid 4-[2-(4-hydroxy-4-phenyl-piperidin-1-yl)-ethoxy]-phenyl ester;
Phenyl-carbamic acid 4-(2-dibutylamino-ethoxy)-phenyl ester;
Phenyl-carbamic acid 4-[2-(cyclopropylmethyl-propyl-amino)-ethoxy]-phenyl ester;
Phenyl-carbamic acid 4-[2-(4-benzyl-piperidin-1-yl)-ethoxy]-phenyl ester;
Phenyl-carbamic acid 4-[2-(4-hydroxymethyl-piperidin-1-yl)-ethoxy]-phenyl ester;
Phenyl-carbamic acid 4-[2-(4-hydroxy-piperidin-1-yl)-ethoxy]-phenyl ester;
Phenyl-carbamic acid 4-{2-[4-(4-chloro-3-trifluoromethyl-phenyl)-4-hydroxy-piperidin-1-yl]-ethoxy}-phenyl ester;
Phenyl-carbamic acid 4-(2-azepan-1-yl-ethoxy)-phenyl ester;
Phenyl-carbamic acid 4-{2-[4-(4-bromo-phenyl)-4-hydroxy-piperidin-1-yl]-ethoxy}-phenyl ester;
Phenyl-carbamic acid 4-{2-[4-(4-chloro-phenyl)-4-hydroxy-piperidin-1-yl]-ethoxy}-phenyl ester;
Thiophen-3-yl-carbamic acid 4-(2-piperidin-1-yl-ethoxy)-phenyl ester;
Thiophen-2-yl-carbamic acid 4-(2-piperidin-1-yl-ethoxy)-phenyl ester;
Phenyl-carbamic acid 4-{2-[4-(2-hydroxy-acetylamino)-piperidin-1-yl]-ethoxy}-phenyl ester;
Methyl-phenyl-carbamic acid 4-{2-[4-(2-hydroxy-acetylamino)-piperidin-1-yl]-ethoxy}-phenyl ester;
Phenyl-carbamic acid 4-[2-(4-methanesulfonylamino-piperidin-1-yl)-ethoxy]-phenyl ester;
Methyl-phenyl-carbamic acid 4-[2-(4-methanesulfonylamino-piperidin-1-yl )-ethoxy]-phenyl ester;
Phenyl-carbamic acid 5-{2-[4-(2-hydroxy-acetylamino)-piperidin-1-yl]-ethoxy}-pyridin-2-yl ester;
Phenyl-carbamic acid 5-[2-(4-acetylamino-piperidin-1-yl)-ethoxy]-pyridin-2-yl ester; and mixtures thereof.

53. A compound selected from:
N-[1-(4-Phenethyloxy-benzyl)-piperidin-4-yl]methanesulfonamide;
1-(6-Phenethyloxy-pyridin-3-ylmethyl)-piperidine-4-carboxylic acid;
1-(4-Phenethyloxy-benzyl)-piperidine;
1-(4-Phenethyloxy-benzyl)-piperidine-4-carboxylic acid;
1-[4-(3-Phenyl-propoxy)-benzyl]-piperidine;
1-[4-(4-Phenyl-butoxy)-benzyl]-piperidine;
1-[1-(4-Phenethyloxy-benzyl )-piperidin-4-yl]-pyrrolidin-2-one;
8-(4-Phenethyloxy-benzyl)-2,8-diaza-spiro[4.5]decan-1-one;
1-(4-Phenethyloxy-benzyl)-piperidine-4-carboxylic acid amide;
1-(4-Phenethyloxy-benzyl)-piperidine-3-carboxylic acid amide;
1-(4-Phenethyloxy-benzyl)-piperidin-4-ol;
1-(4-Phenethyloxy-benzyl )-4-(1H-tetrazol-5-yl)-piperidine;
1-(4-Phenethyloxy-benzyl)-piperidin-4-ylamine;
1-[4-(3-Phenyl-propoxy)-benzyl]-piperidine-4-carboxylic acid ethyl ester;
1-{1-[4-(3-Phenyl-propoxy)-benzyl]-piperidin-4-yl}-pyrrolidin-2-one;
1-[4-(3-Phenyl-propoxy)-benzyl]-piperidin-4-ol;
1-[4-(3-Phenyl-propoxy)-benzyl]-piperidin-3-ol;
1-[4-(3-Phenyl-propoxy)-benzyl]-piperidine-4-carboxylic acid amide;
1-[4-(3-Phenyl-propoxy)-benzyl]-piperidine-3-carboxylic acid amide;
1-[4-(3-Phenyl-propoxy)-benzyl]-piperidine-4-carboxylic acid;
N-[1-(4-Phenethyloxy-benzyl)-piperidin-4-yl]-acetamide;
[1-(4-Phenethyloxy-benzyl )-piperidin-4-yl]-urea;
[1-(4-Phenethyloxy-benzyl )-piperidin-4-yl]-carbamic acid methyl ester;
1-[4-(3-Phenyl-propoxy)-benzyl]-piperidin-4-ylamine;
N-{1-[4-(3-Phenyl-propoxy)-benzyl]-piperidin-4-yl}-methanesulfonamide;
N-{1-[4-(3-Phenyl-propoxy)-benzyl]-piperidin-4-yl}-acetamide;
{1-[4-(3-Phenyl-propoxy)-benzyl]-piperidin-4-yl}-carbamic acid methyl ester;
{1-[4-(3-Phenyl-propoxy)-benzyl]-piperidin-4-yl}-urea;
2-Hydroxy-N-{1-[4-(3-phenyl-propoxy)-benzyl]-piperidin-4-yl}-acetamide;
2-Hydroxy-N-[1-(4-phenethyloxy-benzyl)-piperidin-4-yl]-acetamide;
N-{1-[6-(3-Phenyl-propoxy)-pyridin-3-ylmethyl]-piperidin-4-yl}-acetamide;
N-{1-[6-(3-Phenyl-propoxy)-pyridin-3-ylmethyl]-piperidin-4-yl}-methanesulfonamide;
1-{1-[6-(3-Phenyl-propoxy)-pyridin-3-ylmethyl]-piperidin-4-yl}-pyrrolidin-2-one;
N-[1-(3-Fluoro4-phenethyloxy-benzyl )-piperidin-4-yl]-acetamide; and mixtures thereof.

54. A compound selected from:
1′-[2-(4-Phenethyloxy-phenyl)-ethyl]-[1,4′]bipiperidinyl-3-carboxylic acid ethyl ester;
1′-[2-(4-Phenethyloxy-phenyl)-ethyl]-[1,4′]bipiperidinyl-3-carboxylic acid;
1′-{2-[4-(2-Oxo-2-phenyl-ethoxy)-phenyl]-ethyl}-[1,4′]bipiperidinyl-2-one;
1′-{2-[4-(2-Hydroxy-2-phenyl-ethoxy)-phenyl]-ethyl}-[1,4′]bipiperidinyl-2-one
1-[2-(4-Phenethyloxy-phenyl)-ethyl]-piperidine-4-carbonitrile;
1-[2-(4-Phenethyloxy-phenyl)-ethyl]-4-(1H-tetrazol-5-yl )-piperidine;
1-[2-(4-Phenethyloxy-phenyl)-ethyl]4-(1H-[1,2,3]triazol-4-yl)-piperidine;
Cyclopropyl-[2-(4-phenethyloxy-phenyl)-ethyl]-amine;
4-{Cyclopropyl-[2-(4-phenethyloxy-phenyl)-ethyl]-amino}-butyronitrile;
3-{Cyclopropyl-[2-(4-phenethyloxy-phenyl)-ethyl]-amino}-propionic acid ethyl ester;
3-{Cyclopropyl-[2-(4-phenethyloxy-phenyl)-ethyl]-amino}-propionic acid trifluoroacetic acid salt;
1′-[2-(4-Phenethyloxy-phenyl)-ethyl]-[1,4′]bipiperidinyl-2-carboxylic acid ethyl ester;
1-[2-(4-Phenethyloxy-phenoxy)-ethyl]-piperidine;
2-{4-[2-(Cyclohexyl-ethyl-amino)-ethyl]-phenoxy}-1-phenyl-ethanone;
2-{4-[2-(Cyclohexyl-ethyl-amino)-ethyl]-phenoxy}-1-phenyl-ethanol;
1-{2-[4-(2-Oxo-2-phenyl-ethoxy)-phenyl]-ethyl}-piperidine-4-carboxylic acid methyl ester;
1-{2-[4-(2-Hydroxy-2-phenyl-ethoxy)-phenyl]-ethyl}-piperidine-4-carboxylic acid methyl ester;
1-[2-(4-Phenethyloxy-phenyl)-ethyl]-piperidine-4-carboxylic acid methyl ester;
1-{2-[4-(2-Hydroxy-2-phenyl-ethoxy)-phenyl]-ethyl}-piperidine-4-carboxylic acid amide;
1-[2-(4-Phenethyloxy-phenyl)-ethyl]-piperidine-4-carboxylic acid amide;
1′-[2-(4-Phenethyloxy-phenyl)-ethyl]-[1,4′]bipiperidinyl-2-one;
1-[2-(4-Phenethyloxy-phenyl)-ethyl]-piperidine-4-carboxylic acid;
4-[2-(4-Phenethyloxy-phenyl)-ethyl]-piperazin-2-one;
3-[2-(4-Phenethyloxy-phenyl)-ethylamino]-propionic acid ethyl ester;
3-{Methyl-[2-(4-phenethyloxy-phenyl)-ethyl]-amino}-propionic acid ethyl ester;
3-{Cyclohexyl-[2-(4-phenethyloxy-phenyl)-ethyl]-amino}-propionic acid ethyl ester;
3-{Methyl-[2-(4-phenethyloxy-phenyl)-ethyl]-amino}-propionic acid;
3-{Cyclohexyl-[2-(4-phenethyloxy-phenyl)-ethyl]-amino}-propionic acid;
3-{(1-Acetyl-piperidin-4-yl)-[2-(4-phenethyloxy-phenyl)-ethyl]-amino}-propionic acid ethyl ester;
3-{(1-Acetyl-piperidin-4-yl)-[2-(4-phenethyloxy-phenyl)-ethyl]-amino}-propionic acid;
1-{2-[4-(2-Hydroxy-2-phenyl-ethoxy)-phenyl]-ethyl}-piperidine-4-carboxylic acid;
2-[4-(2-Piperidin-1-yl-ethyl)-phenoxy]-indan-1-one;
Chloro-phenyl-acetic acid 4-(2-piperidin-1-yl-ethyl)-phenyl ester;
Indan-2-carboxylic acid 4-(2-piperidin-1-yl-ethyl)-phenyl ester;
2-[4-(2-Piperidin-1-yl-ethyl)-phenoxy]-indan-1-ol;
1-{2-[4-(3-Phenyl-propoxy)-phenyl]-ethyl}-piperidine-4-carboxylic acid;
2-Hydroxy-N-{1-[2-(4-phenethyloxy-phenoxy)-ethyl]-piperidin-4-yl}-acetamide;
2-Hydroxy-N-(1-{2-[4-(3-phenyl-propoxy)-phenyl]-ethyl}-piperidin-4-yl)-acetamide;
2-Hydroxy-N-{1-[2-(4-phenethyloxy-phenyl)-ethyl]-piperidin-4-yl}-acetamide;
N-{1-[2-(4-Phenethyloxy-phenoxy)-ethyl]-piperidin-4-yl}-methanesulfonamide;
N-(1-{2-[4-(3-Phenyl-propoxy)-phenyl]-ethyl}-piperidin-4-yl)-methanesulfonamide;
N-{1-[2-(4-Phenethyloxy-phenyl)-ethyl]-piperidin-4-yl}-methanesulfonamide;
N-{1-[2-(6-Phenethyloxy-pyridin-3-yl)-ethyl]-piperidin-4-yl}-methanesulfonamide;
2-Hydroxy-N-{1-[2-(6-phenethyloxy-pyridin-3-yl)-ethyl]-piperidin-4-yl}-acetamide;
1-{1-[2-(6-Phenethyloxy-pyridin-3-yl)-ethyl]-piperidin-4-yl}-pyrrolidin-2-one; and mixtures thereof.

55. A compound selected from:
Carbonic acid phenyl ester 4-(2-piperidin-1-yl-ethoxy)-phenyl ester;
Phenyl-acetic acid 4-(2-piperidin-1-yl-ethoxy)-phenyl ester;
2-Phenyl-propionic acid 4-(2-piperidin-1-yl-ethoxy)-phenyl ester;
1H-Indole-2-carboxylic acid 4-(2-piperidin-1-yl-ethoxy)-phenyl ester;
1-Phenyl-2-[4-(2-piperidin-1-yl-ethoxy)-phenoxy]-ethanone;
3-{2-[4-(2-Piperidin-1-yl-ethoxy)-phenoxy]-ethyl}-phenol;
1H-Indole-3-carboxylic acid 4-(2-piperidin-1-yl-ethoxy)-phenyl ester;
1-{2-[4-(Indan-2-yloxy)-phenoxy]-ethyl}-piperidine;
1-(2-{4-[2-(2-Fluoro-phenyl)-ethoxy]-phenoxy}-ethyl)-piperidine;
1-Phenyl-2-[4-(2-piperidin-1-yl-ethoxy)-phenoxy]-ethanol;
4-{2-[4-(2-Piperidin-1-yl-ethoxy)-phenoxy]-ethyl}-phenol;
1-[2-(4-Phenethyloxy-phenoxy)-ethyl]-piperidine-4-carboxylic acid;
1-{2-[4-(3-Phenyl-propoxy)-phenoxy]-ethyl}-piperidine-4-carboxylic acid ethyl ester;
1-{2-[4-(3-Phenyl-propoxy)-phenoxy]-ethyl}-piperidine-4-carboxylic acid;
Chloro-phenyl-acetic acid 4-(2-piperidin-1-yl-ethoxy)-phenyl ester;
Indan-2-carboxylic acid 4-(2-piperidin-1-yl-ethoxy)-phenyl ester;
2-[4-(2-Piperidin-1-yl-ethoxy)-phenoxy]-indan-1-ol;
2-[4-(2-Piperidin-1-yl-ethoxy)-phenoxy]-indan-1-one;
2-Hydroxy-N-(1-{2-[4-(3-phenyl-propoxy)-phenoxy]-ethyl}-piperidin-4-yl)-acetamide;
N-(1-{2-[4-(3-Phenyl-propoxy)-phenoxy]-ethyl}-piperidin-4-yl )-methanesulfonamide;
N-{1-[2-(6-Phenethyloxy-pyridin-3-yloxy)-ethyl]-piperidin-4-yl}-methanesulfonamide;
1-{2-[6-(3-Phenyl-propoxy)-pyridin-3-yloxy]-ethyl}-piperidine-4-carboxylic acid;
1-(4-Phenethyloxy-phenoxy)-3-piperidin-1-yl-propan-2-ol;
2-Hydroxy-N-(1-{2-hydroxy-3-[4-(3-phenyl-propoxy)-phenoxy]-propyl}-piperidin-4-yl)-acetamide;
N-{1-[2-(3-Fluoro-4-phenethyloxy-phenoxy)-ethyl]-piperidin-4-yl}-2-hydroxy-acetamide;
1-(2-{4-[2-(3-Fluoro-phenyl)-ethoxy]-phenoxy}-ethyl)-piperidine;
1-(2-{4-[2-(4-Fluoro-phenyl)-ethoxy]-phenoxy}-ethyl)-piperidine; and mixtures thereof.

56. A pharmaceutical composition comprising at least one compound of claim 12.

57. A method for preventing or treating a disease selected from the group consisting of: asthma, chronic obstructed pulmonary disease (COPD), atherosclerosis, rheumatoid arthritis, multiple sclerosis, inflammatory bowel diseases, Crohn's disease, ulcerative colitis, and psoriasis, comprising administering to a mammal suffering therefrom at least one compound of claim 12.

58. A method for preventing or treating a disease selected from the group consisting of: cystic fibrosis, arthritis, and cardiovascular disease with an inflammatory component, comprising administering to the subject a therapeutically effective amount of a pharmaceutical composition comprising at least one compound of claim 12.

59. A method for preventing or treating a disease selected from the group consisting of: myocardial infarction, aortic aneurysm, ischemia reperfusion, and stroke, comprising administering to the subject a therapeutically effective amount of a pharmaceutical composition comprising at least one compound of claim 12.

60. The method of claim 58, wherein said disease is cardiovascular disease with an inflammatory component.

61. The method of claim 60, wherein said disease is at least one of myocardial infarction, aortic aneurysm, ischemia reperfusion, and stroke.
